(19) United States
(12) Patent Application Publication
(10) Pub. No.: US 2008/0182236 A1
(43) Pub. Date: Jul. 31, 2008

(54) Asthma Susceptibility Locus

(75) Inventors: Tarja Laitinen, Jarvenpaa (FI); Juha Kere, Stockholm (SE); Lauri Laitinen, Espoo (FI); Anne Polvi, Espoo (FI); Siru Makela, Helsinki (FI); Johanna Vendelin, Espoo (FI); Ville Pulkkinen, Helsinki (FI); Paula Salminkangas, Klaukkala (FI)

Correspondence Address: BIRCH STEWART KOLASCH & BIRCH, PO BOX 747, FALLS CHURCH, VA, 22040-0747, US

(73) Assignee: Geneos OY, Helsinki Finland (FI)

(21) Appl. No.: 10/539,565

(22) PCT Filed: Dec. 19, 2003
(86) PCT No.: PCT/FI03/00973

§ 371 Date: Sep. 27, 2006

Related U.S. Application Data
(60) Provisional application No. 60/435,846, filed on Dec. 20, 2002.
(60) Provisional application No. 60/437,895, filed on Jan. 3, 2003.
(60) Provisional application No. 60/458,767, filed on Mar. 26, 2003.
(60) Provisional application No. 60/486,000, filed on Jul. 9, 2003.

(51) Int. Cl. C12Q 1/68; A01K 67/027; C12P 21/06; C07H 21/04; C07K 14/74; C07K 16/28

(52) U.S. Cl. 435/6; 435/69.1; 435/320.1; 435/325; 530/350; 530/388.22; 514/12; 800/14; 536/23.5

(57) ABSTRACT

The present invention describes a susceptibility locus which is functionally related to asthma. The locus maps within human chromosome 7p15-p14. The invention also describes a novel human gene, GPRA. The invention provides diagnostic methods and materials for analysing allelic variation in said locus and the GPRA gene. The invention also provides polypeptides encoded by GPRA gene and antibodies binding to said polypeptides. The invention further provides pharmaceutical compositions for the treatment of asthma, other IgE-mediated disease, chronic obstructive pulmonary disease or cancer.

### FIELD OF THE INVENTION

The present invention resides in the field of molecular genetics.

### BACKGROUND OF THE INVENTION

We have previously mapped a susceptibility locus for asthma and high total serum Immunoglobulin E level (IgE) to a large region spanning ˜19 cM of chromosome 7p15-p14. This locus was the only locus that reached genome wide significance level among the Finnish asthma families (Laitinen, 2001). Linkage was further confirmed among the French-Canadian families recruited based on asthma and in another independent data set from Finland recruited based on high IgE level (Laitinen, 2001). Daniels et al. (1996) have reported among Australian and British families six regions of possible linkage including 7p14-p15 and established by simulations that at least some of them are likely to be true positives. Fine mapping of the region revealed bimodal linkage to bronchial hyperreactivity and blood eosinophil count at D7S484 (P=0.0003) and D7S669 (P=0.006) 63 cM apart (Leaves et al. 2002). German (Wjst et al. 1999), French (Dizier et al. 2000), and Italian families (Malerba et al. 2000) have shown some evidence of linkage, but there are also genome scans with inconclusive results (Ober et al. 2000; Xu et al. 2000; Mathias et al. 2001), some of which have been done in ethnic populations other than Caucasian (Yokouchi et al. 2000; Xu et al. 2001). By comparing genome scans in different immune disorders, it has been suggested that clinically distinct autoimmune diseases may be controlled by a common set of susceptibility genes (Becker et al. 1998). 7p15-p14 has also been linked to diseases such as multiple sclerosis (Sawcer et al. 1996) and inflammatory bowel disease (Satsangi et al. 1996), and genomic regions homologous to human 7p15-p14 have been linked to insulin dependent diabetes (Jacob et al. 1992) and inflammatory arthritis (Remmers et al. 1996) in rat models.

### SUMMARY OF THE INVENTION

The present invention provides an isolated, purified asthma locus-1 (AST1) nucleic acid and a complement or a fragment thereof. The invention also provides nucleic acids comprising at least one single nucleotide polymorphism and/or deletion/insertion polymorphism in different positions in asthma locus-1. One object of the invention is to provide vectors, host cells, primers, and probes comprising asthma locus-1 nucleic acid according to the invention. The present invention is also related to a method for the diagnosis of a single nucleotide polymorphism or a deletion/insertion polymorphism in asthma locus-1 according to the invention in a human, which method comprises determining the sequence of the nucleic acid of the human at one or more of positions in asthma locus-1 and determining the status of the human by reference to polymorphism in asthma locus-1. The invention is also related to a kit for use in the diagnostics of asthma and other IgE mediated allergic diseases or in assessing the predisposition of an individual to asthma and other IgE mediated allergic diseases. The invention is further related to a method for identifying a mutation, which increases individual's susceptibility to develop asthma and other IgE mediated allergic diseases. The invention also provides a transgenic animal comprising asthma locus-1 nucleic acid according to the invention.

The invention provides an isolated GPRA polypeptide comprising an amino acid sequence that has at least 90% sequence identity to an amino acid sequence selected from the group consisting of SEQ. ID NOS: 3, 5, 7, 9, 11, 13 and 15 over the entire length of the selected SEQ ID No: when compared using the BLASTP algorithm with a wordlength (W) of 3, and the BLOSUM62 scoring matrix.

The invention further provides an isolated GPRA polypeptide comprising at least 10 contiguous amino acids from amino acids 343-377 of B-long (SEQ ID NO:5).

The invention further provides an isolated GPRA polypeptide comprising an amino acid sequence that has at least 80% sequence identity to an amino acid sequence selected from the group consisting of SEQ. ID NOS: 3, 5, 7, 9, 11, 13 and 15 over a sequence comparison window of at least 40 amino acids when compared using the BLASTP algorithm with a wordlength (W) of 3, and the BLOSUM62 scoring matrix provided that the polypeptide includes a variant amino acid encoded by a variant form shown in Table 7.

The invention further provides an isolated GPRA polypeptide comprising the amino acid sequence of SEQ ID NO:5 provided that the sequence contain as an amino acid substitution of Asn instead of Ile at codon position 107, Arg instead of Ser at codon position 241, and/or Thr instead of Ile at codon position 366.

The invention further provides an isolated nucleic acid encoding a GPRA polypeptide as defined above.

The invention further provides an isolated nucleic acid that hybridizes under highly stringent conditions to any of SEQ ID NOS: 1, 4, 6, 8, 10, 12, and 14 without hybridizing under the same highly stringent conditions to SEQ ID NO:2, wherein the highly stringent conditions are 6× NaCl/sodium citrate (SSC) at about 45° C. for a hybridization step, followed by a wash of 2×SSC at 50° C.

The invention further provides an isolated nucleic acid having a sequence that is at least 90% identical to a nucleic acid having a sequence selected from the group consisting of SEQ ID NO: 4, 6, 8, 10, 12, and 14 over the entire length of the selected SEQ ID NO when compared using the BLASTN algorithm with a wordlength (W) of 11, M=5, and N=−4.

The invention further provides an isolated nucleic acid having a sequence that is at least 80% identical to a nucleic acid having a sequence selected from the group consisting of SEQ ID NO: 1, 2, 4, 6, 8, 10, 12, and 14 over a sequence comparison window of at least 100 nucleotides when compared using the BLASTN algorithm with a wordlength (W) of 11, M=5, and N=−4 provided that the nucleic acid includes a polymorphic site occupied by a variant form as shown in Table 3 or Table 7.

The invention further provides an isolated nucleic acid having a sequence that is at least 80% identical to a nucleic acid having a sequence selected from the group consisting of SEQ ID NO: 1, 2, 4, 6, 8, 10, 12, and 14 over a sequence comparison window of at least 100 nucleotides when compared using the BLASTN algorithm with a wordlength (W) of 11, M=5, and N=−4 provided that the nucleic acid includes a polymorphic site occupied by a reference form designated with a * in Table 7.

The invention further provides an isolated genomic DNA molecule or a minigene having at least one intronic sequence and encoding a GPRA polypeptide that has at least 80% sequence identity to an amino acid sequence selected from the group consisting of SEQ. ID NOS: 3, 5, 7, 9, 11, 13 and 15 over a region at least 40 amino acids in length when compared using the BLASTP algorithm with a wordlength (W) of 3, and the BLOSUM62 scoring matrix.

The invention further provides an antibody that specifically binds to an epitope within amino acids 343-377 of B-long (SEQ ID NO:5) or amino acids 332-366 of B-short (SEQ ID NO:7).

The invention further provides a method of preventing or treating asthma, other IgE mediated disease or cancer. The method comprises administering to a patient suffering from or at risk of asthma, other IgE mediated disease or cancer an effective amount of a modulator of a GPRA polypeptide comprising an amino acid sequence selected from the group consisting of SEQ. ID NOS 3, 5, 7, 9, 11, 13.

The invention further provides a method of identifying a modulator of a GPRA polypeptide. The method comprises contacting a cell expressing a GPRA polypeptide with an agent; and determining whether the agent modulates expression of the GPRA polypeptide and/or signal transduction through the GPRA polypeptide, wherein the GPRA polypeptide is defined as above.

The invention further provides an method of determining risk of asthma, other IgE mediated disease or cancer. The method comprises determining whether the individual has a variant polymorphic form in a GPRA gene, wherein presence of the variant polymorphic form indicates risk of asthma, other IgE mediated disease or cancer.

The invention further provides a method for identifying a polymorphic site correlated with a disease selected from the group consisting of asthma, other IgE-mediated disease and cancer or susceptibility thereto. The method comprises identifying a polymorphic site within a GPRA gene; and determining whether a variant polymorphic form occupying the site is associated with the disease or susceptibility thereto.

The invention further provides a primer or probe nucleic acid that hybridizes under highly stringent conditions to a segment of SEQ ID NO:1, 2 or 4 or a variant form thereof differing from SEQ ID NO: 1, 2 or 4 at a position shown in Table 3 or Table 7, wherein the segment includes or is immediately adjacent to a polymorphic site shown in Table 3 or Table 7.

The invention further provides a transgenic animal comprising a GPRA and/or AAA1 nucleic acid.

The invention further provides a transgenic animal disposed to develop a characteristic of asthma, other IgE-mediated disease or cancer in which an endogenous a GPRA gene encoding a cognate form of a GPRA polypeptide defined by any of SEQ ID NOS: 3, 5, 7, 9, 11, 13 and 15 is functionally disrupted to prevent expression of a gene product.

The invention further provides a kit for use in diagnosing or assessing predisposition to asthma, other IgE-mediated disease or cancer. The kit comprises a container; and in the container, a compound, preferably labeled, capable of detecting a polymorphic form at a polymorphic site in a susceptibility locus for asthma as defined by SEQ ID NO:1, 2 or 4.

The invention further provides an isolated AAA1 polypeptide comprising an amino acid sequence that has at least at least 80% sequence identity to an amino acid sequence selected from the group consisting of SEQ. ID NOS: 17, 19, 21, 23, 25, 27, 29, 31, 33, 35, 37, 39 and 41 over the entire length of the selected SEQ ID No: when compared using the BLASTP algorithm with a wordlength (W) of 3, and the BLOSUM62 scoring matrix.

The invention further provides an isolated nucleic acid encoding the AAA1 polypeptide as defined above.

The invention further provides an isolated nucleic acid having a sequence that is at least 80% identical to a nucleic acid having a sequence selected from the group consisting of SEQ ID NO: 16, 18, 20, 22, 24, 26, 28, 30, 32, 34, 36, 38 and 40 over the entire length of the selected SEQ ID NO when compared using the BLASTN algorithm with a wordlength (W) of 11, M=5, and N=−4.

The invention further provides an isolated nucleic acid having at least 20 contiguous nucleotides from a sequence selected from the group consisting of SEQ ID NOS: 16, 18, 20, 22, 24, 26, 28, 30, 32, 34, 36, 38 and 40.

The invention further provides an isolated genomic DNA molecule or a minigene having at least one intronic sequence and encoding an AAA1 polypeptide that has at least 80% sequence identity to an amino acid sequence selected from the group consisting of SEQ. ID NOS: 17, 19, 21, 23, 25, 27, 29, 31, 33, 35, 37, 39 and 41 over the entire length of the selected SEQ ID NO. when compared using the BLASTP algorithm with a wordlength (W) of 3, and the BLOSUM62 scoring matrix.

The invention further provides an antibody that specifically binds to a polypeptide selected from the group consisting of SEQ ID NOS:17, 19, 21, 23, 25, 27, 29, 31, 33, 35, 37, 39 and 41.

The invention further provides a method of preventing or treating asthma, other IgE-mediated disease or cancer. The method comprises administering to a patient suffering from or at risk of asthma, other IgE-mediated disease or cancer an effective amount of a modulator of an AAA1 polypeptide comprising an amino acid sequence selected from the group consisting of SEQ. ID NOS: 17, 19, 21, 23, 25, 27, 29, 31, 33, 35, 37, 39 and 41.

The invention further provides a method of identifying a modulator of an AAA1 polypeptide. The method comprises contacting an AAA1 polypeptide with an agent; and determining whether the agent binds to the AAA1 polypeptide, modulates expression of the AAA1 polypeptide or modulates activity of the AAA1 polypeptide, wherein the AAA1 polypeptide comprises an amino acid sequenced as defined by any of SEQ ID NOS: 17, 19, 21, 23, 25, 27, 29, 31, 33, 35, 37, 39 and 41.

The invention further provides a method of determining risk of asthma, other IgE mediated disease or cancer, comprising determining whether the individual has a variant polymorphic form in an AAA1 gene, wherein presence of the variant polymorphic form indicates risk of asthma, other IgE mediated disease or cancer.

The invention further provides a method for identifying a polymorphic site correlated with a disease selected from the group consisting of asthma, other IgE mediated disease or cancer or susceptibility thereto. The method comprises identifying a polymorphic site within an AAA1 gene, and determining whether a variant polymorphic form occupying the site is associated with the disease or susceptibility thereto.

The invention provides a primer or probe nucleic acid of nucleotides that hybridizes under highly stringent conditions to a segment of SEQ ID NO: 16, 18, 20, 22, 24, 26, 28, 30, 32, 34, 36, 38 and 40 or a variant form thereof differing from SEQ ID NOS. 16, 18, 20, 22, 24, 26, 28, 30, 32, 34, 36, 38 and 40 at a single polymorphic site.

The invention further provides a transgenic animal disposed to develop a characteristic of asthma, other IgE-mediated disease or cancer in which an endogenous a AAA1 gene encoding a cognate form of an AAA1 polypeptide defined by any of SEQ ID NOS: 17, 19, 21, 23, 25, 27, 29, 31, 33, 35, 37, 39 and 41 is functionally disrupted to prevent expression of a gene product.

### BRIEF DESCRIPTION OF THE DRAWINGS

FIG. 1: Physical map across the linkage region showing the organization of exons of known genes and microsatellite markers genotyped (above), and the organization of genomic BAC clones and contigs (below).

FIG. 2: Physical map across the critical (SNP509783-SNP638799, a total of 129,017 bp) and the flanking regions showing the organization of all markers genotyped in phase three (above), and the organization of genomic BAC clones (below).

FIG. 3: Genomic localization of the susceptibility haplotype (AST1, gray region) of 129 kb for asthma related traits in relation to verified exons of GPRA (G protein coupled receptor for asthma susceptibility). The cDNA probe used in the Northern blotting is shown in shaded gray. Locations of PCR primers used in the identification of the gene structures and nested PCR primers (in bold) used in cloning of the full length cDNA of splice variants A and B are shown. SNPs in the exons of GPRA and their potential amino acid changes are shown below. Both ends of the susceptibility haplotype are shown with arrows as physical positions in the genomic contig NT_{—}000380.

FIGS. 4A, 4B1, 4B2, 4C, 4D, 4E, and 4F: Deduced cDNAs (SEQ ID NOS: 2, 4, 6, 8, 10, 12, and 14) and amino acid sequences (SEQ ID NOS: 3, 5, 7, 9, 11, 13, and 15) of the alternative splice variants (A-F) of GPRA. Exon borders are indicated by vertical lines. The initiation and stop codons are shown in bold.

FIGS. 5A, 5B, 5C, 5D, 5E and 5F: Genomic structures of the alternative splice variants (A-F) of GPRA. The exons (gray boxes) are depicted by numbered boxes (E1-E9), the length of the exons are indicated below, and the length of the separating introns above. The translation-initiation site and the stop codons are indicated by arrows. The white boxes indicate the 5′ and 3′ UTR regions and the shaded box in the B variant exon 3 shows the alternative slicing of the long and short form.

FIGS. 6A, 6B, 6C, 6D, 6E and 6F: Predicted 7TM structures of the alternative splice variants of GPRA (SEQ ID NOS: 3, 5, 7, 9, 11, 13, and 15). For the splice variants A and B_{long}, cytoplasmic loops (=cytoloop) and extracellular loops (=exoloop) are indicated above the amino acid chain, transmembrane regions are underlined and the conserved amino acids, characteristic for the GPR family A, are bolded. For the other splice variants (Bₛₕₒᵣₜ₋F) the number of predicted transmembrane regions varied from one to six.

FIG. 7: Predicted topology of the seven transmembrane structure of the protein encoded by the GPRA A splice variant by TMpred.

FIG. 8: Expression of GPRA variants in the human lung epithelial carcinoma cell line NCI-H358. RT-PCR was performed by using variant A, B, and C specific primers. PCR-products were separated on ethidium bromide stained 1.5% agarose gel.

FIG. 9: Expression of GPRA by Northern blot analysis with a 470 bp cDNA probe (comprising the exons E2a and E2b) in human placental tissue. Four transcripts were detected that were approximately 6.0 kb, 4.5 kb, 1.9 kb, and 1.0 kb in size. The expression of β-actin is shown as control.

FIGS. 10A, 10B, 10C, 10D, 10E, and 10F: Immunohistochemical analysis of GPRA A in human bronchial (10A, 10B), lung (10C), and colon (10D, 10E), and skin (10D) tissues. Paraffin sections were stained using immunoperoxidase technique with GPRA variant A specific antibody (10A, 10C-10D) or pre-immune sera (10B). Strong immunostaining of GPRA A in bronchus (10A) and colon (10E) was recorded in smooth muscle cell layer in bronchial walls (thick arrows) and in arterial walls (thin arrows) and subepithelially (arrowheads) in colon, respectively. In alveolar walls and alveolar macrophages (asterisk) intense staining was detected (10C). In colon (10D) and skin (10F) tissues, basal cell layer of the epithelium (white arrows) stained positive for GPRA A. Staining of the corresponding sections with the pre-immune serum did not show specific reactivity (10B). Original magnification ×400.

FIGS. 11A, 11B, 11C, 11D, 11E, and 11F: Immunohistochemical analysis of GPRA B in human bronchial (11A, 11B), lung, small intestine (11D), colon (11E), and skin (11F) tissues. Paraffin sections were stained with the immunoperoxidase technique with GPRA variant B specific antibody (11A, 11C-D) or preimmune serum (11B). Strong immunostaining of GPRA B was recorded in the epithelium of the bronchial wall (11A), small intestine (11D), colon (11E), and skin (11F) tissues (arrows). In lung intense staining of alveolar wall and alveolar macrophages (asterisk) was detected (11C). Staining of the corresponding sections with the preimmune serum did not show specific reactivity (11B). Original magnification ×400.

FIGS. 12A and 12B: In Western blot analysis of several human tissues, the A variant specific antibody recognized four major polypeptide bands at approximately 40, 42, 44, and 50 kDa (12A). 50 kDa band was detectable in skeletal muscle whereas uterine muscle, colon epithelium and prostate showed similar expression patterns of GPRA A at approximately 50, 44, 40 kDa. The most intensive GPRA A expression was recorded in colon muscle at 42 kDa. The B variant specific antibody recognized two major polypeptide bands at approximately 25 and 39 kDa with the most intensive GPRA expression in kidney (12B).

FIG. 13: Genomic structure of AAA1 showing both the exon (size below) and intron (size above) structures of the gene. AAA1 (asthma associated alternatively spliced gene 1) shows complex splicing, two alternative starting methionines and six alternative stop codons for the predicted polypeptide was identified. Location of the primers used in cloning of different splice variants are shown below. Gray area shows the location of AST1.

FIG. 14: I-XII different splice variants of AAA1, I-XI are full length cDNAs.

FIG. 15: Sequence alignments of the predicted polypeptides encoded by the I-XI splice variants of AAA1 (CLUSTAL W program). The conserved region of the AAA1 encoded polypeptides is in bold.

FIG. 16: Human multiple tissue expression array (BD, Clontech) hybridized with the probe specific for AAA1 (a mixture of multiple splice variants) (upper panel) and location of studied RNAs in the dot blot (lower panel).

FIG. 17: Human fetal multiple tissue northern blot (BD, Clontech) hybridized with the probe specific for AAA1 (a mixture of multiple splice variants) identifies several alternative transcripts (arrows).

FIG. 18: Tissue specific expression AAA1. RT-PCR with the variants I, IV, VI, X, and XI specific primer pairs in liver (Li), lung (Lu), testis (Te), and kidney (Ki) shows different patterns of transcripts in different tissues.

FIG. 19: Variable alternative splicing for AAA1 depending on genotype. RT-PCR spanning exons 6 to 10b of AAA1 was performed on lymphoblastic RNA samples genotyped for AST1. Only the non-carrier of AST1 processes normal amount of the exon 6-10b transcript, whereas a homozygote and heterozygotes show either absent transcript or smaller splice variants. Beta-actin was used as control in parallel amplifications.

FIG. 20: Nucleic (SEQ ID NOS:16, 18, 20, 22, 24, 26, 28, 30, 32, 34, 36, 38 and 40) and amino acid sequences (SEQ ID NOS: 17, 19, 21, 23, 25, 27, 29, 31, 33, 35, 37, 39 and 41) of splice variants of AAA1.

FIGS. 21A and 21B: 21A. In vitro translation of AAA1 gene using rabbit reticulocyte lysate translation machinery with S³⁵-labelled methionine in the reaction mixture. 21B. Specificity of the AAA1 antibody (anti-AAA1). Glutathione S-transferase (GST)-fusion proteins for AAA1 were expressed in E. coli as GST fusion proteins (GST-AAA1).

### DEFINITIONS

Unless defined otherwise, all technical and scientific terms have the same meaning as is commonly understood by one of skill in the art to which this invention is related. The definitions below are presented for clarity.

“Isolated” when referred to a molecule, refers to a molecule that has been identified and separated and/or recovered from a component of its natural environment and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide sequences of the present invention.

“Nucleic acid”, includes DNA molecules (e.g. cDNA or genomic DNA), RNA molecules (e.g., mRNA), analogs of the DNA or RNA generated using nucleotide analogs, and derivatives, fragments and homologs. The nucleic acid or nucleic acid molecule may be single-stranded or double-stranded, but preferably comprises double-stranded DNA. Preferred nucleic acids of the invention include segments of DNA, or their complements including any one of the polymorphic sites shown in Table 3, 7 or 12. The segments are usually between 5 and 100 contiguous bases, and often range from 5, 10, 12, 15, 20, or 25 nucleotides to 10, 15, 30, 25, 20, 50 or 100 nucleotides. Nucleic acids between 5-10, 5-20, 10-20, 12-30, 15-30, 10-50, 20-50 or 20-100 bases are common. The polymorphic site can occur within any position of the segment. The segments can be from any of the allelic forms of DNA shown in Table 3, 7 or 12. For brevity in Table 3, the symbol T is used to represent both thymidine in DNA and uracil in RNA. Thus, in RNA oligonucleotides, the symbol T should be construed to indicate a uracil residue. Unless otherwise apparent from the context, reference to a SEQ ID NO. of the invention, refers to the strand shown in the sequence listing for that SEQ ID NO., the perfect complementary strand thereof or a duplex of the two. Thus, for example, reference to primers hybridizing to SEQ ID NO:1 includes primers hybridizing to the strand shown in the sequence listing and the complementary strand.

“Isolated nucleic acid” is separated from other nucleic acid molecules which are present in the natural source of the nucleic acid. Preferably, an isolated nucleic acid is free of sequences that naturally flank the nucleic acid (i.e. sequences located at the 5′- and 3′-termini of the nucleic acid) in the genomic DNA of the organism from which the nucleic acid is derived. For example, in various embodiments, isolated asthma locus-1 molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an isolated nucleic acid molecule, such as a cDNA molecule, can be substantially free of other cellular material or culture medium when produced by recombinant techniques, or of chemical precursors or other chemicals when chemically synthesized.

The term “fragment” or “segment” as applied to a longer nucleic acid, may ordinarily be at least about 10 contiguous nucleotides of the longer nucleic acid in length, typically, at least about 20 contiguous nucleotides, more typically, from about 20 to about 50 contiguous nucleotides, preferably at least about 50 to about 100 contiguous nucleotides, even more preferably at least about 100 contiguous nucleotides to about 300 contiguous nucleotides, yet even more preferably at least about 300 to about 400, and most preferably, the nucleic acid fragment will be greater than about 500 contiguous nucleotides in length.

“Oligonucleotide” comprises a series of linked nucleotide residues, which oligonucleotide has a sufficient number of nucleotide bases to be used in a PCR reaction or other application. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid.

“Variant” refers to a polynucleotide differing from the polynucleotide of the present invention, but retaining essential properties thereof. Generally, variants are overall closely similar, and, in many regions, identical to the polynucleotide of the present invention. Allelic variants of a gene refer to variant forms of the same gene between different individuals of the same species. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Cognate forms of a gene refers to variation between structurally and functionally related genes between species. For example, the human gene showing the greatest sequence identity and closest functional relationship to a mouse gene is the human cognate form of the mouse gene. Thus, for example, the invention includes primate, bovine, ovine, murine, and avian cognate forms of the human GPRA and AAA1 genes.

A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele. A set of polymorphisms means at least 2, and sometimes 5, or more of the polymorphisms shown in Table 3, 7 or 12.

“Stringency”. Highly stringency conditions are well-known in the art, e.g. 6× NaCl/sodium citrate (SSC) at about 45° C. is applied for a hybridization step, followed by a wash of 2×SSC at 50° C. or, e.g., alternatively hybridization at 42° C. in 5×SSC, 20 mM NaPO4, pH 6.8, 50% formamide; and washing at 42° C. in 0.2×SSC. These conditions can be varied empirically based on the length and the GC nucleotide base content of the sequences to be hybridized, or based on formulas for determining such variation (See, for example, Sambrook et al, “Molecular Cloning: A Laboratory Manual”, Second Edition, pages 9.47-9.51, Cold Spring Harbor, N.Y.: Cold Spring Harbor Laboratory Press (1989)). A low stringency is defined herein as being in 4-6×SSC/0.1-0.5% w/v SDS at 37-45 degree of C for 2-3 hours. Depending on the source and concentration of nucleic acid involved in the hybridization, alternative conditions of stringency may be employed such as medium stringent conditions which are considered herein to be 1-4×SSC/0.25-0.5% w/v SDS at 45 degree of Celsius for 2-3 hours or highly stringent conditions considered herein to be 0.1-1×SSC/0.1% w/v SDS at 60 degree of Celsius for 1-3 hours.

Hybridization probes are capable of binding in a base-specific manner to a complementary strand of nucleic acid. Such probes include nucleic acids, peptide nucleic acids, as described in Nielsen et al., Science 254, 1497-1500 (1991). “Probes” are nucleic acid sequences of variable length, preferably between at least about 10 nucleotides (nt), 100 nt, or many (e.g., 6000 nt) depending on the specific use. Often probes have 15-50 nucleotides. Probes are used to detect identical, similar, or complementary nucleic acid sequences. Longer length probes can be obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies. Probes also hybridize to nucleic acid molecules in biological samples, thereby enabling immediate applications in chromosome mapping, linkage analysis, tissue identification and/or typing, and a variety of forensic and diagnostic methods of the invention.

The term primer refers to a single-stranded oligonucleotide capable of acting as a point of initiation of template-directed DNA synthesis under appropriate conditions (i.e., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30, 40 or 50 nucleotides. Short primer molecules generally require cooler temperatures to form sufficiently stable hybrid complexes with the template. A primer need not reflect the exact sequence of the template but must be sufficiently complementary to hybridize with a template. The term primer site refers to the area of the target DNA to which a primer hybridizes. The term primer pair means a set of primers including a 5′ upstream primer that hybridizes with the 5′ end of the DNA sequence to be amplified and a 3′, downstream primer that hybridizes with the complement of the 3′ end of the sequence to be amplified.

“Vector” means any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector which is suitable as a delivery vehicle for delivery of the nucleic acid encoding the desired protein, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94:12744-12746). Examples of viral vectors include, but are not limited to, a recombinant vaccinia virus, a recombinant adenovirus, a recombinant retrovirus, a recombinant adeno-associated virus, a recombinant avian pox virus, and the like (Cranage et al., 1986, EMBO J. 5.3057-3063; International Patent Application No. W094/17810, published Aug. 18, 1994; International Patent Application No. W094/23744, published Oct. 27, 1994). Examples of non-viral vectors include, but are not limited to, liposomes, polyamine derivatives of DNA, and the like.

“Recombinant polynucleotide” refers to a polynucleotide having sequences that are not naturally joined together. An amplified or assembled recombinant polynucleotide may be included in a suitable vector, and the vector can be used to transform a suitable host cell.

“Mammal” for purposes of treatment refers to any animal classified as a mammal, including humans, domestic and farm animals, and zoo, sports, or pet animals, such as dogs, horses, cats, or cows. Preferably, the mammal is human.

Linkage describes the tendency of genes, alleles, loci or genetic markers to be inherited together as a result of their location on the same chromosome, and can be measured by percent recombination between the two genes, alleles, loci or genetic markers that are physically-linked on the same chromosome. Loci occurring within 50 centimorgan of each other are linked. Some linked markers occur within the same gene or gene cluster.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair.

Linkage disequilibrium (LD) or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the haplotype ac to occur with a frequency of 0.25 in a population of individuals. If ac occurs more frequently, then alleles a and c are considered in linkage disequilibrium. Linkage disequilibrium may result from natural selection of certain combination of alleles or because an allele has been introduced into a population too recently to have reached equilibrium (random association) with between linked alleles.

A marker in linkage disequilibrium with disease predisposing variants can be particularly useful in detecting susceptibility to disease (or association with other sub-clinical phenotypes) notwithstanding that the marker does not cause the disease. For example, a marker (X) that is not itself a causative element of a disease, but which is in linkage disequilibrium with a gene (including regulatory sequences) (Y) that is a causative element of a phenotype, can be used to indicate susceptibility to the disease in circumstances in which the gene Y may not have been identified or may not be readily detectable. Younger alleles (i.e., those arising from mutation relatively late in evolution) are expected to have a larger genomic segment in linkage disequilibrium. The age of an allele can be determined from whether the allele is shared between different human ethnic human groups and/or between humans and related species.

Unless otherwise apparent from the context, any embodiment, element or feature of the invention can be used in combination with any other.

“Analogs” are nucleic acid sequences that have a structure similar to, but not identical to, the native compound but differ from it in respect to certain components. Usually, an analog has the same or similar function to the native compound. Analogs may be synthetic or from a different evolutionary origin and may have a similar or opposite metabolic activity compared to wild type.

Derivatives and analogs may be full length or other than full length. Derivatives or analogs of the nucleic acids of the invention include, nucleic acids that are substantially identical to an exemplified SEQ ID NO. and/or capable of hybridizing to the complement of the sequence under highly stringent, moderately stringent, or low stringent conditions.

The terms “identical” or percent “identity,” in the context of two or more nucleic acids or polypeptides, refer to two or more sequences or subsequences that are the same or have a specified percentage of nucleotides or amino acid residues that are the same, when compared and aligned for maximum correspondence, as measured using a sequence comparison algorithm such as those described below for example, or by visual inspection.

The phrase “substantially identical,” in the context of two nucleic acids or polypeptides, refers to two or more sequences or subsequences that have at least 80, preferably at least 85%, more preferably at least 90%, 95%, 99% or higher nucleotide or amino acid residue identity, when compared and aligned for maximum correspondence, as measured using a sequence comparison algorithm such as those described below for example, or by visual inspection. Preferably, the substantial identity exists over a region of the sequences that is at least 40 residues (i.e., amino acids or nucleotides) in length, preferably over a longer region than 50 residues, more preferably at least about 90-100 residues, and most preferably the sequences are substantially identical over the full length of the sequences being compared, such as the coding region of a nucleotide for example. For example, when a SEQ ID NO. of the invention serves as a reference for comparison with an object nucleic acid, the comparison is preferably performed over the entire length of the SEQ ID NO. of the invention.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://wwvw.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope of the invention, the default parameters of the BLAST programs are suitable. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix. The TBLATN program (using protein sequence for nucleotide sequence) uses as defaults a word length (W) of 3, an expectation (E) of 10, and a BLOSUM 62 scoring matrix. (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

Another indication that two nucleic acid sequences are substantially identical is that the two molecules hybridize to each other under highly stringent conditions. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence. The phrase “hybridizing specifically to”, refers to the binding, duplexing, or hybridizing of a molecule only to a particular nucleotide sequence under highly stringent conditions when that sequence is present in a complex mixture (e.g., total cellular) DNA or RNA.

The phrases “specifically binds” refers to a binding reaction which is determinative of the presence of the protein in the presence of a heterogeneous population of proteins and other biologics. Thus, under designated conditions, a specified ligand binds preferentially to a particular protein and does not bind in a significant amount to other proteins present in the sample. A molecule such as antibody that specifically binds to a protein often has an association constant of at least 10⁶ M⁻¹ or 10⁷ M⁻¹, preferably 10⁸ M⁻¹ to 10⁹ M⁻¹, and more preferably, about 10¹⁰ M⁻¹ to 10¹¹ M⁻¹ or higher. An antibody that specifically binds to one segment of a protein (e.g., residues 1-10) does not bind to other segments of the protein not included within or overlapping the designated segment.

The term AAA1 gene means nucleotides 163615-684776 of the human genomic contig NT_{—}000380 and allelic or species variants thereof.

The term GPRA gene means nucleotides 471478-691525 of the human genomic contig NT_{—}000380 and allelic or species variants thereof.

A “pharmacological” activity means that an agent exhibits an activity in a screening system that indicates that the agent is or may be useful in the prophylaxis or treatment of a disease. The screening system can be in vitro, cellular, animal or human. Agents can be described as having pharmacological activity notwithstanding that further testing may be required to establish actual prophylactic or therapeutic utility in treatment of a disease.

Pulmonary diseases associated with lower airways obstruction are classified as chronic obstructive pulmonary disease (COPD) or asthma on the basis of the clinical picture. “Asthma” is a chronic inflammatory disease of the airways causing variable airflow obstruction that is often reversible either spontaneously or with treatment. Asthma is highly correlated with other allergic, IgE mediated diseases, such as allergic rhinitis and dermatitis. COPD is characterized by slowly processing, mainly irreversible airway obstruction and decreased expiratory flow rate.

### DETAILED DESCRIPTION OF THE INVENTION

I. General

This invention is based in part on the discovery and characterization of a novel susceptibility locus for asthma and other IgE mediated diseases, two overlapping genes within the locus, termed GPRA and AAA1, and proteins encoded by the genes. The genes have opposite transcriptional orientations and share intronic regions although not exons. The locus maps within human chromosome 7p15-p14.

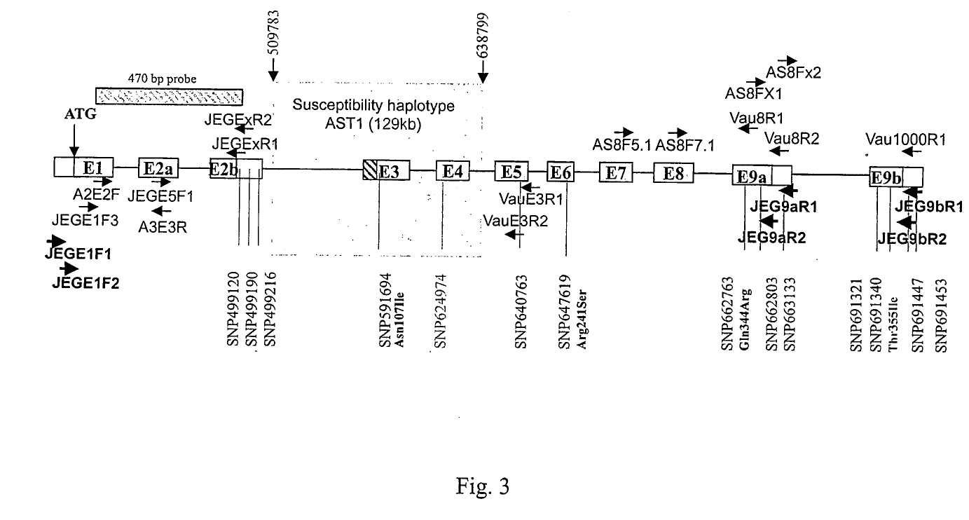

FIG. 3 shows the structure of the human GPRA gene. The gene has nine exons, two of which have alternate forms, and eight introns. The total length of the gene from the first base of the first exon to the last base of the last exon is 220047 bp and the coordinates are 471478 (beginning) 691525 (end) in NT_{—}000380. Part of the genomic sequence of GPRA including part of intron 2, exon 3, intron 3, exon 4 and part of intron 4 defines a sublocus referred to as AST1. This locus is 129,017 bp in length. The locus refers to the part of human chromosome 7 having the 129,017 bp of SEQ ID NO:1 or allelic or species variants thereof.

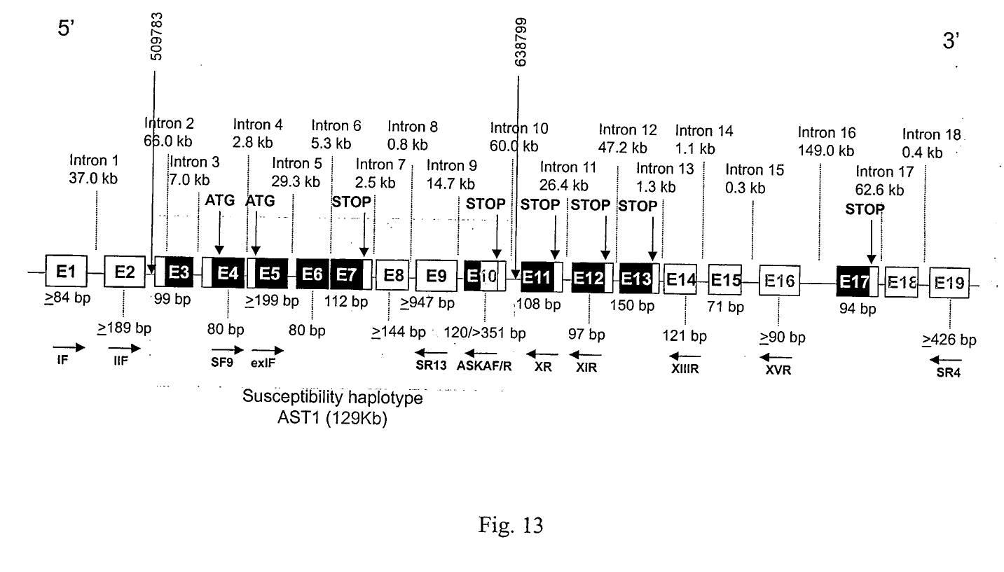

FIG. 13 shows the structure of the human AAA1 gene. The gene has nineteen exons, nine of which have alternate forms, and 18 introns. The total length of the gene from the first base of the first exon to the last base of the last exon is 521,161 bp and the coordinates are 163,615 (beginning) 684776 (end) in NT_{—}000380. Part of the genomic sequence of AAA1 including part of intron 2, exon 3 (a and b), intron 3, exon 4 (a and b), intron 4, exon 5 (a and b), intron 5, exon 6, intron 6, exon 7 (a and b), intron 7, exon 8, intron 8, exon 9, intron 9, exon 10 (a and b) and part of intron 10 occurs within the AST1 locus.

The present application shows that the GPRA and AAA1 genes and AST-1 locus within them are genetically linked and associated with IgE diseases, such as asthma. In addition, the invention provides a collection of polymorphic sites including SNPs (single nucleotide polymorphisms) and DIPs (deletion/insertion polymorphisms) within the locus. Tables 3, 7 and 12 show variant and reference (or wildtype) forms occupying these sites. The variant forms are associated with asthma, other IgE mediated diseases, and/or immune-mediated diseases including autoimmune diseases, and/or cancer and are useful in detection, diagnosis, treatment and prophylaxis thereof.

The AST-1 locus was localized by hierarchical LD mapping in a systemic search for a shared haplotype among the individuals with high IgE serum level across a 19 Mb region that was first identified by a genome wide screen for asthma genes (Laitinen et al. 2001). This analysis defined the AST-1 locus between the markers SNP509783 and SNP638799 and provided statistically significant evidence that specific haplotype of 129 kb within the linkage peak is a risk factor for asthma related traits (SEQ ID NO:1). The best haplotype patterns of polymorphic sites in GPRA (Table 8) were found with the frequency of 13-18% among the high IgE associated chromosomes compared to that of 3-7% among control chromosomes suggesting the risk ratio of 3.9 [95% CI:1.8-8.6] for high IgE level (38/304 among affected vs. 7/220 control chromosomes). The best observe associations for polymorphic sites in AAA1 reached the χ2 values of 8.9-13.6 All markers identified between the positions 509, 783-638, 799 in the genomic contig NT_{—}000380 were found to be in strong LD and therefore useful for diagnostic purposes.

II. GPRA Polypeptides

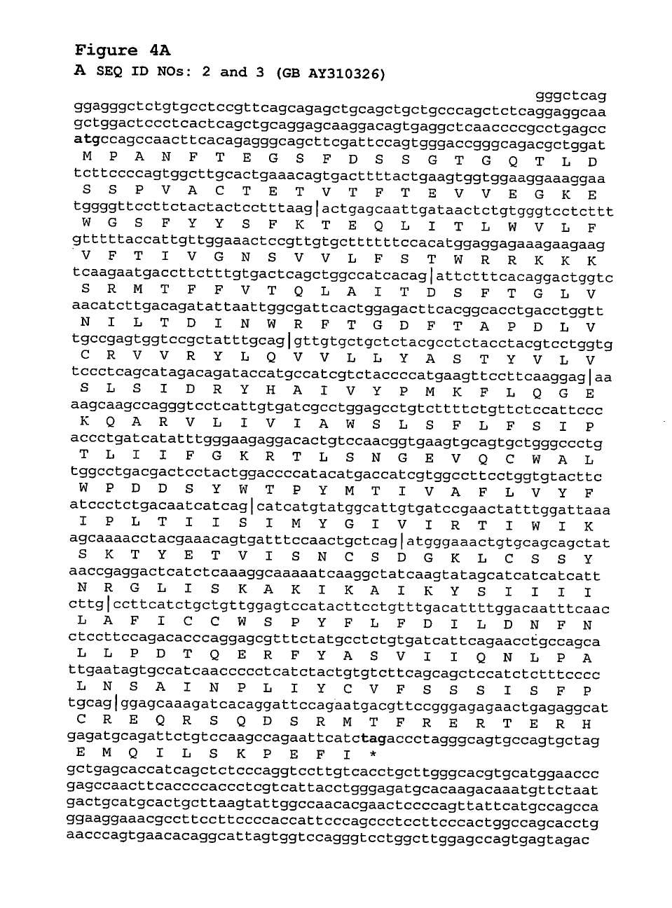

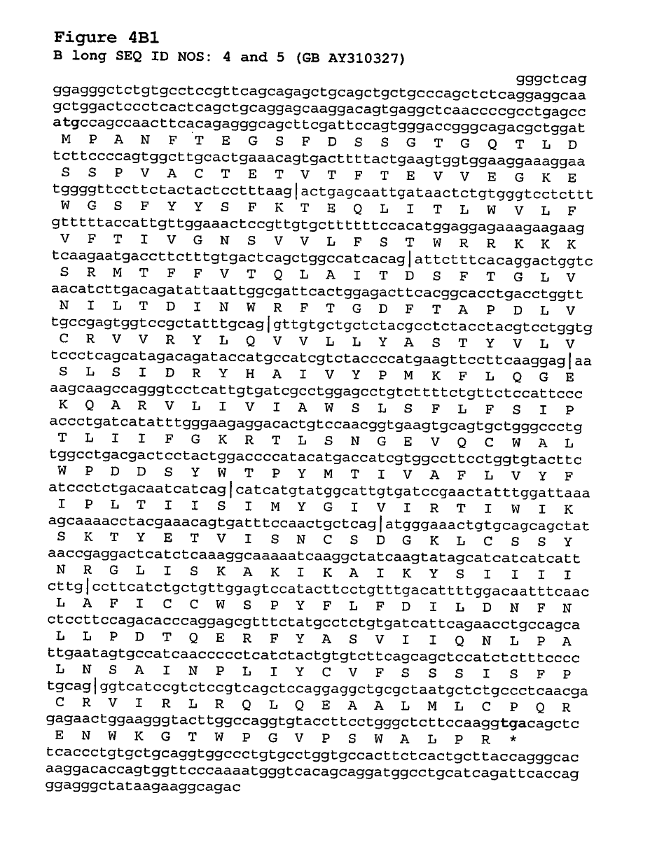

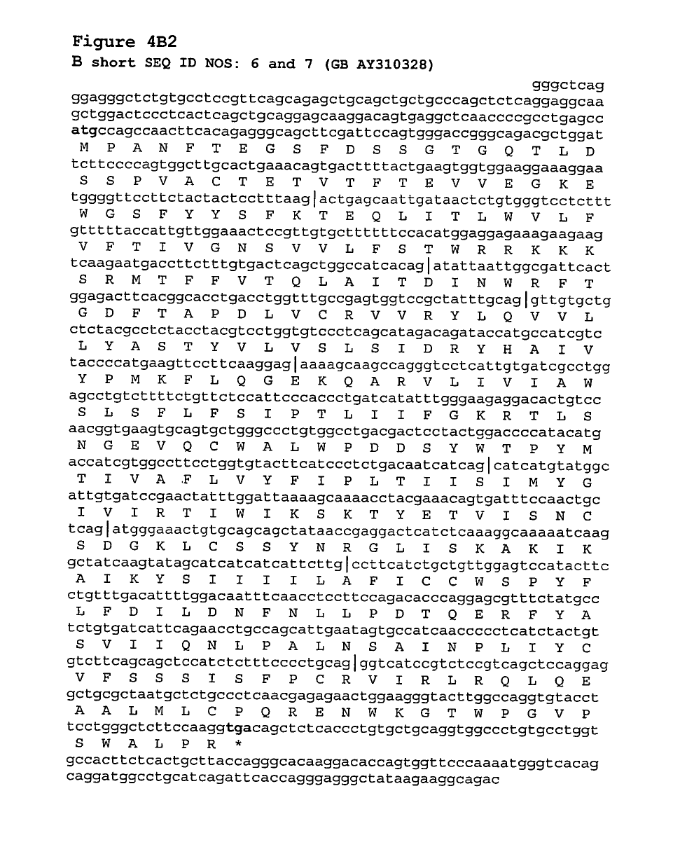

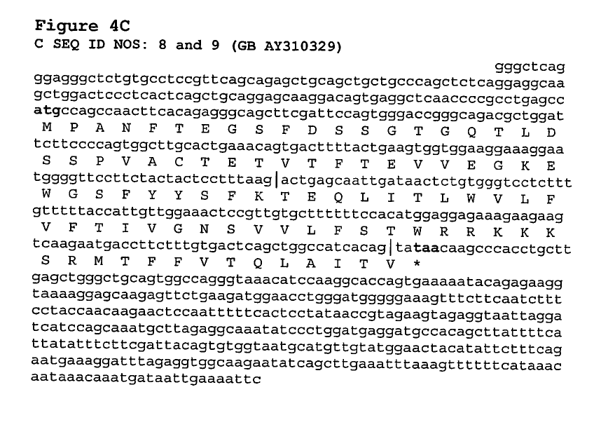

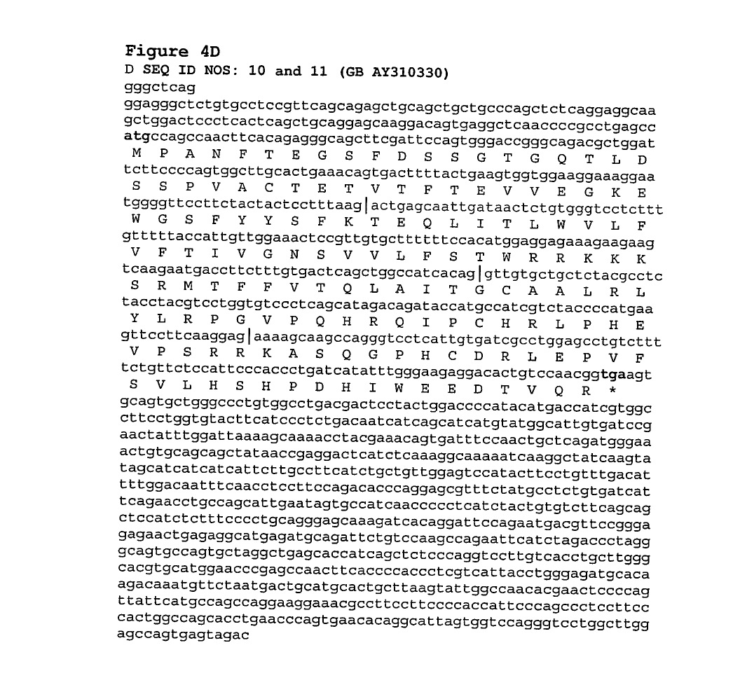

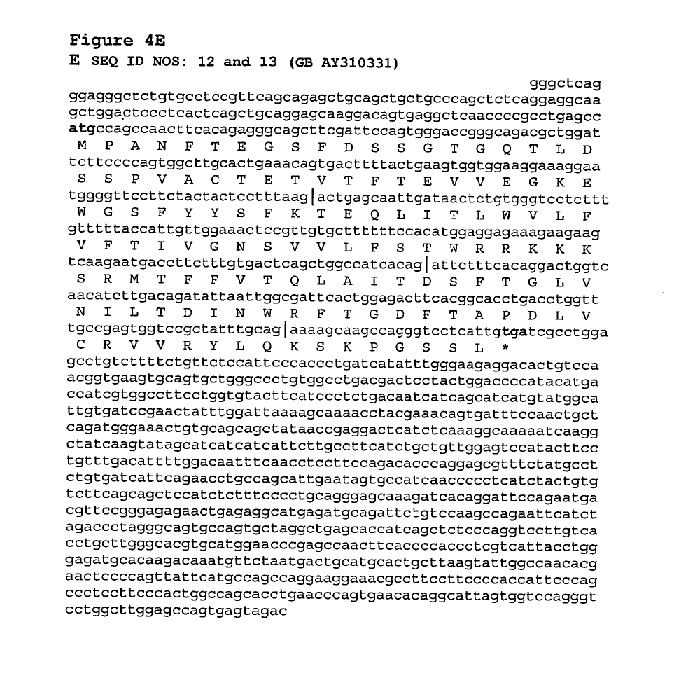

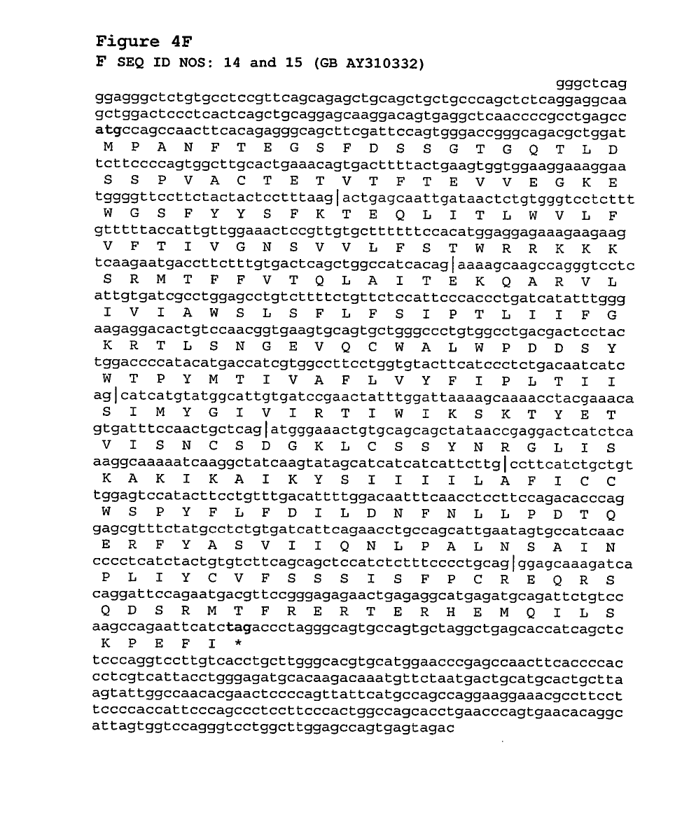

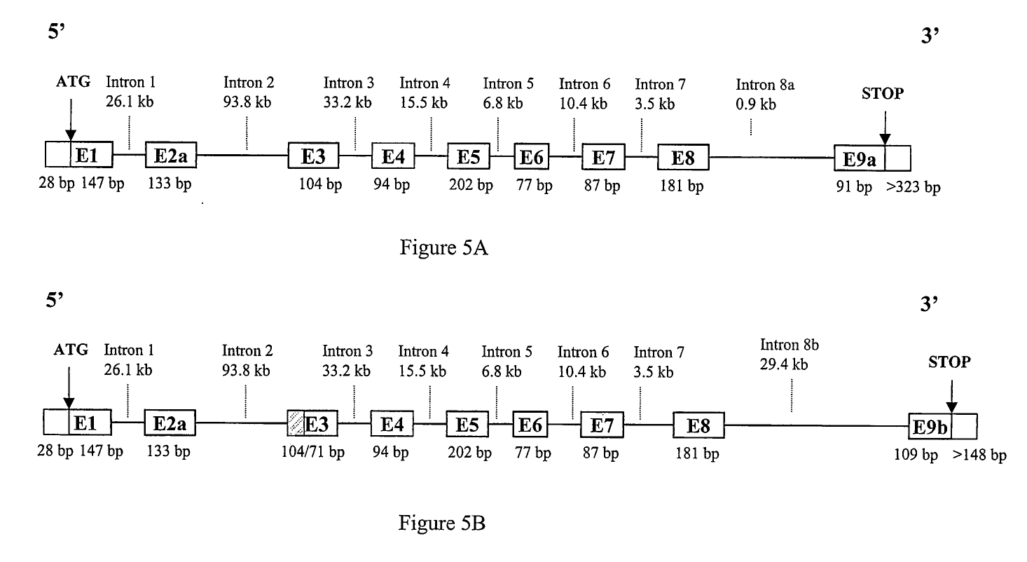

The human GPRA gene can be expressed as at least seven different splice variants termed A, B-long, B-short, C, D, E, and F. The cDNAs encoding each form and amino acid sequence of each form are shown in FIG. 4 (SEQ ID NOS:2-15). The different splicing events that lead to the different forms are shown in FIG. 5. It can be seen that the B-long and B-short forms differ from the A form in that the B forms have a different C-terminus encoded by exon 9b, whereas the C terminus of the A form is encoded by exon 9A. The B-short form (SEQ ID NOS:6 and 7) differs from the B-long form in that the B-short form has a deletion of 10 amino acids encoded by exon III. The C-form (SEQ ID NOS:8 and 9) contains a segment encoded by exon 2b which is not found in other forms. The C-form lacks any segments encoded by exons 3-9. The D form (SEQ ID NOS: 10 and 11) lacks a segment encoded by exon 3, causing a frameshift, whereby exons 4 and 5 are read in a different reading frame causing truncation within exon 5. Form E (SEQ ID NOS: 12 and 13) lacks exon 4 causing a frameshift as a result of which exon 5 is read in a different reading frame causing truncation within exon 5. Form F (SEQ ID NOS: 14 and 15) lacks exons 3 and 4.

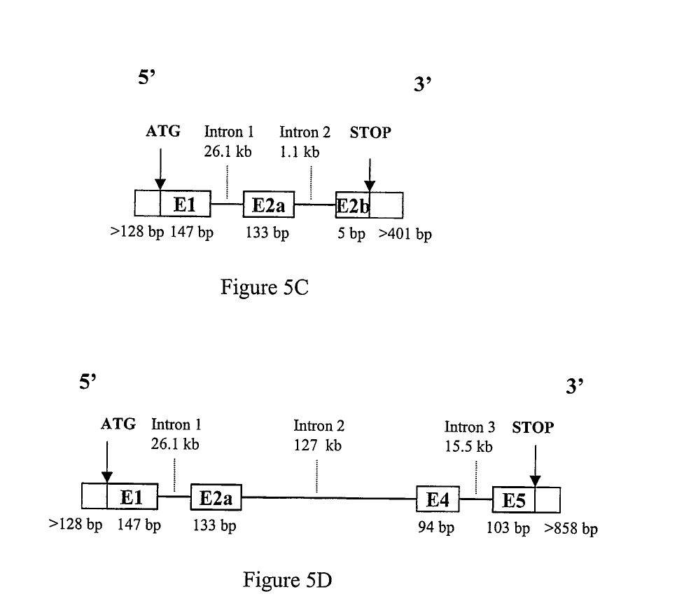

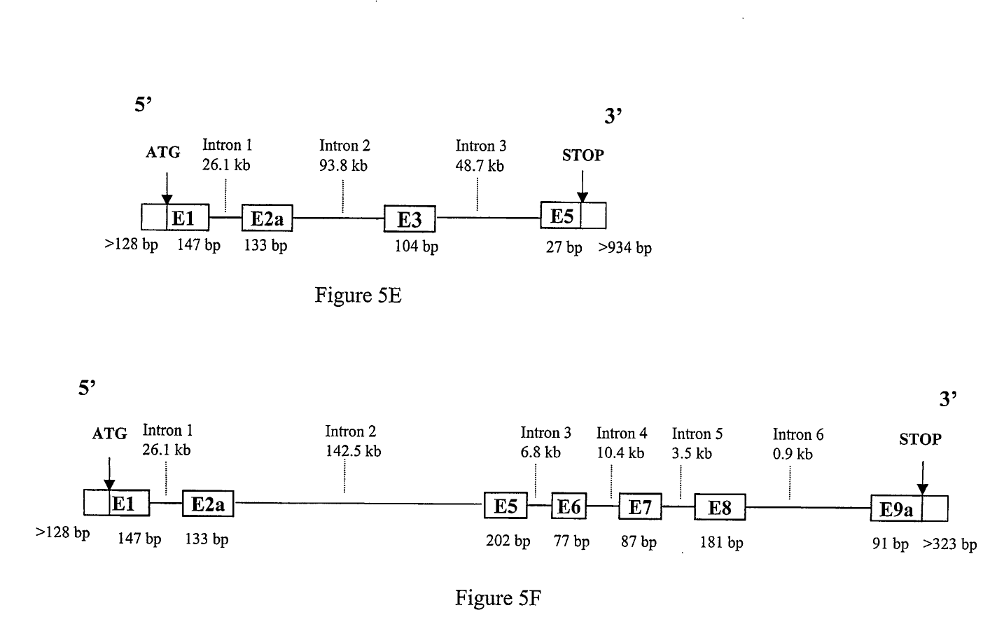

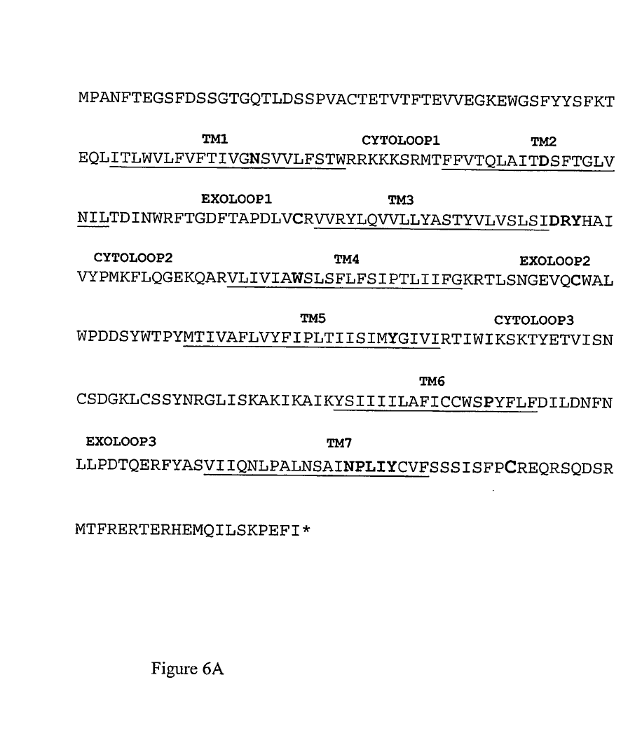

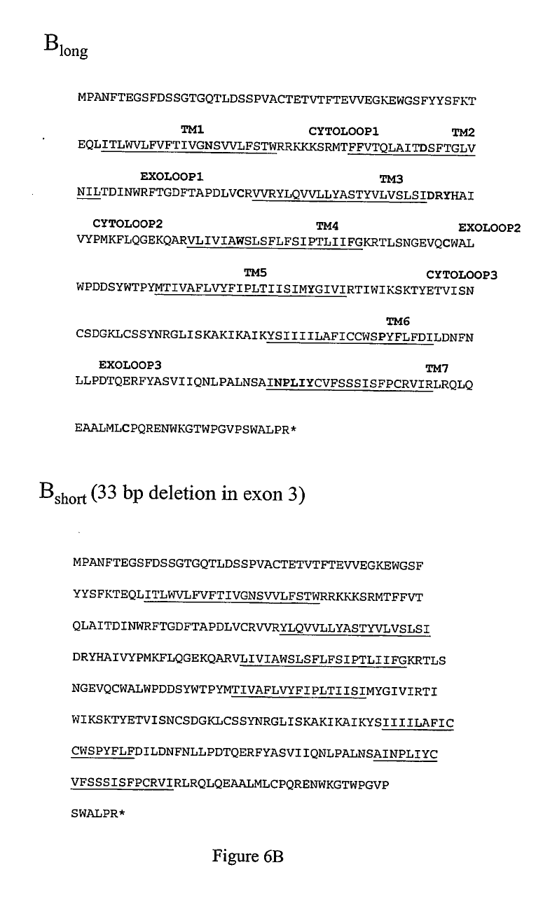

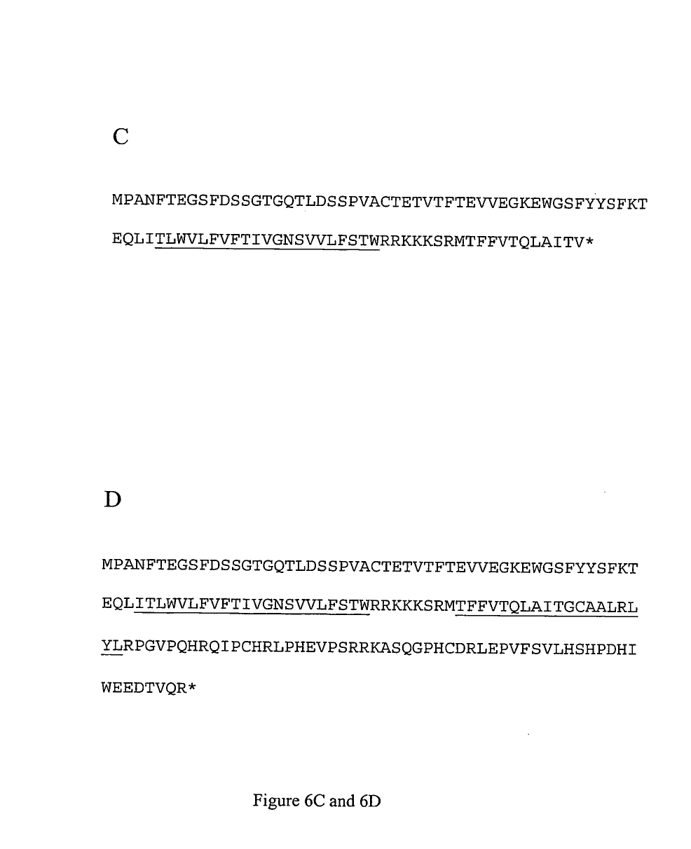

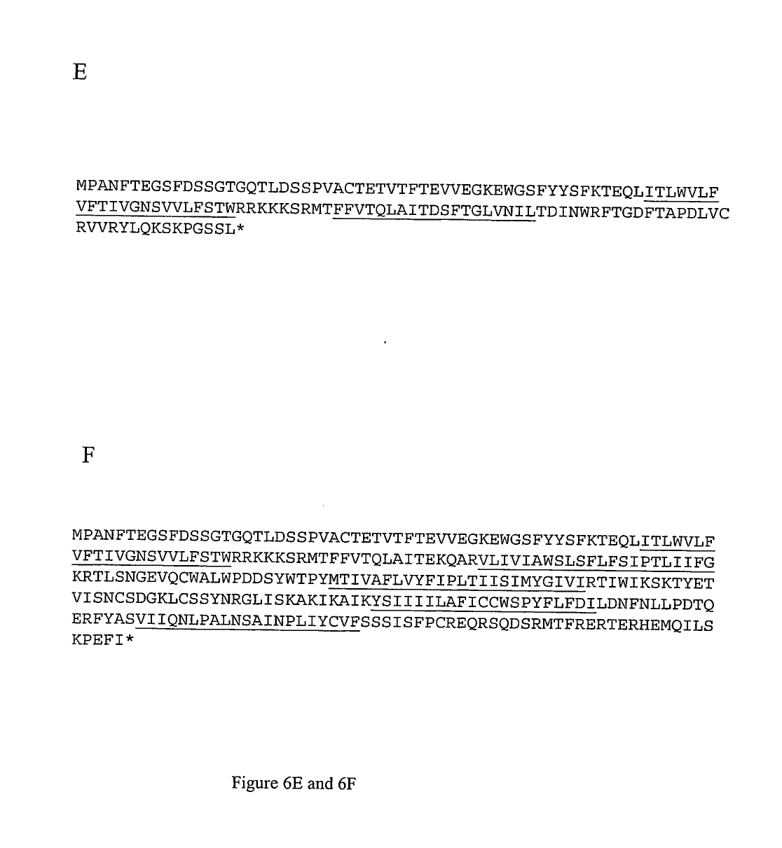

FIG. 6 shows the expected structural characteristics of the different splice variants. The A form includes 371 amino acids and has an extracellular N-terminal domain, seven transmembrane domains and an intracellular C-terminal domains (SEQ ID NO:3). The transmembrane domains are separated by alternating cytoplasmic and extracellular loops. The B-long form (377 amino acids, SEQ ID NO:5) has a similar structure. The predicted size of the extracellular N-terminal domain is about 50 amino acids anticipating suggesting that the endogenous ligand that interacts with GPRA is a small molecule or peptide. The B-short, C, D, E and F variants of GPRA (SEQ ID NOS: 5, 7, 9, 11, 13, and 15, respectively) lack the 7TM structure present in variants A and B-Long.

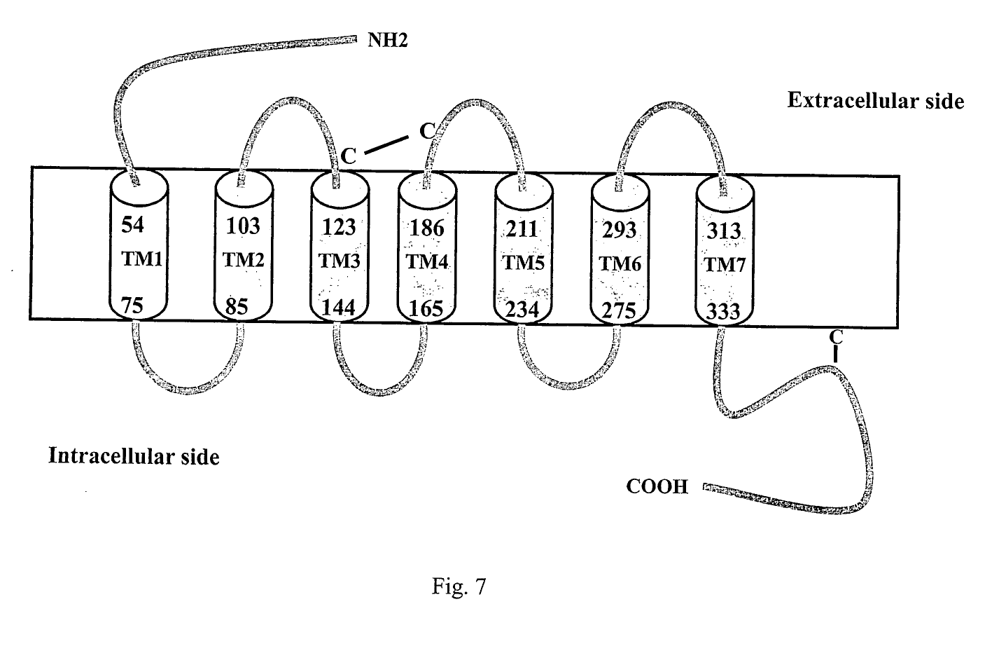

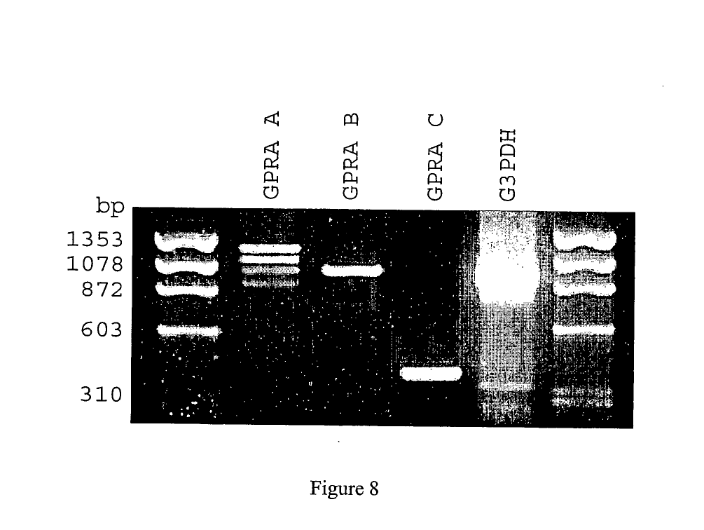

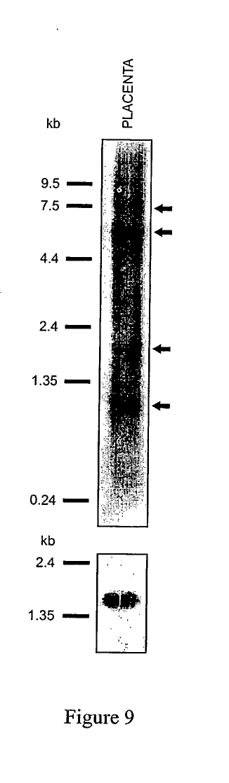

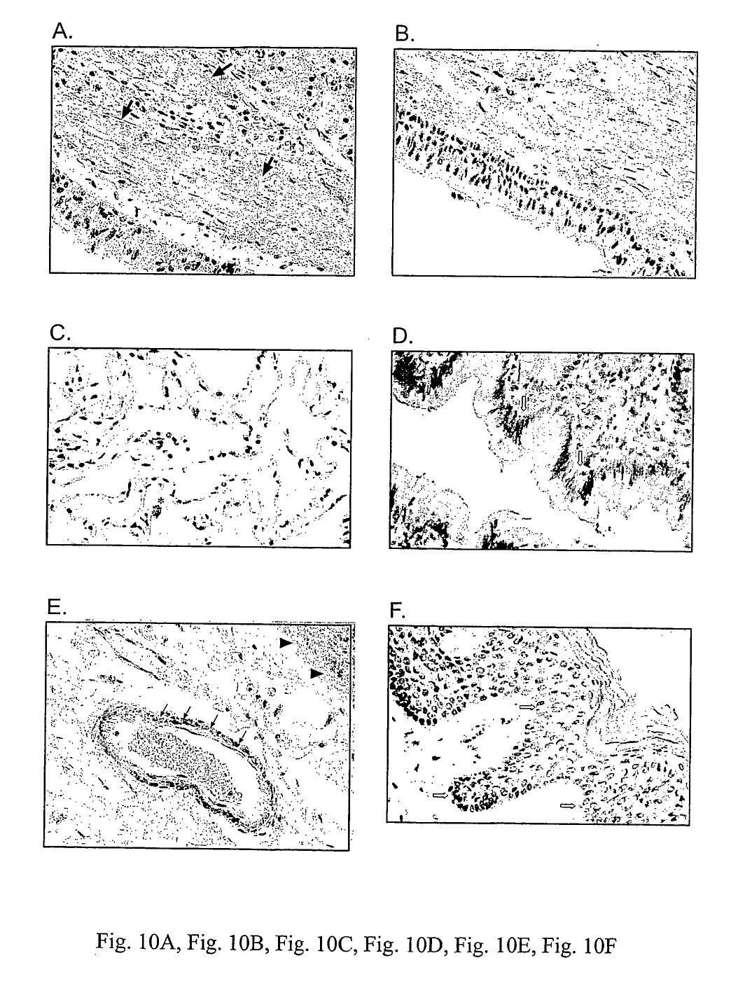

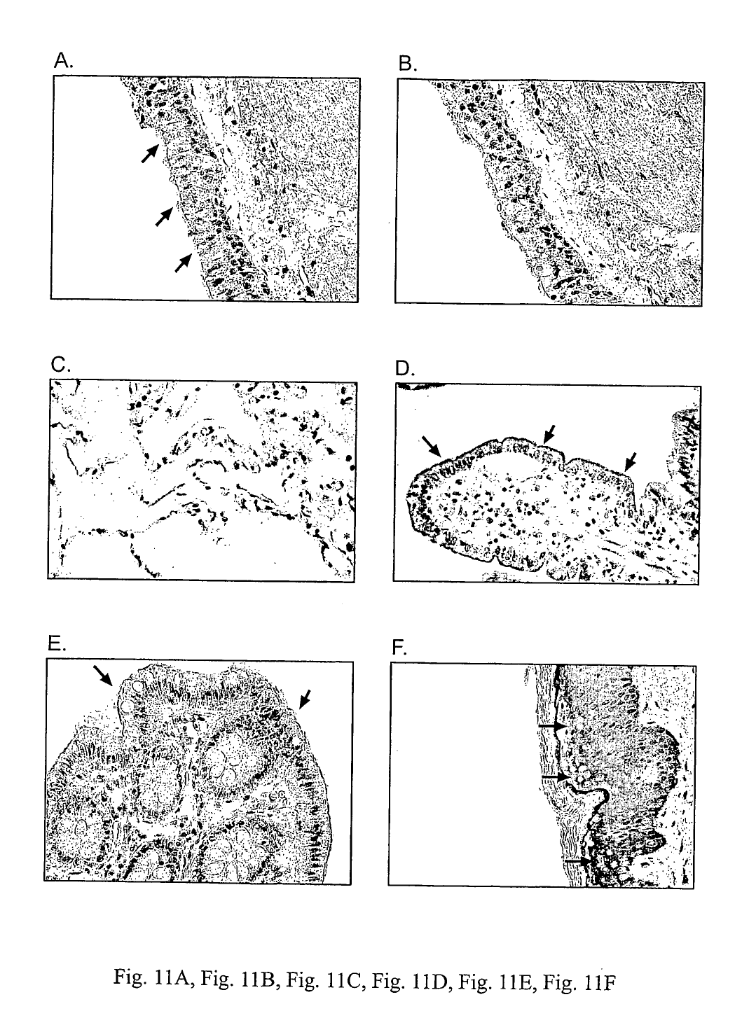

FIG. 7 shows the arrangement of the A or B-Long form in a cell membrane. GPRA falls within the conserved class A of G-protein coupled receptors (GPRs) (Rana et al. 2001, Johnson et al. 2000). GPRs are a large and functionally diverse protein superfamily that form a seven-transmembrane (7TM) helices bundle with alternating extracellular and intracellular loops (see WO 01/18206). Class A contains most well known members of the GPRs such as vasopressin, oxytocin, and bovine rodopsin receptors. For the latter the crystallographic structure is available (Palczewski et al. 2000). GPRA is expressed in human tissues consistent with its role in asthma and IgE mediated disease. Transcripts of GPRA variants A, B. and C were expressed in several tissues including lung and NCI-H358 cell line that represent cells of broncho-epithelial origin (FIGS. 8 and 9). Based on immunohistochemistry, the A variant of the GPRA protein was expressed in smooth muscle cells of bronchi and arterial wall in human lung as subepithelial of smooth muscle cells in colon (FIG. 10) and the B (-long) variant in the epithelium of bronchi and colon (FIG. 11).

A class of GPRA polypeptides can be defined with reference to the exemplary polypeptides defined by SEQ ID NOS: 3, 5, 7, 9, 11, 13 and 15. The class includes allelic, cognate and induced variants of the exemplified polypeptides. Preferred polypeptides shows substantial sequence identity to one or more of the exemplified polypeptides. The class also includes fragments of the exemplified polypeptides having at least 6, 10, 20, 50 100, 200, or 300 contiguous amino acids from one of the above SEQ ID NOS. Some fragments include one or more isolated domains from one of the exemplified polypeptides, e.g., any of the seven transmembrane domains, any of three extracellular loops, any of three intracellular loops, an N-terminal domain and a C-terminal domain. Some polypeptides comprise the amino acid of one of the above SEQ ID NOS. provided that up to 1, 2, 5, 10, 20, 30 or 34 amino acids can be inserted, deleted or substituted relative to the SEQ ID NO. Some GPRA polypeptides comprise an epitope found in one of the exemplified SEQ ID NOS. but not in others. For example, B-Long (SEQ ID NO:5) has a C-terminal epitope of 35 amino acids not found in any of the other forms. Some GPRA polypeptides contain a polymorphic site shown in Table 7 occupied by a variant form.

Preferred GPRA polypeptides are receptors that can be activated to transduce a signal by the same ligand as the exemplified GPRA polypeptide of SEQ ID NOS: 3, 5, 7, 9, 11, 13, and 15. Binding of a ligand or analog to a G-protein receptor induces an alteration in receptor G-protein interaction. The receptor G-protein interaction releases GDP specifically bound to the G protein and permits the binding of GTP, which activates the G protein. Activated G-protein dissociates from the receptor and activates an effector protein, which in turn regulates intracellular levels of second messengers, such as adenyl cyclase, guanyl cyclase, and phospholipase C. Signal transduction can be monitored in cells transfected with a reporter construct consisting of several copies of the consensus cAMP response element (Arias et al., Nature 370, 226-229 (1994)), a minimal tk promoter, and a secreted alkaline phosphatase gene (Clontech) or can be monitored in changes of the concentrations of intracellular calcium.

III. AAA1 Polypeptides

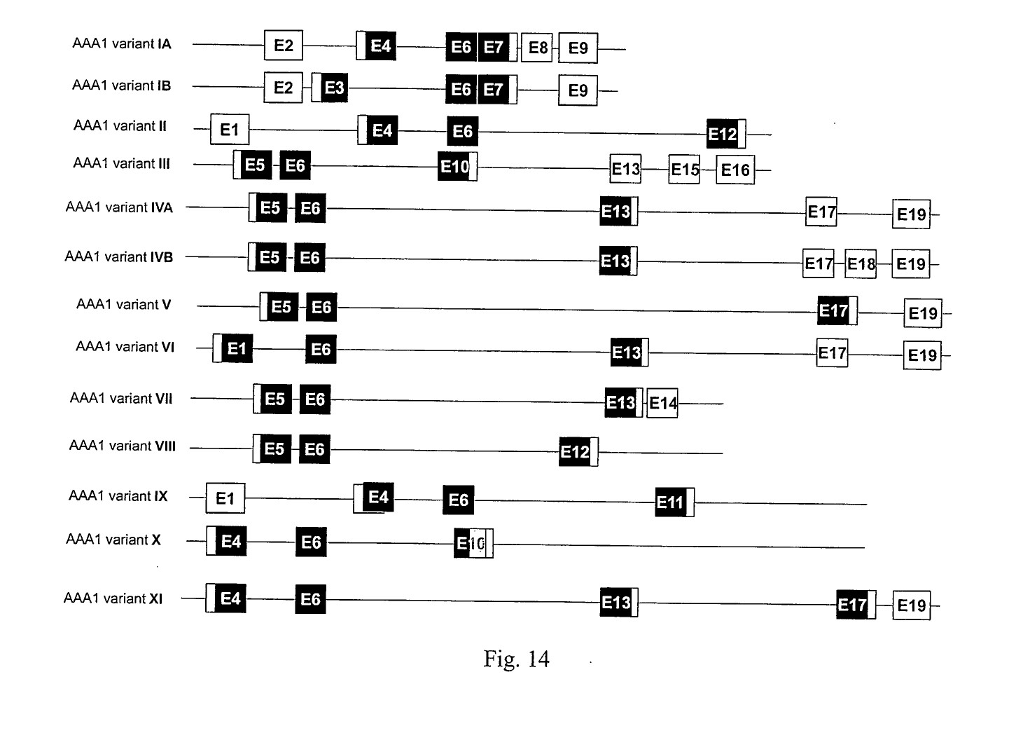

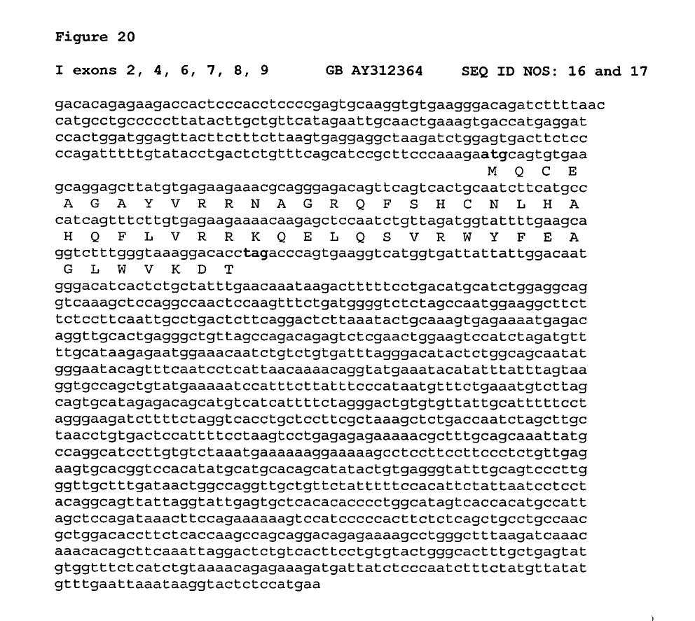

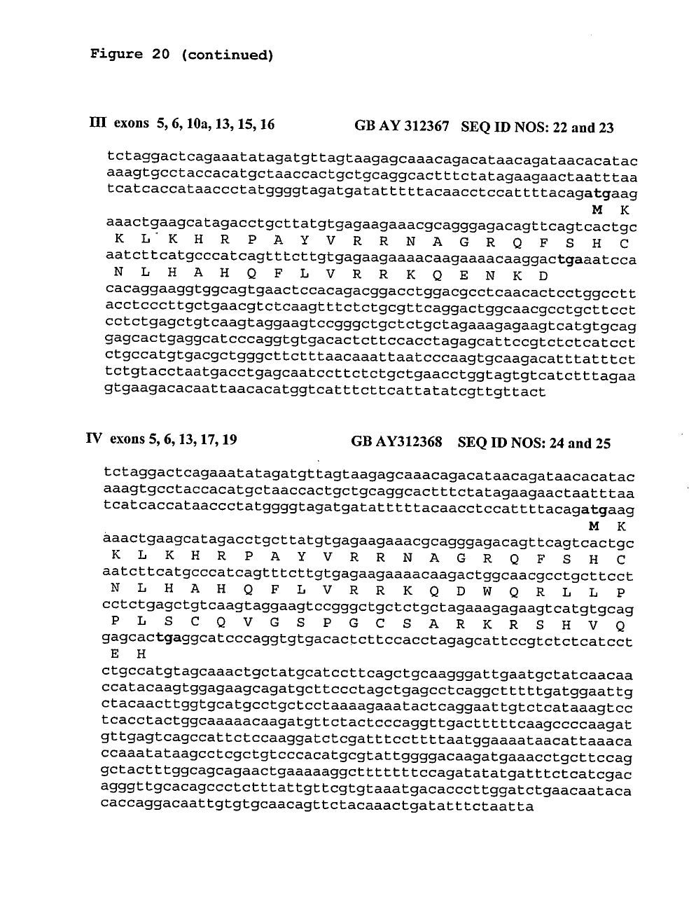

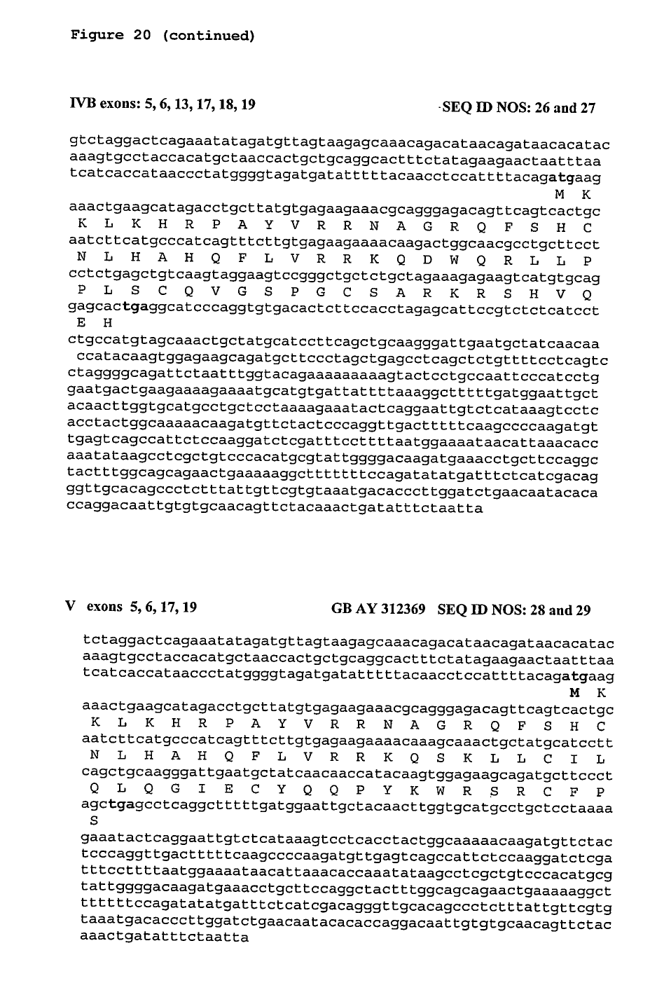

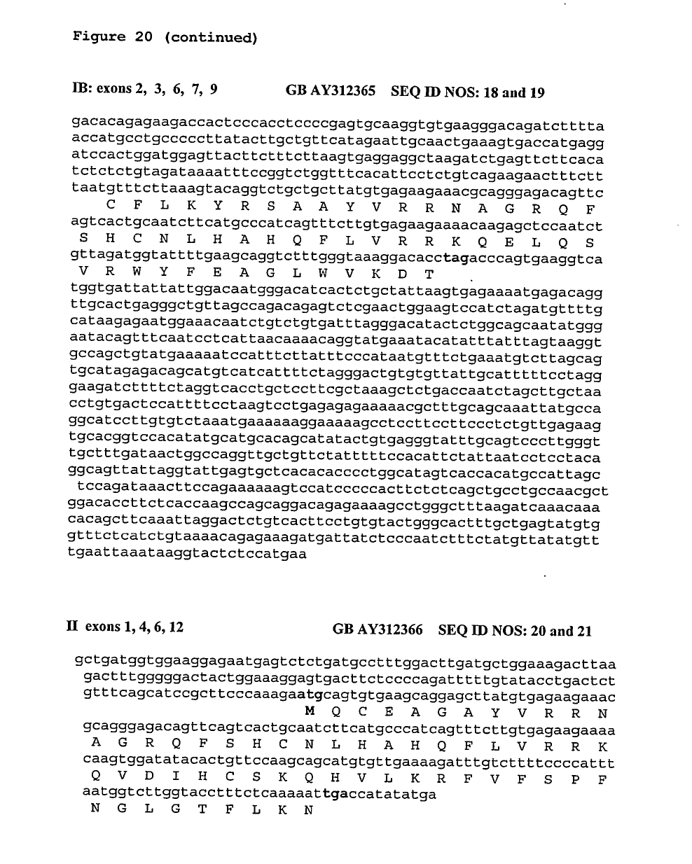

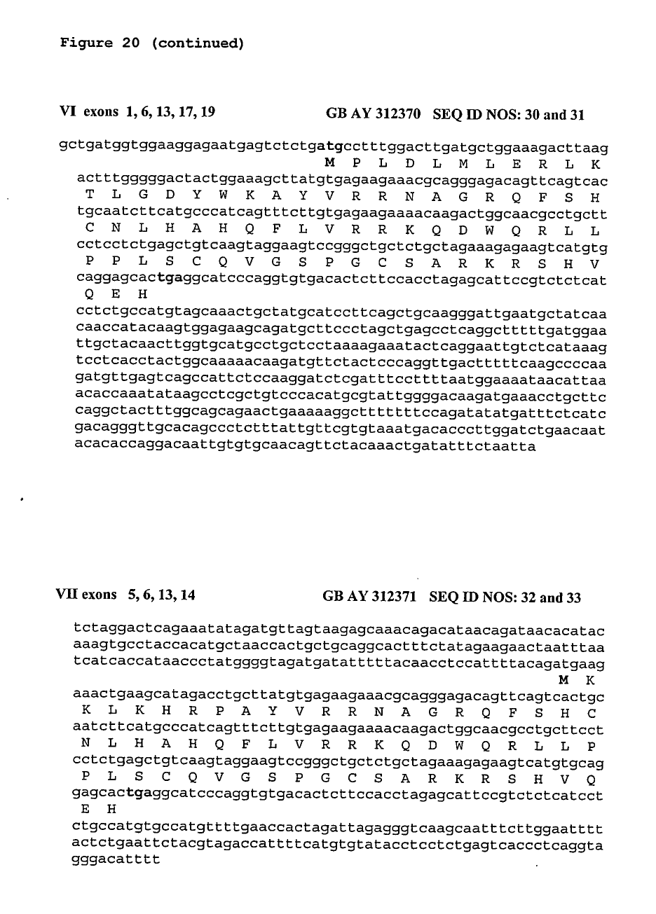

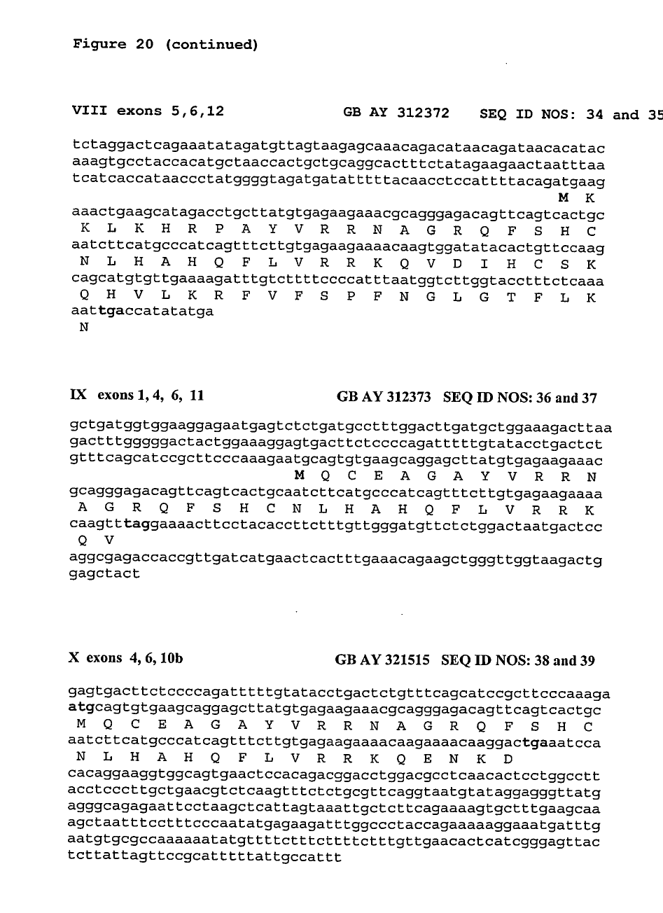

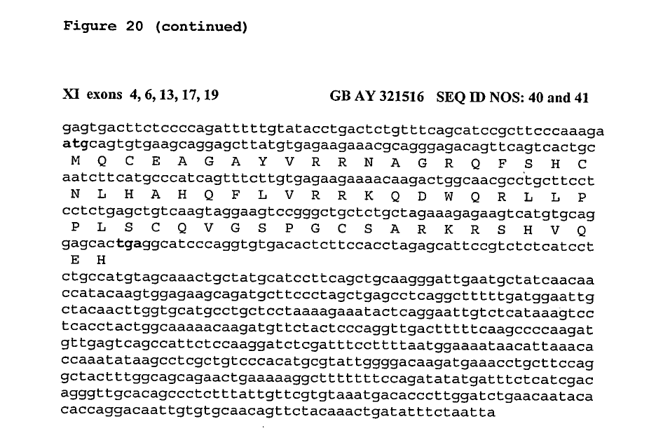

The human AAA1 gene can be expressed as at least thirteen different splice variants termed IA, IB, II, III, IVA, IVB, V, VI, VII, VIII, IX, X, and XI. The cDNAs encoding each form and amino acid sequence of each form are shown in FIG. 20 (SEQ ID NOS:16-41). The different splicing events that lead to the different forms are shown in FIG. 14. It can be seen that all splice variants have exon VI and the same core peptide sequence (AYVRRNAGRQFSHCNLHAHQFLVRRKQ). The AAA1 gene is expressed predominantly in the testis, brain, placenta, lung, heart, skeletal muscle, kidney, liver, fetal liver and fetal lung. In addition to the variants described in FIG. 14, using 3′-RACE we have found also several other variants expressed in different tissues. They all share the exon six.

A class of AAA1 polypeptides can be defined with reference to the exemplary polypeptides defined by SEQ ID NOS:17, 19, 21, 23, 25, 27, 29, 31, 33, 35, 37, 39 and 41. The class includes allelic, cognate and induced variants of the exemplified polypeptides. Preferred polypeptides shows substantial sequence identity to one or more of the exemplified polypeptides. The class also includes fragments of the exemplified polypeptides having at least 6, 10, 20, or 50 contiguous amino acids from one of the above SEQ ID NOS. Some fragments include or consist of the above core peptide sequence. Some polypeptides comprise the amino acid of one of the above SEQ ID NOS. provided that up to 1, 2, 5, 10, or amino acids can be inserted, deleted or substituted relative to the SEQ ID NO. Some AAA1 polypeptides comprise an epitope found in one of the exemplified SEQ ID NOS. but not in others. For example, only the variant 1B contains the peptide sequence encoded by exon III.

IV. GPRA Nucleic Acids

The present invention provides isolated nucleic acids encoding GPRA polypeptides. The nucleic acids can be, for example, genomic, cDNA, RNA or mini-gene (i.e., a hybrid of genomic and cDNA). Genomic and mini-gene nucleic acids contain at least one intronic segment from a GPRA gene, meaning the section of human chromosome 7p15-p14 having the structure shown in FIG. 3, or allelic and cognate variants thereof. Nucleic acids of the invention can include coding regions, intronic regions, 3′ untranslated regions, 5′ untranslated regions, 3′ flanking regions, 5′ flanking regions, enhancers, promoters and other regulatory sequences. A class of GPRA nucleic acids can be defined with reference to exemplified GPRA polypeptide and nucleic acid sequences. The class includes allelic, cognate and induced variants of the exemplified sequences, as well as nucleotide substitutions, which due to the degeneracy of the genetic code, do not effect the amino acid sequence of an encoded polypeptide. The class includes nucleic acids that encode the GPRA polypeptides described above. The class also includes GPRA nucleic acids showing substantial sequence identity to exemplified GPRA nucleic acids (i.e., SEQ ID NOS:1, 2, 4, 6, 8, 10, 12 and 14). SEQ ID NO:1 is a genomic sequence representing the part of a GPRA gene designated Ast-1 and shown in FIG. 3. SEQ ID NO:2 is a 1113 bp cDNA encoding GPRA variant A (FIG. 4A). SEQ ID NO:4 is a 1132 bp cDNA encoding GPRA variant B-Long (FIG. 4B). SEQ ID NOS:6, 8, 10, 12, and 14 encode GPRA variants B-Short, C, D, E and F.

The class also includes GPRA nucleic acids that hybridize under highly stringent conditions with at least one of the exemplified nucleic acids. Some GPRA nucleic acids of the invention hybridize under highly stringent conditions with at least one of the GPRA nucleic acids of the invention without hybridizing under the same conditions to others. For example, some GPRA nucleic acids hybridize to at least one of SEQ ID NOS:1, 4, 6, 8, 10, 12 and 14 without hybridizing to SEQ ID NO:2. Some GPRA nucleic acids include a polymorphic site occupied by a variant form as shown in Table 3 or Table 7. Some GPRA nucleic acids are genomic or minigene. Such nucleic acids contain at least one intronic sequence from any of the intron of a GPRA gene. Inclusion of an intronic sequence can be useful for increasing expression levels of a nucleic acid in cells or transgenic animals. Inclusion of an intronic sequence from introns 2, 3 or 4 is particularly useful for analyzing splice variation of the GPRA gene. Nucleic acids also include fragments of exemplified sequences SEQ ID NOS:1, 4, 6, 8, 10, 12 and 14. The fragments typically contain up to 10, 20, 25, 50, 100, 300, 400, 500 or 1000 or 1500 nucleic acids from any of the above SEQ ID NOS. Optionally, fragments include a polymorphic site shown in Table 3 or 7. The polymorphic site can be occupied by either a reference or variant form.

GPRA nucleic acids can be linked to other nucleic acids with which they are not naturally associated such as vector or a heterologous promoter sequence. Preferred nucleic acids of the invention include or are immediately adjacent to at least one of the polymorphic sites shown in Table 3 or 7. Such nucleic acids are useful as primers or probes for detection of specific alleles. Other nucleic acids are useful for expressing proteins encoded by the AST-1 locus in cells or transgenic animals. Other nucleic acids are useful for achieving gene suppression either by homologous recombination to generate a knockout animal or an antisense or siRNA mechanism. Nucleic acids are also useful for identifying, purifying, and isolating nucleic acids encoding other, non-human, mammalian forms of asthma locus-1.

Nucleic acids of the invention can be isolated using standard molecular biology techniques and the provided sequence information (Ausubel et al, In Current protocols in Molecular Biology, John Wiley and Sons, publishers, 1989); Sambrook et al, supra). Cognate forms (i.e., nucleic acids encoding asthma locus-1 molecules derived from species other than human) or other related sequences (e.g., paralogs) can be obtained by low, moderate or high stringency hybridization with all or a portion of the particular human sequence as a probe using methods well known in the art for nucleic acid hybridization and cloning.

V. AAA1 Nucleic Acids

The present invention provides isolated nucleic acids encoding AAA1 polypeptides. The nucleic acids can be, for example, genomic, cDNA, RNA or mini-gene (i.e., a hybrid of genomic and cDNA). Genomic and mini-gene nucleic acids contain at least one intronic segment from an AAA1 gene, meaning the section of human chromosome 7p15-p14 having the structure shown in FIG. 13, or allelic and cognate variants thereof. Nucleic acids of the invention can include coding regions, intronic regions, 3′ untranslated regions, 5′ untranslated regions, 3′ flanking regions, 5′ flanking regions, enhancers, promoters and other regulatory sequences. A class of AAA1 nucleic acids can be defined with reference to exemplified AAA1 polypeptide and nucleic acid sequences. The class includes allelic, cognate and induced variants of the exemplified sequences, as well as nucleotide substitutions, which due to the degeneracy of the genetic code, do not effect the amino acid sequence of an encoded polypeptide. The class includes nucleic acids that encode the AAA1 polypeptides described above. The class also includes AAA1 nucleic acids showing substantial sequence identity to exemplified AAA1 nucleic acids (i.e., SEQ ID NOS:1, 16, 18, 20, 22, 24, 26, 28, 30, 32, 34, 36, 38 and 40). SEQ ID NO:1 is a genomic sequence representing the part of an AAA1 gene designated Ast-1 and shown in FIG. 13. SEQ ID NOS:16, 18, 20, 22, 24, 26, 28, 30, 32, 34, 36, 38 and 40 encode cDNA splice variants shown in FIG. 20.

The class also includes AAA1 nucleic acids that hybridize under highly stringent conditions with at least one of the exemplified nucleic acids. Some AAA1 nucleic acids of the invention hybridize under highly stringent conditions with at least one of the exemplified nucleic acids (i.e., SEQ ID NOS: 16, 18, 20, 22, 24, 26, 28, 30, 32, 34, 36, 38 and 40) without hybridizing under the same conditions to other exemplified nucleic acids. Some AAA1 nucleic acids include a polymorphic site occupied by a variant form as shown in Table 12. Some AAA1 nucleic acids are genomic or minigene. Such nucleic acids contain at least one intronic sequence from any of the introns of an AAA1 gene. Nucleic acids also include fragments of exemplified sequences SEQ ID NOS:16, 18, 20, 22, 24, 26, 28, 30, 32, 34, 36, 38 and 40. The fragments typically contain up to 10, 20, 25, 50, or 100, nucleotides from any of the above SEQ ID NOS. Optionally, fragments include a polymorphic site shown in Table 12. The polymorphic site can be occupied by either a reference or variant form.

AAA1 nucleic acids can be linked to other nucleic acids with which they are not naturally associated such as vector or a heterologous promoter sequence. Preferred nucleic acids of the invention include or are immediately adjacent to at least one of the polymorphic sites shown in Table 3 or Table 12. Such nucleic acids are useful as primers or probes for detection of specific alleles. Other nucleic acids are useful for expressing AAA1 polypeptides in cells or transgenic animals. Other nucleic acids are useful for achieving gene suppression either by homologous recombination to generate a knockout animal or an antisense or siRNA mechanism. Nucleic acids are also useful for identifying, purifying, and isolating nucleic acids encoding other, non-human, mammalian forms of the AAA1 gene.

As well as or instead of encoding proteins, AAA1 transcripts can have roles in regulating expression of GPRA nucleic acids. A class of noncoding (ncRNA) has been described with regulatory activity in gene transcription and splicing (see e.g. reviews by Michel 2002; Numata et al. 2003).

VI. Antibodies

The GPRA and AAA1 polypeptides of the invention are useful for generating antibodies. The antibodies can be polyclonal antibodies, distinct monoclonal antibodies or pooled monoclonal antibodies with different epitopic specificities. Monoclonal antibodies are made from antigen-containing fragments of the protein by standard procedures according to the type of antibody (see, e.g., Kohler, et al., Nature, 256:495, (1975); and Harlow & Lane, Antibodies, A Laboratory Manual (C.S.H.P., NY, 1988) Queen et al., Proc. Natl. Acad. Sci. USA 86:10029-10033 (1989) and WO 90/07861; Dower et al., WO 91/17271 and McCafferty et al., WO 92/01047 (each of which is incorporated by reference for all purposes). Phage display technology can also be used to mutagenize CDR regions of antibodies previously shown to have affinity for the peptides of the present invention. Some antibodies bind to an epitope present in one form of GPRA or AAA1 but not others. For example, some antibodies bind to an epitope within amino acids 343-377 of GPRA long B (SEQ ID NO:4). Some antibodies specifically bind to a GPRA polypeptide containing a variant form at a polymorphic site shown in Table 7 without binding to polypeptides containing a reference form at the site. The antibodies can be purified, for example, by binding to and elution from a support to which the polypeptide or a peptide to which the antibodies were raised is bound.

Antibodies of the invention are useful, for example, in screening cDNA expression libraries and for identifying clones containing cDNA inserts which encode structurally-related, immunocrossreactive proteins. See, for example, Aruffo & Seed, Proc. Natl. Acad. Sci. USA 84:8573-8577 (1977) (incorporated herein by reference in its entirety for all purposes). Antibodies are also useful to identify and/or purify immunocrossreactive proteins that are structurally related to SEQ ID NOS:3, 5, 7, 9, 11, 13 and 15 and 17, 19, 21, 23, 25, 27, 29, 31, 33, 35, 37, 39 and 41 and fragments thereof used to generate the antibody.

VII. Polymorphisms of the Invention

The present invention provides a collection of polymorphic sites both single nucleotide polymorphisms (SNPs) and deletion/insertion polymorphisms (DIPs) in the GPRA gene. SNPs and DIPs can be used in mapping the human genome and, when a SNP or a DIP is linked with a disease or condition, to clarify genetic basis of the disease or condition, in this particular case, at least of asthma.

Table 3 describes 169 SNPs and 18 DIPs occurring within exons 3 and 4 and introns 2, 3 and 4 of a GPRA gene having variant forms associated with asthma. For AAA1 the corresponding polymorphisms are located in exons 4, 6, 9, 10a/b and introns 2, 3, 4, 5, 6, 7, 8, 9, and 10 (FIG. 13). The locus containing these polymorphisms is also referred to as AST-1 (SEQ ID NO:1). Some of the variant forms of these polymorphism may associate with asthma due to an effect on the splicing of GPRA and/or AAA1 gene in the formation of a transcript. Other polymorphic sites may be in linkage disequilibrium with such sites or sites in coding regions that exert effects through changing the activity of GPRA or AAA1 polypeptides. Seven different haplotypes H1-H7 were identified that explain 94.5% of the genetic variation detected at the AST-1 locus and are described in Table 3. Haplotype H1 was found identical to the reference sequence NT_{—}000380. The first column in Table 3 indicates the types of polymorphism (e.g., SNP or insertion/deletion). The second column indicates the position that the polymorphism occurs in SEQ ID NO:1. The third column indicates the position of the polymorphism occurs in NT_{—}000380. The fourth column indicates several contiguous nucleotides flanking the polymorphic sites and the nature of the variant (i.e., asthma associated) and reference/wildtype polymorphic forms. In all polymorphic sites, the allele present in SEQ ID NO:1 and NT_{—}000380 is indicated first, and the allele from another source second. Columns 5-11 indicate alleles present at that site in haplotypes H1-H7. Column 12 indicates the location of polymorphisms in GPRA and AAA1 exons. In the AST-1 locus, there are coding polymorphism in AAA1 gene (exon 6, Asn->Ser) and one in GPRA gene (exon 3, Asn->Ile). Table 13 shows polymorphisms that are unique for haplotypes H1-H7, respectively, and Table 14 polymorphisms that can be employed to recognize specific haplotype combinations.

The SNPs include the following substitutions with the variant form being indicated before the reference form of SEQ ID NO: 1. Position 1 (preferably T (i.e., variant form) instead of C (reference form)), position 93 (preferably G instead of A), position 918 (preferably A instead of G), position 983 (preferably T instead of A), position 987 (preferably C instead of T), position 1542 (preferably T instead of C), position 1710 (preferably G instead of A), position 1818 (preferably T instead of C), position 1927 (preferably T instead of A), position 2254 (preferably C instead of T), position 2937 (preferably A instead of G), position 3877 (preferably C instead of G), position 4012 (preferably A instead of C), position 4631 (preferably C instead of T), position 4689 (preferably G instead of C), position 4961 (preferably G instead of A), position 5442 (preferably G instead of C), position 5634 (preferably A instead of G), position 5850 (preferably G instead of A), position 6312 (preferably T instead of C), position 6392 (preferably T instead of G), position 6485 (preferably A instead of G), position 6522 (preferably G instead of C), position 6646 (preferably G instead of A), position 6739 (preferably A instead of G), position 6760 (preferably C instead of T), position 7125 (preferably A instead of C), position 7229 (preferably T instead of C), position 7277 (preferably G instead of C), position 7303 (preferably T instead of G), position 7305 (preferably C instead of G), position 7496 (preferably T instead of C), position 7550 (preferably A instead of G), position 8490 (preferably T instead of C), position 9649 (preferably G instead of T), position 10816 (preferably C instead of T), position 11858 (preferably G instead of A), position 12581 (preferably C instead of G), position 16845 (preferably C instead of T), position 16893 (preferably C instead of T), position 16980 (preferably C instead of T), position 17147 (preferably A instead of G), position 17209 (preferably A instead of C), position 17435 (preferably G instead of A), position 18383 (preferably A instead of G), position 18927 (preferably T instead of G), position 18978 (preferably G instead of A), position 19268 (preferably C instead of G), position 19272 (preferably T instead of A), position 19360 (preferably A instead of G), position 19452 (preferably A instead of G), position 19671 (preferably A instead of G), position 19712 (preferably A instead of C), position 19774 (preferably A instead of C), position 20038 (preferably C instead of T), position 20089 (preferably A instead of T), position 20309 (preferably A instead of G), position 20395 (preferably C instead of T), position 20789 (preferably T instead of G), position 21850 (preferably T instead of C), position 22475 (preferably C instead of T), position 22493 (preferably G instead of A), position 22715 (preferably A instead of G), position 22869 (preferably C instead of T), position 22934 (preferably T instead of A), position 24007 (preferably C instead of T), position 24264 (preferably T instead of G), position 24869 (preferably C instead of T), position 26198 (preferably T instead of C), position 26356 (preferably T instead of C), position 26675 (preferably G instead of A), position 27404 (preferably T instead of C), position 28197 (preferably A instead of G), position 28770 (preferably T instead of C), position 28785 (preferably A instead of G), position 28858 (preferably C instead of T), position 28866 (preferably C instead of G), position 31224 (preferably A instead of G), position 31910 (preferably A instead of G), position 32124 (preferably A instead of C), position 32185 (preferably C instead of T), position 32976 (preferably C instead of T), position 33350 (preferably A instead of C), position 33798 (preferably A instead of G), position 34362 (preferably C instead of G), position 34716 (preferably C instead of A), position 35559 (preferably T instead of C), position 36551 (preferably A instead of G), position 36909 (preferably A instead of C), position 37327 (preferably T instead of G), position 37415 (preferably A instead of G), position 37685 (preferably G instead of A), position 37931 (preferably T instead of C), position 37959 (preferably T instead of C), position 39314 (preferably A instead of G), position 39343 (preferably T instead of G), position 39927 (preferably C instead of T), position 45826 (preferably T instead of C), position 50197 (preferably T instead of G), position 50334 (preferably A instead of G), position 50493 (preferably A instead of G), position 50632 (preferably A instead of G), position 50835 (preferably C instead of A), position 50955 (preferably C instead of G), position 51217 (preferably A instead of G), position 51476 (preferably G instead of A), position 51536 (preferably T instead of C), position 51861 (preferably G instead of C), position 51884 (preferably T instead of G), position 51975 (preferably C instead of G), position 52573 (preferably A instead of G), position 52776 (preferably C instead of G), position 53803 (preferably T instead of C), position 53922 (preferably C instead of T), position 54148 (preferably A instead of G), position 54199 (preferably C instead of T), position 54641 (preferably C instead of G), position 54751 (preferably C instead of T), position 55000 (preferably A instead of G), position 55134 (preferably A instead of G), position 56683 (preferably T instead of C), position 56856 (preferably T instead of C), position 57790 (preferably C instead of A), position 60559 (preferably G instead of C), position 60604 (preferably T instead of A), position 61165 (preferably A instead of G), position 64559 (preferably T instead of C), position 65171 (preferably G instead of A), position 65857 (preferably G instead of T), position 66164 (preferably T instead of C), position 66190 (preferably T instead of C), position 66526 (preferably T instead of C), position 66902 (preferably G instead of A), position 67857(preferably A instead of T), position 67919 (preferably C instead of T), position 72270 (preferably A instead of G), position 75115 (preferably A instead of G), position 76080 (preferably T instead of C), position 76101 (preferably C instead of G), position 81912 (preferably T instead of A), position 82203 (preferably G instead of A), position 82332 (preferably T instead of C), position 82922 (preferably A instead of T), position 83552 (preferably T instead of C), position 85227 (preferably C instead of T), position 85271 (preferably A instead of G), position 107610 (preferably C instead of T), position 110989 (preferably C instead of T), position 111012 (preferably T instead of C), position 112030 (preferably C instead of G), position 112037 (preferably G instead of T), position 112283 (preferably T instead of A), position 112726 (preferably A instead of T), position 112859 (preferably C instead of A), position 113428 (preferably G instead of C), position 113645 (preferably T instead of A), position 113944 (preferably G instead of T), position 114945 (preferably G instead of A), position 115192 (preferably G instead of C), position 115628 (preferably C instead of T), position 116032 (preferably A instead of G), position 116464 (preferably A instead of G), position 116515 (preferably A instead of G), position 116926 (preferably C instead of T), position 117276 (preferably A instead of T), position 123667 (preferably A instead of G), position (preferably 123770 instead G of A), position 123788 (preferably C instead of G), and position 129017 (preferably G instead of A).

DIPS of the invention defined relative to SEQ ID NO: 1 include: positions 184-185 (preferably insertion of AAGATA), positions 1202-1206 (preferably deletion of TAAGT), positions 6786-6817 (preferably repeat of (TAAA)₇), position 6821 (preferably deletion of T), positions 7240-7243 (preferably deletion of ACTT), positions 7306-7308 (preferably deletion of TGT), positions 7334-7335 (preferably deletion of AT), positions 9012-9035 (preferably repeat of (CT)₁₀), positions 9199-9201 (preferably deletion of TCT), position 9355-9356 (preferably insertion of T), positions 9782-9785 (preferably deletion of GTCT), positions 22122-22123 (preferably deletion of TG), positions 26929-26968 (preferably repeat of (TAAA)₁₁), positions 28495-28564 (preferably repeat of (CA)₁₂), position 34909 (preferably deletion of T), positions 38850-38852 (preferably deletion of CTC), positions 51022-51049 (preferably repeat of (CA)₈), and positions 52286-52287 (preferably insertion of CC).

Table 7 provide 13 additional polymorphisms occurring within exonic regions of a GPRA gene. The next eight columns indicate the position of a polymorphic site in SEQ ID NO:1, 2, 4, 6, 8, 10, 12 or 14 respectively. The next column indicates a polymorphic site and flanking nucleotides. The form of the polymorphic site present in one or more of SEQ ID NOS: 1, 2, 4, 6, 8, 10, 12, and 14 is shown first and the form present in another source is indicated second. In all cases except those indicated by a *, the polymorphic form indicated first is the wildtype or reference form and the polymorphic form indicated second is the asthma or IgE associated form. In the three polymorphic sites indicated with a *, the variant form of the polymorphic site is found in NT_{—}000380 (and SEQ ID NO:1, 2, 4, 6, 8, 10, 12 and/or 14), and the reference or wildtype allele is found in a different individual. The last column of the table shows whether an amino acid change occurs due to the polymorphisms. The positions of these polymorphisms defined with respect to SEQ ID NO:2 are: position 448 (T (variant) preferably to A (wildtype or reference), position 776 (C preferably to T), position 851 (C preferably to G), position 1159 (G preferably to A), position 1199 (T preferably to C), and position 1529 (C preferably to T); or in any one of the following positions as defined by SEQ ID NO:4: position 1206 (A preferably to C), position 1225 (T preferably to C), position 1330 (T preferably to A), and position 1338 (G preferably to A); or in any one of the following positions as defined by SEQ ID NO:8: position 585 (T preferably to C), position 655 (C preferably to A), and position 681 (A preferably to G).

Table 8 shows the five polymorphic sites within Table 7 that showed the highest association with high serum IgE level. Two of the polymorphic sites involved nonconservative amino acid changes, one within the important region of exon 3 within AST-1. Two of the polymorphic sites were within coding regions but did not cause amino acid changes. These sites may show strong association as a result of linkage disequilibrium with causative polymorphic sites. A fifth polymorphic site is within the 3′ UTR of exon 9A. This site may show a strong association for the same reason or because the nucleotide substitution has an effect on mRNA stability.

Table 12 shows associations for five polymorphic sites occurring within the AAA1 gene. At each site, the wildtype polymorphic form is indicated first, and the variant form second. The table shows relative frequency of associating haplotypes in individuals with high serum IgE level compared to those in control individuals with low serum IgE level. All haplotypes shown in the table gave chi square values of at least 8.9 (P<0.01) for association.

VIII. Analysis of Polymorphisms

A. Preparation of Samples

Polymorphisms are detected in a target nucleic acid from an individual being analyzed. For assay of genomic DNA, virtually any biological sample (other than pure red blood cells) is suitable. For example, convenient tissue samples include whole blood, semen, saliva, tears, urine, fecal material, sweat, buccal, skin and hair. For assay of cDNA or mRNA, the tissue sample must be obtained from an organ in which the target nucleic acid is expressed.

Many of the methods described below require amplification of DNA from target samples. This can be accomplished by e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202 (each of which is incorporated by reference for all purposes).

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

B. Detection of Polymorphisms in Target DNA

The identity of bases occupying the polymorphic sites shown in Table 3, 7 or 12 can be determined in an individual (e.g., a patient being analyzed) by several methods, which are described in turn.

1. Single Base Extension Methods

Single base extension methods are described by e.g., U.S. Pat. No. 5,846,710, U.S. Pat. No. 6,004,744, U.S. Pat. No. 5,888,819 and U.S. Pat. No. 5,856,092. In brief, the methods work by hybridizing a primer that is complementary to a target sequence such that the 3′ end of the primer is immediately adjacent to but does not span a site of potential variation in the target sequence. That is, the primer comprises a subsequence from the complement of a target polynucleotide terminating at the base that is immediately adjacent and 5′ to the polymorphic site. The hybridization is performed in the presence of one or more labeled nucleotides complementary to base(s) that may occupy the site of potential variation. For example, for a biallelic polymorphisms two differentially labeled nucleotides can be used. For a tetraallelic polymorphism four differentially labeled nucleotides can be used. In some methods, particularly methods employing multiple differentially labeled nucleotides, the nucleotides are dideoxynucleotides. Hybridization is performed under conditions permitting primer extension if a nucleotide complementation a base occupying the site of variation in the target sequence is present. Extension incorporates a labeled nucleotide thereby generating a labeled extended primer. If multiple differentially labeled nucleotides are used and the target is heterozygous then multiple differentially labeled extended primers can be obtained. Extended primers are detected providing an indication of which bas(s) occupy the site of variation in the target polynucleotide. The methods are particularly useful for SNPs.

2. Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment (corresponding segments being defined by maximum alignment of the sequences being compared) from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15 mer at the 7 position; in a 16 mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

3. Tiling Arrays

SNPs and insertion deletion polymorphism can be detected by hybridizing target nucleic acids to arrays of probes tiling a reference sequence or one or more variant forms of the reference sequence as described by WO 95/11995 (incorporated by reference in its entirety for all purposes).

4. Allele-Specific Amplification Methods

An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. In some methods, the mismatch is included in the 3′-most position of the oligonucleotide aligned with the polymorphism because this position is most destabilizing to elongation from the primer. See, e.g., WO 93/22456.

5. Direct-Sequencing

The direct analysis of the sequence of polymorphisms of the present invention can be accomplished using either the dideoxy-chain termination method or the Maxam-Gilbert method (see Sambrook et al., Molecular Cloning, A Laboratory Manual (2nd Ed., CSHP, New York; 1989); Zyskind et al., Recombinant DNA Laboratory Manual, (Acad. Press, 1988)).

6. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

7. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

8. Other Methods

Include oligonucleotide ligation assay or restriction fragment length polymorphism (RFLP) (see, for example, Current Protocols in Molecular Biology, eds. Ausubel et al, John Wiley & Sons: 1992, and Landegren et al, “Reading Bits of Genetic Information: Methods for Single-Nucleotide Polymorphism Analysis”, Genome Research 8:769-776).

VII. Methods of Use

A. Discovery of New Polymorphic Sites in AST-1 Locus

Additional polymorphic sites beyond those listed in Tables 3, 7 and 12 within GPRA and AAA1 genes and particularly, the AST-1 locus thereof can be identified by comparing a GPRA or AAA1 nucleic acid from different individuals with a reference sequence, such as SEQ. ID NO:1, 2, 4, 6, 8, 10, 12 or 14 or SEQ ID NOS:16, 18, 20, 22, 24, 26, 28, 30, 32, 34, 36, 38 or 40. Preferably, the different individuals suffer or at risk of an immune disorder, such as autoimmune disease, an IgE-mediated disorder, such as asthma, chronic obstructive pulmonary disease or cancer. If a new polymorphic site (e.g., SNP) is found, then the link with a specific disease can be determined by comparing the frequency of variant polymorphic forms occupying this site in populations of patients with or without a disease being analyzed (or susceptibility thereto). A polymorphic site or region may be located in any part of the locus, e.g., exons, introns and promoter regions of genes.

B. Diagnostic and Detection Methods

The present invention further provides means for prognostic or diagnostic assays for determining if a subject has or is increasingly likely to develop a disease associated with the variation or dysfunction of the asthma locus of the invention. Such diseases include in addition to asthma, other IgE mediated diseases, autoimmune diseases, atopic and other immune diseases, chronic obstructive pulmonary disease and cancer. Basically, such assays comprise a detection step, wherein the presence or absence of a variant polymorphic form in the asthma locus-1 relative to SEQ ID NO:1, 2, 4, 6, 8, 10, 12, or 14 or SEQ ID NO: 16, 18, 20, 22, 24, 26, 28, 30, 32, 34, 36, 38 and 40 is determined in a biological sample from the subject. The detection step can be performed by any of the methods described above. Analogous assays can be performed in which variations in protein sequence relative to SEQ ID NOS:3, 5, 7, 9, 11, 13 or 15 or SEQ ID NO: 17, 19, 21, 23, 25, 27, 29, 31, 33, 35, 37, 39 and 41 are determined.

In particular, the present invention is directed to a method of determining the presence or absence of a variant form of a polymorphic site of the invention in a biological sample from a human for diagnosis of asthma or for assessing the predisposition of an individual to asthma, other IgE-mediated disease, chronic obstructive pulmonary disease or cancer. The method comprises determining the sequence of the nucleic acid of a human at one or more the above SNP or DIP positions shown in Tables 3, 7 and 12. Optionally, the sample is contacted with oligonucleotide primers so that the nucleic acid region containing the potential single nucleotide polymorphism is amplified by polymerase chain reaction prior to determining the sequence.

The polymorphic sites can be analyzed individually or in sets for diagnostic and prognostic purposes. The conclusion drawn from the analysis depends on the nature and number of polymorphic sites analyzed. Some polymorphic sites have variant polymorphic forms that are causative of disease. Detection of such a polymorphic form provides at least a strong indication of presence or susceptibility to disease. Other polymorphic sites have variant polymorphic forms that are not causative of disease but are in equilibrium dislinkage with a polymorphic form that is causative. Detection of noncausative polymorphic forms provides an indication of risk of presence or susceptibility to disease. Detection of multiple variant forms at several polymorphic sites in a GPRA and or AAA1 gene and particularly the parts of these genes within the AST-1 locus thereof provides an indication of increased risk of presence or susceptibility to disease. The results from analyzing the polymorphic sites of the invention can be combined with analysis of other loci that associate with the same disease (e.g., asthma) as polymorphic sites of the invention. Alternatively, or additionally risk of disease can be confirmed by performing conventional medical diagnostic tests of patient symptoms.

C. Clinical Trials

The polymorphisms of the invention are also useful for conducting clinical trials of drug candidates for the diseases noted above particularly asthma. Such trials are performed on treated or control populations having similar or identical polymorphic profiles at a defined collection of polymorphic sites. Use of genetically matched populations eliminates or reduces variation in treatment outcome due to genetic factors, leading to a more accurate assessment of the efficacy of a potential drug.

Furthermore, the polymorphisms of the invention may be used after the completion of a clinical trial to elucidated differences in response to a given treatment. For example, the set of polymorphisms may be used to stratify the enrolled patients into disease sub-types or classes. It may further be possible to use the polymorphisms to identify subsets of patients with similar polymorphic profiles who have unusual (high or low) response to treatment or who do not respond at all (non-responders). In this way, information about the underlying genetic factors influencing response to treatment can be used in many aspects of the development of treatment (these range from the identification of new targets, through the design of new trials to product labeling and patient targeting). Additionally, the polymorphisms may be used to identify the genetic factors involved in adverse response to treatment (adverse events). For example, patients who show adverse response may have more similar polymorphic profiles than would be expected by chance. This allows the early identification and exclusion of such individuals from treatment. It also provides information that can be used to understand the biological causes of adverse events and to modify the treatment to avoid such outcomes.

VIII. Kits

The invention also features diagnostic or prognostic kits for use in detecting the presence of a SNP in a GPRA and/or AAA1 gene or particularly an AST-1 locus thereof in a biological sample. The kit provides means for the diagnostics of asthma, other IgE-mediated disease, chronic obstructive pulmonary disease or cancer or for assessing the predisposition of an individual to a disease mediated by variation or dysfunction of a GPRA and/or AAA1 gene. The kit can comprise a labeled compound capable of detecting the nucleic acid of a GPRA and/or AAA1 gene in a biological sample. The kit can also comprise nucleic acid primers or probes capable of hybridizing specifically to at least of portion of a GPRA gene. Preferably, the primer is a minisequencing primer specific to any one of above polymorphisms. The kit can be packaged in a suitable container and preferably it contains instructions for using the kit.

Often, the kits contain one or more pairs of allele-specific oligonucleotides hybridizing to different forms of a polymorphism. In some kits, the allele-specific oligonucleotides are provided immobilized to a substrate. For example, the same substrate can comprise allele-specific oligonucleotide probes for detecting at least 10, 50 or all of the polymorphisms shown in Tables 3, 7, 12, 13, and 14. Optional additional components of the kit include, for example, restriction enzymes, reverse-transcriptase or polymerase, the substrate nucleoside triphosphates, means used to label (for example, an avidin-enzyme conjugate and enzyme substrate and chromogen if the label is biotin), and the appropriate buffers for reverse transcription, PCR, or hybridization reactions.

IX. Transgenic Animals and Cells

The present invention also makes available plasmids and vectors comprising the nucleic acid of the invention, which constructs can be used when studying biological activities of the AST-1 locus. The selecting of a suitable plasmid or vector for a certain use is within the abilities of a skilled artisan. As the host cell may be any prokaryotic or eukaryotic cell, a plasmid or vector comprising the locus can be used in the duplication of the locus (see, for example, Sambrook et al, “Molecular Cloning: A Laboratory Manual”, Second Edition, Cold Spring Harbor, New York: Cold Spring Harbor Laboratory Press (1989)).

Nucleic acids of the invention can be used to generate either transgenic animals or “knock out” animals which, in turn, are useful in the development and screening of therapeutically useful reagents. A transgenic animal (e.g., a bovine, pig, sheep, rabbit, rat, mouse or other rodent) is an animal having cells that contain a transgene, which transgene was introduced into the animal or an ancestor of the animal at an embryonic (e.g., one-cell) stage. Often all or substantially all of the somatic and germline cells of the transgenic animal have a copy of the transgene in their genome. A transgene is a DNA which is integrated into the genome of a cell from which a transgenic animal develops. Methods for generating transgenic animals, particularly animals such as mice, have become conventional in the art and are described, for example, in U.S. Pat. Nos. 4,736,866 and 4,870,009. Typically, a GPRA and/or AAA1 nucleic acid is operably linked to a promoter and optionally an enhancer. The promoter and enhancer can be selected confer tissue specific expression. Transgenic animals that include a copy of a transgene introduced into the germ line of the animal at an embryonic stage can be used to examine the effect of increased expression of GPRA and/or AAA1 polypeptides. Such animals can be used as tester animals for reagents thought to confer protection from, for example, diseases related to GPRA and/or AAA1. Such a transgenic animal can be treated with agent and a reduced incidence of a characteristic of disease compared to untreated animals (i.e., without the agent) bearing the transgene indicates a potential therapeutic intervention for the disease. Results from animal models can be confirmed in placebo-controlled clinical trials on patients with asthma, other IgE mediated disease, chronic obstructive pulmonary disease or cancer.

Transgenic offspring are identified by demonstrating incorporation of the microinjected transgene into their genomes, preferably by preparing DNA from short sections of tail and analyzing by Southern blotting for presence of the transgene (“Tail Blots”). A preferred probe is a segment of a transgene fusion construct that is uniquely present in the transgene and not in the mouse genome. Alternatively, substitution of a natural sequence of codons in the transgene with a different sequence that still encodes the same peptide yields a unique region identifiable in DNA and RNA analysis. Transgenic “founder” mice identified in this fashion are bred with normal mice to yield heterozygotes, which are backcrossed to create a line of transgenic mice. Tail blots of each mouse from each generation are examined until the strain is established and homozygous. Each successfully created founder mouse and its strain vary from other strains in the location and copy number of transgenes inserted into the mouse genome, and hence have widely varying levels of transgene expression or activity.

Knockout animals or cells can also be made by functionally disrupting an endogenous cognate form of one or more genes within the AST-1 locus (i.e., GPRA and/or AAA1). Functional disruption means that the transgenic animal is incapable of making a functional GPRA or AAA1 polypeptide and/or transcript encoding the same. Inactivation of a protein can be achieved by forming a transgene in which a cloned variant gene is inactivated by insertion of a positive selection marker within the coding region. See Capecchi, Science 244, 1288-1292 (1989). Inactivation of a transcript can be achieved by mutation within a promoter region. The transgene is then introduced into an embryonic stem cell, where it undergoes homologous recombination with an endogenous variant gene. Mice and other rodents are preferred animals. Such animals provide useful drug screening systems.

X. Methods of Screening for Drugs

The GPRA and AAA1 polypeptides and nucleic acids of the invention of the invention are useful for screening for agents that modulate (e.g., agonize or antagonize) their function (e.g., as a G-protein coupled receptor). Such agents can be useful as drugs in compensating for genetic variations affecting GPRA and/or AAA1 function. For example, an agonist of GPRA and/or AAA1 function is useful in a patient having a genetic variation that decreases endogenous GPRA function, and an antagonist is useful in a patient having a genetic variation that increases endogenous GPRA and/or AAA1 function. GPRA and AAA1 polypeptides can also be used to screen known drugs to determine whether the drug has an incidental effect on GPRA and/or AAA1 activity. A drug that has an incidental agonist activity should generally be avoided in patients having atypically high levels of IgE antibodies, and an antagonist should generally be avoided in immunosuppressed patients.

Agents can initially be screened in a binding assay with a GPRA or AAA1 polypeptides. A binding assay reduces the pool of initial agents to a subset that can be screened by a functional assay. Agents can be screened in cells transfected with nucleic acids encoding a GPRA and/or AAA1 polypeptide of the invention or in transgenic animals transfected with a GPRA and/or AAA1 nucleic acids and optionally and a reporter construct containing cAMP response element, a minimal promoter and a coding sequence for secreted alkaline phosphatase. Methods for identifying agents that have a functional activity in transducing a signal through a cellular receptor are described by U.S. Pat. No. 6,309,842. Agents for screening can be obtained by producing and screening large combinatorial libraries, as described above, or can be known drugs, or can be known ligands to related receptors to GPRA.

Agents can also be screened for capacity to inhibit or increase expression of GPRA and/or AAA1 nucleic acids. Suitable agents to screen include antisense polynucleotides, see Antisense RNA and DNA, (1988), D. A. Melton; Ed., Cold Spring Harbor Laboratory, Cold Spring Harbor, N.Y.); Dagle et al., Nucleic Acids Research, 19:1805 (1991); Uhlmann et al., Chem. Reviews, 90:543-584 (1990), zinc finger proteins (see WO 00/00388 and EP.95908614.1), and siRNAs WO 99/32619, Elbashir, EMBO J. 20, 6877-6888 (2001) and Nykanen et al., Cell 107, 309-321 (2001); WO 01/29058.

Agents having pharmacological activity evidenced by cellular or animal testing can be subject to clinical trials in patients suffering from asthma, other IgE-mediated disease, chronic obstructive pulmonary disease or cancer in comparison with a placebo.

XI. Methods of Treatment

The invention provides methods of treating asthma, other IgE mediated disease, chronic obstructive pulmonary disease or cancer. These methods entail administering an effective amount of a GPRA and/or AAA1 polypeptide or an agent, e.g., an antibody, that modulates activity of a GPRA and/or AAA1 polypeptide. The agent can modulate that activity for example, by an effect on signal transduction through the GPRA polypeptide (usually a decrease) or by effecting expression of the GPRA and/or AAA1 polypeptide (also usually a decrease). An agent can be administered for prophylactic and/or therapeutic treatments. A therapeutic amount is an amount sufficient to remedy a disease state or symptoms, or otherwise prevent, hinder, retard, or reverse the progression of disease or any other undesirable symptoms in any way whatsoever. In prophylactic applications, an agent is administered to a patient susceptible to or otherwise at risk of a particular disease or infection. Hence, a “prophylactically effective” amount is an amount sufficient to prevent, hinder or retard a disease state or its symptoms. In either instance, the precise amount of compound contained in the composition depends on the patient's state of health and weight.

An appropriate dosage of the pharmaceutical composition is determined, for example, using animal studies (e.g., mice, rats) are commonly used to determine the maximal tolerable dose of the bioactive agent per kilogram of weight. In general, at least one of the animal species tested is mammalian. The results from the animal studies can be extrapolated to determine doses for use in other species, such as humans for example.

The pharmaceutical compositions can be administered in a variety of different ways. Examples include administering a composition containing a pharmaceutically acceptable carrier via oral, intranasal, rectal, topical, intraperitoneal, intravenous, intramuscular, subcutaneous, subdermal, transdemal, intrathecal, and intracranial methods. The route of administration depends in part on the chemical composition of the active compound and any carriers.

The components of pharmaceutical compositions are preferably of high purity and are substantially free of potentially harmful contaminants (e.g., at least National Food (NF) grade, generally at least analytical grade, and more typically at least pharmaceutical grade). To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions. Compositions for oral administration need not be sterile or substantially isotonic but are usually made under GMP conditions.

### Materials and Methods

Families were recruited in central eastern Finland in 1994 and 1996. The methods for recruitment, control for population stratification, and clinical evaluation have been described previously in detail (Kauppi et al. 1998; Laitinen et al. 1997). We used self-reported asthma as a sampling method. Altogether 253 families were recruited, two thirds of which were trios. Based on retrospective verification of the disease history and the results of diagnostic tests (spirometry, histamine or methacholine challenge test, expiratory peak flow measurements), 87% of the self-reported asthma patients were accepted as verified cases (Kauppi et al 1998). Criteria for asthma were based on the recommendations of the American Thoracic Society (Dantzker, D. R. et al. 1987). In addition to the 86 multiplex pedigrees included already into our previous genome scan, 103 nuclear families with full phase information (a total of 874 study individuals) were included to the association analysis without further selection.

Total serum IgE level was determined by Diagnostics CAP FEIA (Kabi Pharmacia, Sweden) in one batch. Allergy screening was done to all study individuals at the time of the study (Kauppi et al. 1998; Laitinen et al. 1997). Based on total serum IgE level the study individuals were divided into two groups: high IgE responders (IgE>100 kU/L) and low IgE responders (IgE≦100 kU/L).

### EXAMPLES

### Example I

### Construction of the Physical Map

We have ordered the markers used in the analysis and estimated the physical distance between the markers using several publicly available sources as described previously by Polvi et al (2002). Completed chromosome 7 sequence confirms the distance and order of our markers

(http://www.ncbi.nlm.nih.gov/entrez/query.fcgi?cmd=Retrieve&db=Nucleotide&list_uids=22 096428&dopt=GenBank).

### Example II

### Microsatellite Discovery and Genotyping

To create a dense map of polymorphic markers spread evenly across the linkage region, we screened the publicly available genomic sequences for potentially polymorphic tandem repeats (Polvi et al. 2002). Eighty microsatellites were genotyped in the whole data set (874 samples) giving the average marker density of 220 kb (range from 904 bp to 790 kb) across the linkage region. All microsatellite markers and small insertions and deletions (Table 1C) found in the critical region were genotyped using fluorescently labeled primers in gel electrophoresis on the ABI377 sequencer as described previously (Polvi et al. 2002 and http://www.genome.helsinki.fi/eng/research/asthma/detail.htm).

### Example III

### Discovery of Genetic Variation in the Critical Region by Direct Sequencing

To identify all sequence polymorphisms in the critical region, we generated a 129 kb reference sequence by assembling the sequence (genomic clones AC005680, AC005492, AC005174, AC005862.1, and AC005853.1) from the Human Genome Project (in the contig NT_{—}000380 from the position 509,783 to the position 638,799). SNP discovery by re-sequencing was done in four selected patients and in three control individuals who were homozygous across the region and for the flanking markers. Repeat regions such as SINE, LINE, LTR, MER1 and MER2 elements covered 60% of the sequence (a total of 129,017 bp). PCR assays were designed to be 650 bp in length with 100 bp overlap with adjacent assays. Repeat regions were re-sequenced when it was possible to design primers in unique sequence within the range mentioned above.

PCR assays were carried out in 20 μl volumes containing 20 ng of genomic DNA, 1×PCR Buffer II, 0.1 mM dNTPs, 2.5 mM MgCl2, 0.1 μM primer mix, and 0.5 U of DNA polymerase (AmpliTaqGold, Applied Biosystems). The samples were denatured for 10 min at 94° C., followed by 35 cycles each of 30 s at 94 C, 30 s at 58 C, and 30 s at 72° C. Elongation was performed for 10 min at 72° C. Purified PCR fragments (Quickstep 2 PCR purification Kit, Edge BioSystems, MD) were sequenced in both directions using ABI Prism3100 sequencer and dye-terminator chemistry. We assembled the sequence reads using the Gap4 program.

### Example IV

### SNP Genotyping

SNP genotyping was done using two different methods: single base pair extension (SBE) or using altered restriction sites. SBE was done using the chemistry of Molecular Dynamics according to the suggestion made by the manufacturer on a Megabase 1000 sequencer (Molecular Dynamics, CA) using the primers listed in the Table 1A. Allele calling was performed by using the MegaBACE SNP Profiler software (Molecular Dynamics).

Twenty-nine of the SNPs were genotyped using different restriction enzyme digestions. All primers, restriction enzymes, and lengths of the digestion fragments of corresponding allele used in the genotyping are given in Table 1B. If the SNP did not produce a natural site for altered restriction, mutations were induced in PCR-primers (shown in capital letters in primer sequence). To improve allele calling by growing size difference between alleles, in some primers plasmid sequence tail of 20-30 bp was added (shown in bold in the primer sequence). Altered restriction sites of the PCR products stained with ethidium bromide and visualized on agarose gels in UV light were called manually by two independent observers. All the markers were in Hardy-Weinberg equilibrium and observed Mendel errors were less than 0.1%.

### Example V

### Haplotype Association Analysis

The haplotype analysis was done using Haplotype Pattern Mining (HPM) (Toivonen et al. 2000) program. For haplotyping, large pedigrees were divided into trios using an in-house computer program. The program identifies the maximum number of trios that are not overlapping and in which one or two members were affected (not both parents). Trios that included members who had not been genotyped or members with unknown phenotype were excluded. In phase three of hierarchical LD mapping, high density genotyping of the critical region was done in the trios informative for the association analysis (a total of 132 trios, 396 study individuals, and revealing 304 unrelated affected and 220 control chromosomes). Haplotyping was done within each trio and four independent chromosomes were obtained from each trio. In case of ambiguities (missing genotype data, identical heterozygotic genotypes in all of the family members, or Mendel errors), the alleles were discarded. If the child was affected, the transmitted chromosomes were considered disease associated and the non-transmitted chromosomes as controls. If one of the parents was affected, his/her chromosomes were considered disease associated and the spouse's chromosomes as controls. If both the parent and the child were affected, only the non-transmitted chromosome of an unaffected parent was considered as the control and the other three as disease associated. These haplotypes were used as input for HPM.

To estimate the overall significance of detected association, accounting for simultaneous testing of multiple markers, we used a second level permutation test by performing nested permutation tests described in detail elsewhere (Sevon et al. 2001a and b).

### Results

1. Haplotype Association

Our genotyping effort searching for a shared haplotype among individuals who have high total IgE serum level was done in three phases using a hierarchical approach. Based on the initial observation on potential association, the marker map was made denser in the regions of primary interest. In the haplotype analysis we used the HPM algorithm that is known to be powerful in locating a disease-causing gene when one is known to exist (Toivonen et al. 2000). By allowing gaps in the haplotype patterns, the algorithm is robust for genotyping errors, marker mutations, unrecognized recombinations and missing data. The data set consisted of 86 multiplex pedigrees that were included also in the previous linkage study and additional 103 nuclear families recruited solely for the association study. These families revealed 304 unrelated high IgE associated and 220 control chromosomes which were then used as the input data for the HPM analysis.

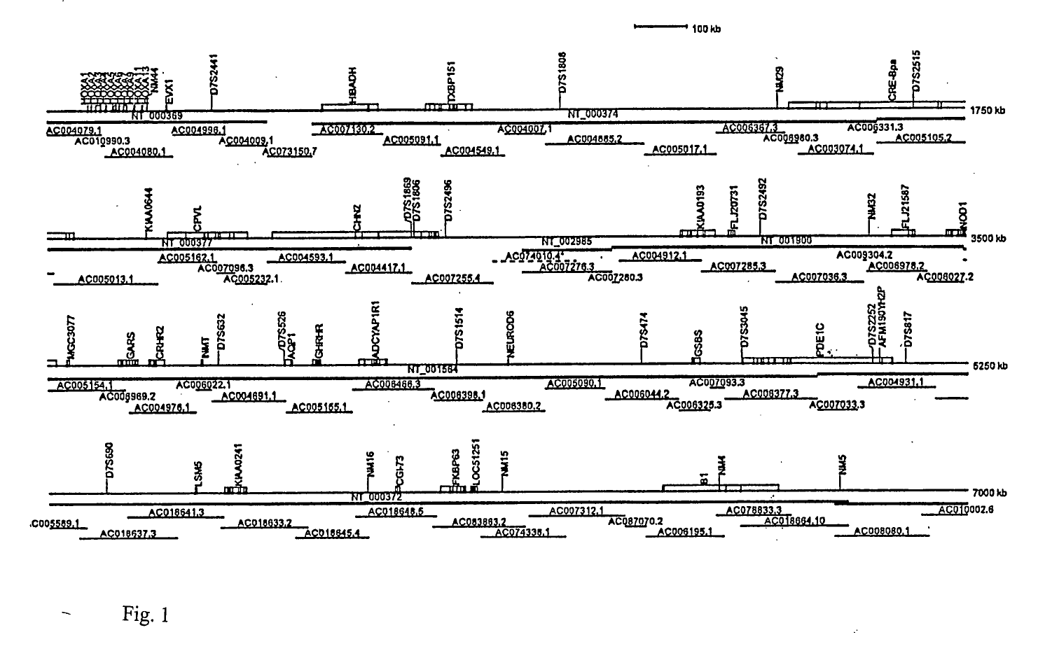

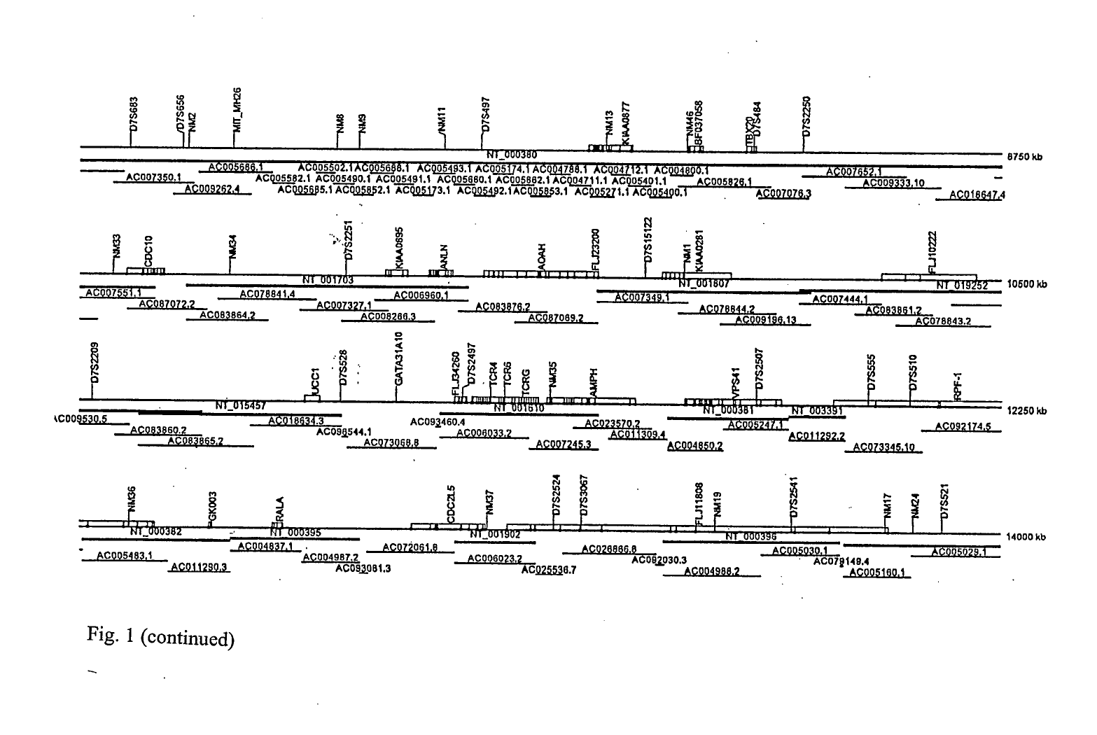

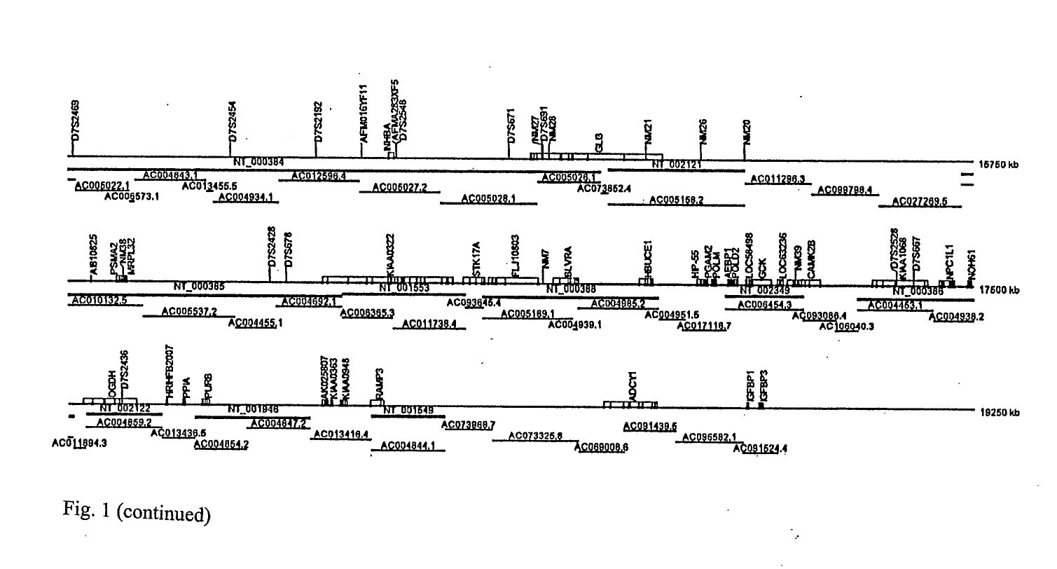

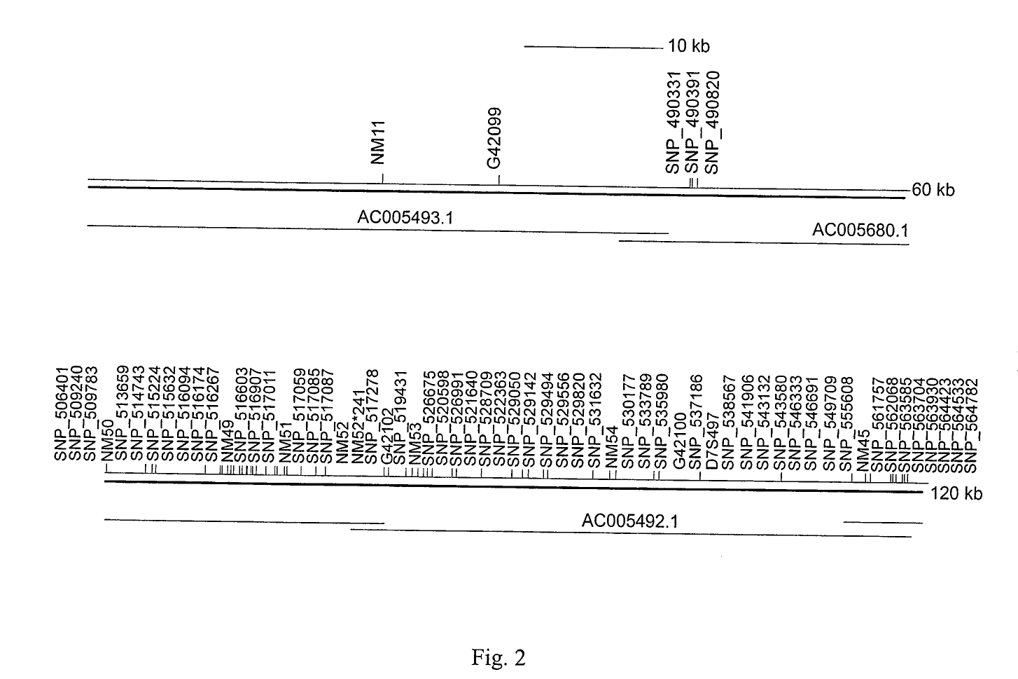

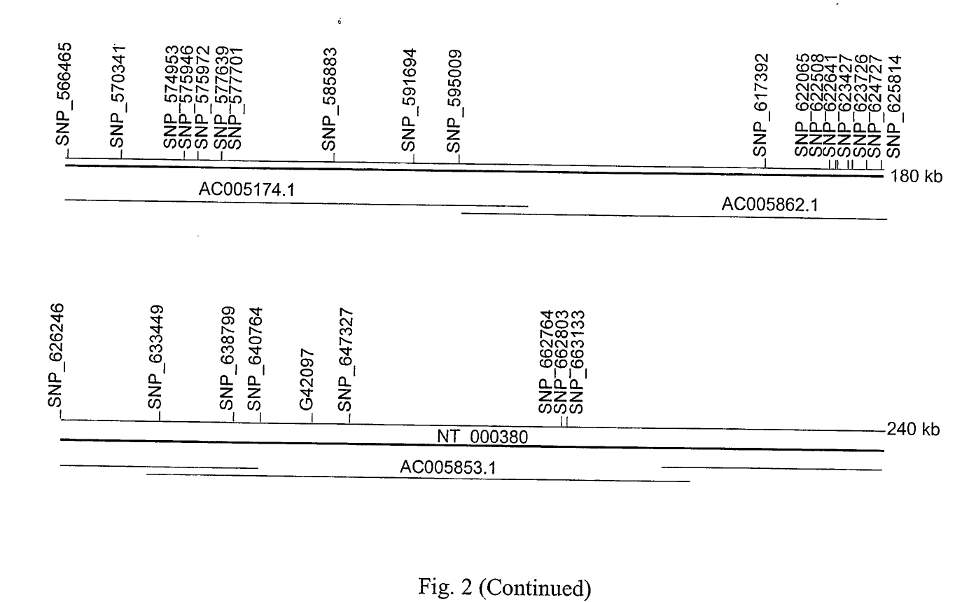

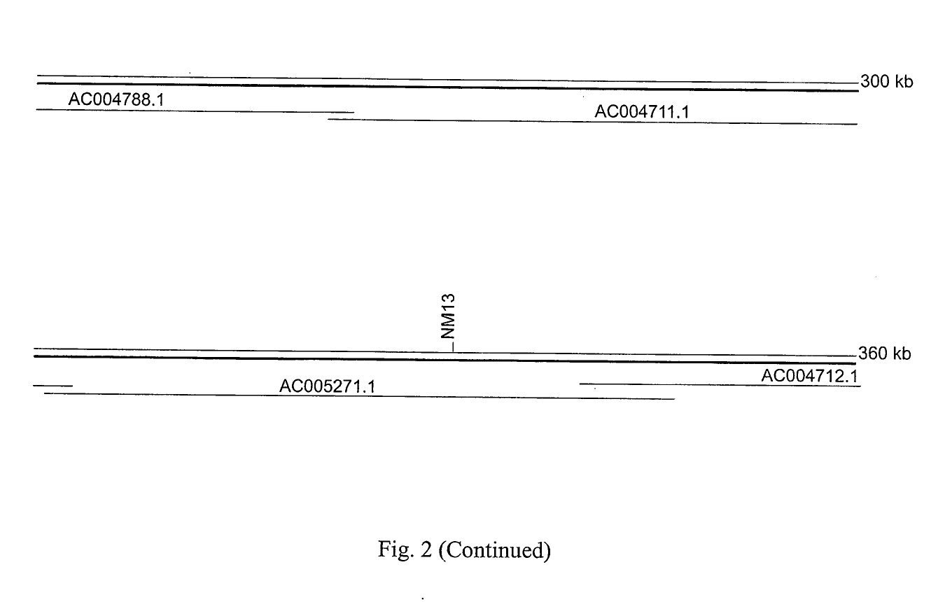

First we built a marker map across the whole linkage region with 80 microsatellite markers (FIG. 1). In the first phase of haplotype association analysis the marker density was approximately 220 kb across the linkage region. The best associations (chi square≧7.4) formed only one cluster of haplotypes located between the markers D7S690-NM13 (˜3.21 cM). In the second phase we added 6 new microsatellites in between D7S690 and NM13. The associated haplotypes (chi square≧8.9) clustered now at the centromeric end of the region, between markers G42099 and NM13 (301 kb) (FIG. 2). HPM algorithm was used to evaluate the overall haplotype distribution among the high IgE associated compared to that among control chromosomes. A permutation test incorporated to the HPM computer package was used to evaluate the statistical significance of our finding by comparing the overall haplotype distribution among the high IgE associated and control chromosomes. Using chi square 7.0 as the threshold for association, 7 markers as the maximum length of the haplotype, and allowing one gap in the haplotype patterns, the best associations were computed for three adjacent markers G42102, D7S497, and G4296 (marker-wise P values 0.05-0.06 based on 10 000 simulations).

Fine mapping was then further continued by adding 49 new markers between G42099 and NM13 giving an average marker density of 6 k-b. By using the HPM algorithm, all associated haplotype (chi square≧13) patterns spanned between SNP509783 and SNP638799 (129,017 bp).

To study if the overall haplotype distribution in the locus differs in high IgE associated compared to that in control chromosomes, we used again a permutation test. HPM analysis was done using the following parameters: maximum pattern length 40 markers, one gap allowed for missing data and possible errors, and chi-square threshold for the association 13.0. The observed scores (=number of qualified haplotype patterns spanning across the marker) for associated haplotypes varied from 0 to 40. Permutation test showed statistically biased haplotype distribution for high IgE. The marker-wise P values≦0.005 were observed for all eleven of the markers between SNP531632 and SNP563930 and the marker wise P values≦0.01 for all twenty-eight markers between NM51 and SNP617392 based on 10 000 simulations. To estimate the effect of simultaneous testing of multiple markers, we used a second level permutation test, yielding a corrected P value of 0.03 (Sevon 2001a and b).

2. Genetic Variation in the Critical Region

Based on haplotype analysis we had determined the critical region in between the markers SNP509783 and SNP638799 (FIG. 2). SNP genotyping resulted in the identification of seven haplotypes H1-H7 that together explain 94.5% of the genetic variation detected at the AST-1 locus. To identify all potentially causative genetic variations on the risk haplotype, we sequenced four patients who carried haplotypes H2/H2, H4/H4, H5/H5, and H7/H7, respectively, and were homozygous across the AST1-locus region. The patients' sequences polymorphisms were compared with the reference sequence (SEQ ID NO:1)

(http://www.ncbi.nlm.nih.gov/entrez/query.fcgi?cmd=Retrieve&db=nucleotide&list_uid s=7382309&dopt=GenBank, human genomic contig NT_{—}000380) and to three population based controls homozygous for the same markers, but with different haplotypes H1/H1, H3/H3, and H6/H6, respectively. The differences are highlighted via SEQ ID NO:1. Genetic variation including small deletions and insertions (≦6 base pairs), variation in the length of microsatellite repeats, and multiple SNPs are presented in Table 3 that can be used as genetic markers of the risk haplotypes. Tables 13 and 14 show the SNPs unique for each haplotype and unique for certain haplotype combinations, respectively. In diagnostic testing these markers identify the haplotypes and the haplotype combinations of an individual without phase information.

### Example VI

### Identification of the GPRA Gene

A. Materials and Methods

1. Exon Prediction

Exon predictions were performed by GENSCAN http://genes.mit.edu/GENSCAN.html) and software using genomic clones from AC005493.1 to AC005826.1 (690 kb) (Polvi et al 2002) as one block. Predictions were performed for both unmasked and masked sequence. In the latter, the repetitive sequences were masked using the Repeat Masker Service (http://repeatmasker.genome.washington.edu/).

2. Reverse Transcriptase PCR

The exon-specific primers were designed based on exon predictions (Table 4, FIG. 3). First strand cDNAs were synthesized from 1 μg of commercially available poly A⁺ RNAs from human brain (Invitrogen), lung, testis, placenta, and thymus (BD Clontech) by the SMART RACE cDNA Amplification Kit (BD Clontech, Palo Alto, Calif.) according to manufacturer's instructions. Alternatively, Marathon-Ready cDNA (Clontech) or cDNA from Multiple Tissue cDNA Panels I and II (Clontech) were used as templates in some PCR amplifications. All the PCR amplifications were performed in 20-50 μl volumes using 2.0-5.0 μl cDNA as template, 2 mM MgCl₂, 0.2 mM dNTPs (Finnzymes), 0.2 mM of each primer, 0.15-0.4 U AmpliTaqGold, and 1×PCR Gold buffer (Applied Biosciences) under the following conditions: 94° C. for 10 min, 35-40 cycles of 94° C. for 30 s, 58° C.-68° C. for 30 s, 72° C. for 1 min 40 s to 2 min followed by 72° C. for 10 min. PCR products were analyzed on 1% agarose gel and extracted from the gel using QIAquick Gel Extraction Kit (Qiagen).

3. Rapid Amplification of cDNA Ends (RACE-PCR)

To generate 3′ and 5′ cDNA ends, rapid amplification of cDNA ends (SMART RACE) was performed using human testis cDNA and Human Marathon-Ready Fetal Thymus cDNA (Clontech) according to the manufacturer's protocol for the SMART RACE cDNA Amplification Kit (BD Clontech). RACE-PCR products were cloned using pGEM-T Easy Vector system (Promega) or TOPO TA Cloning kit (Invitrogen) and plasmid DNA was purified using QIAprep Spin Miniprep Kit (Qiagen).

The purified RT-PCR products and the cloned RACE-PCR products were verified by automated sequencing with dye-terminator chemistry (ABI Prism3100, Applied Biosystems, Inc).

4. Cloning of the Full-Length cDNAs of GPRA

Nested PCR amplification was used in cloning of the full length cDNAs for GPRA splice variants A and B. The first round of PCR for variant A was done with the primer pair JEGE1F1 and JEGE9aR1, and for variant B with JEGE1F1 and JEGE9bR1 (Table 4, FIG. 3). The following inner primers were used in the re-amplification: JEGE1F2 and JEGE9aR2 for variant A; JEGE1F2 and JEGE9bR2 for variant B, respectively. Full length C variant was amplified with the primer pair JEGE1F2 and JEGExR1.

Primary PCR amplifications of variants A and B were performed in 25 μl volumes using 2.5 μl Human Brain Marathon-Ready cDNA (Clontech) as template, 1× DyNAzyme EXT buffer (containing 1.5 mM MgCl₂), 0.2 mM dNTPs (Finnzymes), 0.52 μM of each primer, 5% DMSO and 0.5 U DyNAzyme EXT polymerase (Finnzymes) under the following conditions: 94° C. for 4 min, 38 cycles of 94° C. for 30 s, 65° C. for 30 s, 72° C. for 1 min followed by final extension of 72° C. for 10 min. The aliquot of primary PCR product was re-amplified by 30 PCR cycles under the same conditions as above. PCR amplification of variant C was performed in the above conditions. NCI-H358 cDNA was used as template.

PCR products were cloned into pCR 2.1 TOPO-vector using TOPO TA cloning kit (Invitrogen) according to manufacturer's instructions and plasmid DNA were purified using QIAprep Spin Miniprep Kit (Qiagen). The cloned RT-PCR products were verified by automated sequencing with dye-terminator chemistry (MegaBACE 1000, Molecular Dynamics).

5. Cell Culture and Isolation of Poly A⁺ RNA

In addition to commercially available poly A⁺ RNAs and Marathon-Ready cDNAs, human lung epithelial carcinoma cell line NCI-H358 (ATCC) was cultured as mRNA source for expression studies. Cells were cultured in RPMI 1640 medium (Gibco BRL) supplemented with 1 mM sodium puryvate (Gibco BRL), 10% FCS (Biological Industries), and 1% Penicillin/Streptomycin (GibcoBRL). Poly A⁺ RNA was isolated by Dynabeads mRNA DIRECT Kit (Dynal) according to the manufacturer's instructions.

6. Northern Blot Hybridization

The GPRA C specific (comprising the nucleotides from 58 to 527 of the GPRA C variant) 470 bp probe was generated by RT-PCR using human testis cDNA as template. The probe was radiolabelled with α[³²P]-dCTP using RediPrime Kit (Amersham Biosciences) according to the manufacturer's instructions. A Human Multiple Tissue 8-lane Northern blot (BD Clontech) was prehybridized in ExpressHyb solution (BD Clontech) for 1 h at 68° C. followed by hybridization with the specific probe for 1 h 15 min at 68° C. Herring sperm DNA (100 μg/ml) was used as the blocking reagent. The filter was washed with 2×SSC and 0.05% SDS at room temperature and then exposed to X-ray film at −20° C. for 6 days.

7. Western Blot and Immunohistochemistry

Two specific antibodies against the alternative carboxy terminals of GPRA were raised by immunizing rabbits with the following peptides: CREQRSQDSRMTFRERTER (corresponding to the nucleotides 1148-1205 of the variant A) and CPQRENWKGTWPGVPSWALPR (corresponding to the nucleotides 1196-1259 of the variant B of GPRA). GPRA A peptide synthesis and antibody production were purchased from Sigma-Genosys Ltd (London Road, Pampisford, Cambridge). GPRA B peptide synthesis was purchased from the University of Helsinki and antibody production from the University of Oulu. A total of 6 immunizations were performed at 2 weeks intervals using the total of 2 mg of KLH-conjugated peptide purified by gel filtration. GPRA antibodies were purified from whole serum by affinity chromatography with the peptide coupled to iodoacetyl on a crosslinked agarose support according to the manufacturer's (Pierce, Meridian Road, Rockford Ill., USA) instructions.

For western blot analysis, human tissue lysates from spleen, skeletal muscle, uterine muscle, colon muscle, kidney, colon epithelium, testes, and prostate were obtained by mechanically homogenizing the frozen tissue samples in 10 mM Tris HCl 100 mM NaCl 2% Triton X-100 buffer with proteinase inhibitors. 50 μg of the protein lysates were run on reducing 12.5% SDS-PAGE gels and electroblotted to the PVDF membrane according to standard procedures. Nonspecific protein binding was prevented by incubating the membrane with 5% milk in 0.1% Tween 20 in TBS (TBST). Antigenic sites were revealed by incubating the membrane with the anti-GPRA C-terminal antibodies or pre-immune serum followed by the alkaline phosphatase-conjugated goat anti-rabbit secondary antibody (Jackson ImmunoResearch Laboratories Inc., West Baltimore Pike West Grove, Pa., USA) in 5% milk in 0.1% TBST. The color reaction was revealed by the NBT/BCIP method (Pierce). Negative controls did not show specific reactivity.

Formalin fixed, paraffin-embedded specimens of normal adult human bronchus, skin and colon, and human normal tissue array slides (MaxArray, Zymed Laboratories Inc., CA) containing 30 different tissues were used for immunohistochemistry. For pre-treatment, slides were deparaffinized by xylene-treatment followed by decreasing alcohol series. The slides were heated in microwave oven in 10 mM citrate buffer, pH 6.0 for 5 minutes. Immunohistochemical analyses were performed using the ABC method (Vectastain Elite ABC kit, Vector Laboratories, Burlingame, Calif.). Omission of primary antibody and staining with the nonimmunized sera was used as negative control for parallel sections. Neither of these controls showed any immunoreactivity.

8. Transient Transfections and Elisa

Cos-1 cells were cultured in Dulbecco's modified Eagle's medium supplemented with 10% fetal calf serum, 1% PS and 5% non-essential amino acids. Cos-1 cells were transiently transfected with pCMV-myc-GPRA A, B, C, D, E, F or Bshort separately using Fugene6 transfection reagent (Roche) following the manufacturer's protocol. After 24 hours, the transfected cells from one well of 6 well plate were divided into 16 wells of 96 well plate. After 48 hours of transfection, the cells were fixed with 3.5% PFA. Half of the cells were permeabilized with 0.5% TX-100 for 10-15 min, blocked with TBS containing 2% milk and 1% goat normal serum at 37° C. for 30 min, incubated with 1:1000 dilution of myc-specific primary antibody (Babco) for 1 h at 37° C., washed three times with TBS and thereafter incubated with a dilution of 1:2000 of HRP-conjugaded anti-mouse IgG antibody for 30 min at room temperature and washed three times with TBS. TMB-subrate (Sigma) was added to cells and the reaction was let to proceed for 3 to 6 minutes after which the reaction was stopped by adding 1.5 M HCL. Absorbance was measured at 450 nm.

9. Computational Protein Sequence Analysis

The transmembrane topology and putative N-glycosylation sites of GPRA protein were predicted using PredictProtein (http://cubic.bioc.columbia.edu/predictprotein/submit_def.html#top) and TMpred (http://www.ch.embnet.org/software/TMPRED_form.html) softwares. In protein homology comparisons the BlastP (http://www.ncbi.nlm.nih.gov/blast/) and T-Coffee

(http://www.ch.embnet.org/software/TCoffee.html) softwares were used.

10. Family Collection

Genotyping of GPRA was done in a family collection recruited in central eastern Finland. The methods for recruitment, control for population stratification, and clinical evaluation have been described previously in detail (Kauppi et al. 1998; Laitinen et al. 1997). We used self-reported asthma as a sampling method. Total serum IgE level was determined by Diagnostics CAP FEIA (Kabi Pharmacia, Sweden) in one batch for all the participants. Based on total serum IgE level the study individuals were divided into two groups: high IgE responders (IgE>100 kU/L) and low IgE responders (IgE≦100 kU/L).

Altogether 253 families were recruited, two thirds of which were trios. Based on retrospective verification of the disease history and the results of diagnostic tests (spirometry, histamine or methacholine challenge test, expiratory peak flow measurements), 87% of the self-reported asthma patients were accepted as verified cases (Kauppi et al. 1998). Criteria for asthma were based on the recommendations of the American Thoracic Society (Dantzker, D. R. et al. 1987). 86 large pedigrees were included into our previous genome scan (Laitinen et al. 2001). For the association study, those pedigrees were divided into trios using a in-house computer program. Non-overlapping trios with full phase and phenotype information and additional 103 trios were included to the association analysis without further selection.

11. Screening for Exon Polymorphisms

Genomic DNA of four asthma patients and one healthy control from above mentioned study group were screened for sequence variation with 12 primer pairs covering all the verified exons of GPRA (Table 5). All the patients were homozygous for microsatellites G42099, G42100, D7S497, and G42097 (from the NT_{—}000380 position 476,213 to 644,425, a total of 168 kb) and expressed the susceptibility haplotype (patent 1). The control individual showed homozygosity for the same markers, but to a different, non-associating haplotype.

PCR assays were carried out in 20 μl volumes containing 20 ng of genomic DNA, 0.1 mM dNTPs (Finnzymes), 2.5 mM MgCl2 (Applied Biosystems), 0.1 μM primer mix, and 0.5 U of DNA polymerase, and 1×PCR Buffer II (AmpliTaqGold, Applied Biosystems). The samples were denatured for 10 min at 94° C., followed by 35 cycles each of 30 s at 94° C., 30 s at 58° C., and 30 s at 72° C. Elongation was performed for 10 min at 72° C. Purified PCR fragments (Quickstep 2 PCR purification Kit, Edge BioSystems) were sequenced from both directions using ABI Prim3100 (Applied Biosystems) sequencer and dye-terminator chemistry. We assembled forward and reverse sequence reads using the Gap4 program (Staden Package software).

12. Genotyping

Genotyping of five exonic SNPs in GPRA was done using two different methods. One marker was genotyped using single base pair extension (SBE) with the chemistry of Molecular Dynamics on a Megabase 1000 sequencer (Molecular Dynamics) according to the suggestion made by the manufacturer (Table 6A). Allele calling was performed by using the MegaBACE SNP Profiler software (Molecular Dynamics).

Four of the SNPs were genotyped using different restriction enzyme digestions. All primers, restriction enzymes, and lengths of the digestion fragments of corresponding allele used in the genotyping are given in Table 6B. If the SNP did not produce a natural site for altered restriction, mutations were induced in PCR-primers. To improve allele calling by growing size difference between alleles, in one primer plasmid sequence tail was added. Altered restriction sites of the PCR products were visualized on ethidium bromide stained agarose gels in UV light and called manually by two independent observers. All the markers were in Hardy-Weinberg equilibrium and observed Mendel errors were less than 0.1%.

13. Haplotype Association Analysis

The haplotype analysis was done using Haplotype Pattern Mining (HPM) (Toivonen et al. 2000) program. The data set provided a total of 132 informative trios, 396 study individuals, and revealing 304 unrelated affected and 220 control chromosomes. Haplotyping was done within each trio and four independent chromosomes were obtained from each trio. In case of ambiguities (missing genotype data, identical heterozygotic genotypes in all of the family members, or Mendel errors), the alleles were discarded. If the child was affected, the transmitted chromosomes were considered disease associated and the non-transmitted chromosomes as controls. If one of the parents was affected, his/her chromosomes were considered disease associated and the spouse's chromosomes as controls. If both the parent and the child were affected, only the non-transmitted chromosome of an unaffected parent was considered as the control and the other three as disease associated. These haplotypes were used as input for HPM.

### Results

1. Characterization GPRA at RNA and Protein Level

A. Genomic Structure of Different Splice Variants of GPRA

The full-length cDNA sequence of GPRA was assembled by using RT-PCR with primers designed for predicted exons and by using RACE-PCR to generate the 5′ and 3′ ends of the gene. The genomic location of the primers is shown in the FIG. 3 and sequence in the Table 4. Nested PCR amplification was used to produce full length cDNAs of different splice variants in one fragment. Using different (Marathon-Ready Brain cDNAs, RNA extracted form NCI-H358 cell line) template three splice variants were identified repeatedly (A, B_{long}, and C variants in the FIGS. 3 and 4). All variants possess the same initiation site, but used alternative exons to encode the 3′ end of the gene (exons 9A, 9B and 2B, respectively). The sequence flanking the putative ATG translation initiation site (GCCATGC) contains the −3 purine, but not the +4 guanine residue, of the Kozak consensus sequence. In two of the variants, the open reading frame was distributed across 9 exons producing cDNAs of 1116 bp and 1134 bp in size (variants A and B_{long}, FIGS. 5A and 5B) and encoding 371 and 377 amino-acid proteins, respectively. The C variant is a shorter transcript of GPRA4 including only the exons E1, E2a and E2b (FIG. 5C). GPRA spanned 0.2 Mb of the genomic contig NT_{—}000380. The second intron is the largest, comprising of 93.8 kb.

When nested PCR specific for the B variant was applied in the cloning process a new splice variant was observed (Bₛₕₒᵣₜ in FIGS. 4B and 5B). A 33 bp deletion was observed in the 5′ prime end of the exon 3.

B. Expression Profiling of GPRA Using RT-PCR and Northern Blot Hybridization

RT-PCR analysis showed that different variants of GPRA were expressed in several human tissues. These tissues include testis, brain, pituitary gland, placenta, lung, heart, fetal thymus, and fetal heart. In addition to commercially available cDNAs, we analyzed the GPRA expression profile in NCI-H358 cell line that represents broncho-epithelial origin. Variants A (JEG5F1 and Vau8R2), B (JEG5F1 and Vau1000R1) and C (JEG5F1 and JEGEXR1) specific primer pairs were used in amplifications (FIG. 8, Table 4). All forms were expressed and using A variant specific primers, we found several transcripts. PCR fragments were cloned and sequenced. Sequence verification revealed variants A, D, E, and F (FIGS. 4 and 5). Northern blot hybridization was done using a 470 bp cDNA probe comprising exons E1, E2A, and E2B (FIG. 3). On Human 8-Lane MTN blot, the probe detected multiple transcripts sized approximately 6.5 kb, 6.0 kb, 1.8 k-b, and 1.0 kb as shown for placental tissue in FIG. 9.

C. The Predicted Structure of the GPRA Protein

Both TMpred and PredictProtein softwares predicted that the variants A and B_{long} encode a 7TM protein with an extracellular N-terminus (approximately 50 amino acids) and intracellular C-terminus (FIGS. 6A-6F). Their structure shared the common features of GPR family A with 16 conserved amino acids in the TM, loop, and C-terminal regions. These include among others two conserved Cys residues in exoloops 1 and 2 that potentially form a disulfide bridge; the Asp-Arg-Tyr sequence (DRY motif) in the proximity of TM3; Asn-Pro-X-X-Tyr motif in TM7; and a Cys residue in the C-terminal region (FIGS. 6A-6F). The putative N-glycosylation site Asn⁴ was predicted by PredictProtein. All the other variants lacked the 7TM structure. For variant Bₛₕₒᵣₜ a 6TM structure was predicted (FIG. 6B). Variant C encodes 94 amino acid protein that has potentially only one TM region (FIG. 6C). For the variants D and E, deletions of the exons 3 or 4 caused a shift in the ORF, presumably producing truncated forms of the protein. When both exons were deleted (variant F), the ORF remained intact, but only five-transmembrane regions were predicted (FIG. 6F).

D. Sequence Searches

Determined by BlastP comparisons, the GPRA protein shows 31% amino acid identity to human vasopressin receptor 1B, 28% identity to human vasopressin receptor 2, 32% identity to human oxytocin receptor, and 43% amino acid sequence identity to gene product CG6111 of Drosophila melanogaster which is considered as an orthology to human vasopressin/oxytocin receptor family (Park et al. 2002). The best sequence identity between proteins was found in TM regions and in the first exoloop. Based on T Coffee homology comparisons, GPRA1 showed 20% amino acid identity to the bovine rhodopsin receptor. Sequence comparisons between GPRA and mouse ESTs BB638128, BB632343, BB625809, BB228269, and publicly available mouse genomic sequence suggest the existence of a mouse orthology.

2. Expression Profiling of GPRA Variants A and B Using Western Blot Analysis and Immunohistochemistry

Immunostaining of normal adult human tissue samples with GPRA A variant specific antibody showed strongest expression in smooth muscle cells (SMC), such as SMC layer in bronchial and arterial walls in human lung and colon shown in FIGS. 10A and E. In alveolar wall and alveolar macrophages of lung (FIG. 10C) intense staining of GPRA A was detected. In colon epithelium (FIG. 10D), strong basal staining was observed, whereas in the bronchial epithelium (FIG. 10A) and keratinocytes (FIG. 10F), only mild/weak immunostaining was detected.

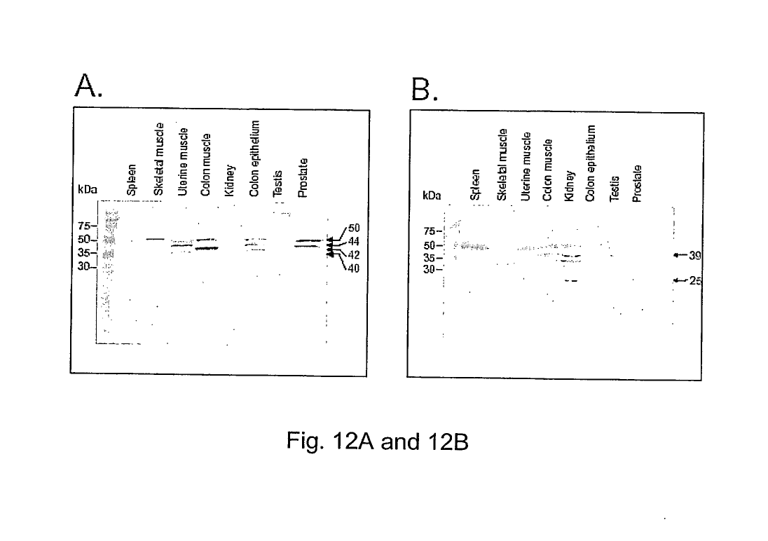

Western blot analysis with GPRA A variant specific antibody revealed four intensive bands corresponding to molecular weights of approximately 50, 44, 42 and 40 kDa (FIG. 12A). In skeletal muscle only 50 kDa band was detectable whereas uterine muscle, colon epithelium and prostate showed similar expression patterns at 50, 44, and 40 kDa. The most intensive GPRA A expression was recorded in colon muscle with the major band at 42 kDa. Weak or nonexistent bands were found in spleen, kidney, and testis.

Immunostaining with GPRA B variant specific antibody revealed most intense expression of the protein in the epithelium of several mucosal tissues including bronchus, small intestine, and colon as shown in FIGS. 11A, D, E. Contrary to the A variant, the expression of the B variant was strongest in the apical surface of the epithelium (FIGS. 11A, D, E, and F). Alveolar walls and alveolar macrophages (FIG. 11C) showed strong and SMCs in several tissues mild immunostaining of the variant B.

With GPRA B variant specific antibody, two sharp polypeptide bands corresponding to molecular weights of approximately 39 and 25 kDa were revealed by Western blot analysis with the strongest expression in kidney (FIG. 12B). The specificity of the antibody was confirmed by the blocking experiment with 10× molar excess of the peptide used in immunization.

Cos-1 cells were transiently transfected with pCMV-myc GPRA A, B, Bshort, C, D, E, or F in order to study translocation of different GPRA variants in vitro. After 48 hours, cells were fixed and studied by cell based ELISA assay using myc antibody. 71% of recombinant GPRA A and 52% of recombinant GPRA B were translocated to the plasma membrane while all the other variants were located in cytoplasm (Table 15).

3. Characterization of GPRA as a Genetic Regulator of Asthma Related Traits

A. Detection of GPRA Polymorphisms Associated to Asthma Related Traits

To detect sequence variations in GPRA associated to asthma related traits, we sequenced its verified exons and exon-intron boundaries in four patients homozygous for the haplotype significantly associated with high serum total IgE level among the Finnish asthma families. Comparison between patients and one control individual from the same data set were made to the reference sequence (genomic contig NT_{—}000380) in the public data base.

A total of 14 SNPs were found distributed in seven exons of GPRA (Table 7). Four of the SNPs were predicted to cause a non-conservative amino acid change: Asn>Ile located in the first extracellular loop, Ser>Arg in the third cytoloop, Gln>Arg and Thr>Ile in the C termini, one in each alternative splice variant. Six of the SNPs were located in the 3′ UTRs of the A, B, and C variants of the gene.

B. Association Analysis

We have previously defined a susceptibility haplotype that significantly associates with asthma related phenotypes (best marker-wise P value based on 10,000 permutations 0.001) (FIG. 3, gray area). The region covers the genomic region between GPRA exons 2b and 5. We replicated the association analysis with five SNPs genotyped in the exons of GPRA in the same data set (304 disease associated and 220 control chromosomes). Best haplotype associations of GPRA for high serum IgE level when one gap was allowed in the haplotype patterns are shown in Table 8. The best observed associations reached χ2 values of 5.7-10.3. Only one of markers (SNP591694) was part of the previously determined susceptibility haplotype and the same amino acid change, 107Ile, showed also significant association to high IgE level together with other GPRA SNPs.

To study whether the overall haplotype distribution in GPRA differs in high IgE associated compared to that in control chromosomes, we used a permutation test. HPM analysis was done using the following parameters: maximum pattern length 5 markers, one gap allowed for missing data and possible errors, and chi-square threshold for the association >5.0. The observed scores (=number of qualified haplotype patterns spanning across the marker) for associated haplotypes varied from 6 to 14. Permutation test showed statistically biased haplotype distribution for high IgE. Based on 10,000 simulations, the best marker-wise P value≦0.01 was observed for a silent SNP (in position 640,764) in exon 5. P≦0.02 was observed for three markers in the middle of the haplotype and P<0.04 for all the markers in the haplotype.

### Example VII

This example describes a second gene occurring partially within the AST-1 locus and overlapping the GPRA gene.

A. Materials and Methods

1. Exon Prediction

Exon predictions were performed by GENSCAN (http://genes.mit.edu/GENSCAN.html) software using genomic clone NT_{—}000380 (Polvi et al. 2002). Predictions were performed for both unmasked and masked sequence. In the latter, the repetitive sequences were masked using the Repeat Masker Service (http://repeatmasker.genome.washington.edu/).

2. Reverse Transcriptase PCR

The exon-specific primers were designed based on exon predictions (Table 9, FIG. 13). First strand cDNAs were synthesized from 1 μg of commercially available poly A⁺ RNAs from human lung, testis, kidney and fetal liver (BD Clontech) by the SMART RACE cDNA Amplification Kit (BD Clontech, Palo Alto, Calif.) according to manufacturer's instructions. Alternatively, Marathon-Ready cDNA (Clontech) or cDNA from Multiple Tissue cDNA Panels I and II (Clontech) were used as templates in some PCR amplifications.

All the PCR amplifications were performed in 25-50 μl volumes using 2.0-5.0 μl cDNA as template, 2 nM MgCl₂, 0.2 mM dNTPs (Finnzymes), 0.2 mM of each primer, 0.15-0.4 U AmpliTaqGold, and 1×PCR Gold buffer (Applied Biosciences) under the following conditions: 94° C. for 5 min, 35-40 cycles of 94° C. for 30 s, 58° C.-62° C. for 40 s, 72° C. for 1 min 30 s followed by 72° C. for 10 min. PCR products were analyzed on 1% agarose gel and extracted from the gel using QIAquick Gel Extraction Kit (Qiagen). Purified PCR products were analyzed by automated sequencing with dye-terminator chemistry (Megabase 1000 sequencer, Molecular Dynamics).

3. Cell Culture and Isolation of Poly A⁺ RNA

In addition to commercially available poly A⁺ RNAs, RNA from Epstein Barr virus infected lymphoblast cell lines of the patients who were homozygous, heterozygous, or non-carriers of AST1 were cultured for expression studies. Lymphoblasts were cultured in RPMI 1640 medium (Gibco BRL) supplemented with 1 mM sodium pyruvate (Gibco BRL), 10% FCS (Biological Industries), and 1% Penicillin/Streptomycin (GibcoBRL). Poly A⁺ RNA was isolated by Dynabeads mRNA DIRECT Kit (Dynal) according to the manufacturer's instructions.

4. Northern Blot Hybridization

The AAA1 specific probe (mixture of the variants I, III, IV, VII, and X) was generated by RT-PCR using human lung, testis, kidney and fetal liver poly A⁺ RNAs as templates. The probe was radiolabelled with α[³²P]-dCTP using RediPrime Kit (Amersham Biosciences) according to the manufacturer's instructions. A Human Multiple Tissue 12-lane Northern blot, Fetal Multiple Tissue Northern blot, and Human Multiple Tissue Expression Array 2 (BD Clontech) were prehybridized in ExpressHyb solution (BD Clontech) for 1 h at 68° C. followed by hybridization with the specific probe for 2-5 h at 68° C. Herring sperm DNA (100 μg/ml) was used as the blocking reagent. Filters were washed with 2×SSC and 0.05% SDS at room temperature and exposed to X-ray film at −20° C. for 1-5 days.

5. Genotyping

Genotyping of four SNPs that are located either in the coding region or near the exon-intron boundaries of AAA1 was done 1) using altered restriction sites (SNP_{—}538567 or SNP_{—}574953, Table 1B) or 2) single base pair extension (SBE) with the chemistry of Molecular Dynamics on a Megabase 1000 sequencer (Molecular Dynamics) according to the suggestion made by the manufacturer (Table 10). Allele calling was performed by using the MegaBACE SNP Profiler software (Molecular Dynamics.

All the markers were in Hardy-Weinberg equilibrium and observed Mendel errors were less than 0.1%.

6. In Vitro Translation of the AAA1 and Characterization of AAA1 Antibody

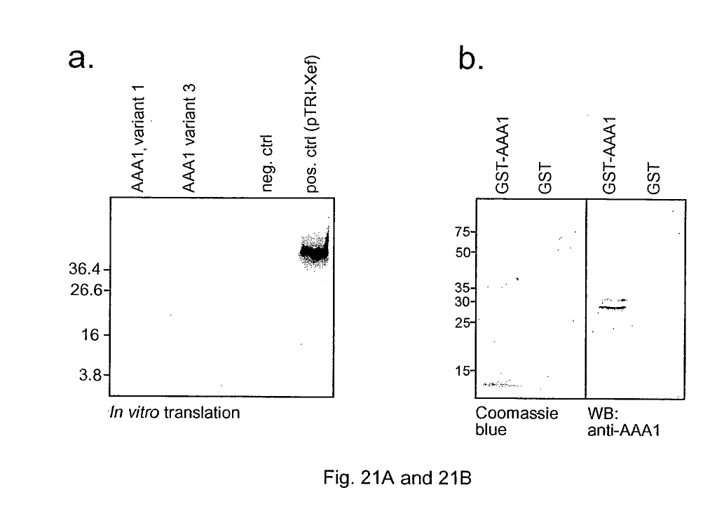

In order to investigate whether AAA1 is translated to any polypeptide, in vitro translation experiments were performed (FIG. 21). Capped RNAs of AAA1 gene variants defined by SEQ ID NOS: 16 and 22 were transcribed from DNA constructs with the aid of T7 RNA polymerases (mMESSAGE mMASCHINE system, Ambion, USA). Translation was performed with rabbit reticulocyte lysate translation machinery (Riboprobe in vitro translation system, Promega) in the presence of S³⁵-labelled methionine in the reaction mixture. The Xenopus elongation factor α (pTRI-Xef) DNA template was used as a positive control for transcription and translation. In negative control, water was used instead of DNA. The translated polypeptides were detected by autoradiography after Tris-Tricine SDS-PAGE.

B. Results

1. Characterization of AAA1 at RNA Level

A. Genomic Structure of Different Splice Variants of AAA1

The human AAA1 gene spans 520 kb of the genomic contig NT_{—}000380 (nucleotides 163615-684776) and it is divided into 18 exons (FIG. 13 and Table 11). The cDNA sequences of AAA1 splice variants were assembled by using RT-PCR with primers designed for predicted exons. The genomic location of the primers is shown in the FIG. 13 and the sequence in the Table 9. Using cDNAs from human lung, kidney, testis and fetal liver as templates, twelve splice variants were identified repeatedly (variants I-XII in the FIG. 14). All variants share exon 6, but use alternative exons to encode the 5′ and 3′ ends of the transcript. In six variants out of ten, the sequence flanking the putative ATG translation initiation site (GCCATGC) contains the −3 purine, but not the +4 guanine residue, of the Kozak consensus sequence.

B. Expression Profiling of AAA1 Using RT-PCR and Northern Blot Hybridization

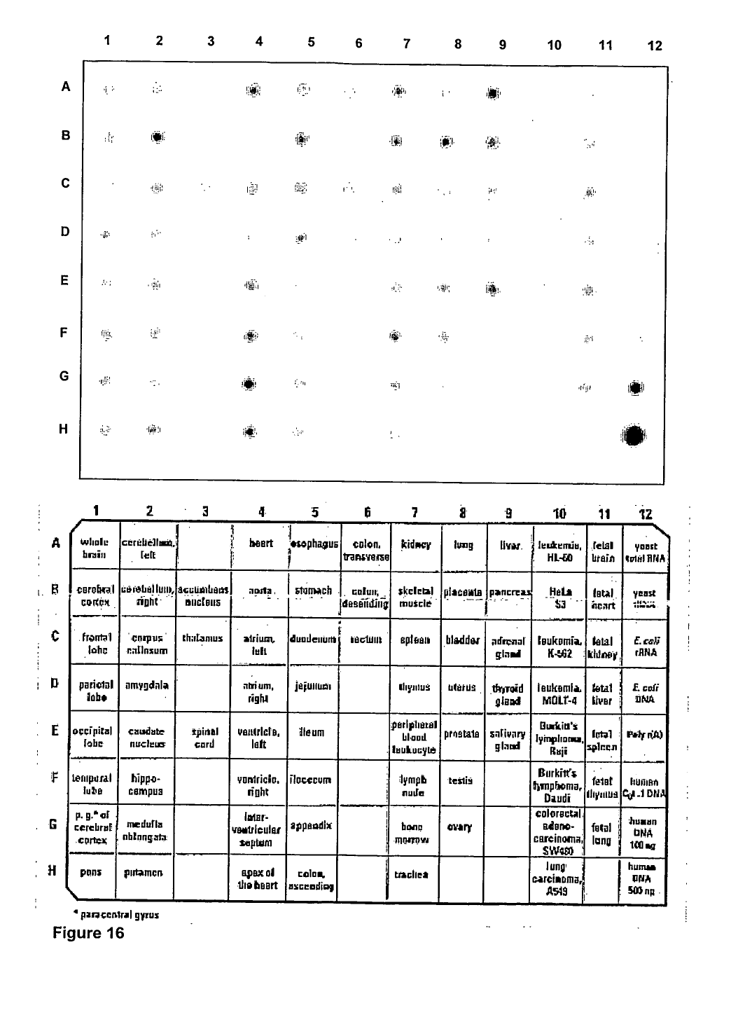

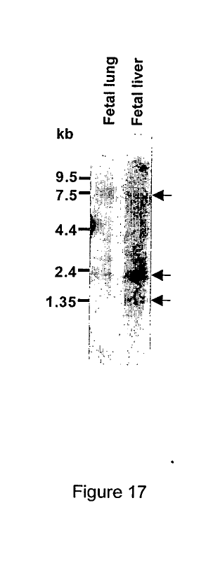

Northern blot analysis of a multiple tissue expression array showed that AAA1 is expressed in several human tissues (FIG. 16). Strong expression was seen in testis, brain, placenta, lung, heart, skeletal muscle, kidney, liver, fetal liver, and fetal lung. In multiple tissue northern blots, two main transcripts (2.4 and 7.5 kb in size) were detected (FIG. 17). Additionally, all sample lanes showed extensive label streaming which can be an indication of several low-abundant transcripts with different sizes.

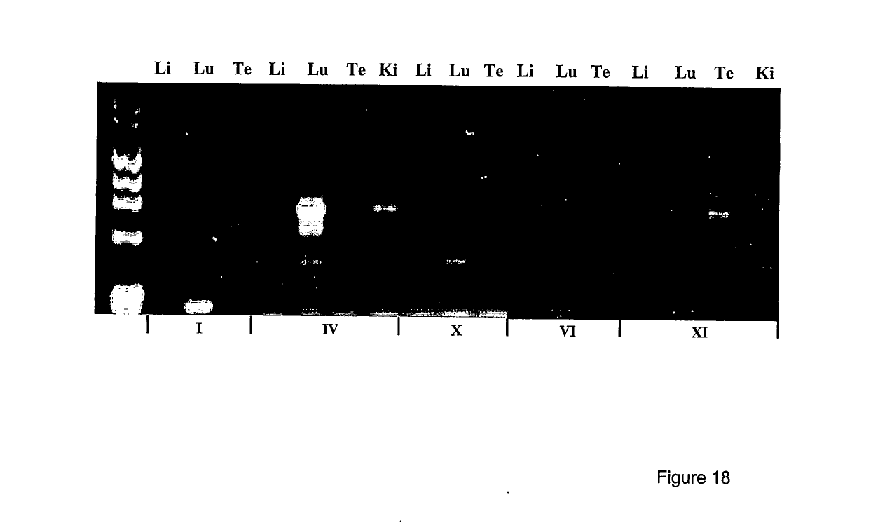

By RT-PCR exceptionally strong splicing and tissue specific differences in AAA1 expression could be found (FIG. 18). For example, expression of splice variants I and IV was particularly strong in the lungs consistent with the association between AAA1 and asthma.

In the in vitro translation experiment, the Xenopus elongation factor α (pTRI-Xef) DNA template used as a positive control resulted in the synthesis of a 50-kDa polypeptide as expected. However, neither of the two investigated constructs was translated into polypeptides strongly arguing that AAA1 functions as a non-coding RNA gene (FIG. 21).

To further investigate the translation of the AAA1 gene, a polyclonal antibody against the constant region (YVRRNAGRQFSHC) of the gene product was produced in rabbits. AAA1 peptide synthesis and antibody production were purchased from Sigma-Genosys Ltd (London Road, Pampisford, Cambridge). To test the specificity of the antibodies, Glutathione S-transferase (GST)-fusion proteins for AAA1 were produced with the pGEX 4T-3 GST fusion expression vector (Amersham Biosciences) according to the manufacturer's instructions. (FIG. 21). The antibody displays high affinity against the recombinant AAA1 protein produced in bacterial lysate with no cross-reactivity between the GST construct alone. In spite of that, the antibody did not reveal any reactivity either in Western blots (spleen, skeletal muscle, uterine muscle, colon muscle, colon epithelium, kidney, testis and prostate) or in immunohistochemistry (bronchial tissue, HepG2 cell line) (data not shown).

In addition, formalin fixed, paraffin-embedded specimens of normal adult human bronchus tissue and HepG2-cells (positive for AAA1 RT-PCR) were used for immunohistochemistry. Immunohistochemical analyses were performed using rabbit AAA1 antibody and the ABC method (Vectastain Elite ABC kit, Vector Laboratories, Burlingame, Calif.). Finally, AAA1 does not appear to have a mouse counterpart. Concluding from our data, AAA1 may be a non-coding RNA gene and is unlikely to encode a functional protein.

C. The Predicted Structure of the AAA1 Peptides

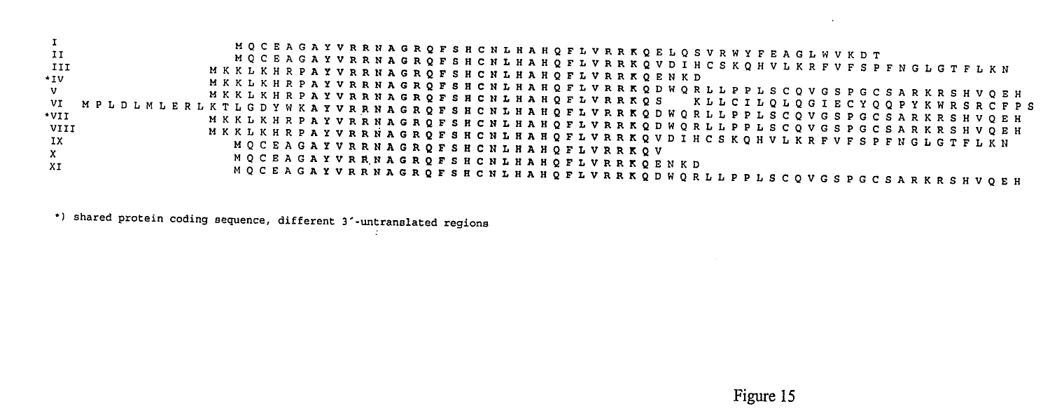

The cDNA sequences of twelve AAA1 splice variants are shown in FIG. 20. All predicted AAA1 proteins are small peptides (size from 34 aa to 74 aa) that do not show significant identity to any known modular structures or motifs. All isoforms contain the same core sequence (AYVRRNAGRQFSHCNLHAHQFLVRRKQ) flanked by alternative amino- and carboxyterminal tails (FIG. 15).

2. Characterization of AAA1 as a Genetic Regulator of Asthma Related Traits

A. Association Analysis

AST1 covered the exons 3 to 10 of AAA1 (FIG. 13). For the association analysis we chose one exonic SNP(SNP 517278) and four additional SNPs near exon-intron boundaries of AAA1 (Table 12). All the polymorphisms were identified previously (Table 3). The analysis for high total serum IgE level was done in the same data set as previously (304 disease associated and 220 control chromosomes, Table 2 and 8). Best haplotype associations of AAA1 are shown in Table 12. The best observe associations reached the χ2 values of 8.9-13.6 and the permutation test showed statistically biased haplotype distribution for high IgE. Based on 10,000 simulations, the best marker-wise P value≦0.0001 was observed for SNP_{—}517278 in exon 10. Corrected P value for testing multiple markers simultaneously was 0.0002.

B. Variable Alternative Splicing for AAA1 Depending on Genotype

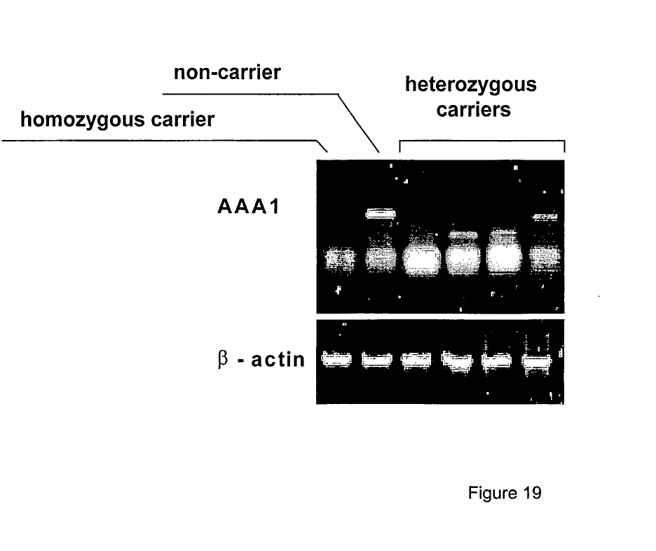

To study further whether AST1 effects on the expression of AAA1 we studied lymphoblast cell lines from the asthma patients who were homozygous, heterozygous, or non-carriers of AST1. Using RT-PCR with the primer pair SCF10 and ASKAR (Table 9) significant differences were found between patients and the differences were dependent on genotype (FIG. 19). Only the non-carrier of AST1 processes normal amount of the exon 6-10b transcript, whereas the homozygote and heterozygotes show either an absent transcript or smaller splice variants. Differences in expression patterns suggest that AST1 can effect splicing of the gene and thereby, increase the risk of asthma related diseases among AST1 carriers compared to that among AST1 non-carriers.

### REFERENCES

Dantzker, D. R. et al. 1987. Standards for the diagnosis and care of patients with chronic obstructive pulmonary disease (COPD) and asthma. Am. Rev. Respir. Dis. 136: 225-243.
Becker, K. G. et al. 1998. Clustering of non-major histocompatibility complex susceptibility candidate loci in human autoimmune diseases. Proc. Natl. Acad. Sci. U.S.A. 95: 9979-84.
Daniels, S. E. et al. 1996. A genome-wide search for quantitative trait loci underlying asthma. Nature 383: 247-50.
Dizier, M. H. et al. 2000. Genome screen for asthma and related phenotypes in the French EGEA study. Am. J. Respir. Crit. Care Med. 162: 1812-8.
Jacob, H. J. et al. 1992. Genetic dissection of autoimmune type I diabetes in the BB rat. Nat. Genet. 2: 56-60.
Laitinen, T., et al. 2001. A susceptibility locus for asthma-related traits on chromosome 7 revealed by genome-wide scan in a founder population. Nat. Genet. 28: 87-91.
Leaves, N. I. et al. 2002. A detailed genetic map of the chromosome 7 bronchial hyper-responsiveness locus. Eur. J. Hum. Genet. 10:177-82.
Malerba, G. et al. 2000. Linkage studies to asthma and atopy phenotypes on chromosomes 7, 12, and 19 in the Italian population. Am. J. Hum. Genet. 67: 330.
Mathias, R. A. et al. 2001. Genome-wide linkage analyses of total serum IgE using variance components analysis in asthmatic families. Genet. Epidemiol. 20: 340-55.
Ober, C. et al. 2000. A second-generation genomewide screen for asthma-susceptibility alleles in a founder population. Am. J. Hum. Genet. 67: 1154-62.
Polvi A et al. 2002. Physical map of an asthma susceptibility locus in 7p15-p14 and an association study of TCRG. Eur. J. Hum. Genet. 10, 658-665 (2002).
Remmers, E. F. et al. 1996. A genome scan localizes five non-MHC loci controlling collagen-induced arthritis in rats. Nat. Genet. 14: 82-5.
Satsangi, J. et al. 1996. Two stage genome-wide search in inflammatory bowel disease provides evidence for susceptibility loci on chromosomes 3, 7 and 12. Nat. Genet. 14: 199-202.
Sawcer, S., et al. 1996. A genome screen in multiple sclerosis reveals susceptibility loci on chromosome 6p21 and 17q22. Nat. Genet. 13: 464-8.
Sevon, P. et al 2001a. TreeDT: Gene mapping by tree disequilibrium test. In KDD-2001. Proceedings of the Seventh ACM SIGKDD International Conference on Knowledge Discovery and Data Mining, pp 365-370. Editors: Provost, F. and Srikant, R. Publisher ACM press, San Francisco, Calif. USA (www.acm.org/sigkdd/kdd2001)
Sevon, P. et al. 2001b. TreeDT: Gene mapping by tree disequilibrium test (extended version). In the publication series Report C-2001, Nr 32:6-7 published by Department of Computer Science, University of Helsinki
Toivonen, H. T. et al. 2000. Data mining applied to linkage disequilibrium mapping. Am. J. Hum. Genet. 67:133-45.
Wjst, M., et al. 1999. A genome-wide search for linkage to asthma. German Asthma Genetics Group. Genomics 58: 1-8.
Xu, J. et al. 2001. Genomewide Screen and Identification of Gene-Gene Interactions for Asthma-Susceptibility Loci in Three U.S. Populations: Collaborative Study on the Genetics of Asthma. Am. J. Hum. Genet. 68: 1437-1446.
Xu J et al (2000). Major genes regulating total serum immunoglobulin E levels in families with asthma. Am J Hum Genet. 67:1163-73.
Yokouchi, Y. et al. 2000. Significant evidence for linkage of mite-sensitive childhood asthma to chromosome 5q31-q33 near the interleukin 12 B locus by a genome-wide search in Japanese families. Genomics 66: 152-60.
Daly, M. J. et al. 2001. High-resolution haplotype structure in the human genome. Nat. Genet. 29: 229-32.
Dantzker, D. R. et al. 1987. Standards for the diagnosis and care of patients with chronic obstructive pulmonary disease (COPD) and asthma. Am. Rev. Respir. Dis. 136: 225-243.
Johnson, E. N. et al. 2002. Heterotrimeric G protein signaling: role in asthma and allergic inflammation. J Allergy Clin Immunol. 109: 592-602.
Kauppi, P. et al. 1998. Verification of self-reported asthma and allergy in subjects and in their family members volunteering for gene mapping studies Resp. Med. 92: 1281-1288.
Laitinen, T. et al. 1997. Genetic control of serum IgE levels and asthma: Linkage and linkage disequilibrium studies in an isolated population. Hum. Molec. Genetics 6: 2069-2076.
Laitinen, T. et al. 2001. A susceptibility locus for asthma-related traits on chromosome 7 revealed by genome-wide scan in a founder population. Nat. Genet. 28: 87-91.
Michel U 2002. Non-coding ribonucleic acids—a class of their own? Int. Rev. Cytol. 218: 143-219.
Numata et al. 2003. Identification of Putative Noncoding RNAs Among the RIKEN Mouse Full-Length cDNA Collection. Genome Res. 13: 1301-06.
Palczewski, K. et al. 2000. Crystal structure of rhodopsin: A G protein-coupled receptor. Science 289: 739-45.
Park, Y. et al. 2002. Identification of G protein-coupled receptors for Drosophila PRXamide peptides, CCAP, corazonin, and AKH supports a theory of ligand-receptor coevolution. PNAS 88: 11423-28.
Rana, B. K. et al. 2001. Genetic variations and polymorphisms of G protein-coupled receptors: functional and therapeutic implications. Annu. Rev. Pharmacol. Toxicol. 41: 593-624.

Although the foregoing invention has been described in some detail for purposes of clarity and understanding, it will be clear to one skilled in the art from a reading of this disclosure that various changes in form and detail can be made without departing from the true scope of the invention. The above examples are provided to illustrate the invention, but not to limit its scope; other variants of the invention will be readily apparent to those of ordinary skill in the and are encompassed by the claims of the invention. The scope of the invention should, therefore, be determined not with reference to the above description, but instead should be determined with reference to the appended claims along with their full scope of equivalents. Any embodiment, feature, step or element described above can be used in combination with any other unless otherwise apparent from the context. All publications, references, and patent documents cited in this application are incorporated by reference in their entirety for all purposes to the same extent as if each individual publication or patent document were so individually denoted.

TABLE 1
Primers that were used in SNP genotyping with SBE method (A); primers, restriction
enzymes, and the length of the digested PCR product of corresponding alleles used in SNP
genotyping with altered digestion (B); primer pairs and the length of the corresponding PCR
product used in genotyping of the small insertions and deletions (C).
TABLE 1A

| | Nucleotide | | | | |
|---|---|---|---|---|---|
| SNP ID | NT 000380.3 | Type¹ | Fw primer | Rev primer | PE PRIMER |
| dbSNP: | 506401 | C/T | CCCCCTGCTCTCTCTCTCTT | GGGATTTATCCATTTCCTCCA | ATGCCAGCATCATACTTCCTG |
| 323926 | | | | | |
| dbSNP: | 509240 | G/G | GATCTTCCATTTCACCAAGCA | GATGGATATTTAGCTCCCCCTTA | CACACTAGCAAAGTCAGGCAAT |
| 323906 | | | | | |
| dbSNP: | 515224 | C/G | TGAGTTGATGAATCTGCTGGA | GGCAATGCCAATTAAGAGGA | CACGAGCCCTGGATTACCTAC² |
| 323917 | | | | | |
| dbSNP: | 515632 | A/G | CAATGGATCACTGCAGCCTA | TTCCCACCCATTAGTTTTTCC | AGAACAAGCTATGCCAGAGACTG² |
| 143266 | | | | | |
| dbSNP: | 529556 | A/C | AACACAGCCCTCCAGACACT | ATACCTGGCCAGCGGTTTA | TGGAAAAATTTGAACATTATTGGG |
| 324377 | | | | | |
| dbSNP: | 531632 | A/G | CTATTGGGCCCTGGAGACTT | ATGCAGCTTGCATTGTTCAC | GGTCTCTGGGACCCCTTC² |
| 324373 | | | | | |
| dbSNP: | 543580 | C/T | AGGGATCATGTCTCCAGCAC | TTAAATGCCCACTAGCACCA | GAGCAGAGATCAGAAACAGGCT² |
| 182718 | | | | | |
| dbSNP: | 555608 | A/G | TGTGTCTCCTTCTTTGTGTTCATA | TGAAAGCAGGGCTACCATTT | GCAGGAACAGAAAACGAAATACC² |
| 324384 | | | | | |
| dbSNP: | 563704 | C/T | CACAGGGAGGAAAGGAACAG | GATGGGATTGAGGAGCTTGA | ACAGTGGATGGTTTCTTGGC |
| 324396 | | | | | |
| dbSNP: | 591694 | A/T | GGCCATCTGATAAAGCAGGA | TGATGCATAGGAATGCAAGG | AGTCTCCAGTGAATCGCCAA² |
| 324981 | | | | | |
| dbSNP: | 617392 | C/T | GATGAAAAGGGAACCATTCCT | TGCTTTTCCTGTGTCCATTG | ATGCCCTAATTCTAAAACCAATGA |
| 325456 | | | | | |
| dbSNP: | 638799 | C/T | TGACATGTTCCCTGCATTTG | AGAGGGCTTTGTGCTCTCTG | CACCCTCCCTGGTGACCT² |
| 62911 | | | | | |

¹Polymorphism detected in assays employing primers specified in this table
²extension primers (Pe) designed in reverse orientation

Primer, restriction enzymes, and sizes of the corresponding
alleles after digestion used in SNP genotyping of AST1.
TABLE 1B

| Marker | Forward primer | Reverse primer | Enzyme | Product size* |
|---|---|---|---|---|
| SNP_490331 | gcctgccctagagacatcag | tggctgccttcttcatctttg | HpyCH4V | 180 + 110 |
| SNP_490390 | gcctgccctagagacatcag | tggctgccttcttcatctttg | HpyCH4V | 180 + 110 |
| SNP_490820 | ccaattctcatccaaaaggc | taaactaggtgcgtcacatttgct | Dralll | 330 |
| | | cactggagatggc | | |
| SNP_513659 | aactgactaaactaggtgccacgtcg | tggatttgcatttcgtctaatg | Mnll | 220 |
| | tatcctgacacagggatttaagc | | | |
| SNP_514743 | ggaaactggaagtcaacacca | tctccctcagaaaagccaga | Sspl | 420 |
| SNP_516094 | cctgcaataaacattctcataattaaa | ggtgggattacaggcatgag | Faul | 400 |
| SNP_516174 | gaagtggacttactgcatcaaaga | ggggtttcaccgtgttagc | Sfcl | 400 |
| SNP_516267 | gctctagaacccgctagcat | gcctcagcctctggagtagc | BsrBl | 360 |
| SNP_520598 | gcaaaagttgagggggattc | accatgcctcgccatagtaa | Nlalll | 300 |
| SNP_521640 | aggaacccttcccagcag | cccaaagccctgagaaaaat | Blpl | 360 |
| SNP_522363 | tgcaccagctttttgaggta | tgtaacatcccccagggact | EcoNl | 380 |
| SNP_526991 | ctttagtgggaaggctgtgg | aagccactaatatttgggatgg | BsaWl | 400 |
| SNP_528709 | tgctctttttgacagccaatc | ggctgctttgatctgaggac | Ncol | 360 |
| SNP_529142 | ccataagcctcgcctatttt | gtgggcactgaggcatagat | Taql | 410 |
| SNP_529820 | caaatcctcgttctcctgct | tacccagaggctgagaaagg | BsaAl | 400 |
| SNP_530177 | cctttctcagcctctgggta | gtgagctgagttcgcgctat | Styl | 380 |
| SNP_538567 | ctcagaacactgctgccaga | gctctgggatgcgttaactt | EcoNl | 380 |
| SNP_541906 | ttgatctggccaaatggaac | ctaaacaaaaaggggctgcaa | Aflll | 350 |
| SNP_546333 | gcatgtgaatacagcacttgg | ctcccgagttcaagcaattc | Haelll | 290 + 90 |
| SNP_563585 | ccctacaagttaccctgcaca | cagaggtttggatgggattg | Hindlll | 375 |
| SNP_563930 | tcaagctcctcaatcccatc | gtggaggaagtgccttttca | Stul | 330 + 40 |
| SNP_564782 | aaaggcagccagtgtctgtt | ggcagagagtcccacagtga | Mspl | 400 |
| SNP_574953 | tctcagcctgctacccctta | aagcacaagcatcaggttca | Ahdl | 365 |
| SNP_585883 | tcaacagaggcatgattgga | gcagatggaggtagagctgttt | Sfcl | 370 |
| SNP_640764 | aactgactaaactaggtgccacgtcg | tagcacaatgcctgccctat | Sapl | 340 |
| | tccatacatgaccatcgtgctctt | | | |
| SNP_647327 | aggcaagtcacctgctcttc | gctgcagaaagaaccaaagg | BstEll | 455 |
| SNP_662764 | tgtctacctgttggcctgtg | gtcttgtgcatctcccaggt | Mboll | 455 |
| SNP_662803 | catggagaaggaaggtcagg | ctagcactggcactgcccta | Ddel | 270 |
| SNP_663133 | atgactgcatgcactgctta | tctgcaaaccgaggctatct | SfaNl | 400 |

| Marker | Allele 1 | Product size* | Allele 2 | Note |
|---|---|---|---|---|
| SNP_490331 | c | 180 + 90 + 20 | a | special combinations of fragments when |
| | | | | heterozygous for the SNPs 490331 or 490390 |
| SNP_490390 | a | 180 + 80 + 30 | g | |
| SNP_490820 | t | 250 + 80 | g | plasmid tail with the Dralll restriction site |
| SNP_513659 | g | 170 + 50 | c | plasmid tail |
| SNP_514743 | g | 210 + 210 | a | |
| SNP_516094 | t | 210 + 190 | c | |
| SNP_516174 | t | 200 + 200 | g | |
| SNP_516267 | a | 180 + 200 | g | |
| SNP_520598 | c | 130 + 170 | t | |
| SNP_521640 | a | 180 + 180 | g | |
| SNP_522363 | g | 220 + 160 | a | |
| SNP_526991 | c | 230 + 170 | a | |
| SNP_528709 | t | 180 + 160 | g | |
| SNP_529142 | a | 240 + 170 | g | |
| SNP_529820 | t | 220 + 180 | c | |
| SNP_530177 | t | 230 + 150 | c | |
| SNP_538567 | g | 220 + 160 | a | |
| SNP_541906 | c | 180 + 170 | a | |
| SNP_546333 | a | 190 + 100 + 90 | g | |
| SNP_563585 | c | 190 + 185 | t | |
| SNP_563930 | g | 150 + 180 + 40 | a | |
| SNP_564782 | a | 170 + 230 | g | |
| SNP_574953 | a | 180 + 165 | g | |
| SNP_585883 | g | 180 + 190 | c | |
| SNP_640764 | t | 290 + 50 | c | plasmid tail |
| SNP_647327 | c | 210 + 245 | t | |
| SNP_662764 | a | 240 + 215 | g | |
| SNP_662803 | c | 220 + 50 | t | |
| SNP_663133 | t | 140 + 260 | c | |

TABLE 2
HPM permutation test done using the following parameters:
maximum pattern length 40 markers, one gap allowed for missing
data and possible errors, and chi-square threshold for association 13.0.
The observed scores (=number of qualified haplotype patterns
spanning across the marker) and marker wise P values based on 10 000
permutations are shown. The critical region (=AST1) is highlighted.

| Marker | HPM Score | Marker wise P value |
|---|---|---|
| NM4 | 0 | 1 |
| NM5 | 0 | 1 |
| D7S683 | 0 | 1 |
| D7S656_2 | 0 | 1 |
| NM2 | 0 | 1 |
| MIT_MH26 | 0 | 1 |
| NM8 | 0 | 1 |
| NM9 | 0 | 1 |
| NM11 | 0 | 1 |
| G42099 | 0 | 1 |
| SNP_490331 | 0 | 1 |
| SNP490391 | 0 | 1 |
| SNP_490820 | 0 | 1 |
| SNP_506401 | 0 | 1 |
| SNP_509240 | 0 | 1 |
| NM50 | 0 | 1 |
| SNP_513659 | 0 | 1 |
| SNP_514743 | 0 | 1 |
| SNP_515224 | 0 | 1 |
| SNP_515632 | 0 | 1 |
| SNP_516094 | 0 | 1 |
| SNP_516174 | 0 | 1 |
| SNP_516267 | 0 | 1 |
| NM49_516568 | 0 | 1 |
| NM51_517022 | 2 | 0.0139 |
| G42102_518794 | 7 | 0.0105 |
| NM53_519564 | 12 | 0.0091 |
| SNP_520598 | 15 | 0.0083 |
| SNP_521640 | 20 | 0.0079 |
| SNP_522363 | 23 | 0.0075 |
| SNP_526991 | 23 | 0.0076 |
| SNP_528709 | 30 | 0.007 |
| SNP_529142 | 33 | 0.0066 |
| SNP_529556 | 40 | 0.0062 |
| SNP_529820 | 33 | 0.0061 |
| SNP_530177 | 34 | 0.0059 |
| SNP_531632 | 34 | 0.0054 |
| D7S497_538277 | 40 | 0.0052 |
| SNP_538567 | 38 | 0.0052 |
| SNP_541906 | 36 | 0.0054 |
| SNP_543580 | 35 | 0.0054 |
| SNP_546333 | 35 | 0.0054 |
| SNP_555608 | 35 | 0.0051 |
| NM45_560804 | 35 | 0.0049 |
| SNP_563585 | 35 | 0.0049 |
| SNP_563704 | 28 | 0.0046 |
| SNP_563930 | 23 | 0.005 |
| SNP_564782 | 16 | 0.0063 |
| SNP_574953 | 10 | 0.0071 |
| SNP_585883 | 4 | 0.0093 |
| SNP_591694 | 3 | 0.0098 |
| SNP_617392 | 3 | 0.0085 |
| SNP_638799 | 0 | 1 |
| SNP_640764 | 0 | 1 |
| G42097 | 0 | 1 |
| SNP_647327 | 0 | 1 |
| SNP_662764 | 0 | 1 |
| SNP_662803 | 0 | 1 |
| SNP_663133 | 0 | 1 |
| NM13 | 0 | 1 |
| NM46 | 0 | 1 |
| D7S484 | 0 | 1 |

TABLE [symbol] [symbol]
genomic contig NT₁₃ 000380) and corresponding alleles in haplotypes H1-H7

| | | | |
|---|---|---|---|
| Type of | Position in | Position in | |
| polymorphism | SEQ ID NO:1 | NT₁₃ 000380 | Polymorphism and flanking sequence |

| | | | |
|---|---|---|---|
| SNP | 1 | 509783 | ACAATACAATAGTTAAACTTAG[C>T]GGCAAATTAAATGTAGC |
| SNP | 93 | 509875 | TTGAAGCATGAAAAAAGAAAGGAC[A>G]AAAAATACAAAAAGA |
| insertion | 184-185 | 509966-67 | GATAGAAACAACACTT[AAGATA]ATAGTGGCCAATAATTTTCCCAAACTCT |
| (NM50) | | | |
| SNP | 918 | 510700 | TCCCCTGCCAATACAAAAATTCGAG[G>A]ATGCTTAAGTCCCTTACATAAA |
| SNP | 983 | 510765 | CTACATACACCCTCCTGTAT[A>T]CTTTAAGTCATCTCTAAATTACTT |
| SNP | 987 | 510769 | CTACATACACCCTCCTGTATACTT[T>C]AAGTCATCTCTAAATTACTT |
| deletion | 1202-1206 | 510984-88 | ATATTATATAAAATCAATC[TAAGT]TAAGACAGGAGAGAAAAAATATTA |
| SNP | 1542 | 511324 | TATCAAAGTTTATAAATTTGCTGTGATT[C>T]TTGGAATCACTTGTAGCTTTATC |
| SNP | 1710 | 511492 | CACCAAATTATATACTTATGATTTTGT[A>G]TACTTTTCTTTATGTGTTTTAT |
| SNP | 1818 | 511600 | CAATGGCTTTAGGAATCAAGCAGG[C>T]AATGAATGTAAAXATGTAAA |
| SNP | 1927 | 511709 | AAGACATTAAAATCAATTTTTGTC[A>T]AACACTGTGCTGATAAACAAAT |
| SNP | 2254 | 512036 | ATTTTCTTCTCCTAATGTGTT[T>C]ATTATTGTTTTTATTGTATAAT |
| SNP | 2937 | 512719 | CAAACAACAGAAATTTATTTCCTC[G>A]CAGTCCTGTAGGCTGAAAGTTT |
| SNP | 3877 | 513659 | TCACACAGGGATTTAAGCCT[G>C]AAGTTTTTTTCAGAGAAGA |
| SNP | 4012 | 513794 | TTCACACCTTCAAGAATGGCTATTCC[C>A]CTATAAAATAATATACTAA |
| SNP | 4631 | 514413 | CAACAGCAAATGCAAGAGAGACTTT[T>C]TCGGAACTCAGCTGCCTAAAA |
| SNP | 4689 | 514471 | CAAAAGAAATTCAATTGCCATATATC[C>G]TCTCCCTGGGAGTTTTATTAA |
| SNP | 4961 | 514743 | CCTGTGGCACCCCATGAACATAAT[A>G]TTAAAATGAATAT |
| SNP | 5442 | 515224 | TATCTTATGTAAAGAAGTCCGGA[C>G]GTGTAGGTAATCCAGGGCTCGTGCTG |
| SNP | 5634 | 515416 | CAGGAAGGAAGAAGGGAGGAA[G>A]GACAGAGGGGCTCATATCAGCTCTTTG |
| SNP | 5850 | 515632 | AGAAATAGAATTAAGGA[A>G]CAGTCTCTGGCATAG |
| SNP | 6312 | 516094 | AATGTCGGGGGACAAATGCTCTAGAA[C>T]CCGCTAGCATAGAC |
| SNP | 6392 | 516174 | ACTTGCAGTAGCTACA[G>T]AATCATAACTGGTTTTTT |
| SNP | 6485 | 516267 | AATTTGCTTATTATCAAAAG[G>A]AGCGGCCAGGCGCGGTGG |
| SNP | 6522 | 516304 | TGGCTCATGCCTGTAATCCCA[C>G]CACTTTGGGAGGCCGGCGGG |
| SNP | 6646 | 516428 | GGCGTGGTGGCGGGCGCG[A>G]TGGCAGGCGCCTGTAGTCCCAGC |
| SNP | 6739 | 516521 | CGGAGCTCGCAGTGAGCCGAGATCGC[G>A]CCACTGCCCTCCAGCCTGGG |
| SNP | 6760 | 516542 | ACTGCCCTCCAGCCTGGG[T>C]GACAGAGCAAGACTCC |
| [symbol] AAA- | 6786-6817 | 516568-99 | AGCAAGACTCCGTCTCAAAA[TAAA)8>(TAAA)7]GCATTTTTTTTTTCACTTTAG |
| repeat | | | |
| (NM49) | | | |
| [symbol] eletion | 6821 | 516603 | ATAAATAAATAAATAAAGCA[T]TTTTTTTTTCACTTTAGCCAGC |
| SNP | 7125 | 516907 | TGAAGCCACTATTTGG[C>A]ACATTCTGTAATTCTTAGAA |
| SNP | 7229 | 517011 | CCAATTAAAATTATTCATTTC[C>T]CATCTTTAAGACTTACTTGTCTGTTTTA |
| [symbol] eletion | 7240-7243 | 517022-25 | CCAATTAAAATTATTCATTTACCATCTTTAAG |
| (NM51) | | | |
| SNP | 7277 | 517059 | ATGCAATAAAAATG[C>G]GGAACTAATAAGAGTAA |
| SNP | 7303 | 517085 | AAGATAACTCCCGAT[G>T]AGTGTTCAACAAAGAAAAGAAAGAAAA |
| SNP | 7305 | 517087 | AAGAGTAACTCCCGATGA[G>C]TGTTCAACAAAGAAAAGAAAGAAAA |
| [symbol] eletion | 7306-7308 | 517088-90 | AAGAGTAACTCCCGATGAG[TGT]TCAACAAAGAAAAGAAAGAAAA |
| (NM52) | | | |
| [symbol] eletion | 7334-7335 | 517116-17 | CAACAAAGAAAAGAAAGAAAACAT[TA]TTTTTGGCGCACATTCAAATC |
| (NM52241) | | | |
| SNP | 7496 | 517278 | ACCCTCCTATACATTACCTGAA[C>T]GCAGAGAAACTTGAGACGT |
| SNP | 7550 | 517332 | AGGTAAGGCCAGGAGTGTTGAGGC[G>A]TCCAGGTCCGTCTGTGGAGTT |
| SNP | 8490 | 518272 | CTCTATATTGTCTCTTTTTATATA[C>T]ATATTTTTTGCCTTTTAACGTATG |
| [symbol] T-repeat | 9012-9035 | 518794-817 | TGTTTGTGTATGTATGTTGAT[(CT)12>(CT)10]CATACACACACATAAGTCTTTT |
| (G42102) | | | |
| [symbol] eletion | 9199-9201 | 518981-83 | TTTTGTAACTGTTTTGGCATTTA[TCT]TCTTCATTGTGACTTTACTTGAAA |
| [symbol] sertion | 9355-9356 | 519137-38 | TTGGGTAGTATTTTTTTTTT[T]CAAGAAGTCATTGGATAAGA |
| [symbol] NP | 9649 | 519431 | TCTATTTTTTGGTCGTCTGTTATG[T>G]GAGTTCTCTGAGCTGCCATA |
| [symbol] eletion | 9782-9785 | 519564.67 | CTTCAGGAACTCTAACTGTCT[GTCT]ATATGTTAGTAATTTTGGCCTTT |
| (NM53) | | | |
| [symbol] NP | 10816 | 520598 | AGAAATAGCTGGCTTCTGGCCCA[T>C]GCAGAAGGAATGTAGAAAATAGA |
| [symbol] NP | 11858 | 521640 | TTCCCCAAAACACATGGGCTGA[A>G]CAGGTAAGATGGATACTT |
| [symbol] NP | 12581 | 522363 | GAATTTGGCGGCCTCATTAC[G>C]TTTCTCAGGAGTCCCTGGGGG |
| [symbol] NP | 16845 | 526627 | TCTAGAATTCCTCCTTTGTAGGCCATCAG[T>C]AATCTCACTGAAAATTAGAC |
| [symbol] NP | 16893 | 526675 | TATAAATAATAATATAA[T>C]ATTATGCATATTTTAAATATT |
| [symbol] NP | 16980 | 526762 | CAGAAATGAAGGCCAATCTTAG[T>C]GGGAAGGCTGTGGGAGCT |
| [symbol] NP | 17147 | 526929 | TGGCAAAGCTCTGCATAGTCC[G>A]TGACTGGGAGATGGGAAA |
| SNP | 17209 | 526991 | TAGGCCCATCCCAGGCCTGT[C>A]CCGGATCCCTCCAGAAATCTCAAGAAA |
| SNP | 17435 | 527217 | GGGTCAAGAATTTAAGTGGAATAC[A>G]GTGAGGTGGCTCATCTITTC |
| SNP | 18383 | 528165 | AGAAACACCACCCCACATCCCAGGCTG[G>A]TATACTTCACAGTAATGATGTG |
| SNP | 18927 | 528709 | CATGTTCACAATATCCCATG[G>T]ATGGGGTCATGTGATGTG |
| SNP | 18978 | 528760 | TGAAATGTGAGTTGAAGGGACT[A>G]TGTCACTCCCAGACTGAGACTGTGAAA |
| SNP | 19268 | 529050 | GAGCAGCACCTGGGTG[G>C]TGAATAGCACCTGGGAAGC |
| SNP | 19272 | 529054 | CATGAGGAGCAGCACCTGGGTGGTGA[A>T]TAGCACCTGGGAAGCTGAACC |
| SNP | 19360 | 529142 | ACTACCATGAGAAGAAAGTC[G>A]AGGGAAGATAAAAGTCAGAA |
| SNP | 19452 | 529234 | CCTTGGGGAAAGAGTGGAAAGAGGGT[G>A]GCCTCCAGAGCTGAGTCCA |
| SNP | 19671 | 529453 | CACTGCCATTCATCTTCCAAAAGAG[G>A]AAATGGACAGACATATAATTACTA |
| SNP | 19712 | 529494 | AGCCCATAAAATGATGG[C>A]AAATAACACAGCCCTCCAGA |
| SNP | 19774 | 529556 | TTTGAACATTATTGGG[C>A]AATCACTGCCTTCTGAC |
| SNP | 20038 | 529820 | CTTAGGACCATGTCTGATA[T>C]GTGAGTCCTCAATGAA |
| SNP | 20089 | 529871 | TTGAATATTATGATATCACCAGG[T>A]TTTTTTAAACCGCTGGCCAGGTATCC |
| SNP | 20309 | 530091 | CTGCTTTGACCAAGACCAGAGT[G>A]TCAGGGGCAGATTACAGGAGTCCTAC |
| SNP | 20395 | 530177 | GCGAGGCAGTGCCTGC[T>C]TTGGAATGCTTTAATAAACA |
| SNP | 20789 | 530571 | CTCCCGATCTCAGGTGATCTGCC[G>T]CCTCAGCCTCCCAAAGTGCTGGGATT |
| SNP | 21850 | 531632 | TTTTGACTTTGCAGATCCCC[C>T]GAAGGGGTCCCAGAGACCC |
| deletion | 22122-22123 | 531904-5 | TCTTTGCTGAGCCTC[TG]TGTTTGTGAGTCTGGAACCC |
| (NM54) | | | |
| SNP | 22475 | 532257 | TGTCCTGCTGGCTTGGTGAGAAGG[T>C]GTCCAGCGTTGGCAGGCAGC |
| SNP | 22493 | 532275 | GTGAGAAGGTGTCCAGCGTTGGCAGGC[A>G]GCTGAGAGAAGTGGGGGA |
| SNP | 22715 | 532497 | GTGCATGCATATGTGGACC[G>A]TGCACTTCTCAACAGAGGGA |
| SNP | 22869 | 532651 | AGCTAGATTGGTCAGAGCTTTAG[T>C]GAAGGAGCAGGTGACC |
| SNP | 22934 | 532716 | AAAAATGCAATAACACACAGTCCCTAGA[A>T]AATGATGACATGCTGTCTCTA |
| SNP | 24007 | 533789 | TGTGTGATGTGTGGGGTT[T>C]GGTGTGTGTGATGGGGGAT |
| SNP | 24264 | 534046 | TCACATATCTGCTTCTAATCATCAAGTC[G>T]GAAAAGAAATCTGGATACTAT |
| SNP | 24869 | 534651 | GGGCTCACCATTTTCAAGCACAG[T>C]AATTAGAACGAGACCC |
| SNP | 26198 | 535980 | CTGCAGATAATGACAGTTC[C>T]GTATGTCAGCATCAACATGGA |
| SNP | 26356 | 536138 | GTGAGGAAGGCCATTTTAGC[C>T]TGAAGTGAGTGTCCCATTA |
| SNP | 26675 | 536457 | AGCACAGTTACTATTTCTGGGAAGATTTC[A>G]TTTGACCCCAATTACTTACA |
| TAAA | 26929-26968 | 536711-50 | CTAAATAGCA[(TAAA)10>(TAAA)11]GATGAATTGTAATTATCT |
| repeat | | | |
| (G42100) | | | |
| SNP | 27404 | 537186 | TTATCCATTTGGTTGAAC[C>T]GAAACCCATCTATCTTCCA |
| SNP | 28197 | 537979 | GTGATATAAGAAAAGTGCTTA[G>A]CATAGTATCTAGGTTACAGTAGGTGACC |
| CA-repeat | 28495-28564 | 538277-346 | CTTCTAAGTACC(CA)6GACT[(CA)13>(CA)12]GA(CA)6CT(CA)6GAGCTCTC |
| (07S497) | | | |
| SNP | 28770 | 538552 | AATTTACTATGTCCCATCAACTGT[C>T]CCAAATCCTCTACCG |
| SNP | 28785 | 538567 | CCCAAATCCTCTACC[G>A]GGATAAGATCACGCTATCTT |
| SNP | 28858 | 538640 | TTGAGACATCTACAATTGAGGA[T>C]ACTGAAGGACAGAGACACTAGGTAATC |
| SNP | 28866 | 538648 | TTGAGACATCTACAATTGAGGATACTGAAG[G>C]ACAGAGACACTAGGTAATC |
| SNP | 31224 | 541006 | TTGGGAGCACCTCACCACACATTAAAGT[G>A]CCCTTTCTTCCACTTCAACTT |
| SNP | 31910 | 541692 | AGCTCAGAAGCTCCAGGTAGAGCA[G>A]ACGCTCTTGCCTTAATCTTTAAAAA |
| SNP | 32124 | 541906 | AATAACCCCTCTGATGGGCTT[C>A]AGACCCATGATAGCTATA |
| SNP | 32185 | 541967 | TTCTCACAAGAAACTGATGGGCATGAAGA[T>C[TGCAGTGACTGAACTGTCTC |
| SNP | 32976 | 542758 | AATTATCAATTTGGAAATGA[T>C]ATGATTAAGAATAAAAAACAAGATTGTTT |
| SNP | 33350 | 543132 | CCCACCCACCACACCTGGCT[C>A]AATGTTTATTTTTTAGTAGCAA |
| SNP | 33798 | 543580 | CTGAACAGTCTTCCCTCA[G>A]AGCCTGTTTCTGATCTCTGCT |
| SNP | 34362 | 544144 | AGAGGAGAGGGGCAGGAGAGAATATA[G>C]TAAAATAGGTTAAAGTTTCCAAT |
| SNP | 34716 | 544498 | ACAAGTAAGATACACACAAA[A>C]TGCAATTCACCAGTAAACC |
| deletion | 34909 | 544691 | AATGAAATATTTAAAAGTACCCC[T]GGTTACATTTTGCTTTTTGGTACTT |
| SNP | 35559 | 545341 | GTTTGTAGAATTCACAACTGATA[C>T]TAAAAGTCTATCATGTAACAGGGCTC |
| SNP | 36551 | 546333 | ATTTAAGAATCCATGCA[G>A]GCCAGGTGCAGTGGCTCA |
| SNP | 36909 | 546691 | CAAGGCTTTGCAATAGC[C>A]TATCCTGCAAAATAAAAGTTAGTT |
| SNP | 37327 | 547109 | ATAGAAAACTGGGAGGGGGAA[G>T]GGACAGAGATGATATTGA |
| SNP | 37415 | 547197 | AGAGGAGTCATAGTCATGG[G>A]AGTGACAGAATCAGATTGGT |
| SNP | 37685 | 547467 | GAGTGACATCTGGGTATCA[A>G]TTTTTTAAAGGTCCCCAGAT |
| SNP | 37931 | 547713 | AAAAAAAACACTAAGGTATAAGTTC[C>T]TCAAATCTAACCTGCCCTCTCCAG |
| SNP | 37959 | 547741 | ATCTAACCTGCCCTCTCCAGGCC[C>T]ACAGACTGGGAGTGGCAATCAGC |
| deletion | 38850-38652 | 548632-34 | TTATCCTGGAGCCCTCTTCCCCTC[CTC]TGTTTTGCTGACCTTAATAAATG |
| SNP | 39314 | 549096 | AACTATTGACAATAAAATTGTAATA[G>A]TATAAAAAAATCTCAACATGAATAAA |
| SNP | 39343 | 549125 | ATAAAAAAACTCAACATGAATAAAAA[G>T]CAGAATAGAAAATTATATATGC |
| SNP | 39927 | 549709 | CTGCTTTTTCTCTCTTTGTACCCACAC[T>C]GGGCGAACAAAGCTGGAAACAAA |
| SNP | 45826 | 555608 | AGCTCACACTTGTAAGTGGGAACATG[C>T]GGTATTTCGTTTTCTGTTCCTGC |
| SNP | 50197 | 559979 | GCTACCTGGATCCAAGAATGG[G>T]AAAGAGTGAGGAGTATGC |
| SNP | 50334 | 560116 | AGGTGGATCACGAGGTCAAGAGATC[G>A]AGACCATTCTGGCCAACGTGG |
| SNP | 50493 | 560275 | GTGGAGGTTGCAGTGAGCCGAGACTGC[G>A]CCACTGCACTCCAGCCTGG |
| SNP | 50632 | 560414 | AGGATTTGGGGCATGAAATAGGAGCT[G>A]CAGGTTGGAGAACATCACAA |
| SNP | 50835 | 560617 | CCTGCAGCCTGGTGCAGGGATGCAG[A>C]AGAATGTACTTATTCTGTGC |
| SNP | 50955 | 560737 | GCTGACAGCTAAGTGCAAG[G>C]TGGAATCAGAGATATCTTAATAGAT |
| CA-repeat | 51022-51049 | 560804-31 | AAGAACCAGGCATCTTAGA[(CA)6TA(CA)7>(CA)8]CGTGCACGAGCACCCACGC |
| (NM45) | | | |
| SNP | 51217 | 560999 | TTAACTTCAGTTTTAGAACAC[G>A]ACAAATCTTATTTTTATTATACAACTAC |
| SNP | 51476 | 561258 | GCTCTTGCCAGCTCACAGCA[A>G]AAGGGCCTCTAATTCAGAGAAAAAGGA |
| SNP | 51536 | 561318 | CCAGTCATGTACCTGTTATG[C>T]TTTCATATAGGCATCAGTTAGACCC |
| SNP | 51861 | 561643 | GACCAGACAGAAAGCAGCCTCAGGA[C>G]GGGGCCAGAGCATGAGACTAG |
| SNP | 51884 | 561666 | GGGCCAGAGCATGAGACTAGG[G>T]GTGGTGGCAGCTGGGTTTTAT |
| SNP | 51975 | 561757 | TGCATGCCATACAACC[G>C[GGAATTCCTACATTACTGA |
| insertion | 52266-52287 | 562068-69 | TAGCATAGGCCATTGCATTCATCTT[CC]CCCCCCAGTACCTGAATCTGTGA |
| SNP | 52573 | 562355 | TGCAATGTTCATGACCCAAACAAAGCT[G>A]AGCCTCTGTGTGGCCACTCTCA |
| SNP | 52776 | 562558 | TTTCAAGGATAAAGGGAAGG[G>C]GGTAGAAAGGGGAAGTGAGGAGAGGT |
| SNP | 53803 | 563585 | GGTTTCTTTTCGGAAGCT[C>T]GCTGCTGGGTGTTCGGA |
| SNP | 53922 | 563704 | GTGGATGGTTTCTTGGC[T>C]GTTGGGGCTCTCTGGC |
| SNP | 54148 | 563930 | CAAGCATCTTGCAA[G>A]GGCCTTGCTCTGTGAGGGG |
| SNP | 54199 | 563981 | ACACACAGTAGGCCAGGTCCTGC[T>C]GCACCTGCTCAGTTTAGGCTGTGTGG |
| SNP | 54641 | 564423 | AGATCCCCAGTGGCAAC[G>C]ATGAGATTAGACCGG |
| SNP | 54751 | 564533 | GCAGAACTTGCCAAGGG[T>C]AGGTGAAGCAGCTGCA |
| SNP | 55000 | 564782 | TTGTGCTCCTGTTCTTCCC[G>A]GAGAACTCGGCGCTG |
| SNP | 55134 | 564916 | AGTGGGGAGCATTGTGGGAGACTCCTTC[G>A]GGTCCCCCTGACTGGTTCTCT |
| SNP | 56683 | 566465 | AATAAAGCCCAGAAATA[C>T]GAGACTGTCTTCCCCAGT |
| SNP | 56856 | 566638 | CCAGGTGTGGTGGTGCACGC[C>T]TGTAGTCCCAGCTATTCAGGAGGCTGAG |
| SNP | 57790 | 567572 | TGGTGGAGAAGGGTGCCTTTCTT[A>C]ATTTTAGTTTTTATTATTTTTATTG |
| SNP | 60559 | 570341 | TATTTGTATTGCACTTA[C>G]AGTGAAAATTATCTAATTATATTA |
| SNP | 60604 | 570386 | CTATTTAATTACAATTGC[A>T]TCAAGCTCCAATAAACAATCC |
| SNP | 61165 | 570947 | AGCTTTCTTCTGGCCATGC[G>A]ACTGTTCTGCTAGTCTGCTC |
| SNP | 64559 | 574341 | CTGCATTCTTTGGGAAGCGGATG[C>T]TGAAACAGAGTCAGGTATACAAAAAT |
| SNP | 65171 | 574953 | CATCCTTTAAAAGAGAA[A>G]ACAAGTTGTCTATGTC |
| SNP | 65857 | 575639 | AAATTTTAATTGGCTAAGTACC[T>G]GACATCAGGTTGGGCCATTCAGTTTTAT |
| SNP | 66164 | 575946 | TCCCTTCAGGAAAGCCAGAGGTCATCTG[C>T]AACACCACAA |
| SNP | 66190 | 575972 | ACCACAATTAAGGATAAAAGGA[C>T]CTTATATTATCAGAAATTTCAATGTCT |
| SNP | 66526 | 576308 | ACTGAGTATCTTGCCAGGCA[C>T]TATGAAAAAAGATAAAACTATAAACATGA |
| SNP | 66902 | 576664 | AAAAATAATAATCTCTGTTATCCCAT[A>G]GTATAGTGGAATAGGAAAGAAATA |
| SNP | 67857 | 577639 | TAAATAAAGTTTTATTGGA[T>A]TACAGCCAGGCCCATCC |
| SNP | 67919 | 577701 | TTCCACTCTACAATGGCAGA[T>C]TTGAGTAGCTGTTA |
| SNP | 72270 | 562052 | AATAAGGTAAAATAAGGAGGATGC[G>A]GTGACTGAGTATTGAGATGTTCTGA |
| SNP | 75115 | 584897 | CAGTGTTGAAGGCAGTCGA[G>A]CACTGACTTCTATAGTATGG |
| SNP | 76060 | 585662 | CAGCCCCCCATTCACCCTTCCACAA[C>T]AGCAGGGGCAGGGCA |
| SNP | 76101 | 585883 | ACAACAGCAGGGGCAGGGCACCCTA[G>C]AGCATAAATAAT |
| SNP | 81912 | 591694 | ACTGGTCAACATCTTGACAGATATTA[A>T]TTGGCGATTCACTGG |
| SNP | 82203 | 591985 | GAGCCACAAATGAGCAATGCTAAGGAC[A>G]TAAAGCATTCATATTTCAAATG |
| SNP | 82332 | 592114 | CTTTCACTTCTCAAATGAACTCAACG[C>T]TGGCTACTGCTCTTTTTTGTAAA |
| SNP | 82922 | 592704 | TTGTATATATATTCATAAGTAAAATT[T>A]TAATTTTTTTCATTGGTTTTATT |
| SNP | 83552 | 593334 | AAGTGGCAACATATTTATGAACTAGT[C>T]TCAAAGGTCATACACCATCACTT |
| SNP | 85227 | 595009 | GCTCACCCATGTTTTTAACA[T>C]CACAGCCAGCACCATAGTAA |
| SNP | 85271 | 595053 | AAGCTAGTTAACACTCCAAGCCCCA[G>A]TGTTAAACTCTGCCTGCATGTGTC |
| SNP | 107610 | 617392 | CCTAATTCTAAAACCAATGC[T>C]ATTAAAAGAAAGGAAAACTA |
| SNP | 110989 | 620771 | AATGCCTTCTTTAATTTTTACTT[T>C]CTTGATATACCTTTGGCCATCCCTCT |
| SNP | 111012 | 620794 | CTTTCTTGATATACCTTTGGCCATCC[C>T]TCTATTTCCACACTTAGATATCT |
| SNP | 112030 | 621812 | TATTCTATGCTCTTCCCTCTTCTT[G>C]AATTCTTAATTTTAATTCAATTCTC |
| SNP | 112037 | 621819 | TCTTCCCTCTTCTTGAATTCT[T>G]AATTTTAATTCAATTCTC |
| SNP | 112283 | 622065 | TTCTTTTAGGTAACTTTTAA[A>T]TTTTTTTACCTGCCTTAAA |
| SNP | 112726 | 622508 | CCAAGAGATAATTTTTTCTT[T>A]TAGTTTTCAGAGGAGCCTTC |
| SNP | 112859 | 622641 | GTTTGTTGTACAGATTATTT[A>C]ATCACCCAGGTATTTTTTTT |
| SNP | 113428 | 623210 | TAATCATTCCTCAGAACCTCCT[C>G]CCTATGGTGGCTTTCACAACATTAGTG |
| SNP | 113645 | 623427 | TCTACTCCCTCTGGATTTGA[A>T]CCTAATGTTACAAAGTGGA |
| SNP | 113944 | 623726 | AGTGGATGAAAAAGGGGAGC[T>G]CTCTGATGGTGTCAGTGCTG |
| SNP | 114945 | 624727 | GGAATACCTGTTCTCATTTA[A>G]GACCACTGGTTTCCAGGCTT |
| SNP | 115192 | 624974 | CATTTCTGTCTTTAGGTTGTGCTGCT[C>G]TACGCCTCTACCTACGTCCTGGT |
| SNP | 115628 | 625410 | CATGCATACCTGTCCAAAGAAA[T>C]GAACTCTCCCACACTAACTTCTCATTT |
| SNP | 116032 | 625814 | GGGCCCTGGGGAGCAGTAAT[G>A]CCCTAGAGGAAGGGTGAGTA |
| SNP | 116464 | 626246 | CTTATTGAAATATAAAATGT[G>A]CAGCCCTTTGATATGACTTC |
| SNP | 116515 | 626297 | TCTGGAGAAACACCCACAC[G>A]TGTGCATGTATACATATGT |
| SNP | 116926 | 626708 | TAGAGCCATGGGCAGTAATA[T>C]GCATCAACATGAACATATC |
| SNP | 117276 | 627058 | ATTTATTTTGATCATGCTTG[T>A]TATGCCTACTGTACAGCATAAGACATATG |
| SNP | 123667 | 633449 | ACATTAAGTGGTATGCATTG[G>A]ATATGTATAGCTTTTTATAT |
| SNP | 123770 | 633552 | TGTTTCCTGGGTTTCTTCTCGGTC[A>G]TTTGGTGTGTCTGTTTTGTGAAGGG |
| SNP | 123768 | 633570 | GGTCATTTGGTGTGTCTGTTTT[G>C]TGAAGGGTTGAGACCTGTACCAAGTTT |
| SNP | 128017 | 638799 | CAGAGAAAGAGGCCTCCCTG[A>G]AGGTCACCAGGGAGGGTGGAAGG |

| | | |
|---|---|---|
| Type of | Alleles detected in haplotypes H1-H7 | Location in GPRA |

| | | | | | | | | | |
|---|---|---|---|---|---|---|---|---|---|
| polymorphism | H1 | H2 | H3 | H4 | H5 | H6 | H7 | and AAA1 exons | Amino acid change |

| | | | | | | | | | |
|---|---|---|---|---|---|---|---|---|---|
| SNP | C | C | C | C | C | C | T | | |
| SNP | A | A | A | A | A | A | G | | |
| insertion | no ins | no ins | ins | ins | ins | ins | ins | | |
| (NM50) | | | | | | | | | |
| SNP | G | G | G | G | G | G | A | | |
| SNP | A | A | A | A | A | A | T | | |
| SNP | T | T | T | T | T | T | C | | |
| deletion | no del | no del | del | no del | del | del | no del | | |
| SNP | C | C | C | C | C | C | T | | |
| SNP | A | A | A | A | A | A | G | | |
| SNP | C | C | C | C | C | C | T | | |
| SNP | A | A | A | A | A | A | T | | |
| SNP | T | T | T | T | T | T | C | | |
| SNP | G | G | A | G | A/G | A | G | | |
| SNP | G | C | G | C | C | G | C | | |
| SNP | C | A | C | A | A/C | C | C | | |
| SNP | T | C | C | C | C | C | C | | |
| SNP | C | C | C | C | C | C | G | | |
| SNP | A | G | A | G | G | A | A | | |
| SNP | C | C | C | C | C | C | G | | |
| SNP | G | A | G | G | G | G | G | | |
| SNP | A | G | A | G | G | A | A | | |
| SNP | C | T | C | T | T | C | C | | |
| SNP | G | T | G | T | T | G | G | | |
| SNP | G | A | G | A | A | G | G | | |
| SNP | C | G | C | G | G | C | C | | |
| SNP | A | G | A | G | G | A | A | | |
| SNP | G | A | G | G | G | G | G | | |
| SNP | T | C | T | C | C | T | T | | |
| [symbol] AAA- | 8 | 7 | 8 | 7 | 7 | 8 | 8 | | |
| repeat | | | | | | | | | |
| (NM49) | | | | | | | | | |
| Z,899 eletion | no del | del | no del | del | del | no del | no del | | |
| SNP | C | C | C | A | A | C | C | | |
| SNP | C | T | C | T | T | C | T | | |
| [symbol] eletion | no del | del | no del | del | del | no del | no del | | |
| (NM51) | | | | | | | | | |
| SNP | C | G | C | G | G | C | C | AAA1 exon 10 | no change (3′UTR) |
| SNP | G | G | T | G | T | T | G | AAA1 exon 10 | no change (3′UTR) |
| SNP | G | C | G | C | C | G | G | AAA1 exon 10 | no change (3′UTR) |
| [symbol] eletion | no del | del | no del | del | del | no del | no del | AAA1 exon 10 | no change (3′UTR) |
| (NM52) | | | | | | | | | |
| [symbol] eletion | no del | no del | no del | del | no del | no del | no del | AAA1 exon 10 | no change (3′UTR) |
| (NM52241) | | | | | | | | | |
| SNP | C | T | C | T | T | C | C | AAA1 exon 10 | no change (3′UTR) |
| SNP | G | A | G | G | G | G | G | AAA1 exon 10 | no change (3′UTR) |
| SNP | C | T | C | T | T | C | C | | |
| [symbol] T-repeat | 12 | 10 | 12 | 10 | 10 | 12 | 12 | | |
| (G42102) | | | | | | | | | |
| [symbol] eletion | no del | del | no del | no del | no del | no del | no del | | |
| [symbol] sertion | no ins | ins | no ins | ins | ins | no ins | nd | | |
| [symbol] NP | T | G | T | G | G | T | T | | |
| [symbol] eletion | no del | del | no del | del | del | no del | no del | | |
| (NM53) | | | | | | | | | |
| [symbol] NP | T | C | T | C | C | T | C | | |
| [symbol] NP | A | G | A | G | G | A | A | | |
| [symbol] NP | G | C | G | C | C | G | C | | |
| [symbol] NP | T | C | T | T | T | T | C | | |
| [symbol] NP | T | T | T | C | T | T | T | | |
| [symbol] NP | T | C | T | T | T | T | T | | |
| [symbol] NP | G | A | G | G | G | G | G | | |
| SNP | C | C | C | A | C | C | C | | |
| SNP | A | G | A | A | A | A | A | | |
| SNP | G | G | G | G | G | G | A | | |
| SNP | G | G | G | T | T | G | G | | |
| SNP | A | A | A | A | A | A | A | G | |
| SNP | G | G | G | C | C/G | G | G | | |
| SNP | A | T | A | A | A | A | A | | |
| SNP | G | A | G | A | A | G | A | | |
| SNP | G | A | G | G | G | G | G | | |
| SNP | G | G | G | G | G | A | | | |
| SNP | C | C | C | A | A | C | C | | |
| SNP | C | A | C | A | A | C | A | | |
| SNP | T | C | T | C | C | T | C | | |
| SNP | T | A | T | T | T | T | A | | |
| SNP | G | A | G | G | G | G | G | | |
| SNP | T | C | T | C | C | T | C | | |
| SNP | G | T | G | G | G | G | G | | |
| SNP | C | T | C | T | T | T | T | | |
| deletion | no del | no del | no del | del | del/no del | no del | no del | | |
| (NM54) | | | | | | | | | |
| SNP | T | T | T | T | T | C | C | AAA1 exon 9 | no change (noncoding exon) |
| SNP | A | A | A | A | A | G | G | AAA1 exon 9 | no change (noncoding exon) |
| SNP | G | G | G | G | G | A | A | AAA1 exon 9 | no change (noncoding exon) |
| SNP | T | T | C | T | T | T | T | AAA1 exon 9 | no change (noncoding exon) |
| SNP | A | A | A | A | A | T | T | AAA1 exon 9 | no change (noncoding exon) |
| SNP | T | T | T | C | C | T | T | | |
| SNP | G | G | G | G | G | T | T | | |
| SNP | T | C | T | T | T | T | T | | |
| SNP | C | C | C | C/T | C | C | C | | |
| SNP | C | C | C | C | C | T | T | | |
| SNP | A | A | A | A | A | G | G | | |
| TAAA | 10 | 11 | 10 | 11 | 10/11 | 11 | 10 | | |
| repeat | | | | | | | | | |
| (G42100) | | | | | | | | | |
| SNP | C | C | C | T | T | C | C | | |
| SNP | G | G | G | G | G | A | A | | |
| CA-repeat | 13 | 13 | 13 | 12 | 12 | 17 | nd | | |
| (07S497) | | | | | | | | | |
| SNP | C | T | C | C | C | T | T | | |
| SNP | G | G | G | A | A | G | G | | |
| SNP | T | T | T | T | T | C | C | | |
| SNP | G | G | G | G | G | G | C | | |
| SNP | G | G | G | G | G | A | A | | |
| SNP | G | A | G | G | G | A | A | | |
| SNP | C | C | C | A/C | C | C | C | | |
| SNP | T | T | T | T | T | C | C | AAA1 exon 6 | Asn>Ser |
| SNP | T | C | T | T | T | T | T | | |
| SNP | C | C | C | A | A | C | C | | |
| SNP | G | G | G | A | A | G | G | | |
| SNP | G | G | G | C | C | G | G | | |
| SNP | A | C | A | A | A | A | A | | |
| deletion | no del | no del | no del | no del | no del | del | del | | |
| SNP | C | C | nd | C | C | T | T | | |
| SNP | G | G | G | A | G | G | G | | |
| SNP | C | A | C | A | A | A | A | | |
| SNP | G | G | G | G | G | T | T | | |
| SNP | G | G | G | G | G | A | A | | |
| SNP | A | A | A | A | A | G | G | | |
| SNP | C | T | C | C | C | T | T | | |
| SNP | C | C | C | C | C | T | T | | |
| deletion | no del | del | no del | no del | no del | no del | no del | | |
| SNP | G | A | G | G | G | A | A | | |
| SNP | G | G | G | G | G | T | T | | |
| SNP | T | C | T | C | C | C | C | | |
| SNP | C | T | C | T | T | T | T | | |
| SNP | G | T | G | G | G | T | T | | |
| SNP | G | A | G | G | G | A | A | | |
| SNP | G | G | G | G | G | A | A | | |
| SNP | G | A | G | G | G | A | A | | |
| SNP | A | A | A | A | A | C | C | | |
| SNP | G | C | G | G | G | G | G | | |
| CA-repeat | wt | wt | wt | wt | wt | (CA)8 | (CA)8 | | |
| (NM45) | | | | | | | | | |
| SNP | G | A | G | G | G | A | A | | |
| SNP | A | G | A | A | A | A | A | | |
| SNP | C | C | C | C | C | T | T | | |
| SNP | C | G | C | C | C | G | G | | |
| SNP | G | G | G | G | G | T | T | | |
| SNP | G | C | C | C | C | C | C | | |
| insertion | no ins | no ins | no ins | ins | ins | no ins | no ins | | |
| SNP | G | A | G | G | G | G | G | | |
| SNP | G | G | G | G | G | G | C | | |
| SNP | C | C | C | T | T | C | C | | |
| SNP | T | C | C | C | C | C | C | | |
| SNP | G | G | G | A | A | A | A | | |
| SNP | T | T | T | T | T | C | T | | |
| SNP | G | G | G | C | C | C | C | | |
| SNP | T | T | T | C | C | C | C | | |
| SNP | G | G | G | A | A | G | G | | |
| SNP | G | A | G | G | G | G | G | | |
| SNP | C | C | C | T | T | C | C | | |
| SNP | C | T | C | C | C | C | C | | |
| SNP | A | A | A | A | A | C | A | | |
| SNP | C | C | C | G | G | G | G | | |
| SNP | A | A | A | A | A | T | T | | |
| SNP | G | G | G | G | G | A | A | | |
| SNP | C | C | C | C | C | T | C | AAA1 exon 4 | no change (5′UTR) |
| SNP | A | G | A | G | G | G | G | | |
| SNP | T | G | T | T | T | T | T | | |
| SNP | C | C | C | T | T | T | T | | |
| SNP | C | C | C | T | T | T | T | | |
| SNP | C | T | C | C | C | C | C | | |
| SNP | A | G | A | A | A | A | A | | |
| SNP | T | T | T | A | A | A | A | | |
| SNP | T | T | T | C | C | T | T | | |
| SNP | G | A | G | G | G | G | G | | |
| SNP | G | G | G | G | G | A | A | | |
| SNP | C | C | C | C | C/T | C | C | | |
| SNP | G | G | C | C | G | G | | | |
| SNP | A | T | A | T | A/T | T | T | GPRA exon 3 | Asn>Ile |
| SNP | A | G | A | G | A/G | G | G | | |
| SNP | C | C | C | T | C/T C | C | | | |
| SNP | T | T | T | T | T | A | A | | |
| SNP | C | C | C | C | C | T | T | | |
| SNP | T | C | T | C | C/T | C | C | | |
| SNP | G | G | G | G | G | A | A | | |
| SNP | T | C | C | C | C/T | C | C | | |
| SNP | T | T | T | T | T | C | C | | |
| SNP | C | T | C | C | C | C | C | | |
| SNP | G | G | G | G | G | C | C | | |
| SNP | T | G | T | T | T | T | T | | |
| SNP | A | T | A | T | T | T | T | | |
| SNP | T | T | T | A | A | T | T | | |
| SNP | A | C | A | C | C | C | C | | |
| SNP | C | C | C | C | C | G | G | | |
| SNP | A | T | A | T | A/T | T | T | | |
| SNP | T | T | T | G | G | T | T | | |
| SNP | A | A | A | G | A/G | A | A | | |
| SNP | C | G | C | C | C | C | C | GPRA exon 4 | no change |
| SNP | T | T | T | T | T | C | C | | |
| SNP | G | G | G | A/G | G | G | G | | |
| SNP | G | G | G | A | A/G | G | G | | |
| SNP | G | G | G | G | G | A | A | | |
| SNP | T | T | T | T | T | C | C | | |
| SNP | T | T | T | T | T | A | A | | |
| SNP | G | G | G | A | A | G | G | | |
| SNP | A | G | A | A | A | A | A | | |
| SNP | G | C | G | G | G | G | G | | |
| SNP | A | A | A | G | nd | G | nd | | |

^{nd}no sequencing/genotyping result is available for the site
del = deletion
ins = insertion

TABLE 4
Primers used in the cloning of GPRA.

| Primer | Sequence | Exon | Splice variant |
|---|---|---|---|
| JEGE1F1 | tctgtgcctccgttcagcag | 5′UTR | — |
| JEGE1F2 | aagctggactccctcactcagc | 5′UTR | — |
| JEGE1F3 | agcaaggacagtgaggctcaacc | 5′UTR | — |
| JEGE9aR1 | ctggcatgaataactggggagttc | 3′UTR | A |
| JEGE9aR2 | tttgtcttgtgcatctcccaggta | 3′UTR | A |
| JEGE9bR1 | tatagccctccctggtgaatctga | 3′UTR | B |
| JEGE9bR2 | gtgccctggtaagcagtgagaagt | 3′UTR | B |
| JEGE5F1 | tgagcaattgataactctgtgggtcctc | E2a | — |
| JEGExR2 | AAATAAGCTGTGGCATCCTCATCCAG | 3′UTR | C |
| JEGExR1 | TGGCATCCTCATCCAGGGATATTTGC | 3′UTR | C |
| VAUE3R1 | GGAAGGCCACGATGGTCATGTATGG | E5 | ′ |
| Vau8R1 | ATGACGAGGGTGGGGTGAAGTTGG | 3′UTR | A |
| Vau8R2 | GAATGGTGGGGAAGGAAGGCGTTT | 3′UTR | A |
| Vau1000R1 | ggccatcctgctgtgacccatttt | 3′UTR | B |
| AS8FX1 | ATGAGATGCAGATTCTGTCCAAG | E9a | A |
| AS8FX2 | TGCACAAGACAAATGTTCTAATGA | 3′UTR | A |
| AS8F5.1 | CAGCTATAACCGAGGACTCATCTC | E7 | — |
| AS8F7.1 | TGTTGGAGTCCATACTTCCTGTT | E8 | — |
| VAUE3R2 | AAAAGACAGGCTCCAGGCGATCACA | E5 | — |
| A2E2F | gattcttccccagtggcttgcactgaa | E1 | — |
| A3E3R | TGATGGCCAGCTGAGTCACAAAGAAGG | E2a | — |

TABLE 5

Primer pairs used in re-sequecing of the exons
and exon/intron bondaries of GPRA

| | | |
|---|---|---|
| Primer | Forward primer | Reverse primer |

| | | |
|---|---|---|
| GPRA ex1 | actcagctgcaggagcaag | tgacactcttaagttccagcagtc |
| GPRA ex2a | aggaggaagaaatccagcct | tgaccgatgcgttacatttt |
| GPRA ex2b.1 | gccatacattgttagtaacctgaaa | cctcatccagggatatttgc |
| GPRA ex2b.2 | ttcctaccaacaagaactccaa | cgatgatgaattagaacatacaacttt |
| GPRA ex3 | taagtcaaagaactcctaccttgc | agcaaaggaaatacattaaaaatcaaa |
| GPRA ex4 | ctgccctctttcacccagta | agccacccaccttccttagt |
| GPRA ex5 | gcttctgttcaagcttcccttt | gtgtggtcctgtcctgacg |
| GPRA ex6 | tggatcctcatggtcactttc | tctctgctggcatagcttga |
| GPRA ex7 | ttgagagagtctgagcattcca | ccaaattattcaacccatagcc |
| GPRA ex8 | tgacatcaatgctccaaacaa | tgtcatgattaaggcggtttc |
| GPRA ex9a | ttaacatgtctacttgccttttca | tctgcaaaccgaggctatct |
| GPRA ex9b | gcagagctgtcacccaaaat | agcctgggcaacaagagtaa |

TABLE 6A
Primers used in GPRA SNP genotyping.

| | Nucleotide | | | | |
|---|---|---|---|---|---|
| SNP ID | NT_000380.3 | Type | Fw primer | Rev primer | Pe primer |
| dbSNP: | 591694 | A/T | GGCCATCTGATAAAGCAGGA | TGATGCATAGGAATGCAAGG | AGTCTCCAGTGAATCGCCAA |
| 324981 | | | | | |

TABLE 6B

Primer pairs, rectrction enzymes, and corresbonding allele
sizes used in genotyping of GPRA exonic SNPs.

Six digit number in the name of the SNP shows its
position in the genomic contig NT_000380.

| | | | | Product | | Product | |
|---|---|---|---|---|---|---|---|
| Marker | Forward primer | Reverse primer | Enzyme | size* | Allele 1 | size* | Allele 2 |
| SNP_640764 | aactgactaaactaggtgccacgtc | tagcacaatgcctgccctat | Sapl | 340 | t | 290 + 50 | c |
| | gtccatacatgaccatcgtgctctt | | | | | | |
| SNP_662764 | tgtctacctgttggcctgtg | gtcttgtgcatctcccaggt | Mboll | 455 | a | 240 + 215 | g |
| SNP_662803 | catggagaaggaaggtcagg | ctagcactggcactgcccta | Ddel | 270 | c | 220 + 50 | t |
| SNP_663133 | atgactgcatgcactgctta | tctgcaaaccgaggctatct | SfaNl | 400 | t | 140 + 260 | c |

*product sizes are approximitations

TABLE 7
SNPs found in the exons of six splice variants of GPRA.

Position of the SNPs

| | | seq2, | seq4, | seq6, | seq8, | seq10, | seq12, | seq14, |
|---|---|---|---|---|---|---|---|---|
| Exon | seq1 | A variant | B long variant | B short variant | C variant | D variant | E variant | F variant |
| ex2b | — | — | — | — | 585 | — | — | — |
| ex2b | — | — | — | — | 655 | — | — | — |
| ex2b | — | — | — | — | 681 | — | — | — |
| ex3 | 81912 | 448 | 448 | 415 | — | — | 448 | — |
| ex4 | 115192 | 524 | 524 | 491 | — | 420 | — | — |
| ex5 | — | 776 | 776 | 743 | — | 672 | 682 | 578 |
| | — | | | | | | | |
| ex6 | — | 851 | 851 | 818 | — | — | — | 653 |
| ex9a | — | 1159 | — | — | — | — | — | 961 |
| ex9a | — | 1199 | — | — | — | — | — | 1001 |
| ex9a | — | 1529 | — | — | — | — | — | 1331 |
| ex9b | — | — | 1206 | 1173 | — | — | — | — |
| ex9b | — | — | 1225 | 1192 | — | — | — | — |
| ex9b | — | — | 1330 | 1297 | — | — | — | — |
| ex9b | — | — | 1338 | 1305 | — | — | — | — |

| Exon | Sequence around the polymorphism | Amino acid change in different variants |
|---|---|---|
| ex2b | TTTTTCACTCCTATAA[C > T]CGTAGAAGTAGAG | no change (3′UTR of the C variant) |
| ex2b | CCTGGATGAGGATGCC[A > C]CAGCTTATTTTCA | no change (3′UTR of the C variant) |
| ex2b | TTTTCATTATATTTCTTC[G > A]ATTACAGTGTGGTAATG | no change (3′UTR of the C variant) |
| ex3 | TTGACAGATATTA[A > T]TTGGCGATTCACT | Asn > Ile (A, B and E variants) |
| ex4 | CTGTCTTTAGGTTGTGCTGCT[C > G]TACGCCTCTACCTACGTCC | no change (coding region of the A, |
| | | B and D variants) |
| ex5 | TACATGACCATCGTGGCCTT[C > T]CTGGTGTACTTCATCCCTC* | no change (coding region of the A, |
| | | B and F variants |
| | | or in the 3′UTR of the D and E |
| | | variants |
| ex6 | TATTTGGATTAAAAG[C > G]AAAACCTACGAAACAGT* | Ser > Arg (A, B and F variants) |
| ex9a | CAGGGAGC[A > G]AAGATCACAGGATTCCAGAATG | Gln > Arg (A and F variants) |
| ex9a | GACGTTCCGGGAGAGAAC[T > C]GAGAGGCATGAGATGCAGATTC* | no change (coding region of the A and |
| | | F variants) |
| ex9a | CCAGTGAACACAGGCAT[T > C]AGTGGTCCAGGGTCCTGGCTT | no change (3′UTR of the A and F |
| | | variants) |
| ex9b | CTAATGCTCTGCCCTCAA[C > A]GAGAGAACTGGAAGGGTA | no change (coding region of the B |
| | | variants) |
| ex9b | AGAGAACTGGAAGGGTA[C > T]TTGGCCAGGTGTACCTTCCTGG | Thr > Ile (B variants) |
| ex9b | TCTCACTGCTTACCAGGGCACA[A > T]GGACACC | no change (3′UTR of the B variants) |
| ex9b | GGACACC[A > G]GTGGTTCCCAAAATGGGTC | no change (3′UTR of the B variants) |

*at these sites contig NT_00380 contains an allele that associates with high serum lgE level and/or asthma

TABLE 8
Best haplotype associations of GPRA for high serum IgE level in the data set of 304 disease
associated and 220 control chromosomes when one gap (marked with an asterisks) was allowed in the
haplotype patterns.

| | | | | GPRA markers | | | | |
|---|---|---|---|---|---|---|---|---|
| | | | | SNP591694 | SNP640763 | SNP662763 | SNP662803 | SNP663133 |
| High IgE | Control | Conf | χ² | Exon 3 | Exon 5 | Exon 9A | Exon 9A | Exon 9A |
| 51 | 16 | 0.761 | 10.3 | — | C | — | — | — |
| 44 | 14 | 0.759 | 8.5 | T | C | — | — | — |
| 40 | 12 | 0.769 | 8.5 | — | C | * | T | — |
| 39 | 12 | 0.765 | 7.9 | — | C | * | T | C |
| 39 | 12 | 0.765 | 7.9 | — | C | G | — | — |
| 39 | 12 | 0.765 | 7.9 | — | C | G | T | — |
| 38 | 12 | 0.76 | 7.3 | — | C | G | * | C |
| 38 | 12 | 0.76 | 7.3 | — | C | G | T | C |
| 35 | 11 | 0.761 | 6.8 | T | C | * | T | — |
| 34 | 11 | 0.756 | 6.2 | T | C | * | T | C |
| 34 | 11 | 0.756 | 6.2 | T | C | G | — | — |
| 34 | 11 | 0.756 | 6.2 | T | C | G | T | — |
| 33 | 11 | 0.75 | 5.7 | T | C | G | * | C |
| 33 | 11 | 0.75 | 5.7 | T | C | G | T | C |

High IgE, number of disease-associated haplotypes with a specific pattern;
Control, number of control haplotypes with a specific pattern;
Confidence, percentage of haplotypes with the specific pattern that is associate with disease,
chi-square value for disease association of the specific haplotype pattern.

TABLE 9

Primers used for cloning of full
length cDNAs for AAA1.

| | | |
|---|---|---|
| Primer | Exon | Sequence |

| | | |
|---|---|---|
| IF | 1 | 5′AGAATGAGTCTCTGATGACTTT 3′ |
| IIF | 2 | 5′ACTTGCTGTTCATAGAATTGCAA 3′ |
| SF9 | 4 | 5′TGACTTCTCCCCAGATTTTTGTAT 3′ |
| exIF | 5 | 5′ACACATACAAAGTGCCTACCACAT 3′ |
| SR13 | 9 | 5′TTGAAACTGTATTTCCCATATTGC 3′ |
| ASKAF/R | 10b | 5′AAATGCAATAAAAATGCGGAACTA 3′ |
| XR | 11 | 5′GAGTCATTAGTCCAGAGAACAT 3′ |
| XIR | 12 | 5′CTGCTTGGAACAGTGTATATC 3′ |
| XIIIR | 14 | 5′TGGTCTACGTAGAATTCAGAGTA 3′ |
| XVR | 16 | 5′CATGTGTTAATTGTGTCTTCACT 3′ |
| SR4 | 19 | 5′GGGTGTCATTTACACGAACAATAA 3′ |
| SF10 | 6 | 5′CAGTTCAGTCACTGCAATCTTCAT 3′ |

TABLE 10
Primers used in SNP genotyping with SBE method.

| | | | |
|---|---|---|---|
| SNP | forward | reverse | extension |

| | | | |
|---|---|---|---|
| 517278 | TTATGAGCTAAAGTGCCAATTAAA | AGGAAGGTGGCAGTGAACTC | CCCTCCTATACATTACCTGAA |
| 549709 | TCCCCGTCTCCTCTAGTCTTC | TGAGATGCTGTCTCTAAAATAAATAGA | TTCTCTCTTTGTACCCACAC |
| 570341 | TGTTGGTTCGATGAGAGCAT | AAGTGGGGAACAAACACTGG | GTACCTATATTTGTATTGCACTTA |

TABLE 11
Exon-intron structures and splice junction sites of AAA1. Gray area shows the exons and introns located in AST1.

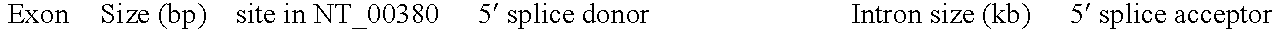

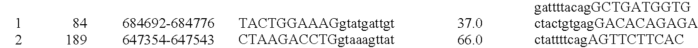

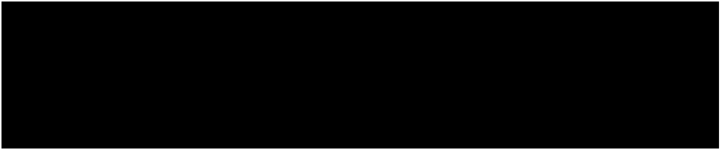

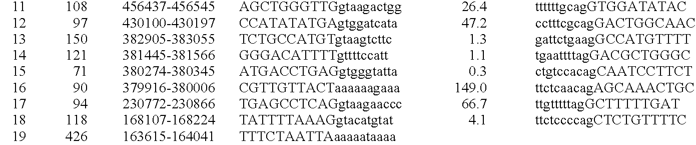

TABLE 12
Best haplotype associations of AAA1 for high serum IgE level in the data set of 304 disease associated and 220 control
chromosomes when one gap (marked with an asterisks) was allowed in the haplotype patterns.
Only the markers from the coding region or close to intron-exon boundaries were included.

| | | | Markers of the AST1 locus | | | | |
|---|---|---|---|---|---|---|---|
| | | | SNP_517278 | SNP_538567 | sNP_549709 | SNP_570341 | SNP_574953 |
| High | | | (C > T) | (G > A) | (T > C) | (C > G) | (A > G) |
| IgE | Control | X² | Exon 10a/10b | Intron 6 | Intron 5 | Intron 5 | Intron 3 |
| 59/187 (24%) | 18/179 (10%) | 13.548 | T | A | — | — | — |
| 49/190 (21%) | 14/177 (7.9%) | 12.5479 | T | A | * | G | — |
| 49/190 (21%) | 14/177 (7.9%) | 12.5479 | T | A | * | G | G |
| 117/130 (47%) | 55/181 (30%) | 12.5318 | T | — | — | — | — |
| 53/189 (22%) | 16/177 (9.0%) | 12.2929 | T | A | C | * | G |
| 53/189 (22%) | 16/177 (9.0%) | 12.2929 | T | A | C | — | — |
| 47/180 (21%) | 14/174 (8.0%) | 12.2377 | T | * | C | G | — |
| 47/180 (21%) | 14/174 (8.0%) | 12.2377 | T | * | C | G | G |
| 47/190 (20%) | 14/177 (7.9%) | 11.4627 | T | A | C | G | G |
| 47/190 (20%) | 14/177 (7.9%) | 11.4627 | T | A | C | G | — |
| 49/190 (21%) | 15/177 (8.4%) | 11.3004 | — | A | * | G | G |
| 103/136 (43%) | 47/174 (27%) | 11.2636 | T | * | C | — | — |
| 50/187 (21%) | 16/177 (9.0%) | 10.9929 | — | A | * | G | — |
| 47/189 (20%) | 15/177 (8.4%) | 10.3762 | — | A | C | G | — |
| 47/190 (20%) | 15/177 (8.4%) | 10.2633 | — | A | C | G | G |
| 60/184 (25%) | 23/179 (13%) | 9.0248 | — | A | — | — | — |
| 53/189 (22%) | 19/177 (11%) | 8.957 | — | A | C | * | G |

High IgE, number of disease-associated haplotypes with a specific pattern,
Control, number of control haplotypes with a specific pattern,
chi-square value for disease association of the specific haplotype pattern

TABLE 13
Haplotype specific AST1 polymorphisms for diagnostic testing.These markers can be
used for the haplotype identification of an individual without phase information.

| | Type of | Position in | Position in | Allele present in the | Allele present in |
|---|---|---|---|---|---|
| Haplotype | polymorphism | SEQ ID NO: 1 | NT_000380 | spesific haplotype | other haplotypes |
| H1 | SNP | 4631 | 514413 | T | C |
| H1 | SNP | 51975 | 561757 | G | C |
| H1 | SNP | 53922 | 563704 | T | C |
| H2 | SNP | 5634 | 515416 | A | G |
| H2 | SNP | 6739 | 516521 | A | G |
| H2 | SNP | 7550 | 517332 | A | G |
| H2 | deletion | 9199-9201 | 518981-83 | deletion of TCT | no deletion |
| H2 | SNP | 16980 | 526762 | C | T |
| H2 | SNP | 17147 | 526929 | A | G |
| H2 | SNP | 17435 | 527217 | G | A |
| H2 | SNP | 19272 | 529054 | T | A |
| H2 | SNP | 19452 | 529234 | A | G |
| H2 | SNP | 20309 | 530091 | A | G |
| H2 | SNP | 20789 | 530571 | T | G |
| H2 | SNP | 24869 | 534651 | C | T |
| H2 | SNP | 32976 | 542758 | C | T |
| H2 | SNP | 34716 | 544498 | C | A |
| H2 | deletion | 38850-52 | 548632-34 | deletion of CTC | no deletion |
| H2 | SNP | 50955 | 560737 | C | G |
| H2 | SNP | 51476 | 561258 | G | A |
| H2 | SNP | 52573 | 562355 | A | G |
| H2 | SNP | 55134 | 564916 | A | G |
| H2 | SNP | 56856 | 566638 | T | C |
| H2 | SNP | 65857 | 575639 | G | T |
| H2 | SNP | 66526 | 576308 | T | C |
| H2 | SNP | 66902 | 576684 | G | A |
| H2 | SNP | 72270 | 582052 | A | G |
| H2 | SNP | 111012 | 620794 | T | C |
| H2 | SNP | 112037 | 621819 | G | T |
| H2 | SNP | 115192 | 624974 | G | C |
| H2 | SNP | 123770 | 633552 | G | A |
| H2 | SNP | 123788 | 633570 | C | G |
| H3 | SNP | 22869 | 532651 | C | T |
| H4 | deletion | 7334-35 | 517116-17 | deletion of AT | no deletion |
| H4 | SNP | 16893 | 526675 | C | T |
| H4 | SNP | 17209 | 526991 | A | C |
| H4 | SNP | 26198 | 535980 | C/T* | C |
| H4 | SNP | 32124 | 541906 | A/C* | C |
| H4 | SNP | 36551 | 546333 | A | G |
| H4 | SNP | 116032 | 625814 | A/G* | G |
| H6 | SNP | 54199 | 563981 | C | T |
| H6 | SNP | 57790 | 567572 | C | A |
| H6 | SNP | 64559 | 574341 | T | C |
| H7 | SNP | 1 | 509783 | T | C |
| H7 | SNP | 93 | 509875 | G | A |
| H7 | SNP | 918 | 510700 | A | G |
| H7 | SNP | 983 | 510765 | T | A |
| H7 | SNP | 987 | 510769 | C | T |
| H7 | SNP | 1542 | 511324 | T | C |
| H7 | SNP | 1710 | 511492 | G | A |
| H7 | SNP | 1818 | 511600 | T | C |
| H7 | SNP | 1927 | 511709 | T | A |
| H7 | SNP | 2254 | 512036 | C | T |
| H7 | SNP | 4689 | 514471 | G | C |
| H7 | SNP | 5442 | 515224 | G | C |
| H7 | SNP | 18383 | 528165 | A | G |
| H7 | SNP | 18978 | 528760 | G | A |
| H7 | SNP | 19671 | 529453 | A | G |
| H7 | SNP | 28866 | 538648 | C | G |
| H7 | SNP | 52776 | 562558 | C | G |

*A rare polymorphism found only in H4 haplotype

TABLE 14
AST1 polymorphisms specific for the different haplotype combinations for diagnostic
testing. These markers can be used for the identification of certain haplotype combinations of an
individual without phase information.

| | | | | Allele present in | |
|---|---|---|---|---|---|
| Haplotype | Type of | Position in | Position in | the spesific haplotype | Allele present |
| combination | polymorphism | SEQ ID NO: 1 | NT_000380 | combination | in other haplotypes |
| H4 + H5 | SNP | 7125 | 516907 | A | C |
| H4 + H5 | SNP | 18927 | 528709 | T | G |
| H4 + H5 | SNP | 19268 | 529050 | C | G |
| H4 + H5 | SNP | 19712 | 529494 | A | C |
| H4 + H5 | deletion | 22122-23 | 531904-5 | deletion of TG | no deletion |
| H4 + H5 | SNP | 24007 | 533789 | C | T |
| H4 + H5 | SNP | 27404 | 537186 | T | C |
| H4 + H5 | SNP | 28785 | 538567 | A | G |
| H4 + H5 | SNP | 33350 | 543132 | A | C |
| H4 + H5 | SNP | 33798 | 543580 | A | G |
| H4 + H5 | SNP | 34362 | 544144 | C | G |
| H4 + H5 | insertion | 52286-87 | 562068-69 | insertion of CC | no insertion |
| H4 + H5 | SNP | 53803 | 563585 | T | C |
| H4 + H5 | SNP | 55000 | 564782 | A | G |
| H4 + H5 | SNP | 56683 | 566465 | T | C |
| H4 + H5 | SNP | 67919 | 577701 | C | T |
| H4 + H5 | SNP | 76101 | 585883 | C | G |
| H4 + H5 | SNP | 82332 | 592114 | T | C |
| H4 + H5 | SNP | 112726 | 622508 | A | T |
| H4 + H5 | SNP | 113944 | 623726 | G | T |
| H4 + H5 | SNP | 114945 | 624727 | G | A |
| H4 + H5 | SNP | 116464 | 626246 | A | G |
| H4 + H5 | SNP | 123667 | 633449 | A | G |
| H2 + H4 + H5 | SNP | 4012 | 513794 | A | C |
| H2 + H4 + H5 | SNP | 4961 | 514743 | G | A |
| H2 + H4 + H5 | SNP | 5850 | 515632 | G | A |
| H2 + H4 + H5 | SNP | 6312 | 516094 | T | C |
| H2 + H4 + H5 | SNP | 6392 | 516174 | T | G |
| H2 + H4 + H5 | SNP | 6485 | 516267 | A | G |
| H2 + H4 + H5 | SNP | 6522 | 516304 | G | C |
| H2 + H4 + H5 | SNP | 6646 | 516428 | G | A |
| H2 + H4 + H5 | SNP | 6760 | 516542 | C | T |
| H2 + H4 + H5 | deletion | 6821 | 516603 | deletion of T | no deletion |
| H2 + H4 + H5 | deletion | 7240-43 | 517022-25 | deletion of ACTT | no deletion |
| H2 + H4 + H5 | SNP | 7277 | 517059 | G | C |
| H2 + H4 + H5 | SNP | 7303 | 517085 | T | G |
| H2 + H4 + H5 | SNP | 7305 | 517087 | C | G |
| H2 + H4 + H5 | deletion | 7306-8 | 517088-90 | deletion of TGT | no deletion |
| H2 + H4 + H5 | SNP | 7496 | 517278 | T | C |
| H2 + H4 + H5 | SNP | 8490 | 518272 | T | C |
| H2 + H4 + H5 | SNP | 9649 | 519431 | G | T |
| H2 + H4 + H5 | deletion | 9782-9785 | 519564-67 | deletion of GTCT | no deletion |
| H2 + H4 + H5 | SNP | 11858 | 521640 | G | A |
| H2 + H4 + H5 + H7 | SNP | 3877 | 513659 | C | G |
| H2 + H4 + H5 + H7 | SNP | 7229 | 517011 | T | C |
| H2 + H4 + H5 + H7 | SNP | 10816 | 520598 | C | T |
| H2 + H4 + H5 + H7 | SNP | 12581 | 522363 | C | G |
| H2 + H4 + H5 + H7 | SNP | 19360 | 529142 | A | G |
| H2 + H4 + H5 + H7 | SNP | 19774 | 529556 | A | C |
| H2 + H4 + H5 + H7 | SNP | 20038 | 529820 | C | T |
| H2 + H4 + H5 + H7 | SNP | 20395 | 530177 | C | T |
| H6 + H7 | SNP | 22475 | 532257 | C | T |
| H6 + H7 | SNP | 22493 | 532275 | G | A |
| H6 + H7 | SNP | 22715 | 532497 | A | G |
| H6 + H7 | SNP | 22934 | 532716 | T | A |
| H6 + H7 | SNP | 24264 | 534046 | T | G |
| H6 + H7 | SNP | 26356 | 536138 | T | C |
| H6 + H7 | SNP | 26675 | 536457 | G | A |
| H6 + H7 | SNP | 28197 | 537979 | A | G |
| H6 + H7 | SNP | 28858 | 538640 | C | T |
| H6 + H7 | SNP | 31224 | 541006 | A | G |
| H6 + H7 | SNP | 32185 | 541967 | C | T |
| H6 + H7 | deletion | 34909 | 544691 | deletion of T | no deletion |
| H6 + H7 | SNP | 37327 | 547109 | T | G |
| H6 + H7 | SNP | 37415 | 547197 | A | G |
| H6 + H7 | SNP | 37685 | 547467 | G | A |
| H6 + H7 | SNP | 37959 | 547741 | T | C |
| H6 + H7 | SNP | 39343 | 549125 | T | G |
| H6 + H7 | SNP | 50493 | 560275 | A | G |
| H6 + H7 | SNP | 50835 | 560617 | C | A |
| H6 + H7 | CA-repeat | 51022-49 | 560804-31 | (CA)8 | (CA)6TA(CA)7 |
| H6 + H7 | SNP | 51536 | 561318 | T | C |
| H6 + H7 | SNP | 51884 | 561666 | T | G |
| H6 + H7 | SNP | 60604 | 570386 | T | A |
| H6 + H7 | SNP | 61165 | 570947 | A | G |
| H6 + H7 | SNP | 75115 | 584897 | A | G |
| H6 + H7 | SNP | 82922 | 592704 | A | T |
| H6 + H7 | SNP | 83552 | 593334 | T | C |
| H6 + H7 | SNP | 85271 | 595053 | A | G |
| H6 + H7 | SNP | 110989 | 620771 | C | T |
| H6 + H7 | SNP | 112030 | 621812 | C | G |
| H6 + H7 | SNP | 113428 | 623210 | G | C |
| H6 + H7 | SNP | 115628 | 625410 | C | T |
| H6 + H7 | SNP | 116515 | 626297 | A | G |
| H6 + H7 | SNP | 116926 | 626708 | C | T |
| H6 + H7 | SNP | 117276 | 627058 | A | T |
| H2 + H6 + H7 | SNP | 28770 | 538552 | T | C |
| H2 + H6 + H7 | SNP | 31910 | 541692 | A | G |
| H2 + H6 + H7 | SNP | 37931 | 547713 | T | C |
| H2 + H6 + H7 | SNP | 39314 | 549096 | A | G |
| H2 + H6 + H7 | SNP | 50197 | 559979 | T | G |
| H2 + H6 + H7 | SNP | 50334 | 560116 | A | G |
| H2 + H6 + H7 | SNP | 50632 | 560414 | A | G |
| H2 + H6 + H7 | SNP | 51217 | 560999 | A | G |
| H2 + H6 + H7 | SNP | 51861 | 561643 | G | C |
| H4 + H5 + H6 + H7 | SNP | 54148 | 563930 | A | G |
| H4 + H5 + H6 + H7 | SNP | 54641 | 564423 | C | G |
| H4 + H5 + H6 + H7 | SNP | 54751 | 564533 | C | T |
| H4 + H5 + H6 + H7 | SNP | 60559 | 570341 | G | C |
| H4 + H5 + H6 + H7 | SNP | 66164 | 575946 | T | C |
| H4 + H5 + H6 + H7 | SNP | 67857 | 577639 | A | T |
| H2 + H7 | SNP | 16845 | 526627 | C | T |
| H2 + H7 | SNP | 20089 | 529871 | A | T |
| H2 + H4 + H5 + H6 + H7 | SNP | 21850 | 531632 | T | C |
| H2 + H4 + H5 + H6 + H7 | SNP | 36909 | 546691 | A | C |
| H2 + H4 + H5 + H6 + H7 | SNP | 39927 | 549709 | C | T |
| H2 + H4 + H5 + H6 + H7 | SNP | 45826 | 555608 | T | C |
| H2 + H4 + H5 + H6 + H7 | SNP | 65171 | 574953 | G | A |
| H2 + H4 + H5 + H6 + H7 | SNP | 112283 | 622065 | T | A |
| H2 + H4 + H5 + H6 + H7 | SNP | 112859 | 622641 | C | A |

TABLE 15
The cellular location of transiently expressed GPRA A, B, B-short, C, D, E, and F variants.
Results are means at least from two independent experiments.

| | GPRA A | GPRA B | GPRA B-short | GPRA C | GPRA D | GPRA E | GPRA F |
|---|---|---|---|---|---|---|---|
| cytoplasm | 29% | 48% | 100% | 100% | 100% | 100% | 100% |
| Plasma | 71% | 52% | 0% | 0% | 0% | 0% | 0% |
| membrane | | | | | | | |

What is claimed is:

1. An isolated GPRA polypeptide comprising an amino acid sequence that has at least 90% sequence identity to an amino acid sequence selected from the group consisting of SEQ. ID NOS: 3, 5, 7, 9, 11, 13 and 15 over the entire length of the selected SEQ ID NO. when compared using the BLASTP algorithm with a wordlength (W) of 3, and the BLOSUM62 scoring matrix.

2. The isolated GPRA polypeptide of claim 1 comprising an amino acid sequence selected from the group consisting of B-Long (SEQ ID NO: 5), B-Short (SEQ ID NO:7), C (SEQ ID NO:9), D (SEQ. ID NO:11), E (SEQ ID NO:13) and F (SEQ ID NO:15) provided that up to 34 amino acids can be substituted, deleted or inserted relative to the selected SEQ ID NO.

3. The isolated GPRA polypeptide of claim 1 comprising the amino acid sequence selected from the group consisting of B-Long (SEQ ID NO: 5), B-Short (SEQ ID NO:7), C (SEQ ID NO:9), D (SEQ. ID NO:11), E (SEQ ID NO:13) and F (SEQ ID NO:15)

4. An isolated GPRA polypeptide comprising at least 10 contiguous amino acids from amino acids 343-377 of B-long (SEQ ID NO:5).

5. An isolated GPRA polypeptide comprising an amino acid sequence that has at least 80% sequence identity to an amino acid sequence selected from the group consisting of SEQ. ID NOS: 3, 5, 7, 9, 11, 13 and 15 over a sequence comparison window of at least 40 amino acids when compared using the BLASTP algorithm with a wordlength (W) of 3, and the BLOSUM62 scoring matrix provided that the polypeptide includes a variant amino acid encoded by a variant form shown in Table 7.

6. An isolated GPRA polypeptide of claim 1 comprising the amino acid sequence of SEQ ID NO:3 provided that the sequence contains an amino acid substitution of Asn 107Ile, Arg241Ser, or Gln344Arg.

7. An isolated GPRA polypeptide of claim 1 comprising the amino acid sequence of SEQ ID NO:5 provided that the sequence contain as an amino acid substitution of Asn instead of Ile at codon position 107, Arg instead of Ser at codon position 241, and/or Thr instead of Ile at codon position 366.

8. An isolated nucleic acid encoding the GPRA polypeptide of any of the preceding claims.

9. The isolated nucleic acid of claim 8 that hybridizes under highly stringent conditions to any of SEQ ID NOS: 4, 6, 8, 10, 12, and 14 wherein the highly stringent conditions are 6× NaCl/sodium citrate (SSC) at about 45° C. for a hybridization step, followed by a wash of 2×SSC at 50° C.

10. An isolated nucleic acid of claim 8 that hybridizes under highly stringent conditions to any of SEQ ID NOS: 1, 4, 6, 8, 10, 12, and 14 without hybridizing under the same highly stringent conditions to SEQ ID NO:2, wherein the highly stringent conditions are 6× NaCl/sodium citrate (SSC) at about 45° C. for a hybridization step, followed by a wash of 2×SSC at 50° C.

11. An isolated nucleic acid of claim 8 having a sequence that is at least 90% identical to a nucleic acid having a sequence selected from the group consisting of SEQ ID NO: 4, 6, 8, 10, 12, and 14 over the entire length of the selected SEQ ID NO when compared using the BLASTN algorithm with a wordlength (W) of 11, M=5, and N=−4.

12. An isolated nucleic acid of claim 8 having a sequence that is at least 80% identical to a nucleic acid having a sequence selected from the group consisting of SEQ ID NO: 1, 2, 4, 6, 8, 10, 12, and 14 over a sequence comparison window of at least 100 nucleotides when compared using the BLASTN algorithm with a wordlength (W) of 11, M=5, and N=−4 provided that the nucleic acid includes a polymorphic site occupied by a variant form as shown in Table 3 or Table 7.

13. The isolated nucleic acid of claim 12, wherein the variant form is at a polymorphic site not designated by a * in Table 7.

14. An isolated nucleic acid of claim 8 having a sequence that is at least 80% identical to a nucleic acid having a sequence selected from the group consisting of SEQ ID NO: 1, 2, 4, 6, 8, 10, 12, and 14 over a sequence comparison window of at least 100 nucleotides when compared using the BLASTN algorithm with a wordlength (W) of 11, M=5, and N=−4 provided that the nucleic acid includes a polymorphic site occupied by a reference form designated with a * in Table 7.

15. An isolated genomic DNA molecule or a minigene having at least one intronic sequence and encoding a GPRA polypeptide that has at least 80% sequence identity to an amino acid sequence selected from the group consisting of SEQ. ID NOS: 3, 5, 7, 9, 11, 13 and 15 over a region at least 40 amino acids in length when compared using the BLASTP algorithm with a wordlength (W) of 3, and the BLOSUM62 scoring matrix.

16. The isolated genomic DNA molecule or minigene of claim 15, comprising at least exons III-IV of SEQ ID NO: 2.

17. The isolated genomic DNA molecule or minigene of claim 15, wherein the intronic sequence is from intron 2 or 3 of a GPRA gene.

18. The isolated genomic DNA molecule or minigene of claim 15, wherein the GPRA polypeptide includes amino acids 343-377 of B-Long (SEQ ID NO:5)

19. The isolated genomic DNA molecule or minigene of claim 15, wherein the polypeptide is SEQ. ID NOS: 3, 5, 7, 9, 11, 13 or 15.

20. The isolated nucleic acid, genomic DNA molecule or minigene of any of claims 8-19, linked to a second nucleic acid with which it is not naturally associated.

21. The isolated nucleic acid, genomic DNA molecule or minigene of claim 20, wherein the second nucleic acid includes a heterologous promoter operably linked to a gene within the isolated nucleic acid.

22. A vector comprising the isolated nucleic acid, genomic molecule or minigene nucleic acid of any of claims 8-21.

23. A host cell comprising the vector of claim 22.

24. An antibody that specifically binds to an epitope within amino acids 343-377 of B-long (SEQ ID NO:5) or amino acids 332-366 of B-short (SEQ ID NO:7).

25. A method of preventing or treating asthma, other IgE mediated disease, chronic obstructive pulmonary disease or cancer, comprising
administering to a patient suffering from or at risk of asthma, other IgE mediated disease, chronic obstructive pulmonary disease or cancer an effective amount of a modulator of a GPRA polypeptide comprising an amino acid sequence selected from the group consisting of SEQ. ID NOS: 3, 5, 7, 9, 11, 13, and 15.

26. The method of claim 25, further comprising administering an effective amount of a modulator of a AAA1 polypeptide comprising an amino acid sequence selected from the group consisting of SEQ ID NOS: 17, 19, 21, 23, 25, 27, 29, 31, 33, 35, 37, 39 and 41.

27. The method of claim 25, wherein the modulator binds to the GPRA polypeptide.

28. The method of claim 25, wherein the modulator inhibits expression of the GPRA polypeptide.

29. The method of claim 25, wherein the modulator is a transcript of an AAA1 gene or an inhibitor of expression of an AAA1 gene.

30. The method of claim 26, wherein the modulator of the AAA1 polypeptide binds to the AAA1 polypeptide.

31. The method of claim 26, wherein the modulator of the AAA1 polypeptide inhibits expression of the AAA1 polypeptide.

32. A method of identifying a modulator of a GPRA polypeptide, comprising
contacting a cell expressing a GPRA polypeptide with an agent;
determining whether the agent modulates expression of the GPRA polypeptide and/or signal transduction through the GPRA polypeptide, wherein the GPRA polypeptide is defined by any of claims 1-7.

33. The method of claim 32, wherein the cell further expresses an AAA1 polypeptide.

34. A method of determining risk of asthma, other IgE mediated disease, chronic obstructive pulmonary disease or cancer, comprising;
determining whether the individual has a variant polymorphic form in a GPRA gene, wherein presence of the variant polymorphic form indicates risk of asthma, other IgE mediated disease, chronic obstructive pulmonary disease or cancer.

35. The method of claim 34, wherein the variant form occurs in a noncoding region of the GPRA gene

36. The method of claim 34, wherein the variant form occurs in a coding region of the GPRA gene

37. The method of claim 34, wherein the variant form occurs between introns 1 and 4 of the GPRA gene.

38. The method of claim 34, wherein the determining comprises determining whether the individual has a variant form relative to SEQ ID NO: 1 (AST-1 locus).

39. The method of claim 34, wherein the determining comprises determining whether the individual has a variant form relative to any of SEQ ID NOS: 1, 3, 5, 7, 9, 11, 13, and 15.

40. The method of claim 34, wherein the variant form is a variant form shown in Table 3.

41. The method of claim 40, wherein the variant form is at a polymorphic site shown in Table 3.

42. The method of claim 34, wherein the variant form is a variant form shown in Table 7.

43. The method of claim 34, wherein the variant form is a variant form at a polymorphic site not designated * in Table 7.

44. The method of claim 34, wherein the determining comprising determining whether the individual has variant polymorphic forms relative to SEQ ID NO:1 at each of a plurality of polymorphic sites within the AST-1 locus, the presence of variant polymorphic forms at two or more of the plurality of polymorphic sites indicating increased risk of asthma, other IgE mediated disease, chronic obstructive pulmonary disease or cancer.

45. The method of claim 34, further comprising determining whether the individual has a variant polymorphic form in an AAA1 gene, wherein presence of the variant polymorphic form indicates risk of asthma, other IgE-mediated disease, chronic obstructive pulmonary disease or cancer.

46. The method of claim 45, wherein the variant polymorphic form occurs in the coding region of the AAA1 gene.

47. The method of claim 34, further comprising amplifying at least part of SEQ ID NO:1 (AST-1) locus including the polymorphic site before the determining step.

48. The method of claim 34, wherein the determining is performed by allele specific amplification, allele specific hybridization, single strand conformation polymorphism (SSCP), oligonucleotide ligation assay, single-base extension assay, or restriction fragment length polymorphism (RFLP).

49. A method for identifying a polymorphic site correlated with a disease selected from the group consisting of asthma, other IgE-mediated disease, chronic obstructive pulmonary disease and cancer or susceptibility thereto, comprising;
identifying a polymorphic site within a GPRA gene,
determining whether a variant polymorphic form occupying the site is associated with the disease or susceptibility thereto.

50. The method of claim 49, wherein the variant form occurs in a noncoding region of the GPRA gene

51. The method of claim 49, wherein the variant form occurs in a coding region of the GPRA gene

52. The method of claim 49, wherein the variant form occurs between introns 2 and 4 of the GPRA gene.

53. The method of claim 49, wherein the determining comprises determining whether the individual has a variant form relative to SEQ ID NO: 1 (AST-1 locus)

54. The method of claim 49, wherein the determining comprises determining whether the individual has a variant form relative to any of SEQ ID NOS: 1, 3, 5, 7, 9, 11, 13, and 15.

55. The method of claim 49, wherein the determining is performed by comparing the frequency of the variant polymorphic form in individuals with and without the disease.

56. A primer or probe nucleic acid that hybridizes under highly stringent conditions to a segment of SEQ ID NO:1, 2 or 4 or a variant form thereof differing from SEQ ID NO: 1, 2 or 4 at a position shown in Table 3 or Table 7, wherein the segment includes or is immediately adjacent to a polymorphic site shown in Table 3 or Table 7.

57. The primer or probe of claim 56, wherein the position is a position other than a position designated * in Table 7.

58. The primer or probe of claim 56 that is perfectly complementary to a segment of SEQ ID NO:1, 2 or 4.

59. The primer or probe of claim 56 that is perfectly complementary to a variant form of a segment of SEQ ID NO:1, 2 or 4 shown in Table 3 or Table 7.

60. The primer or probe of claim 56 that specifically hybridizes to the segment of SEQ. ID NO: 1, 2 or 4 without hybridizing to a corresponding segment of an allelic variant shown in Table 3 or Table 7.

61. A primer of claim 56 for conducting a single-base extension reaction, whereby the primer is perfectly complementary to a segment that is immediately adjacent to but does not include the polymorphic site.

62. A transgenic animal comprising a nucleic acid according to any one of claims 8-21.

63. The transgenic animal of claim 62, further comprising a nucleic acid encoding an AAA1 polypeptide.

64. A transgenic animal of claim 62 disposed to develop a characteristic of asthma, other IgE-mediated disease, chronic obstructive pulmonary disease or cancer in which an endogenous a GPRA gene encoding a cognate form of a GPRA polypeptide defined by any of SEQ ID NOS: 3, 5, 7, 9, 11, 13 and 15 is functionally disrupted to prevent expression of a gene product.

65. The transgenic animal of claim 64 in which an endogenous AAA1 gene encoding a cognate form of an AAA1 polypeptide defined by an of SEQ ID NOS: 17, 19, 21, 23, 25, 27, 29, 31, 33, 35, 37, 39 and 41 is functionally disrupted to prevent expression of a gene product of the AAA1 gene.

66. A kit for use in diagnosing or assessing predisposition to asthma, other IgE-mediated disease, chronic obstructive pulmonary disease or cancer, comprising;
a container; and in the container:
a compound, preferably labeled, capable of detecting a polymorphic form at a polymorphic site in a susceptibility locus for asthma as defined by SEQ ID NO:1, 2 or 4.

67. The kit according to claim 66, wherein the polymorphic site occurs at a position shown in Table 3, Table 7, Table 12, Table 13 or Table 14.

68. The kit according to claim 66, wherein the compound is a primer or probe.

69. The kit according to claim 66, wherein said primer is the primer of claim 56.

70. An isolated AAA1 polypeptide comprising an amino acid sequence that has at least 80% sequence identity to an amino acid sequence selected from the group consisting of SEQ. ID NOS: 17, 19, 21, 23, 25, 27, 29, 31, 33, 35, 37, 39 and 41 over the entire length of the selected SEQ ID No: when compared using the BLASTP algorithm with a wordlength (W) of 3, and the BLOSUM62 scoring matrix.

71. The isolated AAA1 polypeptide of claim 70 comprising an amino acid sequence that has at least 90% sequence identity to the selected amino acid sequence.

72. The isolated AAA1 polypeptide of claim 70 comprising an amino acid sequence selected from the group consisting of SEQ ID NOS: 17, 19, 21, 23, 25, 27, 29, 31, 33, 35, 37, 39 and 41.

73. The isolated AAA1 polypeptide of claim 70 comprising at least 10 contiguous amino acids from an amino acid sequence selected from the group consisting of SEQ ID NOS: 17, 19, 21, 23, 25, 27, 29, 31, 33, 35, 37, 39 and 41.

74. An isolated nucleic acid encoding the AAA1 polypeptide of any of claims 70-73.

75. The isolated nucleic acid of claim 74 that hybridizes under highly stringent conditions to any of SEQ ID NOS: 16, 18, 20, 22, 24, 26, 28, 30, 32, 34, 36, 38 and 40, wherein the highly stringent conditions are 6× NaCl/sodium citrate (SSC) at about 45° C. for a hybridization step, followed by a wash of 2×SSC at 50° C.

76. An isolated nucleic acid of claim 74 having a sequence that is at least 80% identical to a nucleic acid having a sequence selected from the group consisting of SEQ ID NO: 16, 18, 20, 22, 24, 26, 28, 30, 32, 34, 36, 38 and 40 over the entire length of the selected SEQ ID NO when compared using the BLASTN algorithm with a wordlength (W) of 11, M=5, and N=−4.

77. The isolated nucleic acid of claim 76, wherein at least one polymorphic site shown in Table 12 is occupied by a variant nucleotide.

78. An isolated nucleic acid having at least 20 contiguous nucleotides from a sequence selected from the group consisting of SEQ ID NOS: 16, 18, 20, 22, 24, 26, 28, 30, 32, 34, 36, 38 and 40.

79. An isolated genomic DNA molecule or a minigene having at least one intronic sequence and encoding an AAA1 polypeptide that has at least at least 80% sequence identity to an amino acid sequence selected from the group consisting of SEQ. ID NOS: 17, 19, 21, 23, 25, 27, 29, 31, 33, 35, 37, 39 and 41 over the entire length of the selected SEQ ID NO. when compared using the BLASTP algorithm with a wordlength (W) of 3, and the BLOSUM62 scoring matrix.

80. The isolated genomic DNA molecule or minigene of claim 79, wherein the polypeptide has a sequence selected from the group consisting of SEQ. ID NOS: 17, 19, 21, 23, 25, 27, 29, 31, 33, 35, 37, 39 and 41.

81. The isolated nucleic acid, genomic DNA molecule or minigene of claim 79 or 80 linked to a second nucleic acid with which it is not naturally associated.

82. The isolated nucleic acid, genomic DNA molecule or minigene of claim 79, wherein the second nucleic acid includes a heterologous promoter operably linked to a gene within the isolated nucleic acid.

83. A vector comprising the isolated nucleic acid, genomic molecule or minigene nucleic acid of any of claims 74-82.

84. A host cell comprising the vector of claim 83.

85. An antibody that specifically binds to a polypeptide selected from the group consisting of SEQ ID NOS: 17, 19, 21, 23, 25, 27, 29, 31, 33, 35, 37, 39 and 41.

86. A method of preventing or treating asthma, other IgE-mediated disease, chronic obstructive pulmonary disease or cancer, comprising
administering to a patient suffering from or at risk of asthma, other IgE-mediated disease, chronic obstructive pulmonary disease or cancer an effective amount of a modulator of an AAA1 polypeptide comprising an amino acid sequence selected from the group consisting of SEQ. ID NOS: 17, 19, 21, 23, 25, 27, 29, 31, 33, 35, 37, 39 and 41.

87. The method of claim 86, wherein the modulator binds to the AAA1 polypeptide.

88. The method of claim 86, wherein the modulator inhibits or induces expression of the AAA1 polypeptide.

89. A method of identifying a modulator of an AAA1 polypeptide, comprising
contacting an AAA1 polypeptide with an agent;
determining whether the agent binds to the AA1 polypeptide, modulates expression of the AAA1 polypeptide or modulates activity of the AAA1 polypeptide, wherein the AAA1 polypeptide comprises an amino acid sequenced as defined by any of SEQ ID NOS:17, 19, 21, 23, 25, 27, 29, 31, 33, 35, 37, 39 and 41.

90. The method of claim 89, wherein the AAA1 polypeptide is expressed from a cell.

91. A method of determining risk of asthma, other IgE mediated disease, chronic obstructive pulmonary disease or cancer, comprising;
determining whether the individual has a variant polymorphic form in an AAA1 gene, wherein presence of the variant polymorphic form indicates risk of asthma, other IgE mediated disease, chronic obstructive pulmonary disease or cancer.

92. The method of claim 91, wherein the variant form occurs in a noncoding region of the AAA1 gene

93. The method of claim 91, wherein the variant form occurs in a coding region of the AAA1 gene

94. The method of claim 91, wherein the determining comprises determining whether the individual has a variant form relative to SEQ ID NO: 1 (AST-1 locus)

95. The method of claim 91, wherein the determining comprises determining whether the individual has a variant form relative to any of SEQ ID NOS: 16, 18, 20, 22, 24, 26, 28, 30, 32, 34, 36, 38 and 40.

96. The method of claim 91, wherein the variant form is a variant form shown in Table 12.

97. The method of claim 91, wherein the determining is performed by allele specific amplification, allele specific hybridization, single strand conformation polymorphism (SSCP), oligonucleotide ligation assay, single-base extension assay, or restriction fragment length polymorphism (RFLP).

98. A method for identifying a polymorphic site correlated with a disease selected from the group consisting of asthma, other IgE mediated disease, chronic obstructive pulmonary disease or cancer or susceptibility thereto, comprising;
identifying a polymorphic site within an AAA1 gene,
determining whether a variant polymorphic form occupying the site is associated with the disease or susceptibility thereto.

99. The method of claim 98, wherein the variant form occurs in a noncoding region of the AAA1 gene.

100. The method of claim 98, wherein the variant form occurs in a coding region of the AAA1 gene

101. The method of claim 98, wherein the determining comprises determining whether the individual has a variant form relative to SEQ ID NO: 1 (AST-1 locus)

102. The method of claim 98, wherein the determining comprises determining whether the individual has a variant form relative to any of SEQ ID NOS: 16, 18, 20, 22, 24, 26, 28, 30, 32, 34, 36, 38 and 40.

103. The method of claim 98, wherein the determining is performed by comparing the frequency of the variant polymorphic form in individuals with and without the disease.

104. A primer or probe nucleic acid of nucleotides that hybridizes under highly stringent conditions to a segment of SEQ ID NO: 16, 18, 20, 22, 24, 26, 28, 30, 32, 34, 36, 38 and 40 or a variant form thereof differing from SEQ ID NOS. 16, 18, 20, 22, 24, 26, 28, 30, 32, 34, 36, 38 and 40 at a single polymorphic site.

105. The primer of probe of claim 104, wherein the polymorphic site is one shown in Table 3 or 12.

106. The primer or probe of claim 104 that is perfectly complementary to a segment of SEQ ID NOS:16, 18, 20, 22, 24, 26, 28, 30, 32, 34, 36, 38 and 40.

107. A primer or probe of claim 104 for conducting a single-base extension reaction, whereby the primer is perfectly complementary to a segment that is immediately adjacent to but does not include the polymorphic site.

108. A transgenic animal comprising a nucleic acid according to any one of claims 74-82.

109. A transgenic animal of claim 108 disposed to develop a characteristic of asthma, other IgE-mediated disease, chronic obstructive pulmonary disease or cancer in which an endogenous a AAA1 gene encoding a cognate form of an AAA1 polypeptide defined by any of SEQ ID NOS: 17, 19, 21, 23, 25, 27, 29, 31, 33, 35, 37, 39 and 41 is functionally disrupted to prevent expression of a gene product.

110. A pharmaceutical composition comprising an effective amount of a GPRA polypeptide comprising an amino acid sequence selected from the group consisting of SEQ. ID NOS: 3, 5, 7, 9, 11, 13, and 15 or a modulator thereof for the prevention or treatment of asthma or other IgE mediated disease, chronic obstructive pulmonary disease or cancer.

111. The pharmaceutical composition of claim 110, further comprising an effective amount of an AAA1 polypeptide comprising an amino acid sequence selected from the group consisting of SEQ ID NOS: 17, 19, 21, 23, 25, 27, 29, 31, 33, 35, 37, 39, and 41 or a modulator thereof.

112. The pharmaceutical composition of claim 110, wherein the modulator binds to the GPRA polypeptide and/or activates/inhibits expression of the GPRA polypeptide.

113. The pharmaceutical composition of claim 111, wherein the modulator is a transcript of an AAA1 gene or an activator/inhibitor of expression of an AAA1 gene.

114. The pharmaceutical composition of claim 111, wherein the modulator binds to the AAA1 polypeptide and/or activates/inhibits expression of the AAA1 polypeptide.

115. Use of a GPRA polypeptide comprising an amino acid sequence selected from the group consisting of SEQ. ID NOS: 3, 5, 7, 9, 11, and 13 or a modulator thereof for the manufacture of a medicament for the prevention or treatment of asthma or other IgE mediated disease, chronic obstructive pulmonary disease or cancer.

116. The use of claim 115, wherein a GPRA polypeptide comprising an amino acid sequence selected from the group consisting of SEQ. ID NOS: 3, 5, 7, 9, 11, 13, and 15 or a modulator thereof, and an AAA1 polypeptide comprising an amino acid sequence selected from the group consisting of SEQ ID NOS: 17, 19, 21, 23, 25, 27, 29, 31, 33, 35, 37, 39, and 41 or a modulator thereof are used for the manufacture of said medicament.

117. A pharmaceutical composition comprising an effective amount of an AAA1 polypeptide comprising an amino acid sequence selected from the group consisting of SEQ ID NOS: 17, 19, 21, 23, 25, 27, 29, 31, 33, 35, 37, 39, and 41 or a modulator thereof for the prevention or treatment of asthma or other IgE mediated disease, chronic obstructive pulmonary disease or cancer.

118. The pharmaceutical composition of claim 117, wherein the modulator binds to the AAA1 polypeptide and/or activates/inhibits expression of the AAA1 polypeptide.

119. Use of an AAA1 polypeptide comprising an amino acid sequence selected from the group consisting of SEQ ID NOS: 17, 19, 21, 23, 25, 27, 29, 31, 33, 35, 37, 39, and 41 or a modulator thereof for the manufacture of a medicament for the prevention or treatment of asthma or other IgE mediated disease, chronic obstructive pulmonary disease or cancer.


SEQUENCE LISTING (266 sequences)

SEQ ID NO: 1 - DNA, 129017 bp, Homo sapiens
cggcaaattaaatgtagctaaagaagaaatcattgaactggaatataggtccaaagaaaataattagattgaagcatgaaaaaagaaaggacaaaaaatacaaaaagaaaataaaaagcatatggaagtagtgaaaaggactcccagaaaaagataaagcccagactggatagaaacaacacttatagtggccaataattttcccaaactctttaaagacatcaaaacacaaatacataaagcaccataaaggacaacagcaagcaaacaaacaaatataaagagaattaaaacacatgaaatgataagactaagtaaaagccccataatcttgaatttgaaatgaaaatatttatgctagctcatgatgtattttatgcttcaggagtttttaaggatacataccaaaatgtttataggtaaactgataacatgtctgggatttacttcaaaataatttgaggctgggggtgggttgagatgtatagatgtacaaaattgatattgataactgttggaggttggtggtgagtacattggattctttattttttttatactattctctctacttttatgtaagtttaagatttttttcataataaaattcaaaacaaaattatgcatgattataatgaaacaaaataaagagatctgggtagttaaagatgttaaagtccttggattgcctggatgtagtaaaagtactaatttctattagattttaatgtcaaggaggtgtgttaaaatctctaaagtaatcctcccaaattatgaaaactgacattactagcaagctaaagaaggtataaaatgcagtatttgaatatgcaaatatatatgtttatatatagagttattccccagtatccatggaggattagttccagaatcccctgccaatacaaaaattcgaggatgcttaagtcccttacataaaatagatagtatttgcatataacctacatacaccctcctgtatactttaagtcatctctaaattacttataatttctaatgcaatgtaaattctatataaaaatttttatactatattcttaaaatttgtatcattttattattgtattgttattttatactttcttaatatttttaatctgcagttgatagaatccatggatgtggaacccacagataaaaagggccaattataatacatacatattatataaaatcaatctaagttaagacaggagagaaaaaatattagaattattagaacaagtaagaagaataggccagtacacttaacctcaaatgtatcagtaattgcattaattgtaaatggactaaatattcccattaaaataacaagattgtcagagtagatttttaaattacctattttaatttgtttataaaaggtgtacaccataaataaaatgatgcaggttaaaattaaaaggaaaaggatggtaaagtaaaatgataaaaaataatgaaacaaaacaaaacacaaagtaaaagtaagccaatgcagctacatatgtgtatcaaaagtttataaatttgctgtgattcttggaatcacttgtagctttatctgtgtcttagaatttgctttgaaaggaatacaaagacaggagggcttctaaagctagtaatgttctatttcttgatcttgcttgtagttacgtgggcgtgttcctcttgtaataattcaccaaattatatacttatgattttgtatacttttctttatgtgttttatttcaatacaaagtttatttacaaaatatatcacattaatccagggcaggggttgcaatctcaaatggctttaggaatcaagcaggcaatgaatgtaaacatgtaaaagagccaaggggaagagacagaatgaaaacagagataaaaaccagagaatgaatgccctgcctaaagacattaaaatcaatttttgtcaaacactgtgctgatcaaacaaatcacacatgcaggccagatttgacccaagggtgactttcttacaacaactgatatataaaatttaaacatcagaatttgaaaagtgtgccactataaaattctagcccacctctgaagtatgaacccaccttcaatttgtgaggtagaatattaatctttactggtggattgtgagaagtgaggccaagaagttggggaggttaacatttacaatggtttagacaaatcatatgtttgacaaggtttaaaatcccttctgttcaaagaatatcttatattaggattttcttctcctaatgtgtttattattgtttttattagtataatttttaaagaaatctgaactattgcacgcctcagagaccataacagtgagaacaaaaatataaaggaaaattttcaaatgatggaaagtgtaacccatataacccgaaatcttcatgattttcagatgaccatctgattaaggagaaaagtcaagataagcacatattcatatctgcttatttctagaattgttctatcagaagaccatgaattggtcatatgaggggaagaaacactgacatgatactggctctattaggatgatattaaaagaggctgctggctcaagcttagcatggatgagttgttctcacataaagatgctgttattgaccaggtgtggtggctcacgcctataatcccagcactttgggaggctttggtgagtagatcccctgtgtccaggagttcaagaccagcctgagcaacatagtgagaccctgtctctactaaaaaattttttaaattaaaagttaaaaaattaaatgatgttgctattgttaatgcattcctgcaatgctaacatgctttcataaatatacttatgattttataggtgttttcctcttagcctcaagaagggagatgttttagtctagggactgtcataacaaaataatataaactgtgtggctcaaacaacagaaatttatttcctcgcagtcctgtaggctgaaagtttaacattagggtgccagcatggtgtggctctcttcctgggttgcagatgtcaccgtctcactgcatcctcatgtggcctttgtgcaatgcctctgcacaaagagagagaaacagcaagttatgtgccgtctcttcttgtaggctcactaatcctatggaatcagggccccacccttgttaaccttaattatttccacagaggtcatatgttcaaatacagtcacattgggagttagagattcaacatataaattttaggggaatacaaaccttcagtctgtaacagggaacagaggtgctagaagaaatgtagtagtagcaaaaggtggggtggggagataataggagcttcatatgtcttgctaagaagttcaggctttgcttttagcacagcccctcttctgctagctttgttcaggccaccatcacctttgatgcagattactacaaaagccacccaaggtgtatgaaatgagaaaacacatacttaaattaacccatggccaggttttgctggtaacttgggaacacagtaagggaaggaaaataactttgttgtggagagggcaaaccaaggaaggtcagaaagacatagagacagtggaagctcaaggaaccggaggttgtcccaaagtcataaggcggatgtaacgagagcaccagaacaacagagctggaaggaaggaaggaaggaaggatatgtttagaaagcaagatgttcccagatgaatactgcagaggtagtataactcagtgcaacgattgtactgggtaggcaaggtagggtggcagggaaagtcactagcaaacatggccacaataatggaaactaaactttgtgtttaccatgcaccaggctctgtgctacatgctttctgtagatcatctcatttaatcctcacacagggatttaagcctgaagtttttttcagagaagacatacaaatcccaagtaagagcatgaaaagatgttcattagcattagtcattagaggaaatgcaaatccaaagatcagggagatacgacttcacaccttcaagaatggctattcccctataaaataatatactaaatcactctttgaaacataacattttccacaaactcacctctttgaattcacattttgctttgccagtgttttatgttattcagacatgagtttgaatctcagcatttctacttctgagccctatgacctatgaaaagtgacaatttctctgaatatcaatttcccaatccaaaaaatgggaatacctcttggagttgtggtgaggattaaataagataatgaatataaagtgcctagtatagtaagaggcatattattgttcaataaatgctagctgtgaatgctactgctatagttatttcattcactgtagaaatgattaatttaaactccttcaagtaacttactaaagagtgaaagttctccaaaaggcttgaatttcactgcaaacctctttccttgttttgctcttttattcaggacaatacactttgctcaggtgtgtctttgactctcaagcattgctttaaagaatgaccagactttgtcatataccattcttaaaggagttcaggacatgccaccccaaaatatgctgctttcatatattgattattttgagctgatagcacttcaacagcaaatgcaagagagactttttcggaactcagctgcctaaaaacagatcctccaaaagaaattcaattgccatatatcctctccctgggagttttattaatcagcaaagatacacttttatcacaaaacaggaaactggaagtcaacaccacctacagaaaaactttttcataaactgttatatctcccattgattcttctaaaaacccattcatctttccccagaatcatttactccaccctaagtgacttacatcacccctccccatcccctgttaacacagtatgtaagctctcaaatctcttttaggagtattcattattttcctgtggcaccccatgaacataatattaaaatgaatatgccttctctcttattaatctgcctgttgtcagtttatttcatagacacagctatcgaacctaggagagtacagggaaattctttcctcccctatatgcttaaatcataagattcaactgacatcagcaatgaaacaaacattcagaaaactaaattcagagacattatatctggcttttctgagggagaataataagaactaataggcattgtcaggtaaggcaatacagatgtttttaattaaatgatgttttttcaccatgacccacatcttgaaaacaccttcctaattaaagaccagtagtcttgagtgattctttgaagaacatttttacttccatttggtcttttctttattgagttgatgaatctgctggaattagattcagcgcacataacataaaatccaaataatgctggcttaagttcataaggatttatttttatcttatgtaaagaagtccggacgtaggtaatccagggctcgtgctgcattttcttaccattgggaacccaggctcctctgtatttctactgtattaaaggactgtcaccctcaagttcagctcatggtacagatggcggttggttcaagatgactgctagaacatctgtcatcatgcccaaattctagccagcaggaaggaagaagggaggaaggacagaggggctcatatcagctctttgtcctcctcttaattggcattgccagaattcccacccattagtttttccttgcatctcaatggtaacgtctaagtgaaaaaaagcctgggaaatattttcatttattattatttttttaacccaggcccattggccagaataaaaccagcttccttttcctaaggaagaaggagaaatagaattaaggaacagtctctggcatagcttgttctccatacttttacttccatccagaactgagaagtttagacaacaaatctctttttgtccacccattgcctgctttgagaatgcttcttccagagtgctacgctctcttctaatgacttattatcttggcagactgcacttttcacaccctccataggctgcagtgatccattggatgattgtcccattatgtagataagttcctattgctggctgtttatgcttttctcccatttttaggctattagcaatcctgcaataaacattctcataattaaatttgtatgagaattctgtattatttatttagaaaaagtttatagaagtggacttactgcatcaaagaaaggcacttttgtggcttttgaattggctaatgtcttttttggttacccaagttaaaagatgatggaaaatgtcgggggacaaatgctctagaacccgctagcatagacctacactaagtcttattcaaaaaggtccctgtaaacaaaaccctagttgacttgcagtagctacagaatcataactggttttttgagagcagagattctctccgttcctcccttagtgatacttagccagctactggtaatttgcttattatcaaaaggagcggccaggcgcggtggctcatgcctgtaatcccaccactttgggaggccaaggcgggcggatcacgaggtcaggagatcgagaccatcctggctaacacggtgaaaccccgtctctactaaaaatacaaaaaatcagccaggcgtggtggcgggcgcgatggcaggcgcctgtagtcccagctactccagaggctgaggcgggagaatggcgtgaacccgggaagcggagctcgcagtgagccgagatcgcgccactgccctccagcctgggtgacagagcaagactccgtctcaaaataaataaataaataaataaataaataaataaagcattttttttttcactttagccagcgcatcagagaagaacccacacttgattgtcttatttttccccctacaatgcccagagcagtattcacacattcatgaaaagaaaacacatcacttagaacattcgaatagagcacgtgtttttattttgctcttctatgttttacctccacttctccacaattttgatcccttcaaaaaaaaatcttaactaattattctctatatattctattaggaatcttggctgtagacttaatcttgaggttagaaagaaaatagatcttgaagccactattttggcacattctgtaattcttagaatttccattctaacaattttctggatgaatttaataaccgtttatattttatgagctaaagtgccaattaaaattattcatttaccatctttaagacttacttgtctgttttattcaaatgcaataaaaatgcggaactaataagagtaactcccgatgagtgttcaacaaagaaaagaaagaaaacatattttttggcgcacattcaaatcatttcctttttctggtagggccaaatcttctcatattgggaaaggaaattagctttgcttcaaagcactttctgaagagcaatttactaatgagcttaggaattctctgccctcataaccctcctatacattacctgaacgcagagaaacttgagacgttcagcaagggaggtaaggccaggagtgttgaggcgtccaggtccgtctgtggagttcactgccaccttcctgtgtggatttcagtccttgttttcctggattttgagttgcattctcaatacattatttttatttttattcatagtgaaaatattgaagactgaattttttctgaattcatacaatttatatgacatgttattttttattttctaaatattttactgttgaagtagttcctctttcacccaaaaaaactaatagtttttgctgtctttttttaatttctacgtgaccatttctttaatattttgtaattcacatttaattttattgccttgtagtcaaggaatatttctgaaaggacttctgctctttggaacttattaagagtgtctttgaggtttaatatgtaattattatcttaaaaatatttaatgtatactaaaatgtaagacacaatttattgctgtggaattaaacaaagaccatccaaatgagaacaagcaatggctatttactcagagtttgggagtcagcccacagtttcttgtgttgacagagactcaaaaggctgagtggaaaagctttgcaagggggaaaaaaaggcagggtctcagctatgccctgattggaggctgctggcctagggaagttgacctgaagcacagcatcttatcggattgcttaagggtgcatatttacctttctcctattggtcctaagttagaagcaagggaaacaattaaaggagctatcagttatgaaccaagttctgggtttgttgggctaattgctatggtggtattgtttggcttcctgagctggttgctgcaggttgtggataagagttctattttatgtatatggtctggccattgtctatatgttcagtctctcaatgcattatttgtacttattcttttgaatttgcctttactaattatggtctctatattgtctctttttatatacatattttttgcctgtttaacgtatgatagatgactaaaagtgctattaaaaactttcacacttttggttggacgtggtggctcatgcttgtaatcccagcactttgggaggctgaggcgggcagatcactggaggtcaggagttcaagatcagcctggtcaatatggtgaaaccccgtctctattaaaaatataaaaatcagttgggcatgatggtgcacgcccgtaataccagctactcaggaggctgaggtgggaggatcgcttgaatccaggaggtggaggttgcagtgagcagggatcacgccactgcactcctgcactccagcctgggcagcagaatgagactccatatcaaaaaaaaaaaaaaaaaagagagaaaagaaaagcctttcacactttctctcatcttttagtatttttgatcaatgtttgttttatacatttcaatggtttattatttggttcctaaaggctcctgattagcatatctttaatgtatgtttgtgtatgtatgttgatctctctctctctctctctctctctcatacacacacataagtcttttcatttattagctttgctttaaactttgctaatgtgatattaatattatgtttatctatcttctttttattgcacttgtttgccaagcaccgaactaagcattttaaataatatcacatttttgtaactgttttggcatttatcttcttcattgtgactttacttgaaataattatcaatgtgtatttctatatttgagtgtttcaatacttatcaagagttttcttccccattcttggttcctgtgtttcgacacagctctcaactttctgtagtactttgggtagtattttttttttcaagaagtcattggataagattataaacacctggcatttctttatgagcctttttcttcactccacaacaataatgatttagatgggtatagaaatcttgaattcataactcaaatctccatagatgttgctccatttaattctggtattttgtgttaccaaaaaaaaatctaaagtcagtccaagatttctttcttgataggttatccaattttgtcttttgtttctgatttttttttttttagatttcatgctttcaccaggttatgtctattttttggtcgtctgttatgtgagttctctgagctgccatatgctcttttgatctgctgattaatgtgaattcgtaggttattttctttatgtctttgaatacagattacttaccatttattttaatcttatcttcaggaactctaactgtctgtctatatgttagtaattttggcctttctgtcatcatttaaatatcttggtcctattcttctacgtgggaagaataatttatactttattaatcaataatttgattttgattcaatgattagcagtgctaattctactttttattgtttctgaagtatcttaaaggttgctttgtctatgaaaagttttaaaacattcttcctttgtattatctggtgcctttttatttctgtcagcttcttggtgtttcccttctcacaacctatctaatgtcttattatctcgtctttcacctttaattccatactgtcaaagtgtccccaaaatgtaaaaagaatgcaaaatatgcgctaaaatttcagggtttcttgagctaaagtttttttgaatatatattctttttctgatatttttgtgctctttttaactcaatgctaggaaatctctctctgtctcatatgtttcacccatctactgaaagttttttaattggctaacatctgaataaatgcacatatcttttatttggattattcctacactagctgcacaatgccatcattgtttcctccaccaagaagagctgggtgggccatgttcattgagaaaatcagtttctctcaaacaggagaaaaaatggcattgtcctatcgctatgtgaataattcaactaaaaagaataactgttctcccagtaaagctttttctttcttagaaaaattgggtttctttatattccatgcctttgcttcagagcacaatatgtgacctgggaaaggcatttacccttgttaaaccttcattttctcatttctaaaattacaagggttatctccatgatctttaggaagccagaactgatctggttctaatggtcctttgattctaattagaaatttccaactgcaaaagttgagggggattcttacatcttcagaagccaactaattaaacaacttttgctgtaatgagtgtcagtcacctttatgaatcagcttgtcacttaataatagaaatagctggcttctggcccatgcagaaggaatgtagaaaatagactatagtaacaagctgactcccattctcctgttgttccctgcctcactcagtttccacagtcaacttaggacaattccatactgtgattatttactcctgctttgctttcgtaattcaacaaacagtattaaccacttactatggcgaggcatggttccaggtgctaaggacccacagtggatttgattacaaattacaggtaaaataataaagcaataaaaaggataattttgctttcatcatcgaaagctcagtgatagagtaagctttagactttatttgcctcagcaatgcagcagtggtagagggcagaggagaagtctcatcctaagccttgctcccaaggctggttataggatgctgtattagtttgctgggactgccgtaacaaataccaaagattgggcagcttaaacaacagaaatttactttctcacagttctagaagctagaagtcctctatcaaagtgttggctaattttattttgaggcctttctcattggattgtagatggctgtcttctccgtttttttcacatggctttttttctgtccatgtctgtatcctaatcttctcttcttatgaggacaccaccagttatactggattagggcccacccagaattacctcatctttttttcttcattatctctttaaagtccctatctctaagcatgtcacattgtgaaggactggaggttaggactttaacatatgaatttaggggagatgcaattcagcccataacaaatgcctgctagtaactcatgggaactctcttccacatgcaaagagataaaatgtgatttctgcaagttttattagaaatataggaaggaacccttcccagcagggcttgacaagcctgtgcatgtgcctcactgacataactaagttatagggtcatatttgaactaatccatggaacaagacatgcaattacactgatgagcttaggctgaagtcaaatgtctcactcctgaaaattcagacttccccaaaacacatgggctgaacaggtaagatggatacttcaactgaaagccagggtacttaacaaaagcaggagaaatagatgctggggaggtaatcccaacgtccagtgcaaatgctatgagggaaaacgacagagtcccaaacatgctggagcttatagttagaggaaggaaaaattagatttttctcagggctttgggattggggcacctggcagtcagcgcctagattccaaagcaggtcagaagatgcctctgcccaagtattggaaaccttttgaatatctttgagtattaaaaacattttaacactgcctgagaaaagtagtagttatgaagttatacatttagcctatcccattcaactcaggggattgtacacagatcaaataaattgcaattgtgtttccctccctgatattcacataggaaggtgagtggtgagcagtaaacttcaggacctttaaagttatcattgctttcacccttgcaccagctttttgaggtaaatattcccattttctaggagaggataatgaggctcagagagggtaagtggcctccccagggtcttctagctgaattgtaaccagcttgatcttccttttgaattccaccatcttgcctgtgagggaaaaaggaataaggagccaggagttgcagggtcattcttctcctcaagaggcttgagagtaactaagtatctgttcaaaacacttttgagaatttggcggcctcattacgtttctcaggagtccctgggggaggttacacttttaggatccccaggaatagagtgacaaaatacagctaccacaacttggtcctgtgcaactgtgcccttgctcagcaattactgactccattctgactgaactctgccaaaaactcttaacattaataattattacctgttctcaattttaataattaagagttttacaacttccagtgactcttaattcaggtttctttggcttactgttcttggacaataacagtatggtcactgttgcacagacagaagctagacatagtctgtatagctaactccctcatgtccttcaaatctttcttcaaatgtcagtttcttttggggccttctgtgaccaacctgatgaatagagcccaccagccccagaccacagcgttcctgacttcctttactctgcttcacttttccctttttcacagcaattgtcacattctattctactataacttacttatttattgaatttaccactgattgtttgcctaacctcactataagacagggatctttgttttattctccaagaacttagaacagtagttattcaattaatatttgttgaagcaatgaatgaaccaaatgaatgaatgaaagatgaatataaaaatatttatatgtcctaacactctactaagcattatgacagaatatccaccaaaaaaaaaagatgagaaggagaaaccattattcttctgtagcagggctaacagaacatgagcagatggcaaacgtgagattatcagcaccttctgggctagttcctggaaatcagctgatttcctgctgcttccaaaaattcttaaagctttcagactcctgagcaagctcctgcaaaacacctattttggtgctgcaagcacctgcagtagctctctagtcattcaggtacttgttgaccttaggaactcaagccatagcttccacgattttcaatccctgatccctcttcagtctcaaattgctgtatccaaaacccttcctacttgcaatatattagaaaggccaccttctgaccaaatctgaaaagttgggtaggtttgtagaggatgagtttttggattattcccaattcttttgatcctcaccccaacctcctaagactaaagctacagtggcctccctttagggaagaaagcagaggaaaggagcagaggaattattttctgtgaattaactgtttctagattctaatattagtgactaaactgccatttgtacttcctatattcctgttttagcaagagctaaaagagtttcaaagaaggtaaattgtattctaagaagagaattcagaaagtcctttccagaatagatgaatcttaaatgatgggcaggtttcccacagagataggtggggaagccattctaacctagaaaacaggatgcctagaagtatgcagtgttttcagaaaatggcagacacaggcttccttggctagagaacaaagacttcatacaggagtaggttcataaatacaagcacaagagacctttatagcattggggtctagtgattctcaattattctgcctataagcatcaactgcaagaatagttaaaattctattgaagagaagaaatgtttcctgctgtgccagaaacccttaagaaaaagcaaaggaatttcacagagctgaagatcaagcacctgagaaacaagtttgcctcaagagatgcttccaaaggcaagttggaagcttattgatgaaaaagtgaaacactatcacaaggagtacaggcagatgggacagaactgaaattcaaatggctaggatggcaagaaaagctgaccacttctatgtacctgcagaacgcaaattggcatttgtcatcaggatcagagatatcaatgatgtgagcccaaaggtccaaaaggtgttgtagcttcttcgcctttgtcagatcttcaatggaagctttgttaagctcaacaagcttagcatgctgaggattgtagaaccatatattgcacgagggtacccaaacctgaagtcattaaatgaactaatctacaagcattgttatggcaagatcaataagaagagaactgccatgatagataccaatttgattgcttgatctggcttcatctgcgtggaggatctgattcatgaaatttgtactgctggaaacacttcaaagaagcaaataacttcctgtaccccttcaaattatcctctctatgagatggaatgaagagaaaaaccactcattttatagaggtggagatgttggcaacagggaagaccagattaatgggctcattagaaggataaaataaggtgtctgccatgattatttttgtaatcttgtcagttaataaacagtgactgctttcgatttgaaaaaaaaatacatagttaaaatacacctacacattcacaccaaagattctgatttaattggtatgaagccaaaccaagacattagtacttttccagagcttcccaggtgattttaatttgcagccagtgctgagaaacactgagacgatgcacataaaatgcctagcccagagcctgctcaagaaatgttagctgttgctctctactgtcactgtttgggtggttgagacttgtgcccaggcttcctaggggagtgttcctcccattgccccacagttccctgtaagtggtggaaaaggagaccctatagcagcacttctcaacaccggctacacatttgaatcaatagaggagccttgccaaggtttgcaaaagatggctgggtctgtctctaaccaattagaatctctgaaggtgggacccaggtatccccattatttactttccccattaactattccacaaatatcctcatatgatctgatctctatttccccataatgattgactggtatttctctttgccatttcagtattcacaatctgctttgtattaaagctgcttatttacgttatctctcccactgtgaaccactgcagatcagggactggatttgttttgttttgttttagtttttatacaattgctttccccttgcactcagcatacagcaaagctctaacacatgatatatacttacataatattaatttaattaattgtcagaaaccatagatactcaactataagactttcctgccttctccactctacttttccatttttcatgttttctttgttgttgtgcttttctatgccttcttcacactgtagccaaagttattttattcaaaattcaaattaatttaattatggaagctgggtaatgagcctaataatataaacacagtggtgcagtgagcatgcagttagttacaatgttccatctatttttctgttgtgtgaaggcatccacaatttataaagtacctggtgattcaaaaattaagaaccactattctaagagtaggtggaggccagactatggaatcttccaacaaccaggagcaggagttggaatggaataaattagagcactggatcatccctgaaatccctgagcagaggaacaaccgaaacagagatgtatttaagcttataagtctaaataaaagttgggggtttaaaaaggctacttaattacttctgagaaaaaatattttattcatttctcactgggcttctataatcacctcagctctgtctgcagtttcctttcttctgtaattatttttctctccattttcttgccctgatagccacgtgctcatttgggtatccatcaaaagactacatttcagtatattaattctctcactggatggtacagcttgaatttccttctgctagtttcattaaaatctaaatggggggaaaagtcctctacattatctaggagatccatgtgagctgcccagctccattagctgttgtctgttagaggtggattacaagcaggtgtaatcttaaggacaccgcccttctgtgattgttaagaaggaacacagactgagcagagcggctactgttctgatcaggagccaccaccactggttcctacagtatacagaatttcaattgtcagatctgtcctggaggaacaatagaatacaactgtgaattaaataatttagaaaccaaggacataatctcacttttctaaatgagtttgcctcttttaggagtcagtatattcgtagttttgctagcacagtcctgtgatgtaggaggaaaatgcccaaaaatattctagaaaaaacattgagaattcaagttaaaatgtcagatggagcagcctctagaattcctcctttgtaggccatcagtaatctcactgaaaattagacatgtattatttataaataataatataatattatgcatattttaaatattaatgatttaggtcaaacgcagaaggtgaaatttgtaggtgtcagaaatgaaggccaaatctttagtgggaaggctgtgggagctgaagctctgcagagcacagtggggatccagatgcaatatttagaggctggtgttttcactctcctgtgcagaagtcaatgctccaactctagagcatggaactaggcttcccaaataagcctattgctggcaaagctctgcatagtccgtgactgggagatgggaaatctggaaaactaaccatctgacctaggcccatcccaggcctgtcccggatccctccagaaatctcaagaaacaggatctcaaagccacagggcactatgtgcgaggcgccacgtccactggacacattcagggaacatcattcacatgaacatgtgtccttgttctggttattgattgctgtgtaacaaaccatcccaaatattagtggcttaaaacatcagtttattgcctttcatatttctgagggtcaagaatttaagtggaatacagtgaggtggctcatcttttctccatgctgtctgagactcagccagaaatggcttgactggcactgttggcactggtgccagctgtccgctggaagtctggcaaagatcgccaggaaagttcctcagttcctcctcacttgggcctctccagaactccctttctggtgctacctgggttccaagaggaccaggcgaaagctgcaaggactcctatgacccaggttctgaaattatgaaatgtccattctgccgcattccattggttgaggatgagtcccagctccagccagggttcagggagacgggaccacacaagggtgcgaataatatgaaacttggctcattgggggccatctttggagactagtgaccacacgtctggaattgtgcaatgggccagtacttggttgtggttatagaaatcccagtgtaaagcctaaatagaaacatgagttaagaataagtcaatccaatggtgcacttcagaagtgaccgtaaaactctcctgtagaaggactttcacatattagagcatctacaaagctccctcagtttccaaagatgatagatctcctggaagataaacttgaggagaaggtaggtgcccactatcagagctggactgtgagctccaagctgggctcatttacctagtgggggtctacaggttactcatcctagacccagtcaattgcccctgacctgcctcaggagtgaagcagagccagggtcagggctaggtgggtaggtgggctgccccagacttaagctgtgcattgagaagtgccatagataccaggaatcaaatgaatggagatttgctattatttggaaggatgtaagaccacaagcatatctataagaaattgtaccaggatcacaaatctcccatctcagcccagcagcagtgatctctggaaagaaacaccaccccacatcccaggctggtatacttcacagtaatgatgtgtgcccagattaacacaaatgacaaattccatcatactccctctatctgaaggagcacacctgatgtgagaaagagcatgagaggagcctggctgtcagagcaggaattctgcactgtggcaggttctgctctgtctggctgatgcttctgaatacagcagagatatgacggcctcataatttaggttgcttgaaaggcggagcaagaaaagttacctagtttagaaaaatcaggaaactcatatatggaaaggctatgctatcccaagccagcattgctctggaactaagcagatgagaatgtgagcccagaaagtttttctcccaaagaaatgctctttttgacagccaatcaggcccaagtggtttataaggtagcccaagcggcaaaacatttgagtcagaaaaccctcaccttggcacctctaccaaggaattgtcagtcagccctacagaaccttctgtctggccttatcttaaacttgtggcagaagtaatgccacatgttcacaatatcccatggatggggtcatgtgatgtgttcaggccaatgaaatgtgagttgaagggactatgtcactcccagactgagactgtgaaaagcccctattagatggttgtccctctgctttgagctgtgagtttggaggccacgtgttgagatggtagagtcctcagatcaaagcagccctgaatacaaaatagctgtatttggagcttgtgtgctactgcagataccataagcctcgcctattttgttactccaaagatgtgctgggttcatctttgtagctagcccaggggcaccatgatgctcaggctaccatatgggagggcgatccatgaggagcagcacctgggtggtgaatagcacctgggaagctgaacctctgtgttcagcttggaggtagttagcagatggagttctaggatgtactaccatgagaagaaagtcgagggaagataaaagtcagaatgagggagatggtattggttaaattcaaagaaggaaggataaaaaccttggggaaagagtggaaagagggtggcctccagagctgagtccaccaggatgtacaacaaattatgcttgagatgagctgtccaggactcagtatgcaaatgggtgatctatgcctcagtgcccacttattggaaaacacacctgagatgaaaagcatgctctaaactctgtcactggagagtcttgccgcttggaccgtgagatgactataacaaaacactgccattcatcttccaaaagaggaaatggacagacatataattactagcccataaaatgatggcaaataacacagccctccagacactatacttggtgccatggaaaaatttgaacattattgggcaatcactgccttctgacttcaactttgacttaagaagagcagaagagtgtacatattgggagcaggatgaagacacagagtttctggttcaaatcctcgttctcctgctttctattgggagatcttgcaagttaattgcttgacttctttctgcttcaattatttcatctgtcaaatagagataaaagcaattcctttttcatgaggttagtatgaaaattacagtggatcatgaagaaaagttcttaggaccatgtctgatatgtgagtcctcaatgaatgatgaattcattgaatattatgatatcaccaggtttttttaaaccgctggccaggtatcccctcacaatcctttatctctcctcctctgtaaatgtccaacacaggccacttctcccctttgtacacctgctgtgcaattcctgccactttggaatgggagcatcacagcttaagggtcctaattcctttctcagcctctgggtatcccaagtctctaacttgccattgcctgctttgcaccaagaccagagtgtcaggggcagattacaggagtcctacagtcactgctctgctaatgagtgcttcccttagaggtgaagaagcgaggcagtgcctgctttggaatgctttaataaacaccagaaagtcctcactgccagggatattttactcatttactttctaaagcctaaaattctagaagataggacacattcatttgctaaaatacaaaatttccagggagaggatgtataatgagtcttttttgtttgattgttttttgagacagagtttcgctcttgttgcccaggctgcagtgcaatagcgcgaactcagctcaccgcaacctccacttcccgggttcaagcgattctcctgcctcagcctcccgagtagcttcgattacaggcatgtgccaccacccccaagtagttttgtatttttaatagagacagggtttctccatgttggtcatgctggtctcaaactcccgatctcaggtgatctgccgcctcagcctcccaaagtgctgggattacaggcgtgagccactgcgcccggccacataacaagtcttatcagtcagtatacctcagtataaattaatttatcaggctccttttctaaagggttagatgagggggaaatgtaagatggtggaagacttgggcatttgagaagcaattgagagggaaaatgatccaaatgagaaccctgggacccccagagacagtcactaaggagttaccagttggcatagaacttctacttctgctcccaactgtccaagctcaaaatgaagcccagtagggtcttcagggtctcatcttggaaaaggacagcttccaagcctaggatttcagaatgtataatggtcatttgagtgccgctcatcagtgaacaatagctaatcagtgtgcaaattcagcaggcctgagctccatcaccaaaggcattttcatgaggacacatcctgcttggagaattttgagtgccatcagacaagtcaaatcgctgcccttctctttactcaaaggatctggaatcccctgaaccaggaaaaatgccactgtgacccttctgggtgcataagcacaggaataaacacaatagatgggcaaatccaactgtaccttatcctgatgacatattttagggcagcagtttttaactttctagtttacactcttaaaaataatattgagaacccaaaagaatttttgaggatgcagattaagttatctctacagatatttatcattttaaaagttaaagtggaaaattctttaaaaatattatttataaattcattgtaaataacaataataaaatgttaacataaatgtaatacgttatgctaaataactcatattttccaaaatgacaaaaatgcagcttgcattgttcacatttttgcaaatctcttcgatgcttgtttccattcagctgtgatatcactcatgatgccacctctagaaaactccactctacacacaagagagaatgagaatgaaaaagacaaaggacagctcaggattgctgtaaaaatagttttgactttgcagatcccccgaaggggtcccagagacccacaggaggccctgaccctactcaaagacctcttttttagaggattgttttaggtgattgggctgttctgatatgatccattcctaggcttctaaacacctcaggcaagtctccagggcccaataggaaggtcaatacagcctcttccttgttaagggtagacctacattgaagatcaaggaatcttggtgtttgtagaataattatggactagcaaaggacaaagcagttgcaggtttctttgctgagcctctgtgtttgtgagtctggaacccaaaggggcagcagctgaggggcagggactgagaagtagcaggatctgtctcttaatagctgcaccaaaagggaaagtcatgacatttttctgagacttcatttgtaaaatggaaataaagtccaagcacttcatggagagtaccttatttaattcaaacatataacatagaaagattgggagataatcatctttctctgttttacagatgagaaaccacatactcagcaaagtgcccagtacacaggaagtgacagagtcctaatttgaagctgtgtttgtttgatcttaaagcccaggcttttctctgtcctgctggcttggtgagaaggtgtccagcgttggcaggcagctgagagaagtgggggatggacttttttctggaagtttatctggagctaatggcatgtggtgactatgccaggggtgtgtgagcactcaatacctaataactgcctgtaggaggattaatagaatgtggaaaaatagaacagcaacctggccagttatcaaagcaacccaagggactgcaaataccctcacagtatatgctgtgcatgcatatgtggaccgtgcacttctcaacagagggaaggaaggaggctttttccttttttcatttagacacaaggatgcctggcataatttgctgcaaagcgtttttctctctcaggacttaggaaaatggagtcacaggttagcaagctagattggtcagagctttagtgaaggagcaggtgacctagaaaagatcttccctaggaaaaatgcaataacacacagtccctagaaaatgatgacatgctgtctctatgcactgctaagacatttcagaaacattatgggaaataagaaatggatttttcatacagctggcaccttactaaataaatatgtatttcatacctgttttgttaatgaggattgaaactgtattcccatattgctgccagagtatgtccctaaatcacagacagattgtttccattctcttatgcaaaacatctagatggacttccagttcgagactctgtctggctaacagccctcagtgcaacctgtctcattttctcacttctagaagatgggaaaaatgaaaactcattattatggaaaaccaccagtgacagacagtctaatgtcagaatcagagaggaactgacactaattgtaaggcagatggagctgcattaggaaacaccaagccaaggagccctccatgcccatgccacacttagtttcaaaaagaaaaaaaaaaaaggagccaaggccaggcatggtggcgcacgcctgtaatcccagcactttgggaggccgaggcgggtggatcatgaggtcaggagtttgagaccagcctgaccaacatggtgaagccctatctctactaaaaatacaaaaattagccaggtgtagtggtgcacgcctgtagtcccaggtactgaagaggctgagggaggagaatcgcttgaacccgggaggtggatgttgcagtgagccaagattgcacaactacactcccgcctgggcaacagagcaagattccgagaaaaaaaaaaaaagggagccagacaaaggcaaggcagaagcatggagccctgtgtgtcaggatctggcccaccagaccaggggttgctgagctgtagatgctactaggctgtgaggcaagcagaatgtggactctataccagtctccactttctggaagagaaaacaagtcccatggggaaatatgggcaggatgtaggggcagacataagatatgatgcaatgtgtagttccccaaataaagccctgaatatgtgagttgggaaaagttctctgagtaacagtaccatgttgagagttgtgtgtgatgtgtggggtttggtgtgtgtgatgggggatggggagtgagtgtgcatgtacttaactgcagtatttaagagtcctgaagagtcaggcaattgaaggagaagaagccttccattggctagagaccccatcagaaacttggagttggcctggagctttgacctgcctccagatgcatgtcaggaaaaagtcttatttgttcactgtacaatgaacagaaagcaggggcaagagtaatttattcacatatctgcttctaatcatcaagtcggaaaagaaatctggatactatttttgagaaaaaaatagagaaaccataaataatttggaactgtataagtacactttataccaagaaaaaggaacaacatctggaaggctcaaaagaaaaggatatctctgaaaaaaaaattatgtatatgtaaataccttagtgactagaaaaatatatttcaaggaaatgttttactgtcatgcaaagttgtaggaagacataaataggaacagtggcataagcaaaaaggcagggagggtaaaggaataggatggtaagaaagaacaagaatgatcaaggagcaatgcttgaggcaataatgatcaaaacaggtgtaattcatgttgcataacacccaatccattcctggaaagacaaatttgagatgcatttccagaacgtcaaaggaaaggataaggaaatgaaaacaacaaaataggaatggaagatagcaacgagtgttcctggaagagaaaccggaataaatggaaaagaagcagaatcaaacctacaatagaagaaacccttcctgagcttaataaaagacggaggaggagtcttgtgtgaacaaattcccagggctcaccattttcaagcacagtaattagaacgagaccccacctccctcccaccttcagcatcacatcttgccaaagcattcttattcattctctcaactaacacttatttcattccaactgcatgccaggccctgcaattagaaaggttaaagaaaaaggccccaaagtttcaggtagcacaaaattatgtatttataatatgcacagtgactttagatttctttgtaaaaacaaatatatcaaaacatcatggaaaaacatctacagttttaagacaatagtttgtgactcaggaatttcatatgccgccgaatttatgtatgaaagcaacagtttttaaaaagatgacattaacattaaatgcttaaaaatagttaaaggtgttccagaaccaatggagagttggaacagaattaaagtcctaagactaaagaaataatggtatagaaattctgaaattattaaaatatagataagcataattggtacaatgtagctataatgaaatgatattgcaaatattataccaggaaaagaatatatataaatgtataaatccttaactcaaccattccttttattttattttatagagaaaaggagggggaaaaggaggggaaggagaaggaagagaaggagaaagaaaaagatggaacggtttcattagtaaaaaaaaattcccaatgaattatttaaggatgggtataatagtttctaaatgagggtgtaatttgcaatattcattacaaagtgaaaaatgttaaagtatttaagaataaaacaatgatggagttgcttatatggacaaaacatacctataatgaaatacaatagaataatgtggataaaatccatccgtagggtaataaactgtaatatgtggtatatgtaaaaaaaccaagttataatctaatacttagtacaattctactttggaaaaatatgtctatatgtgtataaatagaaaataaggtctagaaatctctgcagctaaatgttaacagttgttatatctggaggaaaagattttgcatgcttattatgttctgttttatattgatttgtttggggctcatctggactttttagtttttctggaagttacatgtaaaggatttaaacaaggctatggtatatttagatttgtaagggtagagacagaattgcaggggccagtatgaaagagaaggtaattgaagcaaaccaagtgagaggtgatggtggtctaaatccagacaatggcaagaagatggacaaaagtgggcagacttgagctattctaagatgaggaactgatggtgttggagatccattagatatgggagtgtggctgcagataatgacagttccgtatgtcagcatcaacatggagggaagtgtcttgagccactagaagatcagtctgggaggagttggcaaggcctcttagaagaaatcatgcctcctttgagctttaaaagataatgaagagtcagccagctggagaggtgaggaaggccattttagcctgaagtgagtgtcccattatttgacctgtgtcacagtggcctttcccagccaaattgcaaacagagtcaccaaattcgaataaacatttttaaagggagaagaaagcactgctccctctggaaactgtgaacaaattaagccagagcaaatccgtccatcctggctgcagcaatcccaagataatgctgctcagttctaggctgcttggcagaaaggggttattctctgttatgtgcctcatttactcatgaatggagaacttctgggaatccaggatcagccaggcacagcacagttactatttctgggaagatttcatttgaccccaattacttacaatagcagagtgatgtcccattgtccaataataatcaccatgaccttcactgggtctaggtgtcctttacccaaagacctgcttcaaaataccatctaacagattggagctcctgtcaaaacaagaagagtcatcacactgacaggatctccctgaggacaccaagtgcctaagaatttgaacaaagcatcctttctgtgcaggaggaagggggcacaagatttctaaatagcataaataaataaataaataaataaataaataaataaataaagatgaattgtaattatctatgtttagcagacatttgaagtagaaataggtcactgggatttccatttcaaatctctattgaaattctttaccaaaagaattcttttgcacttgatctatccagcctttttttgagagggggatctcactatgtttccaaggctagacttgaactcctgtgctcaagcaatcccttcatctcagcctccggagcagggctttgggtgtgcacccccactgcaccccgctcagcactttttatcagctataatttataacaataatagtgataataattataataaaataatatgaatgtttgttaccatctgtcaggctctgttatgaatatatcactgtctttataacaatcacatgaagaagctaactcaatttctacagaggtcagagagaaaaattatccatttggttgaaccgaaacccatctatcttccacactgagttagtcttttattttaaataaatatgttgttatcaagaaaggaaatcatccattatactttcactttcacctttgctttcaaaatcacccaaacctgaattactagctcatttctccaaaagcataattaatattgactttctttttattatctcctataactagatattgaaaaacactatagctagcataaaggaaaacttctgaacagtgtagccatattgccacagaaacagattcagaaaaataaacctaagaactctggagaaagaatgcatgttatataagtttctccctagagtttggcagcctttgttcatagttggatgtaaacttattcatttctacccataagcccacatagctttacttcctgacatctgttctctcctcccaggatcggaaaggtttcttccttctgataaagtgtgaaacatttgtgaggaaaaggtgggttcagagagggagagaaagacgacagcctgattcctagatctgaggtgggacgcaggaagatggatttcttttaattcccccttcaaagttattcctcctcatgtgcttttgggactatgctttgagaatctctggagtggagtatctggtggtctgcacatagtggtgaagagcgtggagtcacagaatttggggtaaaatcccagctctatgtctaatgagctgggtgaacttgacaggtcaattcactggctaagcctcaccttacctgtttttaaaatggagagtggtgatataagaaaagtgcttagcatagtatctaggttacagtaggtgaccaataaatgggagtgataactaccatcattatattacatggaaagtacaaaacaaatagtatcatagtatgtagcaaagtcctcattttgttggaaaagagtgattaaataaaaccgcaaatgtctgtgcctgtggggtgagatgagggagatgttcaacggtaatagacagcaaagtgttaccaagctacccaaatggatgatccatttaatttctacatggaaaaggagcaaaaaaggaaagggacctctgaaacccttctaagtacccacacacacacagactcacacacacacacacacacacacacagacacacacacacactcacacacacacagagctctcagaacactgctgccagagggtgaatgcagatacaactttcataaccattgctgaaatcacatatattccaagaagggaaaatacttgtaaaactgaaacttattaattaagatactatagccaaaatggatgtcacaatgcttgtctgaatacccataataacatttatgtaaaatttactatgtcccatcaactgtcccaaatcctctaccgggataagatcacgctatcttgacagccactctagttggtaagtgttggttttgagacatctacaattgaggatactgaaggacagagacactaggtaatctacccaggctgcacacctcatagacagttgaggcaagactcatatataagttaacgcatcccagagcctgtgctcccaaacacagccctcatacagaactgctcctgtagttaagaacatagtgaggtccccagaatgtgggcaaaatctagtggagagactcttttccgccccataccagctgttttcaccagattgttagtaaaaagaaagtggatcaagagaatgagaagacaagccacaggctgggagaaaatattggcaaaagacagacatcggataaaggactattgatatggtttggctgtgtccccaccaaaatctcaccttgaactgtaataatctccacgtgtcaagggtggtgccgggtggagataattaaatcatgggggcagtttcccccatactgtttctgtggtagtgaataggtctcacgagatctgatggttttataaatgggagttcccctgcacaagctctcttgcctgccgccatgtaagatatgactttgctcctccttcaccttccactatgattgagagacctctaaatcatatgtggagctgtgagtcagttaaacctctttcctttataaattacccaggctcaggtacgtctttattagcagcatgagaacagactaatacaactatggtacaaaatataacagaattcctaagactcaacaataagataacaaatagcccaattaaaaatgggtaaaagacttgaacagacacctcaccaataaagatacacagatggcaaatgaacatatgaaaagatgctccacatcatatgtcatcagggaatgcaaattaaagcaacaatgacatagcactacacatcttttagaatggccaccatccagaacactgacaacaccaaatgctggtggggctgtggaataacaggatttctcattcactgctggtgggaatgcgaaataggactgtcactttggaagacagtttgacagtttctcacaaaattaaatatactcttaccatataattcatcaatcacacttcttggtatttatccaaatgaattggcaactcatgcccacacaagaatctgcacatgaatgtttatagtagttatattcataacagccaaaacttggaagcaaccaaaatgtcctcagtagatgaatggaaaaacaaactatagtacatccagacaatggaatattattcagcactaaaatgagctatcaaaccatgaagatatgaaagaaaattacatgcatattactgattgaaagaagtcaatctgcaaagactacatattatgattcccaagtatatgacattctggaaaaaaacaaaactatgaagacagtaaaaaaaaaaaaaaaaatcagcagctttcagggattagggagggggagggattaatcagcagagcatggaggatttttagggcagtgaaactatttcatatgttattataagggtgtatatgtgtcattgtacatttgtctaaacccataggatgtacaacaccaagagtgaaccctaatgtagacaatggactttgggtgatgatgatgtgtcaatacaggttcatcaatagtaagaaatgtaccattctggtatgggatattgagagcagggagtgggggttgtgcatgtgtgggagcagagagtatatagaaactctgcacttactcgattttgccatgaacttaaaactgctctgaaaaataaggtgttttttttaatgatgcatatttgccaattatattttaataaagctgggagatcaaaaggagtggaggctatcttcaaacacatgtgcagccttttaaaattcatttataaacaggatctccaggagggtgatcatgtttatcttgttcactttttaaaaagtcattttgaaaaagcaataaattcaaaggaaattgcaaaaataaagaggcctgatgtattattgaccaagtttccctcaatggtttttgcataacatttaacgtaactctagtttgatatcaaaatcaggacaatgatgtatgtacaatgtgtgtatagctggacaccactttgctacctggataagttcatgtagcaaccaccacaatatcttgttcacttttgtcatccggtgctgatcatgattctcaacaatactcttaagtgactcagtcaacaaacaaataagcaagtgaatgaacagatgactgaatccatgacatcagtaaagagcatgtggttcttctgatttgcatatgcactcagtcaatctcacagatcatttttatttgtataaagccttgagctgagagcagtctattaaatacaaggattgggagcacctcaccacacattaaagtgccctttcttccacttcaacttgactaattgagcctatgtgagtgtaactgccattctcccaaagatcgttattgaaataatataacataaaagtggaggaaatgattttttaaaaatccctgatgataggaaagagtgctatccatatggttaaaaaaaaaacctgaggctttcttgccagattcagaacaggtgagatggaaaaccaccatctgggaagaaaaacaaaaaactgcctttatttttttttttgaggtgtaagttttttttattattattatactttaagttttagggtacatgtgcacaatgtgcagtttagttacatatgtatacatgtgacatgctggtgtgctgcacccattaactcgtcatttagagatgggaagagatacccatcaaaacttcattctcaacccccaaaccaagagctggaaataaaaggtgtacaaggtgccagttacagaggaaatgcaccagaccctgtcgctggaacagggtatcagaacatcctcccaatctattctgggccaaggaggcatgctggaggcgggctgggctgagctgggtacatgatgtggggtgcacgctggcagagccagttcccacctgagccagtgatgctaactagagaggcgtgcagtgcctcatcccccgacaaagctcagaagctccaggtagagcagacgctcttgccttaatctttaaaaataactggactttgatctggccaaatggaactggagaaaattctccccctccttttgtctgttcacctgaggtttttcaggaaccatccaagcaattaaacaggagtgacattatctctgaagaaaataatttcagttaccagtttggtattatgaaagacccttgggaataacccctctgatgggcttcagacccatgatagctatacgtacttgttttcttctcacaagaaactgatgggcatgaagattgcagtgactgaactgtctccctgcgtttcttctcacataagctgtaaatgggaacaaagagagaagattaaatgtctgtaaattttcattgcagcccctttttgtttagaaagcatttctgcttaccttttatatttctattaaataatagagagaaatttactcctccagctcccaagagaagaatgaaagttaaattagaatttatagtggacccacaatttctgacctaagaacattaatacaggaatccaaacccctcccagctcttctttgctatagtcatcacccactcagaggttcagctctgaacaaaccccttctgagcctctcccaggaagccttatcaacccacagtccagaaccaccttcaccgtttgagaacctcacaaagggtacttgcagtgctagtgatgcctacagactgcttggccttgtacttgagatggagtgaagacgagggaaaccccttgattgttctcagttttcccctttgtgccttgaaatttccatctcattgtgaataaaaaagtcaaagcatattcaatcccagagaggggttgaatccctgaatttcacaagattgaaagatcatgaaaaaattgaccccctctggaaagatgagaaaaatcaaaaagatcaatgaacacatacaaagagtctgtatctatggctcttattaacagtgactaaagaatttatctgagtaatttagctaattcaactcaataaggaaacaacataataaaagatataactgaaaaaatgaaattatcaatttggaaatgatatgattaagaataaaaaacaagattgtttttgaaaaatcattgggcctaataagacatgtaattaaagaagatgattataaaaccaacaaatataaaaagcaactagcttctctgtacactatcaataaccaattagaaaatacaatcttaaaaaacaaaagattaaatattcaagaacaatcaaaatacaaaatgcatatgaacaaacttttttgtttgttttggggacagagtcttgctctgtcacccaggctggagtgcaatggcacgatctcaactcactgcaacctccgcctccccagttcaagtgattcttctgcctcagactcccaagtaactgggactacaggcacccacccaccacacctggctcaatgtttattttttagtagcaatggggtttcaccatgttggccaggctagtctggaactcttgacctcaggtgatctgagtgtcatggcctcccaaagtgctgggattacagacatgagccaccgtgcctggtcatgaataaattttaaaagaattgtgatgacttacatgaagaaaatcgcacaagtttactgagtgacattttaaaactttaataattgaaaatccattcaatgttcctggtaagggaacagaattaatggtcagaaacaaatcaaatatatataagaacctaggatttttttaagtgcatttgaaatgaatagagaaaaaagggatcatgtctccagcacattgatgttcctgcttttctgaacacacttaactctgtgttgcctgaagacctctgtacttgctgttccctctggcctgaacagtcttccctcagagcctgtttctgatctctgctcaatgttcttttaaaggatagcagtgtcttggggtctttataactttggggcttatctatggctagcaaatgtaggacataggtgtgtagagtatgcaaagcaggaaggctctcaatggttaaatatctgtttatctggttatgtttaaaacaattggatgtgtaaaaatttgagcgtggtgctagtgggcatttaagctaacagatctcagcctactatgaagaagtcataaacccaggagtaatatacagaagccttctggctcatgtgcatgacacaccccacctaattctcaagctcctccctcttcattttttattatagcaactatcactctgtgacattataatctatatctatttggttgtgtgtctgttatctgtttttctcattagaatgttaactccatggaggcaggaaccatgtcatccttgttgactgcattttcagtaccaaaaaaaagtgagtgcctggacagaatggaaacctaataaacattcaccaaaggaagtaggagaggagaggggcaggagagaatatagtaaaataggttaaagtttccaatatagccatgaaattatttttaggaaaagaatttcaataactcgcatagaaaaagattttttaagcatgataccaaggcagaaatccttacaaaagactaacagattttactgcaaaatataaaatttacaaaatttaaattgtaaagatttttttaatacaaatagaattataaacggatttaaatgtttagaatataaataatatttacatagctgatcaagagttaatatactcactaaaaaaggactttctacaaatcaataagtagaagataaatacttaatacaagaataaatacaaatataggaacaagtaagatacacacaaaatgcaattcaccagtaaacctgttttaaatgtttaattttgccaaaaactaaataaatgaaaataaaaataacaatgagttaccaaattttccaataagaaaagaaagtaggttttaagaaattggtagtgcattaggccatttttattaaataaacctaaaatggtatcaatgaaatatttaaaagtacccctgggttacattttgctttttggtactttctaattattccaccaatatgtattacttatgtaattcaaagaagtgattttacaagaaaaaataaatagtatggtaaatcgctggctcctagagagtgggccaatcttggaaaacaatgccccagtgctgagaactgtgtaggtaagttgcatctttcagtagcacaaaggagagattggtttttcactactttagatgagggccaggagctgaggggttaatagtgcacagataaatcttgaaggacaaatgaagttttccacatggccaaaggaaggagaaattataggcagatgaatagaacgtgtgaaaagagaggaacacaagacagcatgggccaatgtagcatcataataaaaataagcaacacaaggccaggtgcagcggctcatgcctgtaatcccagcaccttgggaggccaaggcgggcagatcacctaaggtcaggagttcaagaccagcctggccaacatggcgaaactctatctctaccaaaaaaaattagccaggctccacggcattccagcctgggtgacagagtgagacttcatctcaaaaaaaaaaaaaaaaaaaaaaaatagcaacacttggaattctcagtttgtagaattcacaactgatactaaaagtctatcatgtaacagggctccggttgaagttcacctcactggttatgagttaaagacagggtttttgatgagactgacacagaaaatgtcagcaagaaaagaaaggacacatttctctgatctcgcactctggggaatgctggaacaggtcatgagtgtaccttctgcaagaggggcccagctccctttctcactcatcaagaagttgctcaactttctaatatatcagtagataaagcaagtttaccagtgaggggaagctagtggtgggggcaggaactggaccacttgggcaccagagggtatgattttgatgtcatttgtttagctgcttaggaaagccacccactgccagcttaagattcaggggaagttgagatcaggtgcagccccacctcctgacatggggaaaactcattgtcctcactcattcatcatgcaggcaacctggggagaatttccacggttgcttttggactgccctatttgtgataccagagctctatgacatttcattaattttaattttactgaggctgaatttgactcactcatgcttgaaaggtagggtgtataacacatcactaaacaagcaaatgaggctgtggaccaccagcagtcacatctcaactagacttctgttcaggtatttatcagcacatatgcatgctcactcacaaaatgagtagccacaaaggggtgctaattcctaatgatggtggtggccgttaagagtgaaattggtagttcttggacagaaaagagagttctggataaatgacaaagaagaaaaatattaacacatctaaaagtttgcatgtgaatacagcacttggtaaaagtaaaggtgatttgtgaagctgttttaataatttcttgaaccaattcaaaaattgctcatcaacttaaaaattagacaaatgaatgatgaaacggtggttaacagagtgttaacaggtcactgactcatttaagaatccatgcaggccaggtgcagtggctcatgcctgtaatttcagcactttgggaagccacagtgggtggatcacgaggtcaggagttcaagaccagcctggccaacatggtgaaaccccatctctactaaaaatacaaaaattagccgggcacgatggcggttgcctgtaatcccagctactgggaggctaaggcaggagaattgcttgaactcgggaggtggaagttgcagtgagccacgatcacgccactgcactccaccctgggtaacaaagcaagattttgtcttaaaaaaaaaaaaaaaagaaaaagaaaaagaaaaaatgaatccttgcagagcaaaagtactatcaaggctttgcaatagcctatcctgcaaaataaaagttagttcaaatttttaggaaatatttttctttgcagaatttagattctctttgtgaagagacggcttttcatgatcacacctgtatataaatgtggactcaataggaataattaacatcattcatcactcttggtcagtgctatcatagagatgtaatgcaatatttttgtggaggatcttttcatgacaaagtacactcattgctatttaaattacattacttttgcatttatagaatctgtgtggaagtcacagtttttccattataagttatggccttctatgggataaaattacatgactttccagaaaatggagtttcacaactgtcttgagatatttctcttacgaatgccgaggactactttggtactgctatagaaaactgggagggggaagggacagagatgatattgaggagatggacaatgaggttgtctttaaagctatgcagaggttagggtcacagaggagtcatagtcatgggagtgacagaatcagattggtgttttacaaatattcccctggaaatgaatggaggacagatgggaaagaaaccagattggaggaggcaggttgacaacttaggaaacgaatttaatagccctgaaaagatgatgaagatctaaagttaggttcaaatccagtggtcctcaactttagctagatattagagatggctgaagagcttttgaaactacagatgttgaatatcattccagataaattgtatttaagagtgacatctgggtatcaattttttaaaggtccccagatagttgtgatgcaaggtaagaattgagaatgctactctaaaccaaacaaggcaataaatgtggatacattcaaaaagcttttagaaaacagatgatgactgatggatgggaatggagaaacaggcaggcctcaacttccagatatgtgtcttgaacagcatggatgatagggtcgaaaatattaacacatttcaagctttgaaaaaaaacactaaggtataagttcctcaaatctaacctgccctctccaggcccacagactgggagtggcaatcagcaaggaaaacctctattactccaaaaataatcttttctttggttgggaaaagtaagtcttctcaaaactcacaagagaaaaagcactaaaggtgcttgttctgaagggagatctggatgttgaaatagcagcatcccattatgtgagattagattgatgatttcttcaatactgagatggagcgagtgtcctcagaggacagtgtttctcaagaaggtggtctaaatgaccacaaagtttgataataattaacagatttcagtctccatgaaaacccgtgttacctgcaagtgtgttctctgtcacctcctattggccttatatttttctctcttagcttgccctcttggctgtacttttttctgagatggtgttatgcaatctccccacaagggacctcagtagcactaggcttacaccacccttagagtgagtaaccagtcactatggccatggatggactactctggccagccctaggtcataagataaccctggttaaggagaaagagagtcaccaccacctaaaccatgtggatgaagacctggttgggggaaagataattgagatagtgttattagaagaagaaatatttttagggtaggcaaaaacaacagatgatgactaaaacctcaagagagtctctccactcccttggccctcatcagggtcatgcacctgaggaatgattcatgggtgactctatcccctccctggtctccattcatagccatgtgtggaggcagagatgaccatgctggagaagagaccaccccagaagacacagctaaaataaaggcaaatttgttgtgccattcttggcagggccatctctcttgtttattatcctggagccctcttcccctcctctgttttgctgaccttaataaatgaatacatcaatatatgtttggaaatacatctgtttctacataccttatgccttccctccaaagaataaattaagtgattagtagaatataagagctgaaaatgtgagagctatctgctcagaatccagataaaacagaatggggcttatgaaaagtagtgcttgattagttagtttcaaaaatcctaaaacagtgttttcaaatgttcatttctttgaaacagtagttaaggttttgatactctcccctgaggaagtcatctaaaactgctgataaagcttttggcaaagagcttttcattacggggctatttataacaacagaaaaccgtaaacaacttaaatatccagcattgaaagaacagttaaataatagatggtaaatccacacaaataatattatgttccattaaaaactattgacaataaaattgtaatagtataaaaaaatctcaacatgaataaaaagcagaatagaaaattatatatgcaccataaactccagctatataaaaaagtgaagcctcacattaaagacagggatgagaaggatcatttaataatgtaaataagagttgttttgatggtgataaaacatataagattgtcatctcccaactttctactttcctctagtttttatattttgagtaagaataatttacacaaataacttacccatgtttagttctcagtcttcatcttggttaagctgtcagcagaatttgacacatctgatcactccctcctccctgaaacaacctctctgccctccccgtctcctctagtcttcatataataatacaatataataaaggtatctatttatgtgtttgtctgacacacaatactgtgaattcctttacagcaggtactatgctatactcatcttcatctgttttttctctccttacccagattgaattccacagtcatttttataagtctcttttgcacatcctcaacttccttcaccctgcctcttttcatgatattactggcaaaactacaaccctggtaaaatcctgctttttctctctttgtacccacactggcgaacaaagctggaaacaaacacacacatgcacacacacaccctacctactctccttgcttacttacttacttactttatttatctatctatttattttagagacagcatctcactctgttgcccaggctggaatgcagtggtgccatcatagctcactgcaacctggaacttctgggctctagtgatcctcccgcctcagcctcccaagcagctaggactatggtcatgcagtaccacaatcagctcatttttgtattttttatttttgtagacataaggtctctctctccttactttaataacctagaactcaaataagtcacagattctacccagcaaccatatgcttattcccttacggttcattgaattctccaaaaattatcattagaattttttaaatattcgcaattttcacaggataagaaagcagtatttcatcctacatctcgcactgccctgaactctttcttactttctctctcttctaactttttagcacctcctctttcatcctctcctcaaccagatgactttgtttctactccactgagaaagctgaagcaacagaagggaatgtcctcactccctggcctctctatacctttgctgccaatatcagcattcatatcccctgcctctcacctgaaaagtatgagtaaactctccatgtgacaaggttaaaccatcttctcatgttttagacaccaccacctcttacctactctagatcttcgctccaaaaatcttcccccaaccccgattaagttttctctttctgctgattattaaaatccacatttttcatgctgttctttctcaaacactgaaaacacaaacgaaaaagaagaagcttattcatcccacttttccctccatctcgcactgcatttctttaccacccattagaggaaaacttcttgcagtaattgtccatactcactgtagtcaattcctcttctcccattcccattcaaattgtctagccaggcttctgccagtaaagtggctctggtttaagtacccttcagtaactttcatattctaaaccgagggtcgattatttgaacggacagtagtattcaacacaactggccactccctcttcttgaaaatacttttttacttggtgtctagccaccaaactctcctggtctgcttcgatcctatgaattgcttcttctcagtctcttttattggttccttctcttatttctgactttattaatatttctcatattaatagagtccctacccctcagatcaatgcttggatcttcttctgtacttagggacatcatcatgtcttggggcttttaaagtgtctctacacggatgacccctgaagtgatatctccttccaggagttctcttttgaactcaagtttccttagtccaacacagactatgtttactgcctatatcccaccaccggaatggaaactctgtccaaggaagatttttatctgttttattcactgcacctagaaaggtgcctggcacattacggacatttaacaagtgtttttgaacgaaaggataaataaataaatgggaaattggagcattatggttgaaggccaggagagagcatttttggaggcataaagagccaaaatcaaatcaaagcatcttgaacaagaaattcataaaatcttgccttttgtgtactatagtatcttcataaaatcaattaggcaataccttctttcactattctctagaagattttgtaaaagaccaaaatgagatgtttcttgaatgcctggtggaattcacaggtaaaacaatctgggcctgatgctttctttctaggaatattttacactaacagattccacttctttagggttacaaaaacaatcaggttttctatttcttctcaaattatttttggcaagttattttttaggaatgtgtgcattttattaaagcattcaatttactgacataaatgtgtcaatagtgttatactatttttttctgtctctactgtacctatagttgaatgtctgatttttacatcaaatagtttgtttttggcttcttttttttcttgtaatttatatcacaaatactttttcagctggcttgattgatcctttctattatatttggggtttttatttgttccctgcttttgtcttcttcatctcctttcctctgatttaaattgatttatataccattattctttttctaacatctttttattttaattgaggtaaaatttatataacatatcattcatcaaattaaccaagtgtacaattaagcggcagttagtacattcaaaatgtggtcaactataagatctatctagctccaaaacccaaaaggaaacttaataggaaacctcagaaccattaaacagtcacttccccacaccccccatccagccctagcaaccatgagtctgtctatgaatttgcccattatgaatatttcatataaatggaatcatggtagaatatgtggctttttgtttctggcttctttcactttgcataagatgtttaaagttcatccatattgcagcatgtatcaatacttcatttttttatggctgaaaaataatactccattattacatattatgttttatttatcagctcatccactgatggacatttgggttgtttccagcttttggctattgggaatgatgctgctattaacaatcagacataaatatttgtttgaaacctggtttctattcttttaactatatacctaggagtaaaattgctgggtcatatgataattctatgtttaactttttaagtaaatacaaaattgttttctacaatgactgcatctttttatattctcatcagcaatgtacaagagttctaacatctctacatccttgccagcacttgttattttccttttttaaattataatcatcctagtggctgtgaaatggtatctctgtggttttggtttgcattttcctaataattaatgatggtgagcattttttcatgtgcttgttaaccatttgtgtatcatcttcagagaaatgtcacttaagtcctttgttccattttttagttgcatgtcccacagggtattcccttgatgtagtactctccccacttttcctagggatgtggcttcctgagagctgagctctagtgattgttatctctcttttggatctagccaccgagcaggtctaccaggctccaggctggtactgccagagtctgcccagagtcctgtgatgtgaaccatctgtgggtctctcagccatggatactagcacctgctctggtgcaggtggcaggggggtgaaatggactctgtgaagttccttatttttggttgtttaatgcattatttttgtgcttgttggcctctgccaggaggtggtgtttttaagagagcatcagctgtggtagtatggggaggatcaggtggtgggcagggccctagaacacccataagtatacgccgtttgtcttcagctagcagggtgagtaggaaagaaccattagttggggacagggctaagcatgtctgagctcagactctccttgggaagtcttagtgcagctgctgtgggtaattatggtgtggttcccaggtcaatggagttatgttcctgggaggattatgcctgcctctgctgtgtcatgcacattgtcagggaagttggggaaagctggcagtcacaggcctcacccagctcccaagcaacccaaaaggccggtctcactcccaccatgccccgccaacagcactgagtttgtttccaggcagtgggtgagcagggctgagaacctgtcccaggctacctgcctcccagctgtgaaagcaagtagagcttcagtacttccccacctgtggagtctgcataccagatacacaccctcccccaagttctgtccaggagacttctagtttggtagcaattgttacgaagttcagctggaggtttccttctccttgtagccttttcccagtgcctctggcagcactccccaaggacccctgtgaggcaaggcagaaatggcttgttaggggacccacagagcccacagggctttcctgctgcttcctctacccctgtatttcacttggctctctaaattgacttagctccaggtatggtcagaatattctcctgtcatctagacctttaggttacctagtgacggtgtgtgtttgggggtggacaatctccctttcccactttcacagtttgggcactcacaggatttggggtgtctcctaggtcttgcaggaacaatctgcttcctgcagagggtctgtgggttctctcggctttcctgatttattcctgcagtcattctggagcaagagttcatgaagcaagactccacatgctgctctgtccatctaagtgacagctgcaatctagtcctgcctcctgtccaccgtgagcctctgtaaatcctggatatggaattcttgattgacaggattatttttctttcagcaccttaaatatgttatcccattgccttatggcctccataattcctgaagagaaacctactgtcaatcttatggaggcactcttgtacatgacaagtcatttcttttttgttacttttaagattgtctcttagtctttggtctttcagaaatttagtaacatggtgtcttggtatggatatctgtaagtttctccttggagttcatcaggctttttgcatgtgtagatttgtgtcatttgggaaggttttggccattatttctttaaatattctttctgtccctttatctctctgccttcctctaagatacccagaggtgaaagctcagggtcttctcatgttttttttgagagtttattcagtcctgagtatgcatttgatctctttgatttacttgtacatattagtgcttttgaaaattgttattcccccatatatcttctttcccaacctcattcttcctaggattcttgatctattgtttgtcctgactgttatcacctgctccaagctgctgtggacaattcctttggctttaaatattttttagcaaatgtggttcaggaagctctatgaccctgggaatattctaagttaggtaaaacaaaggcacactttgtactgatccttcagggaccctgggaatattctaagttaggtaaaacaaaggcacactttgcactgatccttcagggagctgccagacagtcaaaacacacaaccaaaactcttcaagaataaagtctatactgtcccctgtgacattagcaaactggaacagtattttcatggccaccattactctggggaagaggggtgacaagcaggcaattttaaattctctaacaccctcttaccacaatgcagcagcttctttcttcattaagcttcccctggttgttgttagtttttcactgtgtttcctagttctgtgaaagttgattctaccagtttttgccagcatttgttgctggatgtatggaaccttagagttccctactctgccattgttggcaaaatcaattgcacctttttttaacttgtcaaattagttgcttagatagtttattttcactttcttcttctctaatataagcagttaggaaccataagtttttctcaaggattttttagtttcatcatctcacaaattataatgtgctattctctcaatctctctccttttttttttttaggtttgggggtgcatgtgaatgtttgttacataggtaaactcatgtcacaagggtttgttgtatagattatttcatcacccagatattaagcccagtacccaatagttatcttttctgctcctctccctcctcccaccctccactttcaagtagactacagtgtcttgtgtctccttctttgtgttcataagttctcatcatttagctcacacttgtaagtgggaacatgcggtatttcgttttctgttcctgcattagtttgctaagcataatatcctccagcttcatccatgttcccacaaaagacatgatctcattctttttatgattgcatagtatcccatgatgtatatgtactacatttgcttcatccaatctgtcattgatgggcatttaggttgattctatgtctttgctcttgtgaatagtactgcaatgaacatttgtatgcatgtgtctttatggtagaatgatttatatttctctgggtatatatccagtaatggaaatgctgggtcaaatggtagccctgctttcatctctttgaatggcaatactgcattccacaatggtttacactcccaccaacagtgtataattcttcccttttctctgcaacgtcaccagcatctgttattttttgactttttagtaatagccattctgatggatgtgagatggtatttcactgtgattttgatttgtatttctctaataatcagtgatattgagctttatttcatgtgcttgttggctgcatgtgtcttcttttgaaaagtgtctgttcatgtcttttgcccactttttaatggggttgtttttctcttgtaaatttgtttaagtttcttataagtgctagatattaaaactttgtcaaatgagagtttgaaatattttctcctattctgtagggtgtctttttactctgttgatagtttcttttgcgtgcagaaactcttaagtttaattagatcccacttgtcaattgttgattttgttgtgattccttttggtgtctttatcatgaaatctttgtcccttcctgtatccacgatggtattacttacgttgtcttccgggtttttacagttttgggttttacatttaaatctttaatccatcttgagtttatttttgtatgtggtgtaaggaaggggtccagcttccatcttctgcctatggcttgccagttatctcagcactatttattgaatcaggagtcttttccccatgacttgttttctctctctctctgtcttcttttttttttttgtagatggagtctcactctgtcacccaggctggcgtgcagtggcatgatcttggcgcactgcaacctccgcctcctgggttcaagcaattctctgtctcagtctcccgagtagctgggattacaggcacccactaccacgcctggctaatttttgtatttttagtacagacggggtttaaccatcttggccaggctggtcttgaactcctaacctcgtatcagctttgttgaagattagatcatgtgttattttcattactatctcattatgatcaacaaatctataatttttattatgatttctttttgggcaactgttacttagaagtgtgctttttaattttcaaaagtaccaggcttgtctagttatatttttgttatcattttctagcttactttcctgtatgttttagtttttttgaatcagatgaaatgacatatggtctaatatataataatctgatactcttctatgtacccttgaatacatgtgttcagccgatttgagaagtattagtttgtatgtttacattaggtcaagtttgtttgctgtgtagttaaaatctgctctatccttgctgattattattatgtattccatctttaaataaataagagaacttgctaaaatctgaagttctggtggtggatttatctttttctcccatagttctattattttcttactttatttattctaatgggcttagaattgctatatcttcctggtgaatttaactagtattcaatatagagtatctctctttatctctagtaatgcttttagcattaaaaagatttttatcttacattgatatcactacaggatttttcttaattaatatttgtctggtataccattttattgttgttttaaaattttatgtttttatgttctaggtgcttcctttttcttcttttttgagagcaagtctacatgtgcctaaccttgtgtctggtagcagtgggcctcaggataaaaggggcaggaagagcagaggaaatgggaaggaccccaattttgctgggtgactttctggcacctagcagggattcaatagatgtccattgaactgtcaaaatgaatgcagatggctggacttccttctagtgttacccttggactcagatgaagtctccccattccttcttcctgtttgaaacactagttgtcccattgggaatccctcatccccaaggccttctgctcatgatgaataatcaatcaggtttgtgattcaaaacaacatccatcatctctgtaaaaccagttccccatcctaaagctatgacattgtttgacaagggtatgtattatttaaaaccaggactttgtcatgttttcattatctgagtacagttggccctttgtatccatgggttctgcatttatgaattcaaccaattgtgaattgaaaatattcagaaaaaaatgcatctatactgaacatgttcacactttctccttgtcataattttctaaacaatatagtataacaacaatttatatagaacttacattgtattaggaattaaaagtaatatagagataatttaagcttggttaatatatgcaaacacagcatcattttatttcagggattttggtatccatggaaggctatccatggaaaccaataaccccatatatacaaagggacaactatattggcatcctttactgattatagaacattcctagtcactaccatagtgcctctttcacattctctttattatcttcttctggaactccagtgggatttttgttggactttctcatcctattcattatgcctcttcacttctctttggtctatctctttgtctctttctattgctttttgagtaatttatttggtccaatcatcaagtttactatttctctttttattggttccaaccttataatcaacatattcattgagattctaactttattacaattattttttcatttctaaaagttctactttgttcttaaatttatctagtcaattagagaattcctctttcatcaaacttgcaattttctgcttaatttctttcaatatattatctattaatttatttatttagagatagagttttgctctgtcaccagggctgaagtgcactggcatgatcacagctcactgcagacttgaactcctaggctcaagccatcctcccatctcagcctcccaagtagctggaactataggtgtgcaccaccaagcccagctaatttttttttctggagatagggtctcactctgttgcccaggctggtcttaaactcctggtcacaaacagtcctcctgccttggtctcccaaattgctgggattacaggtgtgagccaccatctccggcctatttttaaatattatgtaatactgtttatctgatattcccaatatatgcaatctttgcacatctgatttctgtgtgtacacaggtgttcatgtttatacatttgagttttccaaaagccatagtacactcatgagaaaatgaaagtgaaaagacaaatacagtcttagtattaattataaaaatagtttttgcctaatggattcagagtcttagtgaccctcagaggtcctcagactacacactcagaaccacttcactaaagcatccatcgatacaatcagttatttctttcctgtgcctccaaaatgtttctacctcaataccaaccttggaaaaattgagaaaatgaaagctcaaattattttccatggagcttaattttctctgcaattctgatcaaaaaagattgtaagcaatgaaagaatcaaactagctgtattcagtagtcctcccttatccatagtttcactttccatggtttcagttaatagtgcaatagagtaagatattttgagagagaatgaccccatattcacataacttttattacagcatatatttatcattatcatatggtatcagtatattattgttgttcatttctttatgtgcctgatttataaattaaattttatcatagttatgtatgtatgtataaaagaaaacatggtatatacagagttcagtactatctgcagttggaactcttcaaacatatccctcacagataaggggagacctgctgtaatatttttgcttcttaaccaagtgttctccctccacccatccctgtcttagcatttctatcattacataattctctatttggaaaactgtatcatgaggtttttactgccacaatgcaggttaattaggaaatgagaaaaacaaagtagcctatttcttggtcttgagtttcaagtgatttagaaggaaaaatgaaaaccaggctacctggatccaagaatgggaaagagtgaggagtatgcagggaggaggagacaagagcttaaacatggaggcatttctggctgggtgcggtgactcttgcctgtaatcccagcactttgggaggccgaggcaggtggatcacgaggtcaagagatcgagaccattctggccaacgtggtgaaaccccgtgtctattaaaaatacaaaaattagctgtgcatggtggaacgcacctgcagtcctagctactcaggagactgaggcaggggagtcacttgaacctgggaggtggaggttgcagtgagccgagactgcgccactgcactccagcctggcgacagagcaagactccaccaaaaaaacaaaaaaaaaaaaaaaaaagaggcatttctaagagagagaccttaaccctaaggcagacactgaagaggatttggggcatgaaataggagctgcaggttggagaacatcacaagcaagggcatctgtaacccaccctacccactttctcaagagacagtccagagagcgagtaaggcacaggggccctcttctgcatattgaaagtgagaagaaatgcctatgcgagtgtctaggcagagagcttgaaacagccacttgtgggtctctgcacctgcagcctggtgcagggatcagaagaatgtacttattctgtgcaacaagagccatgcctttgtgacaaagcccgtggcccaagaaggccctgcatttggaacaaggaggcaccactttgatctgctgacagctaagtgcaaggtggaatcagagatatcttaatagatgtcaatgataacagatgataccaagaaccaggcatcttagacacacacacacatacacacacacacacacgtgcacgagcacccacgcacgcatgtgactggataccgcatgagttttctaaagcttaaagatgactacaaggacaaagagtaaacacttaattgactgcaattaagtttttgatatccagcagagtaggagtttttactagcaattaacttcagttttagaacacgacaaatcttatttttattatacaactacaaacaaatatataatgaatgctcagattccaggaccctatacctggggtgatgggtgggacgagttaaagaagggatctggacactgctgcctctctgccttctttatatggggaccgtatcacaaatgaggctcactgtgcctccagtcatgtttactgtgcagcaaggcttttggccagagaacataattgacatgcacagagctggagctcttgccagctcacagcaaaagggcctctaattcagagaaaaaggagttggacctctgccagtcatgtacctgttatgctttcatataggcatcagttagaccctgatctcagtctgacaaaccagaaaatataaaaccatcaggatcaaggcatactttttactgggagtgttagcctggtaatggagaggaatgttattgctctaagcattcgcataatatctattgatcaaacctctgtgaagctgctattcttcatgccagcgtctggcggcaagggaggactatttcaggtctcaccatcacagccacttcacactcaggccatgccacagtcaccagcacagcttgggattggggcaggaagtccccagggtgaccagacagaaagcagcctcaggacggggccagagcatgagactaggggtggtggcagctgggttttatttactctgggctgtgtgaccttgggcaagctgccagcccattctgttcctctctgcatgccatacaaccgggaattcctacattactgagtcactcctgggccaaggtttgatgtccctattaaaatgctgttgagagagtaagtggcttcagtcactagccctggagaatgagttcaccagtttgatttgtttacaactgagaactctttcagtttgtgtgcgtgaggtgctgggggagcaggtggaagaggctttgctggagtgggggaaatatcctgcctgggagtgggattggcggaagggggcatatttgtcaatcacaatagaatcctagacacttatgtctgggaggcatagcataggccattgcattcatcttccccccagtacctgaatctgtgaccaatggaactgaggatctttacttaacaacctctagtgatgggataatcacctccttctaaggcaagctacttggaaactctaagatattactgagaaaagaccctgagcccagccttgaattagtccaaagtctaatgcttctcccaggtgacagcccttgaaagagtgtgtgacaggcgccatgccccctgaatggtctcttctcccaaataaataatacctgattctccctctgcaatgttcatgacccaaacaaagctgagcctctgtgtggccactctcagtttgatgttgtaccccaaaccttcaacctcagtcttaatgcctgggaatgggggaatgtgtggaaaaaggcatgaaacacagtacacagcaaaatgtcttaacatttctcttgattctctagctccttatcccttttcctaaaaatctctttctaaatctttcaaggataaagggaagggggtagaaaggggaagtgaggagaggtaaaggaagatgttcattattgggagcctataatgttccaaacacgtgggacatttaatcttcacaaagtaagtatacccaccctaattttataaggtagaaactaagacccaagatatctaagtaagttgcccaaagtcacaaagcaattacctggcaaacaatggattcagataatgactatatcataggccaagcccaattacactcaaaataattcacatcttcctaagaggcaagccctgggggcccaagagggggttgttgagtaagggcagaaagtacaagggagacaagttgccaatcacagcttggccctgattattgtaataactgacacaattaaagttcaatttcgtatccttagaaggtttccgtcatattccaaggctgatgagaaatttccattctaagaatggggatctgttgaatgttttaaattcaggcacttaattaaatcttttaagatgactctttctgtgtttgcccttagtcttttatttctatccaatattgttcctggaccaaatagggtcgggctgctgtttcttgtagcccaataatgagatgcagatgaactggggaggaagagagttttttttttaagtatataaaaacatttattcattagaaaacaaggagactggcaaacatatattccaaagtgaaagcagctcaatgcagttcagttaggctaatttaagagaaaggccttgcattttaaagatcgtgtatgtattttttttttttttcaaaaaaggagacaggcaaatattctacaaggggaacagaattagaattctaggtcaccctacaagttaccctgcacagggaggaaaggaacaggcaagatgacttctcaggatctgtgcctgcgagctgatgctctgagaatgggggttattttcttgggtgtcctgtcttctgtcatctaggctaaaaaatcttcctcacttgactcatcacttgagaagacaacttttggtttcttttcggaagctcgctgctgggtgttcggatgccgatgggtagtacgacggggctctggtgtgacgaagtcattgtctgaggctgcagaagccagaggctggtgcctacaaacagtggatggtttcttggctgttggggctctctggctacctgcttggttaatctcaagctcctcaatcccatccaaacctctggcatgacaggcctgaagcttttgctcaaattcatctattatagccttgccctcaggcttggccccatgttgtagcttgcccataactgacaaaaaagaactgaagatggactcatctgacagtttatctccaaaacagtcaaaattgccaagcatcttgcaagggccttgctctgtgaggggcagctgtgacacacagtaggccaggtcctgctgcacctgctcagtttaggctgtgtggaaccgctgacacagcttttccaccaggctttgtctgcttgtctttggtgatgatgtaggagaagaactgcttcataatggtgtgaaaaggcacttcctccacccccagctcggggtctgacaggtggctgatgatatctggaaggagactatagattgcattgcccttgtggaagagctcattgaagaacttcttggccagggcagcaatttgagactcagggttgatgagcagcatggccatctcactcacctgcccctttaccttcaccatgtccttgaggatcaggtggagtcatcaccagccccactgttttccacactggctgagcagggtcctgaaggtgagcatatagatgaggagtccaggggtccaccagattgggaaagtggatggccagatccccagtggcaacgatgagattagaccggacaatgggaagtggagacttttctagcatggtgaacagaagatgaggctgggagttgcagaaagtggtactgatcatgcagaacttgccaagggtaggtgaagcagctgcagagaggtctgggttgctatagaggcctgagttgttgtagactttaagcaagagtggaacaaaggcagccagtgtctgtttgccatccaacagttccatcttgcagaagccgcggattagttctgcctccgtgtcatctgctgccccaaccagccccagctcctcctccatagtggtctcgaaacttgtattcttctccttgggatctttggtcttgtgctcctgttcttcccggagaactcggcgctgacagagctctccactcactgcctgctccaagtggaccagctgctgcagagccacatccccagccagggacaagaggttcatcaacaggaaagtggggagcattgtgggagactccttcgggtccccctgactggttctcttctcttctagcttctccagggcctgttttgcacagccctgccatatctgggcacagatcactgtgggactctctgccagttggtaaatgagggtcactgccacctctttgaatgggatccagagtgggtctggtggacaaagcctttcgggaccgtctcccgcagtcactcaaacaacttgtgttcctgaggcaactggaagtgggggtgacgtttgcccagagaagtctttctactgtcagagatgttggcgatggcatggcacacctgctgggccagccggtagtcctgtggaaacttcggaagagagttttattttctgcaaccggtgacggggagaaggcctggaaattattgccagaccaacttaaaattacaaagttttccagagcttatataccttccaaactatatgtctacgtgtaaggtatgcattcatctaaagatgtaaatggttaacttcttttaatctataaccaaggtctgagtcctaaataccttcctctggagcctcagtaaatttacttaatctaaatgggtccaggtgctggggggattacccttatcttgtctcctgctaaattacagaggttttgggagttccttcagacctccaataaacttgtttgtggaggcctaaggagtttccttagacccccagtgaaacttgtttaatcctaaatgggtcctgttaagaattcctttgttattttgtcatgccttaagtcccaggaaaggcctaggtaaaactcttgatgggcttttgttacattccagccttcgtacaggggcactggcttttaatatttaacttaaccactcagtcagtactgaaagagttgtcagtgacacctggcctgccacaattatgagtgtccagaattttactagatgtttgggggaaggggaatgagaagcctgaattaggaatattctccacgagtctctcacaatctagttggaaagactagtaaactcaatgacatattaaatgataagtaaatgacaacctgtggtccttacttcatgcctttgcacacactgctctctctacctggatgcccttctctcacctgatctctccagcaaagcactactcattcctcaagacacaaagctgaacaggcactctgtgaagcctgcctagtccgcttgcttccttcccagaagattttgccaatcatccctcttttggacaaatatcgtgcaacactgtattttaattatttcccaactccctgaaacactagaatgtgagcaatttgaagataggaatggagtttccttccttgcattctcagcacatagcattgtgtctgaaaccaaatagtcagcaatcaatagtaattgatgagtgtttaaagaagcactacaggaagtggcaaaaatcagggtcctccagggcaacacgatggggacttcacagagtgggtggagcttgtattgggccttagaggatgactaacatttggagatgctgatgctgccaaaagtagaaagatcaggaggaataaaagcccagaaatacgagactgtcttccccagtgttttatggtggtcctagattcaaatatcccatcatgtcacaactttgaaatgaggaaaatactttcaccaagaagttgaatgctattctgaattacaaacccaggtgagtgctcatcaagaacagaagaccagccaggtgtggtggtgcacgcctgtagtcccagctattcaggaggctgaggtgggagtatcacttgagcccaggagctcatagtgcgctatgtggatcggatgtttgtactaagtttggcatcagtatggttacccctatcccaggagcaggtgaccactagattgcctaaggaagcatgaaccagcccaagttggaaacagagcaagtcaaaattcccatgctgatcagcagtgggattatgccagtgaacagccactgcactacaatgtgagcaacataaagagacgcccctgtccaaaaaaacaaagaaggaaacctgcaatccgagcactttgggaggctggggaaggaggattgcttgaagccaggagtccaagaccagccagggcaacatggcgagaccctgtctctacaaaaataaaaataagttaaacataaataaaaaattctaaatgaattttaaaaagaaagaaaaaataatagaaggctttggggatcagggcagcccaaagggattcctggagtttcaaacacaaaatagacatgagagacttaatctggatgtcagaccagcactagaagaaaacctggacattttgaagcccttaaattcataaaactttgacagtttcatgaacatttgttgaaaccttgccatgtccaagaaggtcacctaggatcagaacttccacccatctcctcttctccccctgtccttttctgaggtctacaccccctttgtagtaggtggtcaactggacacaggactaggtttatcaaaacttgctctcgggaaataaaaataagacaagaaatacaatatacacaagaacacatgttcttgtacctcagtctctgaatagcctgtctctagactctgtttgatgtaagagtggttgcttctgaaaaaagaactgagtgactaacaatggttggagaagggtgcctttcttaattttagtttttattatttttattgaagtatgaagttcaatccggtgcacgcaagtatcagctgatgaattttcacaaacagaagataccagtgtaactagcacccaaatcaagaaatgttaccatctgcctctatgccccctcctaccaattcccagtaccacctcttccccccaccaagagtaagccctgaggcccagacaggcttcacctaattttatattttattttttctttttatgaacagctttattaaaatattcacataacaatacagttcacccattaaaagtataaaactgagtggcttttaatatattcacagatatgtataaccatcaccaaagtcaattttaaaatattttcatcacctcaaaaagaaggcaaataccttttagctataactcccacctcacatcctccgtaaccctagccaactgccaatctacttcctatgtctatgaaattgccagttctccacatttcatataaatggaatcatgtaatatattatcttttgtgactggtttatttcacttaacactttcaaggttcatttttgttgtagcacgtatccatatttcattttttatggctaaataatattctatcatatggatatagcacattttgtttatccgttcatcagttgatgattatttcagtggtttttaccttttggctcttaggaataaaatgctgctataaacattcatgtacaactttctgtgtggacagttttttctcttgggtatgtaactagaagcagaattgctgggtcatatggtaacactgtggttagcattctaagcaactacagttttatattcccaccagcagtgtacaaaggtttcaattcctcctcatcctcaccaacatttgttgtttggggattgttcattacaaatacatgaaacagtggttaccagagtcagtcagggtgggagggaggaatgcagagatttaggtcaaggatacaaagtagcaaatacgtagaatgatttaattctttacataaatagaaccttacagtatgtgtgcttttgtttctgacttcttttgctcagcattagattcaggagattcatcaacattgctgcgtatagttgcagacagtttactatccttattgaatggtgttctactgtgtgaacatactacagttgcttttttcattccactagtggtgggcatttgggtcatttccagtttagggctactatgaatactgctgccttgaacagtccaggatacatcattttgtgagctgcttatttttattatttgtgctacctgaattttgtattatgtgcaggtattactttttttaaatgtcctaaaaacacctttaagggcttttaaactaggatcagaaacttagctccttccctactgacaagcaggcagaaaaaaaaaataatagtaatagctagtatttactgaatgctcattgagccacctctttagggaaaaaaatcttttcatatatgtttgcttttaatcctccaaattactcaaagccataggtattctcattatgcccatgttagacctgcagaaactgagaccgaaggaggttaaataacttgtgcaaaaccatacaactagcaagtagggaatcacagtacaaatacaggtctctgaatataaagcctgtgcattcaccctccaattacacagcctctgattacactcagctgggagttcagggagcctgctagacatggggaggagaagctgaaatgagagcacctgaaactgccagctcagtccagctcaacagctcggattgttctccccaccgcccccctacttcctgtcctaattaatcaaatgctcttgaacatgaaagagagtgagattggatttttgttaagatgaaagaaaggaatctgttcccatctggcaccttcaaaactctcctattcaagccagcagggagcatttatgcaacctacatggcagtttaataattccccaagaatgccaggagattaagcaatgtcacaaccgatctggatccaccggcacttgagaataatggaaagagcaggggtctaaggtctaggacaggggtcaaccaaatttctctgtaaaggcccagatattaaatattttaggctttgcaggtcctatgatctctgtgtcaactacccaactctggcattgtaacatgaaagcagccatgagcaaacaggcatggctgtgtaactaaaacttcttttatggcacagatatttgaatttcatataattttcatgtgcctgggcatatatttcttttttaaaaaatattttctaaccagtaaaatatgtaaaagttattcttagtttgaaagctgcacaaaaatggaggacaaagtctagcgacctgaattcaaatcctctctctgccatttgttatttctgtggctttcgacatattacttacattcactaagcttcatttctctcttgagaaaatgagcagagcaggtgccatgcattcagggcctcccttgtggctggcactgagccacatgctttatatgctttactgaattaaatcctacaataaccacaaaaggctgaaattatatgattgccttcttcagatagttacgatgattaagaaattatgtctataattgtgcctgagctggcctccatagttcattgagtttgatgcttcctgttgtacgcaaaaattgccatgttggttcgatgagagcatgcctgctttttctactacctgtggtgttttgagtacctatatttgtattgcacttacagtgaaaattatctaattatattattctatttaattacaattgcatcaagctccaataaacaatccaaatttcactgaactgaaagttgagtgcacactttgaatagtcactccttctgttaggttggtgcaaaagtaattgccattacttttagtagcaaaaacagcaattactttcgcaccaacctaatatatatgtgcacagtaggcagtttggctcaggtacacttattcaaaacattgcattaagtaagataatacatgttaaatacctagcacacagcctgggatgcaggtgtcggccagtgtttgttccccacttccttagtgacaaagattaggcttagctactttttgggaaaggtgagtgccaccagaaagatgaggccaaggccttatcagacacactggaacacagccttcccaaggaataaatccagaatattttgtgtatgagttctggctctgaagcccagcctgttcttacactgaccccccagtttctgccttgatcagccacccactaggctgggcattgcttcctctcactctgctatctagtgacccaacctcggcccaaccctccagctttcttctggccatgcgactgttctgctagtctgctccgcatatctgctcctaccttccgatgcctcctctgagctttggctccactccccactcagcacaaacaacttactttgccccagtatggggtacaacagctcagactcttctagaggtctaatcctggacacctcgtttgctttataagcacaatctggaaagaagagtttcattgctcagagcccctcaggaagcagaagaggagagtggtgaagagagcattcatcctctggaatgaccataactggtgtttcaatcccagctcccctccatactaatacttgggtgagttactcaccaggtctatgcttcagtttcttcatctgtaaaatggaggttgtaaaaatatcatctaccccatagggttatggtgatgattaaattagttcttctatagaaagtgcctgcagcagtggttagcatgtggtaggcactttgtatgtgttatctgttatgtctgtttgctcttactaacatctatatttctgagtcctagacctccccagagagtgtgaagttcatggtgtttcatgggaacagatgtaagcccacttgctgaatctctctctgagctaataattaacaaatacattgcacagtagcttaccaggaaagcctatactggtctgaacaagaagaaacttgctgaacaattctaaagctcacttctcaatataacagcagcccttgctcaacatgttgcccatatcttcagtaccgtgcagaggtcacagccatcatattctgccagctcttcccctatgggttgagcaaccatatgtgtcttcccaaccaggacatttttcatttttagcttcttgttttgacataatttcaggtttacagaaaatcttcaagagtagtacaaatacccccatgtaccctgtacacatattcctgaaatgttaatatttttcatatttgctttatcatttcctctatcagtctatctataatacctacctatctacctatccatccatctaaaaatgtcccaggtaatcagcaaacctgctttaaatgtcaccaattatcccaagaacatcctttttagcacaaaaaaccccgaatcatgtattgtattcagttgccatgtacgtttagtgttctttaacttagaacagttccttggtctttctttgtatcttatgacatcaacatttttaaatagcataggttagatattttgtggagtttctctcactttgggtttgtgtgatgtttccttctgaatagattaaggttatgtgcttttgagagttatacaccaggaatgatgctatgttcctcttagtatatagtatcagcagacacagtatatcaatttgtcccattactagtgatgttattgtcagatttctccactgtaaagatactatttttccttttgcaactgacaagtattttaagagtacatagccatcctgatactccttaaactttcactcacagcacttaccatacagtgtagattcctgtctgaatcaatgctataaaggtttccaaatgatgattttctaatttcattatttcttctatacttattagcagtctctctactttaaggaagaaatttcccttctctcccatttttaaatttacatcataggttcttcttttattctgtgggttataatttgttgccatagttatttctttttatgcctagattgctgactggggtcagcaagagccctacaatttggtgcgtgtttctgttgacatgtttctcagcatttttgggcatgtcctcacttgccagcacaacatgatgttccaggcttatcttgtccttttcccagccccaggccaggaatcagccatctccaatgagccaagtaaaaccatggcatatttaagagaaagggccacaggcataatcatggcgggtataaactgaaactgacctagaaatgctgggtcatattcccaatctaccactagatgtccttccacacctcaatagttcttcacaggaagcagtcctggggcatttggaaatggtgggatatttggagttatcacaaagactggggcagcaggggttacttctggcatttagtaagcacagcccaggcatgccaaatttcctgcacaggaccatctatcctaacacagaattgccccataagtagcaatcctgttaataaatagtggcccaatccctgaactaatctatcttgtgttctagtctctcaaggattggcttagttcttttcaccttatccgccatcagttaatttaatcaccactgtttccatagtgagaatctaatatattagactgatcaagtgtgttaatcaaatatattggattagctggcatgcattatagcaaataaaatagccagtcttcattttagatctagtttaaagaaagcctctacttcttatgtgtcaatttttgcagaaaacattattctagcaagttggcaaggagtctaggacttatgttaagacacaccatctcatccccagttcttgcagctctctgtctagttattggccctattcattgcatcagcaccaacaacagctacccaggttttatagggcaagcattttactaaacattttacataaactatctcatttaaccttcacagtagccctataaatcaggcattcttattacaattttacaaagagccctggattccgtgtggtcagtagcaagcccaagatagcacagctgtcatgtagcagagccaacattcaaaatcccacagtcgggctccagtagccatgctgtgtactgcccatgcatgtaagaacatacccaacacaatgaaaaataagaaatgaaatagaaagggcatggaaaatatcaaatcataactgcagaggaaaaaagtcaaatttaagcttagagttactgcaaagttactgcaacccatcatattttattttgtgcttcctagttagtggctaaaactaagggcaaaactgactaaatataatttgttgtaatcttgagggaaatgcacagcaagatgtttcttgatcaagaaaactgatactacggagctttatattcagtactttgatgaaacagcactaaaatgtcctcaataacatctctacaaagaatcagtgattcttagacctctaagaggagccagtgtgatgcctaaacataggcatgtaggctacttgacagacagactcagcatagcagtcatcatcacgtagtaatttctagcatcctagtagctttctcactggctttagttttgaataaatgggatttttgctaagatcttacattatgcaacagaggcaaccacaaagaccacccctagaaaatgaaatgatcttgggccccttcctcccttacctcctgcttcacactgcattctttgggaagcggatgctgaaacagagtcaggtatacaaaaatctggggagaagtcactcctgtgaaagcaaagggaagaagtaagattgggctggaggacccatcagagcctgacattaacctgacagtctctggcagctcaggaatcctttaggatttccatgttgggtataaatgattagactcttctactataacttagtcattggtagaggttgtaccaagaatagcatgatcccagctaaaaagctgaggcaaaccctgaggaatctaacagctggaggatgtcagcaaaccatatacctcacagctggacagcaaggtctttcttgaagggggatctgagcagcatatctctatgtctgcctcatttccctatgtacaagcgtgtacctgattgcagcagaatgagatgagaaacaaatatttttttgttacaatttaccacttccatctcagcctgctaccccttagctctccttgggcaaagggaaaaagacatttgaagagcctgcctcttggataagtgaaaatggaactctcctctccttctcttggatttctaaacatcccagatcatttctgtgaattctttttattaggaaaatagcatcctttaaaagagaaaacaagttgtctatgtcttatattcagagtcagtttgagccccatgttgtgcaatgatcttgtgcaatgataattacatgctaccatgcctcaagaaatttccccttaagtggagtgaggaggagaaatagtggaggctcaatgtcttgttttaagaaaacacctgttattgctgaacctgatgcttgtgcttatgagcacaggagttttttgtggcccagcaataaccaacaactcaggtaggccagttgcctcatttaatgagcttcttggtttctgaatacaacagaggcaaagagagtaagttttacgaggacttgggtttccttaattagacacaagctttttatttggttgggggaactaatggagaaagggagacactagcggcatttgtaacttgttaagactaattgaaaatcttaggaccaaagctttctgtgaagagagtgctctaatgagaataaatgtgatgattatttacctaattgagcagtaatgactgaggtgtgagccacttccccaggattaataaagagccggggagttgacagtttagaccattaggcttagctggtacttcatcttggcgaaggacacagtgccagaatcatattaatattgtaaaatgagcccaaaacatagacatatacttttggagaaataattttaattggctaagtacctgacatcaggttgggccattcagttttatcattaaacaaagtttcacacaagttcttcaagacttgtgccatttacagtgttctcagcattcagtgtttaaaaaagaaaagtagaagagggacctttatcaatatcaaaaatagttttttgtgcaaccaaaagcaagcaagctattattatatatacacactcgctttaaaaacaacactaaagacattgagttgagctggaagcattgcatactctgagatacacagttgatattgttacccacagtgtcccttcaggaaagccagaggtcatctgcaacaccacaattaaggataaaaggaccttatattatcagaaatttcaatgtcttcataaaaattgtgtttatattcaatatctcccttatttagtgtgacaaatgttcatgtacgagtcatctcaagaaaaagagttactcagcttgctacgtattcattattctatggctctgctatgcaggggaaagtgcaaacagcaactgagagaaagcaggaatctaagacatgggactcaacttgccctttacacagggtgaatcctacactagtgacctttgtgttttaaggaagtttgcaaatatgtttaattggacaacatttcttcctttatctagcaagcactgagtatcttgccaggcactatgaaaaaagataaaactataaacatgagcaaggcacagccactgccaatcagaagcttacaatctagcagagatcttaaaatgcagggagctcttttagaaatctgcttagggatcaaaatttccaatcacacttttccttccagcatctttattctgtaatcattattttaatcttcattgaacgagatgattaatgtggttccatctgctgtatgaaatcacccatgctacagtcagccaatcaagcaacagcagtaataatgtactacttcatttaatggaaatttaccaagctgttactatgtaatatcacagttttagggcctggagaggtagagctgaactaaaaataataatctctgttatcccatagtatagtggaataggaaagaaatacaaaaaaaaataatgtgttcatgtattataggtactataatagtagggtaggaagatacacagaagtgggaataaccaagccaatctcagtactcagaaaaggtgttataaaggaggtaaccttgaattaaaccttgaggaaatggatattcttcaggtagaggtgtataaagagcttcccacaccaatggagcagcataaagaaatggcatggcagagtgaacagttgggtatggccaaggaaactctcaactgcatcttcttcactgcctcaactgatcactcaacgctctccattctctcagcaaacaccctgactaactaaactcccatgactagcaggatattattagacttaacaaacctgtcaatttatatgctttccctttagaaagacttgacttttttttagtgaactgcaatagaactaagctcaggagatgcaactatgaaagatgaaggaacattataaaaatctctgcaatgagattctgccttagattgctttgctggtatgtcaactgattactccaatttaaagaaacccaaactggaaattataaaagataaattgtgagtgtattttatgcaaaaaagaatcaaatcaaatcaaagtcttggctctacattaaacaaacgacaaaggcatgtttgtttttatcaaaatgttttagggtagatttgtatgtatgcgttatttctttaaccaatgttttaatcagttaaacagctgctattattaatgtgctggttgaaagtgtttttcttaaagtacaaatttaaaatagaatgagaaaagatcagacttgagaagaaggaaagtcacagtaggacatttggaatttattctctagaccagaggtctgcaaactccagcctacaggccaaatccatcctgcagcttatttttataaataaagttttattggattacagccaggcccatccagttacagaattgtctatggttgcttccactctacaatggcagatttgagtagctgttacagagactatacgcaagtgtgcaggagagtttagaccacacctacaggaaaggtatgaaatactaccctccttcctagacacttccccaaacaaaaaacttaagtcagtgtgggaacaaacactgttgccccacagagcactaataaaggcagataaatgactgagaaaaccctatcccttggggacggaggccagaaatggcaacaccaataccattggaagtctcctacttctaggtaaggacatgaaattctctcatatgcaacacccatcacagatacaaggcaggagtttgattttcatggggaggaggaattggaacactgagaataccccacccctaaggcccaggcacactggcctatatgggtctgtggctgaactgggacaaaagagaagccctaaaaccaccagtagcaagcattgagtaacaagcagtagcagtattacagcaaaacaggcacagctgaaagcagagagtggggcattaaaacccttcagcgtctcagctactatgctaatagaaaatccaaagaaaaatctgaagcccataggaggctgaggatgaccctactaacaacaaaactcaagtctagctagactcctaagcagattgatttaataccccactctaacaaccttaaagaagtgtgcccatttccatgcataaatactatgtatctcaatatttgatatactctacacaaaatgtttgccatttaatcaaaatgcatgagactcaaaaagaaaatgcaacccactgttaacagccaaagcaatcaacagaaccaaacagagacatgactccaatgttggagctattagacacggactttaaataaaataattacaatgaataagaaaaatacattaaaaatacttcatgaaagaggatatatggatggcaaagaagcacctttaaaagtgttcaacagtttatacactattgatggaaatgtaaattagttcttccactgttggaagcagtctggagagttctcaaaaaacttaactaccatttgaattagcaatcccattactgggcatatacccaaacagaagaaaactgttctcccaaaaggacgcatgcactcatatattcatcatagcactatgtatattcacaataccaaagacatggaatcaacctgaatgccaatcaatggattggattaaaaaatggaatactatgcagtcgtagaaaagaatgaaatcatgtctttacagcaacttggatgcatctggaggccattatcctaagtgaattaatgcagaaacagaaaaccaaatgccacatgttctcatttataagtgggagttaaaccttggatacacatggacataaagatggcaacagtagaggggaaaaggaagaactagagcaagggatgaaaatccagctattgggcattatgctcagtacctgtgtaatgggatcaatcgtaccccaaatctcagcaccacacaatatacccatgtaataagcctgcacatgtaccccctgaatctaaattaaagttgaaatcatttctaaaaaaagaaaagaaaaatgaacaaattgaaaataaaaagatgttcaacatgattagtcattagagaaatgaaaaataaagccatgatgaaatatcactacatatctagtagaacaactaaaagaaaacacactgacagtcctgagtgctagaaagtgtgtgcaaccacagtctctctcatacattgttggtgggagtacacagtggtgcagcaacattggaaaactgctgggcagttttttatgaagttaaacatatatttacttactctgtggcccaacaattccacttctgagtatttgtcctagagaaatgaaaatttatattcacacaaaaacctgtacatgaatgtttatatcagccttgtttgaaataacaaaaaactgaaaataacccaaacatatttcagtgagtgaataaacatattgtaatacatttatacaatttaaaactattcggcaataagaacaaacaaatgatacacacaccttagaagaatttcaaaggcattatgatgaattatttttttttttttgagatggagtctcactctgtttcccaggctggagtgcagtggcgcaacctcagctcactgcaagctccgcctcctgggttcacgccattctcctgcctcagcctcccgagtagctgggattacaggcgccagccaccacgcccggctaatttttttgtatttttagtagagacggggtttcactgtgttagccaggatggtctcgatctcccgacctcgtgatccacccgcctcggcctcccaaagtgctgggattacaggtgtgagccaccgtgcccggcctatgatgaattttttaaagctaatttttaaaggttgcatattttgtaattccatttacgtaagattcttaaaatcacaaaactataatgataaagaacacattagtgtttgctaggaaatgggtgggtagggagtgtggctataaagggatagcacaagggaatttctcgacagttgtagaactgttctctatcccgactgtggtgttagtgacatgaccttatatgtatgtgagattaaatttcattgatatatgacatgcttgtaaaaattggtgatgtgcaaataattctatagtttagttaacagtattataccaatgtcaatttcctggttttcatcattatactgatattgaccaggtagagcgtacatgggaaacttctgtcttatttttgcaactttaatatgggtcttaaatgatttcaaaataaacactttaaaacattctccctactaaataaccatgattaataggttaaagtttccaggggaaaaaagtggacaacatagatgaacagttgagtgatttcacagaaagataaaaactataagcaagattcaagtagaaatgtgtgggggaaataaatcacaatagtaattcacagatttaattctaggaatcgtagactttacacagccagaggaagaattaggtaatctgaagatagatccataaaaattgcccaaagtgaaaatcagtaagaaaaggacaaagaaagtggatgaggagaacaaagtatttaaaaatgatgggacaatatcaaatagtctaaaatatatgtcattgaagttacagaaggagaagagagaatgggcaaaagaaatctctgaatgatagctggaattctccaaaaataatgaaagcatcaaatcatagatccaaaattcttaaataacctgaagcagaataaattttaaaaagctagctataacttatccaaagtaccaaataaaggtaaagaagaaattgaaggaagacagtggaaaatatatgaacatattaaatcagagaaattcttaaagaatacaaaattacagctagatagaactagaataagttctagtgttccatattactgtagaattactatagttaacaataatatatggtttcacataactaaaagaatattgcatgatcctcacaaaaaaatgataaatgctagagatgatagatatgccagttatactctgataactatatgtactgagacatcaccatgtagccccatgaatatgtacacttattttgtcattttaaaaattaaattaatataaagaggtaaagacaagaattatagctgacttcttatctgaagcaattaaagccagatgataaaacatatcttcaaagccagctggggggacaactatcacccagaattctacatctagcaaaaatacctgtttaaaatgaagatacaataaggactattcaaaatttttgtaatgggagaattccttaccagcagacctgtactttaagaaacattaaagaaagttcttctgacagaggaatgtgaaaccagaccggaaattttatctacagagagatgtgaagaactctgaaaatagtaaaaataaagattaatattaaaagccacatttttcttaatgttaaatgactctaaaatgtaattgactacttgaagaaaaattgtagcagtgagatttataacccatgtaaaatttaaatgtctgataacaatagcacaaagtttatgaggaatttgaatgtctaagtcttcacaatatgtaagaatttgtataatattatataaaacatctaatactcagtctttacttgctgcagggaagatcaatttggcaggtccagcactggaattaccatttagattagttaggaaactgcggaagttcaagaaagaaatcatcagtacctagagtatgccaaataatagtaagaatacagagaggattcaaatttgagatctacttaataaggtaaaataaggaggatgcggtgactgagtattgagatgttctgaatgtgaggtagagggaagagtctactatgtattatgggttcctggcttgggtgtccctagcagacagtggtgctatcaagtgagtctgaggagagaggaggataggatcattaatatgaaaagttaatgagtttggattttaactggggtttcaggcaatttacatgtaatgttattattgatgtgtttggattgaaatctaccactggtttcttgcttttatttgttccatctatatttttcttccttttttccttctatttcagcctggattaattgagctttttatgatttcattttgtctccactattggcttattaattatgcctctttttctatttcttttaatgttgtcctaggcttcacaatgtacatcctctctacattcctacagttatttaacatacgtttcttccttgaacttctgcatagtttcctaattaaatggtaattccagtattggacctaacaaattgatcctcaccacagcaactaaagaaatctatttataatacatgtttggagacataaacattcaacaactctcacagtataaaaaaatacatcctcttttaattggcatgtgagacccagaagtctggattcttcttgtttctgcagacccatttttgccacttcctagctcctatttaggcttcagcaataacaaacagatgcttaatatacacaagccaagtagtttctcatctccataactttgttcatgctgttatatgtgcctagaatcaatattctgcattcctttctcacctgaccaatcctcactgaaaagctcagctattgccccattcttgacttcttccttgtccacccgcttttgtgctccaactggaaccccattcatgtttacaagaaacactcaaaggacaataaagcctgttgtgactctacttcggcttttctgagtattaaaatttcctaagatctttcacaggacaaatttttaaaagcaagagtaatacatggaggtgattaagcctttaaagtgtaccctttgttcccaaggtcttgttaattatcatttaggagcagtttatctgcaatgtgctcaattacttaggtaactaaatagctttttactgatcagcattgaacatcatttgcttaatagcaactaacaaatgcggtggcagaattccactgatagtctactttcagaaataggcttatttaagattgagctccaggggaaaaatagattctaacagaatagattattgttatttacataaccacttaggtctagagaaacaaaataatctttccgttttttaaaattaaggaatcagtaagccaaaagagaatatccaagtaactcccaagtccattccgttcattcatccagtccatttgtgccatgaaaatcttcatgacattcctatttgatcaatcacaactcacagctctcaatgactgctcacatctgaccttctcagtaaggacttttcttatagcttagaacagtcataggatttttcctctccaagcttttgacactttggattatagatcccatgtgcttgccaatgctttttttgttaactgtagaaaaataaaaagcaaatacagacaggcgaaagactcagagggtcacataacattgtatggtgaactataccatgtgaatggtcaaggaaagcttctggagcatgggacatattagctaggtgttgaaagaaaaataggaattcaataggctggaaaatggggggagggtatttcaagcagagggaacacctatctagatgcacaaatgtatgatcaatatggtgttcaacattgaaatgccacatggggacctgacaagaggtagttgtattagtttgttttgtgctgctgtgatggaatgccacagactgtataatttataaacagaaatttattaggacacagttctggaggctgggaagtctaaggtccaggtgccagaaagttcagtgtctggttaggtcattctcttccaagatggtgccttgcatgctgcctagatcctcacgtggcagaaggccaaagggagaaagaggatgagcccattcactttttataatggcattaatctattcatgagggctcaacctaaatgcttcccattaggccccactgtcacattgggaattaagtttccaacacaccaattcttgggcacacattcaaactgtagcagtaggcaaagccaaatagtggaaggcttttattctaagctgtgggactcagctttattctgttaacagtgaagatgcagaaagggcgagatgcagtggctcatacctgtaatcctagcactttgagaggttgaagtgagtggatcacttgagcccgggagttcaagaccagcctgggcaacatagtgagatcccgtctctatgaaaatttttaaaaagtaaaagttagctaggtgtggtgtcacgtactgttcccaactactcaggaggctgaggcaagaggatcacttgatcctgggaagttgaggctgcaataagccatgatcacaccactgcactccagcctgagtgacagcaataccctgtaccacttccccaccaaaaaagatgcacaaaagggagatttatctccctagttttctaaaggagcaaaagcaacttctgtgggtggaatagccctataagtagaaggggccaaaagataaaaagggcaaccaaagaagggaatgaggggatgagaagaagacagggactcatatctgcacattagtctagggaataaagaagaaaagacacaacatcaatctaaaaagaaataaatgaaaacaataaaaggcagtgttgaaggcagtcgagcactgacttctatagtatgggttaaagcccagcttcacctctaactttctctgaaatcttaaaatgttctaacctttcttattttgttcatctataaaatggagattttatctcagtttgttgtgagattaaacaagttaatgattctaactacttagaaccttgccaaggatataataactgcttaataacccctcacaatcatgttctgtgttctagagaagtcaggttaagggtagtaattctcttttaaaccatgaagtctacctagagttatgtaggatgtcaccattgggggatgctttgcggtaggcacatgaaacctttctgtaccatttattgtaacatcttgtgagtctctaattatttcaaaataaaagttttattttgttttgtttttcttaaagccttttcaaaccacaatggcaattcattcaagcatgttctcagcacttgctgcatgccataccctgagctccttctgttctaacattagtaaacctcattttccaatattcagaaagcaattaaccaaatgacagtgtgactttccatttttactattgctctttagcagaaacatagcaataaaaataaactattggaaatgaaattatgagtatcctagattaatccccaaagttgcagtttcttttgttgttcagctgtttgattttctaagagcctgaacccagccaagtgcatactctatttctgagcagagttctgcccagttctctgtctgactagaattgcacagaaaccacaaatctctcataaagcctaatggtgtttctcaacagaggcatgattggaattctgagctggataatttaatactagggtacagcccatgggtagtacagaaaagagtcaatacttgccttgtctttgtacaaccaggatgtatgtctgcatgacaataatgaagactcaatttcagcccccattcacccttccacaacagcaggggcagggcaccctagagcataaataatctgaacaaccctgtgttgggggcctgctgaagatgcttaacatccctggccccatccactaattgtctgccatcaatcacaacttgacagttacaagtaaattgcctgtgttctccccatatgcctgcctttctcattgtgtaaaaacagctctacctccatctgctagtcactaccaatcactcttgccaagatggtgactcctcttcttgcctgctagtccctagacacaaagagtccaaagtgccctggtggcagccatagcttttagttcaatggtacctttgctatgttccctggcagaagtacacttcctttggtgaccaggacctccaagttgaagagcacagaggtgcagatgaagaagtgcaaagcccgttagtgggtcaatgggagtgatgttaacttaggccatttcttcctgtaccccttgattctaagacctatgtatacttcctgttagggacccaacaccatgaagtgggctctgatttaacacatatgctgcactttaaaggatgggaccctaaactttcagagcattacctttaagtggcacttcatatgttccttcagtagttcattctagggctctaaaagtacagctacttctgtctggtgaagtatgtaatacaaacagtaggttctaggatcaagagctatcctacacccccttcacaattaagtgggtcccctgatcacatactatgttgtgggatttccattcttgtttggtaatctaaaagcccccagataacagttagctgtgttgactatgactcagagatcaggaaaggaaagcttatacccagaataagtgcatcattggaaggatggacagctggctgatgcaggacagaggggaaccaatgtagtcaacttgccacaaatcaaccagtcgaatctcctgaagacctagtaacctattgagggctccttgttggtctctcttgttagcaagttggacattcaaaatcagcagtagctagatttaccaggataattggtagtccattttattggctaatgagtagtcttgctctctgccactgtggccactcgattcatgtgcccatcatgccagtacttaatccaaagacaaagcctggataatgtcaactggacaagttatcttgtctatttgttttttcagtgcttcttgtgtacttgatgctttccaattagtgttaaacgcatgacacaaaaatcttcacacattgtgcccactcccatagtcccaaccacatgtctctgtctcagactcttgtttccaatctccaagtccctttcattcaggtccctgtccacacagctagactattggctactgtgcagaaacctatacatatattcacaccttaggccaatttttcttccacacgaatagataaccatattcattgcttgcagcttcattcattgggaagaccttccttctccactgtcttccaaggctaccccataattcagctgtacttcagtgatcatctatttttgctttacagctacttaaggaactaacccattgtcagccaggattggccttttacctccctttcagctagtcatagagaacccccatatagccataagtgtgagctatgcaagggcactggtacaaccctggtgggtgacatggaggcatctgtgctacttgctcatgcagcctcttcatgccagtctagtcccatatgggtttgatcccagatgtgccatttccattttccaatagaaggctgtgggacacgtctgagtgtatgacttagtgcattggcaataatctagctcataatggtcagttccaaatacatttggtgccccattatcagctgttccatcttgaccaaggctcagcaacattctaggaagggtttctcaaaaggtgtataactttgtactgaaaatggatggcattgcttcagagccctaggggcctgcattctgattctttcgcttgggcatccatcaactccaaactgtatctttccccactaacacattcagcaccatggggtccaccagatcatatgcctcaagtagtagggttgcttatactgcagcccatacatgctgcagagccctttcttgctttcttgctccaggcccactcaaagctgtgagacttctatgtagttcagtatatgggaccaaaaatattcctagatgtggaatgtgttgcctccaaaacacaaagaggcctaccaggtatcttgctctatttattgagattgagaatgtaagatacagcaatttaacttttacttgaaaatgcagtggtgggagtggtattctagcagactttcactactgaaccactaaaaactttaccgatgtggcatgttcctgaattttcacagggtttgtctctcaccctccagagtgtatgtgtcctaccaatgcctctaatgtattagacacttcttgttgactcttaatgacatctttgatgtaaatggatcaatgtgatattctacagaatgttcagatggtccatgtctcttcagaccttattatgacagatgtttgaagaggtaacatagccatgggcaaagcagaaaacgaatattattgtctgtttcatgtgaatacagactctgattttttggatgtgtctttgtctggttttggtatcaggataatattggcctcgtagaatgagtttggaagtattccctcctcctctatttttcagaatagtttgagtaagatgatattagttcttcattaaatgtttggtataattcagcagagaagccatcagttcctgaacttttctttattggaagacattttattataagttcttgttatttgttattggtcatagttcaatcttggtacattgtatgcatctaggaatttgttcctttcttctgtaatttccaatttatttacaatttatttatttgagtcttctctcttctttattagtctggctaaaggtttgtcaattttgcatgacatttcaaaaatccaactttttgtttcattgatattttgtattgttttcttcatctcaatttcatttatttctgctctgatatttattatttattttctgctactaattttgagtttgttatgctcttccttttctagttctttaagatgcatcattaagttgtttatttaaagtttatcttctttttcaatgaaggtactaatagccataaacttccctcttagtgattttgctgtatcccatagattttggtatgttgtgtttctattatcatttgtttcaagaaacttttaaattttcttcttaatttcttcattgacccaccagtcattcaggagcatatggtttgatttccatgtatttgtatagtttccaaaattcctcttgttattgatttctagttttattccattatggtcagagatgatgcttcatattatattattttattttgttttaatgttttcaggctttttagtgatttaacatatagtctactcttgagaatgacccatgtgctgtggaaaataatgtgtattctacagctgttgtttggtctatagcgcagattaactctcgtgtttctttaccaattttctgtctggaagatctgtccaatggtgaaattgtggtgttgaagtctccagctattattgtatggggtctatatctctctttagctctaacaatatttaatttatatgtctgggtgttccagtgttgggtgcatatatatttacaattgttatattctcttgctgaattgacccctttttcactatatagtaaccttctttatctcttcctatagtttttgtcttgaaatctattttgcctgatataagcataactagccctgctcttttgtggtttccattgacatggaatatcttttcccatccctttattttccgcctacatatgccttatgtctttattggtgaagtgtgtttcttgtaagcaacagatcattgaatcccttttgttttttcaatccgttcagccacttggtgacttttgattggcaagtctcagagtctcatccaaagtcttcaatgtacctgggtattgctgctggttattctgagcccagggactctttagttagcaggtgatgaatgttgccaggactatgtccttcccttcaaggcagtagtttcccttctggcctagggcatgtctagaaatgccatccaggagctagagcttggaaaggcggcctctcagagctgactagtgccctctcctgttgtgactgtgctggtatccaagatgtaagacaaaatccttcctactccttccttctccttcttctcctttccttaagtggaagaaagagacctcttttggagctgtgagaattgcagcctggggttaggggaggggtagtgccagaactcccttagccacccaagctggtatctcagtagtccatgtgcctccccagtgtactggctctgggcccagttcagaactagaacttgcttgaaagttgcactccttgtggcctagactgactttcaagtgtatttagagccctagagcactttagcttgcagtggtaaggcttgtgggaactcaagttctgaccaccaggattggtgatttccttttggctagagctaatataaatgctgtctccatggggtggggcatcagctgagtttggcccagttttcttttatgctataataggacagcactaagttcaatgcctcacaattgctgtgcttttctcccctggcacctaggaatgctctccacaccctgctgccattggtgggggatgggagaggggtggcgtcagagatttaaaactgttttttttttttttctacttcttcagtgcctctttcagcaatatgaagttaaaaccaagcactatgagagctcacctgatttttggtgcttacaaagctactttttttgtgtgtagatagttgctaaattagtgtccttgctgagggaggaagagggaacgatcagtggaaccttctattctgcaaccttgcctgcagcactcttttgagaaagattctggattacgtgataaattagtgatgtctgtcatggaggggtgaggggcagtttttagacatatagcctatcttgccatttctgctataaacatctgcacagcttgtacaatctgtaactatagaattgagtacagatgacctgcccacaagaggaaaaatgtcctgatcttggccttagtgcctctgtaattcaaccaagtatattaattaaatggatagatcatacacttgctgtttcccaagtgtagaggttgaaagtggaaaggagaagaaagcaaagaaaactgggaggaggggcactaagactgggcataaggagaaacaagaggtccaatttgcaaaataatgggtgtagtaaggggaattttgaagtattctccaaagataacgattttgcttttctcaattttgtcctcttgattctcacacagtgctttctttagtacagacctacataccccgaaaacttctgttctgtgacttaaaaagtcaactctatgattagcatgattcctcttcagtcattactgaaattcttcctaggtggttacatttaagtggtaagaagcttcagtaatataagtcaaagaactcctaccttgctttgcatttcctcagtggccatctgataaagcaggaaggaaaaaaattaaaaatgaacctccccaggatttcatttctattgtggcttagctgcaaatttgaaaaataaaaataaaaataaaccctgaatgtaagcacttgtacgtttttgttagattctttcacaggactggtcaacatcttgacagatattaattggcgattcactggagacttcacggcacctgacctggtttgccgagtggtccgctatttgcaggtatgtcacaccttccaaatgtgatgagacaaactgataccagagttagcttttagatttatctaaggaaaatcatataaaaccttgcattcctatgcatcaaactgcccgctttctcctgggtagaagggcaatgaaaatttgatttttaatgtatttcctttgctaaaaatagtatctatataaagaaaacacaaacgagccacaaatgagcaatgctaaggacataaagcattcatatttcaaatgttaaataaaatgtctgggtcaatattaaggattttggcatattgtggataatctccaaaaagtttgttaattatatttacctttcacttctcaaatgaactcaacgctggctactgctcttttttgtaaaccaaggggaatttgttggaacataaccattaactattgaattactgcctaaattatactttttaaagtatctattggcatttgaatttcttcctctcaaatttcctattttcctttttgaatgattttataactgagagactttttagtttttctaagatctatttttagattaaagatattgatgcttcaaatgtcaatctgcttcattgtaagccatatttatgtatcatatgtatatttgatttaaaatattacatatatacatatttacatatatacatacttgttaatatgcattagatgtttataatatagatttagacacttcaactaagtctggaaggaatggattatttcataactggtattaggtttgatgtgctgtttctgagttcttaagattattttttattaatatatttttattctgatgctagtacttcaggtctaaattattatagctttatgtatatataatgcatttctagaaagctttatcctgctagattccatttgttagtagactcttttaggatctttgtatatatattcataagtaaaattttaatttttttcattggttttattattttcaggtcatggtaatgaagttattccatattcagaaaatgaatatggaatagttacaatcttcttttgtgctctgaatataaccttgaaagtttggtatgtctgaaagagccttttctattatgtacggctagtattttctcaggggaaatttttaaaaacatatttcaatttctttctgaggatatttgcttccttgtaaataaatgctttcaattgcttattatattagcaatcaattgctgatacacaaattatgacaaaacatggtggcttaaaacaataaacatttattatctcacagtttctattagtcaagaattcaggaacagcttggctaggcagatctggataaaggcttatcaaaaggttcggtcagatgctttgtggtactgtagtcatcagaacgcttggctgggactggacaagatggatcactcacataggtggtaaattagtactggctttagtggaatactcagttctccacatgggcctctctataggtacattttagtgtcctcacaacatagtagctggcttttccaagaataacctattcaagtgactgaggtggaagtggcaacatatttatgaactagtctcaaaggtcatacaccatcactttcatatgattattttggttgtacaaatcaacttggattcagtatgggaaaagaagaaccacaaaactaggaagtaaggattattttcgacatgttggaggttggcaaccacactatcttcatatttttccaaagcatttgataacctatggtttctcccaattgttaactttaaaatgtcttcttgatttagaaatattcatgtcagtaaaattttttctaactgtatctacaataaatgttaagactatatataaaatatatcttttgacatagggaaaacgatagagaaagaaattctgataattctctcttctataaaggaagcaagaaaattggcaaaaaatatcagaatcaaatttgtaaaactctggaaattaaccaaacacttgcaattactcagggagcatttattcaggaaaatggctgaatctcagtgagaaaaatgagctttgcggtgctttggcttgcattattctcagcccccagtatccaactctgtaacagccttaaaaagtaatagctctcattcatagtgaaaacaaattgtctggcagccactgtagaaagcaaaacagggacagagccctttcaaattcccaagaaattgccattatttgacatgtctggcagttctctggaagactccacttgcaaggctgtctttattttacttaaatcagaactcacccactgtgaaaaacgtttctcctctgggcattcgtcaaaacagttacaggcaattgattaactttgcttatgacaaaggtattggataatagtcagggaaaacaacagactaaccaaagcttaagaacatacgctgagtaatgagatatccataggggatttgaaaatctctgacatactcccaaaaatctagaagaccaggtatatgcccaggactatgctcatcctaaggaaaaaatagagaaggcactgagctgtaacctgtgaatgatcttgaggctttgtgcaagcaggaagtgagggcaaagacagaattgtaagttgcctggctaagtgtcaaaatcatgctccaacattcacacagacttcctctgcaatgaatgggggaattaatagtttctagaatgtaagaaaatctctgtttaggacctatcaccaagatcatggagtaggagatattcatccctccaaagcattcaagtatagacagctattcacaaactaaaatagcccagagaggactcaagggcccattaaagaatttgcagcaacatcgtgaaggaaaaaatggagaataaccacatagaaagaattgctggtgtgattggcacacctaagaggccatgatttggctaggaacaaagaagaaaggtggaagctatcagtattagacacagaaaggaccaacattgtctctagtggcctgctccacaggagacactagcatcttttcccactgagtaaccaacagtcattcctgcaagagaaccccagagggggagatgtggctataccttcctcctatcccccaagaagcagttgctgtcgagctgctttgggaatggggtcaccacctctcccaaccatgtgtaagctccagccctggagccatggccaccctgggaatgcccacactccagactgatagtctgactacactgggctcacccatgtttttaacatcacagccagcaccatagtaagctagttaacactccaagccccagtgttaaactctgcctgcatgtgtccacactccagacactgacttaaccaccatagagagctagccccatccaggcctcagagccattataattctgtgcatacctgtgctctcattcttgtctccttgactaatttatacgtatactattaccaacattacagcaggggcacctgtgccctgggcaacagctcagccatagaaagctacgccttgccccaaccctgaagccactgaaagactgtgttctcagctcccacactactttgcatgtgcactcatgcctcacatactataccatgcagcagaaggggcatctgcaccttgagcaccaatgtcattaccattctaatcccagagccataatctctccatgtgagcccatgcatcagacctcagtttcatggctactccatgagtgtcacccatcagacactggtgatactgccactaagagaggccctacaagccagatctagtaccaagagaaatcttctcagctccagcttccctggtggggggaaagatcagaagaaccttagcagccatcaccactgaagaccctatcaattcttgctgctactgccaatatccactgctttggccactgaggatccttatgatctttatcaactctgacctgagctgaaaaagctacacagagactacaaatctgcaccatcactgatgctaccccacccaacaaaccctccctcatagggcatggtctttccatagtgaactagttcataaaatctggaaggtgattctttttggttttgttgatgtttatctttcccttatattgatgtgtgtgcatttgactgctttaccaaacgcacacacatcaacataaggcaaaaataaacataaaaaaacagacataacatcatcaaaagaacacagtaattttacagtaactggccccaatgaaatagagatatatgcaatgcttgaccaaaaaaatcaaaataatttttttaggaaagcttagtgaacttaaagtacagagaagcaattcaatgaaatcagtaaaacagtacaaccaaaacaaagaattaaagagagagactgaaattattttttaattaaatagaaattctggagctaaaaaacattataaatgaaataataaatgcactagacagcatccagcagaaattatcaagtagacaaagaatttgtgaagtgaaaaacaggttatttgaaaatatacaactaaagaaaaagaaagaaaaaagaatgagaaaagatgaagaaagcttaagagatttatgaggcagcatcaaaagagaaaatgtgcaatgcattagtccattctcatactgctataagaatgtattcaagactgggtaatttataagggaaagagctttagttgactcacagttctacagggttggggaggcctcaggaaacttataatcatggtggaagggaaagcaaacatgtccttcacaaggtggcaggagagagaagaatgagaactgagcaaaaggggaagccccttataaaaccatcagatctcatgagaacttactatcatgagaataccatgggggaaaccacctccatgatttaattacctcccactgaattcctcccatgacatgtagggattatggcaactaaaattcaagatgagatttgggtaggaacatagccaaactatattatacaagttataggagttatagaaggagatgagagagacaaaaaaaatatagattatttaaagaaataatagcagaatagtttgcaaatctggggaaagatacaaatatccaggtgtacaagaaggtcagaagtttctaatcagatttaatgaaaataagactacaccaagacatattataatcaaactgtcaaaaatcaaaaacagagaaaatcctgataacagcaagagagaagaggcaaatcacatataagagagttataacaaggctagcagaaaatttctcagcaaaaaccttgcagaacaggagagactagaagaacatatgtaaggaaggaaaaacaaactgccaaccaaaaatatttacccaacaaagctgtcctttaggaatcaagtagagagaaagatttccccagacaaacaaaagctgaaagagttcatcaccaccaaacctattttccaaaaattctacacagaatcttcaagctaaaggaaaaaaaaaagagcgagctaattagtgacatgaaaatatataaaagtgattttgggctgagacaatggggttttctagatatacaatcatgtcatctgcaaacagggacaatttgacttcctcttttcctaattgaatacccttcatttccttctcctgcctaatggccctggccagaacttccaacactctgttgaataggagtggtgagagagggcatccctgtcttgtgccagttttcaaagggaatgcttccagtttttgcccattcagtatgatattggctgtgggtttgtcatagatagctcttattattttgagatacatcccatcaataccgaatttattgagagtttttagcatgaagggttgttgaattttgtcaaaggccttttctgcatctattgagataatcatgtggtttttgtctttggttctgtttatatgctggattacatttattgatttgcatatattgaaccagccttgcatgccagggatgaagcccacttgatcatggtggataagctttttgatgtgctgctggattcggtttgccagtattttattgaggatttttccatcaatgttcatcaaggatattggtctaaaattctcttttttttgttgtgtctctgccaggctttggtatcaggatgatgctggcctcataaaatgagttagggaggattccctctttttctattgattggaatagtttcagaaggaatggtaccagttcctcagaaaaaatgctcaccatcactggccatcagagaaatgcaaatcaaaaccacaatgagataccatctcacaccagttagaatggcaatcattaaaaagtcaggaaacaacaggtgctggagaggatgtggagaaataggaacacttttacactgttggcagaactgtaaactagatcaaccattgtggaagtcagtgtggcgattcctcagggatctagaactagaaataccatttgacccagccatcccattactgggtatttacccaaaggactataaatcatgctgctataaagacacatgcacacgtatgtttattgtggcactattcacaatagcaaagacttggaaccaacccaaatgtccaacaatgatagacgggattaagaaaatgtggcacatatacaccacggaatactatgcagccataaaaaaggatgagttcatgtcctttgtagggacacggatgaaattggaaatcatcattctcagtaaactattgcaaggacaaaaagccaaacatcgcatgttatcacacttagatgggaactgaacaatgagaacacatggacacaggaaggggaacatcacactctgggcactgttgtggggtggggggaggggggagggatagcattaggagatatacctaatgctaattgatgagttaatgggtgcagcacaccagcatggcacatgtatacatatgtaactaacctgcacattgtgcacacgtaccctaaaacttaaagtataataaaaaaagaaagaaaatatataaaagtatatatcactcactgttaaaagtaagtacatttacaatactctaataatgtaatggtggtgtgtaaatcactgatatcattaggataaaggctaaaagacaaaactattaaaaataataatagctacaatagaggtatacctcagagatattgtgggttcaattccagaatgcctaaataaagcaaatttcacaataaagctaatcacaatatttttgtttcccagtacatataaaagttatgtttacattttactgtagccttttaaatgtgtgataatatgtctaaaaagtatacatgttaatttaaaaatacttaatggctaaaataaaaagattaacagtcatctgagccttcatcaaatattggcctttttactgttggaaggtcttgctttgatgttgatgactgctgacttatcagggtggtggttgccaaaggttgtggtggctgtggcaatttcttaaaatcagacaacaataaagtttgttgcatcaattgacttttccttacatataaaaaattatctgtagtatgcaatactgtttgatagcattttacccagagtagaacttctttcaaaattggagtcaattctctcaaatcttgccactgttttatcaaatatgtttatgtactagtttaaattatttgttatcatttcagcaatgttcacagcattatcaccaagagtagattccatttcaaggaatcactttctttgctcatccataataagcaattcctcatccattctagttttattaagaaagtgaagcaattcagtcacatgtacacactccacttctaattctatttctcttgctatttccaccactttcgcagtacctttctccagtgaaatcttgaacccatcaaagtcattcataagagttggaatcaaccttctacactcctgtaaatgttgatacattcaccttctcccataaatcatgaatgttcttaatgacatctaagtagtgagtccttctggaaggttttcaatttcccttgtcctgatccagcagaggaattactatctatggcagccatggccttacaaaatgtatttcttaaataataacactggaaagtcaaaattactccttgatctatgggatgcaaaatggatgttgtgttagcagaaatgaagacaagtttaatctttttgttcatcgtcatcaaagctcttgggtaacaatgtgcactgtcaatgagcagtgatatttggaaaggaatctttttttctgagcagtagatatcaacagggagcttaaaatattcagtaaaccatgctgtgaacagatgtgctgccctttaggctttgttgttttatttatagagcacaggcacagtagattagtctaattgttaagggccctaggatctttagaatgatcaatgagcattggcttcaactcaaagtcaccacctgcattagctgttaaaaagaatatcatgatgtcctttgaagctttgaagccaggcattgacttctcctctctagctatagaagcgctagatggcatcttcttctagtattttactgtttgtttccactgaaaaactatcgtttactgtagccaccttcatcagttatctaacctcaatattctggataacttaatgcaacttctgtattatcacttgatgctttaccttgcatgtttatattacagagacaacttccatccttaaacctcatgaaacaaactatgctagcttcaaacttttctgctgcagctttcacatctatctcagccttcatagaggtgaagagagtttaagaacttgttctggattaggctttggcttaatgttgcaactagtttgatcatatatccagaccactaaaactttctccatatcagcagtaagcttgtttcattttcttgtaatttgtgtgttcactgaagtagtcattttacttttcttcaagaaagtttcccttacattcacaacttggctaactggcccaaaactaagcttttggtctatcttggtttctgacatgctttttctccctaagctaagtcatttctagcttttgatttaaagtgagacatgtgtgactcttcctatcacttgaacacctaaagacaatttcagggctatttattggcttaatttcaatattactgtgtctcagagaataggaacagctgaggagagggagacagatggaggaacaaccagttggtggagctgtcagtatgcacacaacatttatcagttaagtttgtcaccttatatggatgtgatttacagcaccacaaaacaattacaatagtatcatcaaacatcacttgatcacagatagccttaatggacgtaataataacaataatgaaaaagctcaaaataaccaaaatatgacacagacatgaaataaacacatagtgctggaaaaatgacagacttgctcaacacagggttgccacaaaccttcaatttgtaaaaaaaatgcactatctgtgaagaacaataaagtaaatcatgacaaaatgaggtatatttgtagtgtgttaagggatatactatataaaaaatttaaattgtgacatcaaaaactaaaatgggggtggtagagtaaaagtgtagagtttttgtgtgtaatcagagttaagttatcattttaaaatcgctcgttataagacattcctttgtaagtctcaaggtaaccacaaagcaaaacctgtagtagatacatgaaagataaaaagtaaggaatcaaagcataccattagagaaaagcatcaaccacagaagaagacaggaaaaatagaagaaaggaaaaaaaacacacaaagcagaaagagtggaagacagaaaaaaaatatatgatagccagaatataattattaaaatggcaatttagggcacatataactgaaagtaaaaatggaaaaaagatattccatgcaagtggaaaccaaaatagatgaggggttgctacatttacatcacatagaatagactttaagtcaataacagtaaaacagggcaaataaggtcattgcataataataaacggattatttcatcaggaagaaataacagttataaatatatatggaccctacactggatcacctaaatatataaagcaaatattaatagacctgaagagagaaatagactgcaataaaatcataggtcttcaaaatcccacttttaataaacagattatctagacagataatcagtaaagaacgattggatttaaactacactttagaccaaatggacctaatagacacatatagaacatttcacctagtggcagcagaatatataatcttttgaagcacccaaggaacattctccaggataatcatagagtaagccacaaaacaagtcacaacaaaattaagattgaaatatataaattattttttcaaaaacaatgacatgaaactagtaatcaataatataaggaatttcaaaaatgttacaaacgtgaaaattaaacaagagactcctcaacaatcaataggtcaaagaagaaattaaaagcaaaaatttaaaatatcttaagataaatgaaaatgaaaacacatcataacaaagcttatgggatgcaacaaaagcaggtctaagaggggagtttataccaataaatacctacatcaaacaaaagatttaaaataaataacctaatattacaacttaagtaactagaagaagaagaagagtcagggtttccctttcataaaaagggagaaataataaaaattagagaagaaagaaaacaaatggagaatagaaaaacaatagaaaagatcaatgaagagttagttctttgaaaagataaaattgacaaacctttagctagaataactaggaaaaagaagaaaactcaaataaaattagaaatgaaagctgagacattacaactgataccatagaaacaaaaagcatcataacaagcgactatgaaaaactaaatgtcagccaggcgtagtggctcatgcctgtaatcccagcacttcgggaggctgaggcaggcagatcacctgaggtcaggagtttgacaccagcctgaccaacatgcagaaaccccgtctctactaaagatacaaaaattagccaggtgtggtggcgggcccctgtaatcccaactactcaggaggctgaggcaagagaattgcttgaacccaggaggcagaagttgcagtgcgcaaaaaaaaaaaaaaaaaaaaaaaatagataacctagaggaaatggataagttcctagatacacaaaaactaccaagactttgagtattgaaggaatggaaaatctgaacacaccaatactaaggagatttaatcagtaataaaatgtttccattccaagaaaagaccaggatcagatggcttcactgcaaaattctaccaaacacttagagaagaactaatagcaatctttctcaaagtcttcccccaaaaaagtgaaaaaagtgtttacttccaaactcatgttacaaagtcagcattactctgataacaaagtcagacaagatactatgagaaaattacagggcaataaccctgatgaacataggtttaaaaatcctcaacaaaatactagcaaacatatttcaatagcacacttgaaatattattcaccatgattaagtgtaatttattcctggaatgcaacaatagtttaacatacacaaattaataaatgtgacatgccacattaacaaaatgaaagataaaaggcatatgatcatcttaatagatgcagaaaaacatcgtgacaaaattcaatatctttttgtagtaaaaatgctcaacaaattaggtacagaagagaagtgcttcaacaaataaagaccacatatgacaagtccactgctaacatcacagtcaacagtggtaaactaaaagcttttcttctaagattaggaacaagataagaatgctcactattgcaacttctattcaacatagtactggaagttctagccaaagaagtaaggcaagaaaaaaagaaaagaaaaaagaaaggcattcacatcagaaaggacaaacttaaattttctgtttgaagatgacctgatcctacatatatataaaatatataatttatatataatatgtattatgttataaatatatgttatatgttctatgttatatataatatatgttctatgttatatataatatatgttatatgttatatgttatatataatatatgttatatgttatatataatatatgttatatgttatatatatgttataggttatatgttatatatgttataggttatatgttatgttatatatatgttatatgttatgttatatatgttatatgttatattatatatcatatatgttatatgttatattatatatcatatatgttatatgttatattatatatcatatatgttatatgttatattatatatgttatatgttatattatatatgttatatgttatatgttatattatatatgttatatgttatatattatattatatatgttatatgttatatattatattatatatgttatatgttatatatattatatataatatgttatataatataatatatatgttatatattatatataatatgttatatattatatatgttatatattatattatatatgttatatgttatatattatattatatatgttatatgttatatattatattatatatgttatatgttatatattatatgttataagttatatataatatagataatatataaaatatataatatatatacacaaaacacaaaacaccaccaaaaactgttaaaactaacaaatgaatttactatagttgcaggatacaaaatcaacatgcaaagtcagtaatgtttctatacactaacaatgaactatccaaaaaaaaatagaatctatttacaataactccaaaaagaataaaatacgtagaaatgaagttaatcaagtaggtgatagacttatacaaaaaaactataatatattgatgaaagaaattaaactaggcaaaaattggaaaggcatcctgttttcataaatatacagacttaatactttaaagtgtctataaatcaaagctatctacagattcaatgcattccctatcaaaatcccaacagcatgttttacagaaattaaaaaaaatcaaaaattcaaattaaatcacaaaagtcccagaattgccagtgttaacttgagaaagaacaaagctcaaggcatcacacttcctgcttacaaaaatatattatgaagctacagtaatcgaaacagtgttgtactggcatgaaggcagacatatagacagatgaaacagaataaagaagccagaaataaatccatgtatgtatggtccactgatcttcaacagtgatgccaaaaataaacagtggtgccaaaaataaacaatggggaaaggacagtctcctcaacaaggtgttggaaaactggacatctacatgagaagagtgcaatttaacccttaacttatacaatacagaaaaaaatcaactcaaaacagattaaagacttaaatgtaagaccttaaactataaaattcctagataaaaacaggggaaatgttccttgacatttctcttggcagtgatttttttggataagacaccaaaaactcaagaaacaaaaccacaaatagacaagtgggacaacattaaactaaataactctggtgcaataaagaaaacaatgaatcaaatgaaaaggtgacctatggaaagggagtaaatatttacaagccatacatatgataaagggttaatatccaaaatatattagaaacatctacaactcaatttaaaaaatagtaccaggcatggtggctcacgcatatagtcctagtactttgggaggttgaggctggtgaatggattgagcgcaggtattcaagacccgccagggaaacatggcaaacccctatctctacaaaaacacacacacacacacatatatatatatgtacatatatacacacacacatatatatgtacatatatatatatgtgtgtgtgtatatatatacgtatatatgtgtgtgtatatatgtgtatatgtgtgtgtgtgtgtatgtgtatatatatatgtatatatatatttatatatatgtatatatatatatgtatatatatacgtatatatatatatttatatatatatatatatattagccaggcatggtgggtgtgtgtctgtagtcccagctactcaggaggctgaggtagtaggattgcttgaacccggggtgtcaaggctgcagtgagccatgacagcaccattgcacttcagcctgggcaacagagagagaccctgtctcaaaacaaacaaacaaaaacataggctgtgtgcagtggctcatgcttataataccagcaccttgggaagccaaagcaggaggatttgagaccaggagtttgagaccagcttgggaaacatagcaagaccctgtctctacaaaaagtaaaaataaaaataaataaccaggcatattggcatgagttagtagtcctagctacatgggaggctgaagtgagaggattgcttgagcccaggagttcaagaccagcctgggcaacatagtgagacccccatctaggaaaacaaaaagcacaggcaacaaaagcaaaagtatacaaatgggattgcaggaaactaaaaagcttctgcacagcaaaagaactatcaacagagtaaagatacagtttacagaatgggagactattggcaaactatgcatctgatgaggagttaatatccagaatatatgagaaactcaaacaactcaatagcaaaaaacaaaaaataaacccaatccaaaaaatgggcaaatgacctgaatagacatttctcaaaaaatatatatacaaaagaagacatacaaatgggcaacatgtatacgaaaatatgcttaacatccctagtcatcagggaaatacaaatccaaaccacagtaagataccacctcagtctagttagaatggctactacagaaagacaaatgataacaagtgttggcaaggatgtggagaaaggaaactcttacatattgttggtgggaatgtaaattagaacagtcactataaaaacagtatggaagttccttgaaaaattaaaaattgaactgccatatgatccagcaatcccactaatgggtataaacccaaaagtaatgaaatcagtatgtcaaagagacatctgctgtcctgtgtttattacagcactattcataatagccaagatatggcaacaatctatgtccatcaatggatgaatgaatttaaaaatcttggtatatacataatatgcaatactattcattcataaaaaagaaagaaattgtttcatctgtgacaacatagatgaacctggagaacattgtgtcaagtgaaataagctacacacagaaagaaaactactgcatgatttcactgatatgtggaatcttaaaaagttgatctcatagaagctgagagtagaatggtggttagcagaagctggtgagggtaagtgaaagagggtatgggagaacactggtcaatgagtacaatgttaaagttaaatagaggaacgggttctagtgttctattgcacatcagggtgaatataaaaaacaatagtgtattatatatttcaaaataacttgaagaaaggattttaaatgttctcaccataaagaaatgataaatgtttaagatgatggatattctaattaccctgatttgagcattacacaatatatacacatatcaaaacatcatatcgtaccacataaatatgtacgattattctggctcaattgaaaataaaataaaattttagaaacgttaagaaaggaatattatgaacaatcttatctcacagattttataacctagttaagatggaccaattccttgaaagatacagtctattataactcatacagggagaaataggcaatctaaatactgttaattgtattaaaggaattaattattgaataaacttccaaaacagaaagcatcaagcccagatgggttcactggagtattccaccaaacatttaggaaagaaaactacaccaagtctctacagttgcattgaaaatatagaagcagaggcaatattttctaactcattgtataaggtcagcattacactaataccaaaagcagacaaaaatattgcaaggaaaaaaacacatcaatatatctgatgaatatagatgcaatatttctcaagaaatattagcaagtcaatccaacaaatttataaaaataattctacacttgagataagcctgggcaacatagcaagacactgtacctacaataaatacaaaaattagccaagcatggtggtgcatacctgtactcctagctacttgggaggttgaggcaggaggattatttgagcccaggggttagaggctacagtgagctatgattgtgccactgcactccagcctgggtgacagagtgagaccctatctcttaaaaaaagaaaaaagaattatacaccacaaacaagtaaggcttatcccaaaaaagtaagactggttcaacactggatatcagttaatgtaacccattatatcaataggctaaagaagaaaagtcacaagattataacaacagatttttaaaaagtgtttaacaaattacaacacctattatgatttcaaaaaaaattcagtaaactaggaatagaaagtaatcttcctcaacttgaaaaataacatctacaaaacagccttcatctgacatcttatttaatagtgagaaactatgagctttcttgctaagattaggaagaaaacaaggatttccctctcacattccttttcatcattgtacagtaagtcctagctagtgcaatgagacaaaaaaggaaataaaagacacactaattggaggagagaaataaaatgttgcaggtgacatgattgtctatgcagacaatctaaaataattaaccaaaaaaactctcccagaactaataggcaattaaagcaagattacagaatatgttaatatacaaaagttcattgctttcctatataaccgcaataaacaagtataatttgatattaaaaaacaatattatctgcattagcacacctgaaaatgaaataattaggtataaatttaataaaatatgtgcaagatctatacaaggaaaactacgaaacttgaatgaaagtaatcaaagaaaaactaaataaagagttattccatgtatacataggaagattcaatattgtcaaggtgtcagatcttctcaacttactctatgtagattcaacacagtcccagtcagtaacccagcaagttattttgtggatatcaacaaactgattctaaactttatgtgaaagacaaaacatgcagaatggtcagcataatattgaaggtgaagaacaaatttgaaggactgatacttcccaaaatcaatatttactataaagctatagtaattaagacagtgtgctattagtgaaagaacaaacaggtcaatggaacagaatagcaagcccagaaatagtctggcaaaaatatagtcaaccaatatttacaaaaaagcaaaatacaatggagaaaaggtcataattttaacaaatggtgctgaaacaagaagacatccacacgcctaataataaatctaaacacagatcttatatccatgacaaaaacgaacataaaagaaaacctagatgaccttgagtatggtgatgactttttagatacaacaccaaaggcatgatccatgaaaaaaagaactgataagctggcatttattaaaattaaaattaaaatttctgctctgcaaaaggcaaatgagaagcaaaccacagactaaagagaatatatttgcaaaaggcatacctgataaaagattgttattcaaaatatgcagaagagctcttaaagcacaataggtaaatgaacaatctgattttaaaatggtcaaaaaaacagacacctcaccaaaaaattacacagaaggcaaataagtaaatgaaaagatgctcatatacttatgaatatctttgttattagggaattgcaaattaaaacaatgatatagcactgcatatttattagaataatcaaaatctaaaacattaacaacacaaaatgctattgagaatgtgaagcaacaggaacgttcactcattgctagtgggaatacaaaatagcacagccacttcagcgacagtttagtggtttattccaaaattacattcttaccatgcattccagcaattgtgctcctgggtatttacccaaataagttgaaaacatatattcccacaaacacctgcatacaaacgtttatatcatgtttattcctattttccaaacttggaagcaaccaagatgtcctttggtaggtaaatggagaaataagctgtggtacatccatacaatgaaatattattcagtgctaaaaagaaatgagctgtcaagcttatttaaaggttacgtgtctcatgcaaagacatggaagaaatttatgcatattagtaagcaaaagtgacagtctgagaagctacatactgtagattccaactttatgacattctggaaaaggcaaaactctggagaaagtgaaaacattagtggttgccagggcttggggagagggaggcagagcacagagggggaacaaagggcagtagaactactctgtactatactataacagtggacacatgtctatacttttgtcaaaatacaatgtataacaccaagagtgaatcctaatgtaaactatgaactttgggtgataatgatgtgtcaatgtaggttcatccactgtaacaaatataccactcttgcatggaagcgtgatggtgagtgaggctctgtgtatgggggacagggggtttatgggaaatctgtaacttctgctcaattatgttattaatctaaaactgctctaataataaaatctacttttaaacatgtaatagaattcccaagaactgtgggacaattacacaaagtgtagcacacacatataacagaaataccaaaaaaagaaaagagcagaagaagtatttgaagaaataaaagatgaaaaattttctaaattaatggcaaacaccaatccagagatccaggaagctcagagaacaccaaagcaaaataaatgtcagaaaaaatgtacctctaggcatatcatattcaaactgcagaaaaccaaagacaaagataaaatcctgatagaagttgagggggaaacttacagataaataaacaataataagaatttcctctgacttatcagaaatcagccaagtaagaagatagtataataaaatatttaaagtgggaaaagaatacaaatccccctgacctagaattctctatcaagtgaaattattcttcaaaagtgaagaagaaataaagactttctcagacatacaaaaacaggaaatttctcaaaagtaaacttttcttacaaaaaatgctaaaagaagttcttcagataaaggggaaaatgataaaataataagcacaaatcaatataaaggaagagcaattgagaaggaataaatgaaggcaaattaaagctacttttcttactcttaattgacctatagatagttgctcgacacaaaaatagcaataatgcattggtaatcattattaccacttattaggataagtgaactgaatgacaatattaaaagagatgcagcccaggcacgggtggcccatgcctgtaatcccagaactttgggaggccgaggcaggtggatcacttgaggtcaggagcttgtggccagcctgaccaacatggtgaaaccccgtgtctactaaaaatacaaaaataggccagatgtggtggcacgcacctgtaatcccagctactcgggaggctgaagcaggagaatctcttgaacccggaaggcgaaggttgcagtgatccaagattgtaccactacactccagcctgggtgacagagtgagactctgtctaagaaaaaggggcggggggttgaatgcaaagagaaatacaatttaactgaacagctatcagttaaagtgaactgaactattaattaaatggatctaattggcatttgtagaatacttcatccagtaatagcagaatatatcttcttatcaaacttatatgcgatttccatcaagagaaacttcatatggaccataaaacacattttaacaatgtaaaaaaatagaaattacacaaagtatactctcagaacacacagaatagaactaaaaatcagtaacagaaggatagctgaaaattttcaaactgtttggagattgaacaacacacttctaagtaataagtgaattgaagaagatatctcaagaaatattttgaaataaatgaaaatgtaacatcatagtttgtaggatgcagtaaaagcagttctttgagtgaaatttataacactgaatggatatattagaaaataaaaaaatgtgatattaataaactaatcttaggccttaggaaactagagaaagaaaagaaatgtaggcctaaagaaagcagaataaaagaaatgataaaaattggagcataaattattaaattgaagtagaatttaaattttctctgcagagtaagtatgtgaggtaatgaatatgttaattagctttacttagccattccacatgtatacataaacacgttatacagcataaacgtatattattttgtttttcaattaaaaatttaattaaaatcagaaaaaattaataattaaatttaaaacaggaattcaatactgaaaaatcaatgaaaccaaaagctgtttctttaaaaagataataaaattgataaatttctagccaggctaatcaagaaaagaagaggattcaacattcttaaagaaaacaattttcaacccagaatttcatatccagccaaactaagcatcataagtgaaggagaaataaaatattttacagacaagcaaatgctgagagacttttgtcaccaccaggcctgccctaaaagagctcctgaaggaagcactaaacatgcaaaggaacaaccggtaccagccactgcaaaaccatgacaaaatgtaaagaccatcgagagtaggaagaaactgcatcaactaacgagcaaaataaccagctaacatcataatgacaggatcaaatttacacataacaatattaaccttaaatgtaaatgggctaaatgctccaattaaaagacacacactggcaaattggataaagagtcaggacccatcagtgtgctgtattcaggagactcatctcatgtgcagagatacacataggctcaaaataaagggatggaggaagatctaccaagaaaatggaaagcaaaaaaaagcaggggttgcaatcctagtgtctgataaaatagactttaaaccaacaaagatcaaaagagtcaaagaaggccactgcataatggtaaagggatcaattcaacaagaagagccaactatcctaaatatatatgcacccaacacaggagcacccagattcataaagcaagtccttagagacctacagagagacttagactccaacacaataataatgggagactttaacaccccactgtcaatattagacagatcaacgagacagaaggttaacaaggatattcaggacttgaactcagctcagcaccaagcagacttaatagacatctacagaactcttcaccccaaaacaacagaatatacattcttttcagcaccgcattgcacttattccaaaattgactacataattggaagtaaagcactcctcagcaagtgtaaaagaacagcaatcacaacaaactgtctctcagaccacagtgctatcaaattagacctcaggattaagaaactcactcaaaaccgcacaactacatggaaactgaataacctgctcctgaatgactactgggtacatagtgaaatgaaggcagaaataaagatgttctttgaaaccaataagaacaaagacactatgtaccagattctctgggacacatttaaagtagtgtgtagagggaaatttatagcactaaatgcccacaagagaaagcaggagagatctaaaattgacaccctaacatcacaattaaaagaactagagaagcaagagcaaacaaattcaaaagctagcagaaggcaagaaataactaagatcagagcagaactgaaggagatagagacacaaaaacccttcgaaaagtcaatgaatccaggagctggttttctgaaaagatcaacaaaattgatagactgctagcaagactaataaagaagaaaagagggaagaatcaaatagatgcaataaaaaataataatgtggatatcactaccaatcccgcagaaatacaaactaccatcagagaatactataaacacctccatgcaaataaactagaaaatctagaagaaatggataaattcctggacacatacaccctcccaagactaaaccaggaagaagttgaatctctgaatagaccattaacaggctccaaaattgaggcaataattaataggctaccaaccaaaaaaagtccaggaccagaccaattcacaactgaattctaccaaaggtacaaagaggagttggtaccattccttctgaaactattccaatcaatagaaaaagagggaatcctccctaactcattttataaggccagcatcatcctgataccaaagcctggcagagacaaaacaaaaaaaagagaattttagaccaatatccttgatgaacatcgatgtgaaaatcctcaataaagtactggcaaactgaatcctgcagcacatcaaaaagcttatccaccatgatcaagtcggcttcatccccgggatggaaggctggttcaacatatgcaaatcaataaatgtaatccagtatataaacagaaccaaagacaaaaaccacatgattatctcaatagatgcagaaaaggcctttgacaaaattcaacaacccttcatgctaaaaacactcaataaattaggtattgatgggacatatctcaaaataataagagctatctatgacaaacccacagccaatatcatattgaatgggcaaaaactggaagcattccctttgaaaactggcacaagacagggatgccctctctcaccactcctattcaacatagtgttggaagttctggccagggcaattaggcaggagaaggaaataaagagtattcaattaggaaaagaggaagtcaaattgtccctgtttgcagatgacatgattgtatatctagccccatcatctcagccccaaatctccttaagctgataggcaacttcagcaaagtctcaggatacaaaatcaatgtacaaaaatcacaagcattcttatacaccaataacagacaatcagagagccaaatcatgagggaacacccattcacaattgcttcaaagagaataaaatacctaggaatccaacttacaagtgacgtgaaggacctcttcaaggagaactacaaaccactgctcaatgaaataaaagaggatacaaagaaatggaagaacattccatgctcatgggtaggaagaatcaatatcgtgaaaatgcccatactgcccaaggtattttatagattcaatgccatccccatcaagctacaaatgactttcttcacagaattggaaaaaactactttaaacttcatatggaaccaaaaaagagctcacatcgcgaagtcaatcctaagccaaaagaacaaagctggaggcatcacactacctgacttcaaactatactacaaggctacaataaccaaaacagcatggtactggtaccaaaacagagatatagatcaatggaacagaacagagccctcagaaataacgccgcatatctacaactatctgatctttgacaaacctgagaaaaacaagcaatggggaaaggattccctatttaataaacggtgctgggaaaactggatagccatatgtagaaagctgaaactggatcccttccttacacctcatacaaaaatcaattcaagatggattaaagactaaattgttagacctaaaaccataaaaaccctagaagaaaacctaagcattaccattcaggacataggcatgggcaaggacttcatgtctaaaacaccaaaagcaatggcaacaaaagccataattgacaaatgggatctaattaaactaaagagcttctgcacagcaaaggaaactaccatcagagtgaacaggcaacctacaaaatgggagacaatttttgcaacctactcatctgacaaagggctaatatccagaatctacaatgaactcaaacaaatttacaagaaaaaaacaaacaaccccatcaaaaagtgggcgaaggacaagaacagacacttctcaaaagaagtcatttatgcaggcaaaaaacacatgaaaaaatgctcaccaccactggccatcagagaaatgcaaatcaaaaccacaatgagctaccatctcacaccagttagaatggcaatcattaaaaagtcaggaaacaacaggtgctggagaggatgtggagaaataggaacacttttacactgttggtgggactgtaaactagttcaacccttgtggaagtcagtgtggcgattcctcagggatctagaactagaaataccatttgacccagccatcccattactgggtatttacccaaaggactataaatcatgctgctataaagacacatgcacacgtatgtttattgcggcactattcacaatagcaaagacttggaaccaacccaaatgtccaacagtgatagactggattaagaaaatgtggcacatatacaccatggaatactatgcaaccataaaaaaggatgagttcatgtcctttgtagggacatggatgaaattggaaatcatcattctcagtaaactatcgcaaggacaaaaaatcaaacaccgcaggttcgcactcataggtgggaattggacaatgagaacacatggacacaggaaggggaacatcacactctggggactgttgtggggtgggaggaggggggagggatagcattaggagatatacctaatgctaaatgacaagttagtgggtgcaccacaccagcatggcacacgtatacatatgtaactaacctgcacattgtgtacatgtaccctaaaacttaaagtataatagtaataaaataaaatttaaaaaaagatagcatgagtagaaaaaaaaaaaagaagagggaagccacaagttactaatatcagaaatgaaagaggggtcatcactactgattctgtggacattaaaagaatgataaataaataccataaacaactctatgcccacaagtttgatagcttagatgaaatgaacctattccttgaaaagctcaatctgccaaaactcacataaagtgaaatagataatttgaataggcctatacctactaaagaaactgaattaataattaataatcttccacaaaagaaaacaccaggcccagatgggttcattagtgaattctgtcaatcatttaaaaaaaaaatggtaacaattatctgcagtcttctctagaaaacagatgaaaagggaaccattcctaattcatatacttgctgacttgttccatgaggccagaaatgccctaattctaaaaccaatgatattaaaagaaaggaaaactacagaacaatatccctcatgaacataaatgcaaaaattctcaataaatattagcaacttaaatttttgtttaaaggtataatcatgtatagaaagaattatagtccgtggccaagtgagatttattccaggcatgaaaatctggttaaatatttaaaaatcaattaatataatctatcacacaaacaagataaagaaggaaaattatgtgatttttccatcaatggacacaggaaaagcatttaacaaaattcaacatccatttataataaaaactcagcaaactggaaacaaagagaaactgcctcaacttcattaacagcatctaaaaaaaccatacagttaacctcatatttaatggcaagaaattaggtgcttttgtcctaagactgggaacaaggtaaagatgtcctctctcgccactcccattcaacactgaactagatgtcttagataatttaacaggacaagaaaagtaaataaaaagtttatacattggaaaagatgaaataaaaatgtctttgttaataaatttgtaattttctatattaaaatctgaaagaatagacaagaaaaataaaaacaaacaaaaaagaaacttccagacctagtaggcaaaaaaatggcaacactgaagaatagaaggttaatatacaaaagtcagttgtttttttccagcaattaccaagttgagattgaaattaaaaacacaataccatttatgtgaacaccaaaaaatgaaacttaggtgtaaatctaacaaaatatatacaagatctattgaagacgactacaaaactgatggaaaaaaaagcaaatatctaaataaatgaagagatgttccatgttcatgaataggaagacttaatattgttaagatgtctattctgcccaacgtgatctataaattcaatacaattccaatcaaagtcccaacgagatattttatcaatataaacaaactctaaaatcatatggaaaggcaagagacacagaataaccaatgcaatattaaagaaaaagagcaagtttgaacaaccaacactacctgattttaagacttactataaagcttcataatcaagacagcatggtattggtaaaagaatcaacaagtagattgatgaaacagaacagagagcccagaaatagacatacaaaaagatagtcaactagtctttgacaaatgagcaaaggcattgagagatagccttttcaagaatggctgtcttagttgaaagagtggttgaatatccacatgcaaaaaaaaaaaaaaaaaaaaaacttaatgtagaccttatgcctttcacaaaaaccaactcagtttagatcatagatataacataatgctcaatactattatataaaacgactagaagataacataggagaagatttaagggacctactctttgtctatgagtttttagatacaacaccagaagcataatccatgaaaaatgattgaaatgttggacattatgaaaatttacaactttgcaaaggacactgtttaagagaatgaaaagacagccctcagaatgggagaaaatatctgcagaatatatatttcataaaggatttgtatccaaatatgtacaaagaactcttaaaactcaacaataagaaaacaactcatttcaaaaacggccaaaagatctgaataaacccttcaccaagaagatggcaagtaaacagatagaaagaagctcaaaatcatatgccattagggaattgcaaattaaaacaacagtgagacaacactacacatctattagataatcactaaaattcaaaaaaaactgaaaataccaatggctgatgaggatgaatattaacaggaactttcattcattgctgctgggaattcaaaatagtacagtcactttggaagataatttagcagtttcttacaaaactaagcatggtcttaccatacaacccagcaattgtaaggctatgtatttactcagttgagttgacaacttatatccacagaagaatgtttattgtagctttattcataatcaccaaaaactggaagcaaacaagaggttcctcaaaagctgaatggataagctgcggtacatccattaaatgggatattattcaatgatataacaaaatgggctatccagccatgaaaagacatggaggaatcttaaatgcccattgctaagtgaaagaagccagtctgaaaaagctacatactaaatgattcaactatattacattctcaaaaaggcaaaactgtagaagagcaagaaaccagtgttgccagagatttggagtaggaaagagatgaatagataaaagcagtggatttttaggacagtgaaactcttctgatattataatggtgggtacataacattatgcaatggtcaaaacccatagaactgtccaacacagagtgaaccttaatataaacaatggactttagttaatgataatatatccatattggttcatcagttttaacaaatgaactacaagatgttaatgacaaggaaaagtatatagatgggagggagtcatagggaaaccctctgtgttgtttgcaattttttgtaaatataaaattgttctgaaaaataaagtctattttcttaaagtcagtccagggatcctcctttatatgaatacaatctggcattttgctaagtaaaatttctctgtttccagcatttaagtctcacttctctgtgcaagattttctttagttttatatttcctaggtcatttctgtcagaattaaggggtggggtagtgagaattcagacatatgtcctcaagctgccatctgttctccaaatggtatcccatatgttgttgatgtgtagagttttcattgttgacatttatgaattgactataattgtactttgatttccttcttgactcgagttaattatgagggtgtttaaatttccaagtggtattttttaaaaaaacatgttactaatttctgcttcattgctcattgttttaaatatgtgatttgcgtaagtcttactttttagagctattgagagggttttttttcttggtagtttagtatatcgccaatttttgaaatgtctagatattttatagcaatgtgtactattcttatgttatgaagtctatttttttctatctgtatatttttgtttattcttctgtaaaactttgaacggtttcattatatatttagcattatgttacttgttacatataactttgtgaatatcctattgccatcatgtgttgtatcttttatcaatataaataatcctttatcacatgaaatgctgtttgctttgaatttaatttgtctaatagcaatgttgtaatgaatgccttctttaatttttactttcttgatatacctttggccatccctctatttccacacttagatatctaagtgatgtggtttaaaatttttctcttttaaacagcatagaattagattttagggaagttttaaaataagttattttcttaatgaaagaccttaagtcattcacatgtattgcgacaaatagtgttttggtttggtgtaatttcagttccttattttagtctttctatattaagggttttttgtttgtttgttgctatttacttgttttttctttttatttgattatcaattgtggtaggcttgaaaaaagctccccgcccctaccccccaccaccaccaccaccacaaaatccgtatcctaatccctggaacctgtgatgttaccttatataagaaaagggaacttcacaaatgtaatcaaattaaagatcttaaaactgggagattattctggtttatcccagtgattcccacatgtaatcataacagggtggtagaagggagtgtgacacacagacagaagaggaaaaggcagagcttgggccgatatggtcacacgccaaggactgctggcagtcacccgaagccagaagaggcaaggggcagattctcccctagagtctctggaggactcatgggcttgccaaacttttatttttgcccagtagaactaattttggacttctggtctccagagctgtgcgagaataaatttatgttgttttaagccataaagtttgtagtagtttgttacagccaccataggaaactaattaaaatttctccatttttattttactctatcatagcttatctttaacttttagctttaatatatctctatcaaggttaaaaaacaaaagagagacccaaaatacataacaaaattgaacatgatgtctttctttgtactctgtcccttcactcaactcccttaagtttttcctgaaatgttccatagtcatatttataattattttgtaagcatttcatgtgtttctaacttcattgcttaccatagttgttattattctatgctcttccctcttcttgaattcttaattttaattcaattctcaattagctgaagtatgccaggaagactataaaatattatattttcttccttcttatatatctgagaaaactgttctgtgccttggcacagaaatcatatttacaaagttcttgaatcacaattttttttcaaaactctgaacatgttgttatagtgttttctggcattactttttgtgggaaagaggtatgataatttaagatgcatttcttttaggtaacttttaaatttttttaccctgccttaaatgcctgaagatggttaatctctgaaattaaatactcttttgattgtatctcaaagtcagcatctttttatcaacattgactgctcttctggacactcttatttatggctcttcctcctgctccagattctgaactgtgcctgtcacctccctcaagacagccacagagccccaagatccttataacctcaccatgatgttccactccacagggttctgccattgtgtgctttctttttcatccttctctgatgccacctcctcctctgtttgttgccagccttaataattttaaattcatggatcacaggtagaaaccccagttttgtctaagtagacgatcgattttttttttcttaaagctagtcttccatttttctctttctctatttgtcaccttttcccaagagataattttttcttttagttttcagaggagccttcaatttctttttttaaaccttttttctaacttttattttaagttgaggggtacatgtgcaggtttcttatacaggtaaacttgtgtcatgggggtttgttgtacagattatttaatcacccaggtatttttttttttttttttttttgagacggagtctcgctctgtcgcccaggctggagtgcagtggcgcaatctcggctcactgcaagctctgcctcccgggttcacgccattctcctgcctcagcctcccgagtagctgggactacaggcgcccgccactacgcccggctaattttttgtatttttagtagagacggggtttcaccgttttagccgggatggtctggatctcctgacctcgtgatccacccgcctcggcctcccaaagtgctgggattaagcttcatattcattagttatttttcctgatcctaaccctcctcccaccctacaccctcctataggccccagtgtgttttgttcccctctatgtatctgtgtgttctcatcagttagcttccacttataagtgagaatatacagtattttgttttctgttcttgcattagtttgctgaagataatgccctccatccatgtccatccatgtccctgcaaagcacattatctcattctttcttacgagccttcaatttctaatcattcctcagaacctcctccctatggtggctttcacaacattagtgctgagatgcatttgtagaaataacaaatcataaagaaccacaatgtgcttgattgttatttgtctgagccagagaaagaacaaaaaggcacgaattcagaacaaagataactgccaagaagaaagtgaagtaaatgctctccattagacttcaatctacaagttactcttctactccctctggatttgaacctaatgttacaaagtggagatacagccagactcttgatagaaatgaggctccaagttacctgaaaaatgtccaccttgatcatcccttcaaatatatttcatttaatcacattctaaatatgctcttcacataagagaaattaacttcttaaatcccaatttccctttctgctaattatgcaaaaaaagattgtacacagataattaaaaggtgtgggtaaagttaataaaagagacagtgggaaggaagaggagacccagatcctgagcctaatgaagtggatgaaaaaggggagctctctgatggtgtcagtgctgatgatgataaatgattcaacaccgcacataggctccctaaaaccggaacccttctatcatctcttgactcccactgataaaaagcaaatgaacttttgtttctaattattctggaatgtcaattgtggcagaaaaaattagtttagctttaaaagtcaattccaaacaaatgtaccatgcaaatgtcaaggactgatcctgaaatatggagtttccaagaggccaagggaataagtgagcaagaaagaactgaagcattgtcactcatcatggccagcagtacaaattctagcaccaggaaccaaactctgcttcctggtgtggagatgaaaagggagagttccctggtccatccatcacaggacatgggacaggggtatggctctctgttcagcttcctcaagctcaaactccttataggagggggagcatgcagaagggtaggtgcaggagccagggcaagtgcattgaactccggccccacagtagtgtctggggtgggtgcctgtgactcctgaagccccagtgttatagtgctgttttaactctgccatctgcagatggcttaagtggtaaccagctcagtgccctcttggtggccatgtcctcgtccataggccaggaagagtcaggtcacacacagacttgaaggatgaatgcaggagttttattgagtggtggaggtggttctcagtgggatggatggggaactggaagggggatggagtgggaagatgatcttcccctggagtttggctgtccagggccattctcctgtctgattgtccccagctgaactcttcttagtgttcagaccctccttctcttctctccttctctgccatgctgttctgccattcgtctgctcatctcctcctggagcctgggatttggggtttatatgggtacaggataggggcatgtagctggccaaaaggcaactttttgggcacaaaaacaggaatacctgttctcatttaagaccactggtttccaggcttgagggtggggtctttgctgggaaactgccctctttcacccagtatttccctttctcttgtccatgtaagagagttatttcccttcctaatccaacactcaccccttctttggttcacctctcagatttctaaataacacaaggtgctattctttccattatgtgccttccctcacctctctgaagtcataagcttcctctcatttctgtctttaggttgtgctgctctacgcctctacctacgtcctggtgtccctcagcatagacagataccatgccatcgtctaccccatgaagttccttcaaggaggtgagctggctttaccaggtgctctttcataaagaactgtccttgagtgagggtaggatcattttcactaatattttactcttaataatagtgatcttcctcctctttccttctctttcttctctcttcattctcctcttccacctctttcctcacttcatgggtctgattgtctaaaaagatgtacaaaagaaaacttagatgcctggaaaaatgccgatggttcccatggacagcccagagccactaaggaaggtgggtggctgacccactttgtgaccattcagcttgatgctgatgcttccatctttttcctctagagcagtgttgacatgcatacctgtccaaagaaatgaactctcccacactaacttctcattttccagaaacaaacaccttgttctccttatccaaaaccatagaaataattggaaagatgtttttaattgatcatcgtattgggaaaaaaaaataaggaaactctctttgatccagaaatggttagggatccctaagaaaaggaagcagatgggtcagagggaaggctgagaccacacagcccacaaaacccctaggagatgctgaagtgggatgggaggccttaggagcacttcctgaacaggctgcatcaatgtggagtgtaggagaggacccgaggcaactgacaaggtgatttctgaagtacaacacagaggattaatcacagcaagtttccctggatttgaagaggcagagaggggccctggggagcagtaatgccctagaggaagggtgagtacatattcacaagactagctgcctactgctctgcccacaatcagagaccactctcctccatacaccctgaaagcaaatacaaagccattgtttagcaacctgttctcccttcctccagataacttcgaaaactgataaagaacctaacaaaaaatcttgaccacaaaaatgagtgcaatcaaatattatttaaaaagtgacaaaatccaaactgtagaaaacagataatacctttaaaatctcattccacacaatttggatctagggaagcaaatttaatagaagtagcagaattttaggataaatataattaacgtcatcaaggatctacaagaagtggagtgaaatagggtaatttcatttactattgacaggggtataagtaaacacatcttattgaaatataaaatgtgcagccctttgatatgacttcttggtatctgctctggagaaacacccacacgtgtgcatgtatacatatgtgatgtatattgtggcactgcctacaatagcaggaattcatcgctaaccttggtgtgtctcagtggaggaatggctaactagtctatggaagaagtttcagtaccttctgtttattgattgcatactaggtgtcattcatgattctaactgattcatacatattaaatattttaatattcataatatctctgtgaggtgggctctattattatctgcattttacaggtaaggcaatcgttgttccaagagtgaaagtaatttgcccaaggtcacagctagtaagtgcaggaacaggatttgaacccacacagtctgggtctagctccagcgccctcaaatgctatatagcctgcctctcaatgtggaagtggtagagccatgggcagtaatatgcatcaacatgaacatatccatattgtatgctgagtgacaaaaacaatatgcacaaaaatccatgtgaacgataacattaaagtcaaaaactcaaaacaatgctagatattgtgtacagaaagacagagaggagcagaaagagagaacacacaaaagaaaactccaggaaaaataaaaaactatttgcaaaaacacacaaaacttgacatttgacctcacttctagttatgggagttagaccagtggtgtgaccaccacaggaacttatatgcaaggtggtaatttttacatgaagaacatatcaatgtgttgtgtgcaattaaaatgaatttattttgatcatgcttgttatgcctactgtacagcataagacatatgagtaaaaatactaaattgtatacttaagaatttgctggggatctgttccaagatggccaaataggaacagctctggtctgcagctcccagcatgatcgatgcagaagacaggtgatttctgcatttccaactgaggtgcctggttcatctcactgggactggttggacagtgggtgcagcccatggagggtgagccaaagcagggcagggcatcgcctcacctggggagcacaaagggttgggggatgtccctttcctagccaagggaagctggggcacactgtacctgggacactcctgccaaatactgcacttttcccaaggatttagaaacgagcagacaaggagattctctcccatgcctggctcaggaggtctggagccttgctcactgctagcacagcagtctgaaattgaactgtgaggcagcagcctggctaggggaggagcgtccaccattgctgaggcatgagtaggtaaacaaagtggcccggcagctcaaactgggtggagcccactgcagctcagcaaggcctactgcctctataggctccacctctgtgggcaggacatagctgaacaaaaggcagcagacagcttctgcagacttaaacgtccctgttcgacagctctgaagagcacagtggttcttccagcatggcgtttgagctctgagaatggacagactgcctcctcaagtgggtccctgacacctgtgtatgctaactgggagacatctcccagtaggggccaacagacacctcatataggtgggtgcccctctgggacaaagcttccagaggaaggatcaggcagcaatgtttgctgttctgcaatatttgctgttcacagcctctgctagtgatacccaggcaaacagggtctggagtggatctccagtaaactccaacagacctgcagctgagggacctgactgttacaacggaaactaacaaacagaaaggaatagcaccaacatcaacaaaaaggacatctacaccaaaaccccatctgtaggtcaccaatatcaaagaccaaaggtagataaaaccacaaagatggggagaaaccagagcggaaaagctgaaaattctaaaaatcagagcgcctcttctcctccaaaggactgcaactccccgccagcaatggaacaaagctggatagagaatgactttgacgagttgacagaagtaggcttcagaaggtcggtaataacaaactactcctccgagctaaaggagcatgttcaaacccattgcaaggaagctaaaaaccttgaaaaatggttagacgaatggctaaatagaataaacagtgtagagaagaccttaaatgacctgaaggagctgaaaaccatggcacaagaacttcgtgaggcatgcacaagcttcaatagccaattcaatcaagtggaagaaagggtgtcagtgattgaagatcaaattaatgaaataaagcgagaagacatggttagagaaaaaagattaaaaagaaacaaacaaagcctccaagaaatatggtactatgtgaaaagaccaaatctacatttgattagtgtacctgaaactgatggggcgaacagaaccaagttggaacacactcttcatgatattatccagaacttgcccaaaatagcaaggcaggccaacactcaaattcaggaaatacagagaacaccacaaagatactcctcgagaatagcaaccccaagacatgtagtcagattcaccaaggttaaaatgaaggaaaaaatgttaacggcagccagagagaaaggtcaggttacccacaaagggaagcccatcagactaacagcagatctcttggcagaaaccctacaagccagaagacagtgggggccaatattcaacattcttaaagagaagaattttcaatccagaattccatatccagccaaactaagtttcataagtgaaggagaaataaaatcctttacagaaaaacaaatgctgagagattttgtcaccaccaggcctgccttacaagagctcctgaaggaagcactaaatatggaaaggaacaaccagtaccagccactacaaaaacatgccaaattgtaaagaccattgatgctaggaagaaactgcatcaactaacgggcaaaataaccagctaacatcataatgataggatcaaattcacatataacaatattaaccttaaatgtaaatgggctaaatgctccaattaaaagacacacactggcaaattggataaagagtcaggacccatcagtgtgctgtattcaggagactcatctcatgtgcagagatacacataggctcaaaataaagggatggaggaagatctaccaagaaaatggaaagcaaaaaaaagcaggggttgcaatcctagtgtctgataaaatagactttaaaccaacaaagatcaaaagagtcaaagaaggccactgcataatggtaaagggatcaattcaacaagaagagccaactatcctaaatatatatgcacccaacacaggagcacccagattcataaagcaagtccttagagacctacagagagacttagactccaacacaataataatgggagactttaacaccccactgtcaatattagacagatcaacgagacagaaggttaacaaggatattcaggacttgaactcagctcagcaccaagcagacttaatagacatctacagaactcttcaccccaaaacaacagaatatacattcttttcagcaccgcattgcacttattccaaaattgactacataattggaagtaaagcactcctcagcaagtgtaaaagaacagcaatcacaacaaactgtctctcagaccacagtgctatcaaattagacctcaggattaagaaactcactcaaaaccgcacaactacatggaaactgaataacctgctcctgaatgactactgggtacatagtgaaatgaaggcagaaataaagatgttctttgaaaccaataagaacaaagacactatgtaccagattctctgggacacatttaaagtagtgtgtagagggaaatttatagcactaaatgcccacaagagaaagcaggagagatctaaaattgacaccctaacatcacaattaaaagaactagagaagcaagagcaaacaaattcaaaagctagcagaaggcaagaaataactaagatcagagcagaactgaaggagatagagacacaaaaacccttcgaaaagtcaatgaatccaggagctggttttctgaaaagatcaacaaaattgatagactgctagcaagactaataaagaagaaaagagggaagaatcaaatagatgcaataaaaaataataatgtggatatcactaccaatcccgcagaaatacaaactaccatcagagaatactataaacacctccatgcaaataaactagaaaatctagaagaaatggataaattcctggacacatacaccctcccaagactaaaccaggaagaagttgaatctctgaatagaccattaacaggctccaaaattgaggcaataattaataggctaccaaccaaaaaaagtccaggaccagaccaattcacaactgaattctaccaaaggtacaaagaggagttggtaccattccttctgaaactattccaatcaatagaaaaagagggaatcctccctaactcattttataaggccagcatcatcctgataccaaagcctggcagagacaaaacaaaaaaaagagaattttagaccaatatccttgatgaacatcgatgtgaaaatcctcaataaagtactggcaaactgaatcctgcagcacatcaaaaagcttatccaccatgatcaagtcggcttcatccccgggatggaaggctggttcaacatatgcaaattgataaacgtaatccatcacataaacagaaccaatgacaaaaatcacatgattatctcaatagatacaaaaaaggccttcaacaaaatccaacgcccttcatgctaaaaactgtcaataaactaggtattgattgaacatatctcaaaataataagagctatttatgacaaactcacatccaatatcatactgaatgggcaaaaactggaagcattccctttgaaaaccggcacaagacaaggatgccctctctcaccactcctattcaacatagtgttggaagttctggccagggcaatcaggcaaaaggaagcaataaagcgtattcaattaggaaaagaagaagtcaaattgtccctatttgtagaccacatgattgtatatttagaaaaccccattgtctcagcccaaaatctccttaagctgataagcaacttcaccaaagtctcaggatacaaaatcaatgcgcaaaaatcacaagcattcctatacatgaataagagacaaacagagagccaaatcatgagtgaattcccattcacaattgctacaaagagaataaaatacctaagaatccaacttacaagggatgtgaaggaccttttcaaggagaactacaaaccactgcgcaaggaaataaaagaggacacaaacaaatggaagaacattccatgctcatggataggaagaatccatatagtgaaaatggccatactgcccaaggtattttatagattcagtgccatccccatcaagctaccaatgactttcttcacagaattggaaaaaactactttaaagttcatatggagccataaaagagcctgcattgccaagacaatcctaagcaaaaagaacaaagctggagacatcaagctacctgacttcaaactgtagtacaaggccacagtaaccaaaacagcaaggtacttgtaccaaaacagagatatagaccaatggaacagaacagagccctcagacataacaccacacttctacaaccatctgatctttgacaaacctgacaaaaacaagaaatggggaaacgattccctatttaataaatggtgctgggaaacctggctagccatatggagaaagctgacactggatcccttccttacaccttatacgaaaattaactcaagatggattaaagacttaaatgttaaacctaaaaccataaaaaccctagaagaaaacctaggcaataccattcaggacataggcatgggcaaagacttcatgactaaaacaccaaaagcaatggcaacaaaagccaaaattgacaaatgggatctaattaaactaaagagcttctgcacagcaaaagaaactaccatcagagtgaatgggcagcctacagaatgggagaaaatttttgcaatctacccatctgaaaagggctaatatccagaatctacaaagaactcaaacaaatttacaagagaaaaaacaaccccatcaaaaagtgggcaaaggatatgaacagacacttctgaaaagaagacatctatgcagccaacagacacatgaaaaaatgctcatcatcactgatcatcagagaaatgcaaatcaaaaccacaatgaaaaaccatctcatgccagttagaatagtgaccattaaaaagtcaggaaacaacaggtgctggagaggatgtggagaaataggaacacttttacactgttggtgggagtgtacattagttcaaccattgtggaacacagtgtggcgattcctcaaggatctagaactagaaatactatttgacccagcaatcttattactggtatatacccaaaggattataaatcatgctactataaagacacatgcacacgtatgtttattgctattgcagcactattcacaatagcaaagacttggaaccaacccaaatgtccatcaatgatagactggattaagaaaatatggcacatatacaccatggaatactatgcagccataaaaaaggatgagtttgtgtcctttgcagggacatggatgaagctagaaaccatcattctcagcaaactgtcacaaagacagaaaaccaaacactgcatgttctcactcataggtgggaactgaacaacgaaaacacttggacgcagggtggggaacatcagacaccggggcctgtcagggagtgaggggctgggggagggatagcgttagcagaaatagctaatgtaaatgacaagttgatgggtgcagcaaaccaacatggcacatgtatacctatgtatccaacctgcacattgttcacatgtacaatgataatttaaaaaaaagaatttgctaacaagataaaccttatgttaagtgctcttgccacaaaaggaaaaaactaaacaaaacaatactaaagggggacaggaagaagcctttggaggtgatggatacatttatggcattgcttgtaatgatggttacaggaatgtatacatatctccagacacattaagtggtatgcattggatatgtatagctttttatatgccaattatacatcaataaagtggtttagaagttaattttaaaaatagaaaatatacttgtttcctgggtttcttctcggtcatttggtgtgtctgttttgtgaagggttgagacctgtaccaagttttctgggcactgtaaagccctagttattatgataggggaggcaggaaattgatgaggcaggggagataggagagacatttcgtgacataggtatgcaaaaatgtgtcaatgaagcatcaaattctgtaaaatatttgtagagatttattctgagtgaaatatcagtgacaatggacaaagccctcaggaggtcatgagaatatgtgcccaaggtggtcagggtgcagcttggttttatacattttagggcgacatgaaccttcaatcaaacacatttaagaaatacatagggttggtccgaaatggcagggttgggtagtgggggatggggtgggcgaagcttccagattatagttagatttaaattttttctggttgacaattggttgagtttatctaaagacctgggatcaacaaaaaggaatgtctgagttaagacaaagaattgtggagaccaaacttcttatttgcaggggaaacttttaggtttcctcttcagggagactatgttgtaaaatgtttctcatcagacttaaagtctgtgttgatgttaacgctggagaggtataatgagacacacctgaacccgcactgcccatcatggcttggaacagcctctcaggttaaatcttaaaagagccttggctgaggaggaaatccattcggaaggttggggggccttataactttatttttggtttacattcatggatttgtgatacacttttttttttttttttttttttttttagacagagtctcactctgtcgcccgggctagagcgcagtggcgcgatctcagctcactgcaacttctgcctcccaggttcaagtgattcttgtgcctcagcctcccgagtagctgggattacaggtgtgcatcaccacgcctaatttttgtattttagtagagatggggtttcaccgtgttggccaggctggtctcaaactcctgggctcaagcaatccaccttggccttccaaagtgctgggattataggcatgagacactgcacctggcccgtgatacacttatttatccatctatttgtttatgcctagccgactttagaaagggttgaatgtggtggacaaaaggacatcgacgccaaagtattttaaggtgaagaaatataagtgaaggaaaaataagtataggaaattgcaggagggaagtcccaggaaacttgttagaggtacagacttgagacccatagccagagggtgacatgatgaattacatgtttcaccatattcactaggttaaaaaattagctgtttgctcgtggaaaatatgtctagttctgggagacactgtgtaatatcatcaagagtgtcctaaattatcagaaaggcagcaattaatattatagtgcttttctaaagatgtaaaacaacttattccctcactacaaaattaagagaggctcaacataaggtgcttggaaaatcctcatcatcagtagcttctgggaaagatgaaagcttagaaacacattagccagctgccttcctctgcccaggcccaacgaaagtcactgaagaaatctaaaaacagggggagacctgcatctggaatgagagccaaggaaagaagccatccaaataccagaaacttctaagaattcccagcacacgagggagcagatgaaacacggaagccaagtgtataatgtataattcagcccccgggagaactccctggccttgtagaggatgaactgatactcacaagtttatagggtgagggcagccggtgagaacgcgacacgagaggttccaggaagcccaacgcgggagtcattttagtaccgcagcttgtcagaatttaagggcagctgagaggcagggttcggagtttatgcggctcagctgagagaaagccgtagaaagaagagattccccgagagtggcagggctctctgggagagtcatggagcagggagaaaccaggagggagtcaaatgtagctgtgcaggcacaaggcaatggccgcttaactgaggggaggccatattttatttattttttactgctatttactgaagcagtatttttcttagccagcaactccagaaaagttgacaattttaaaggtcttcaccaaaaatgaatatgagaataattagaaaaatatcaaaataataaagagaaatatgcagtgttatattaaaatgtaatataaaactttgataattaaaacaatgctggaaccagaataacacatataaatcatacaaaatttcaaatcagttggcaaaaaatgaattagtatacggtgttgaaatagctgtctggccttttttcagaatattagatcaccacccaccatagctaataacaaagtaaattttagttggattgaaaaaattaacacttcaaacataaaaggagtaaaagaatatttgggtaacaccttcataatttcagtgaaggaaaggcctttccaggaaccatatcaaaagcagaagcaagaaggaaaatatagttttttactacataaaaatttaaattatttatgtaaataataaatatccataaacaaacttaaaaagcaaaattataaatggaaaatatttttatgtaacaggcaaagaaaaatatcctttttataaagaagtctggcaaagaaataaaaacattctaattgagtaaatgggcaaagggcatgaacagactaacaagaaacaaagaaacataaatgagaccaggcgctgtggcccacgccggtaatcccagcacttcgggaggctgaggtgggtggatcacttgaggtcacaagtttgagaccagcctggccaacatagtgaaaccctgtctctactaaaaaaatacaaaaattagctgggcatggtggcatgcacctgtaatcccgctactcaagaggctgaggcacaagaatcgcttgaacctgggaagcggaggttgcggtgagtggagatggcaccactgcactctagccctctagcattggcaacagagcaagacttcaccagaaaaaaaaaaaaaaaaacaacaccataaatgaatagaaaatgtatagaaatattcattttgactactaattaaagaattgcaaaattgaacatttggatacaaaattttaccaaccacataacaagattaaaaggaagaattatccaactattaggagagtataaggaaattggtacttttgttgttacagtgagagccataaatagtaggcacttttgagggaaatgtaacaatatttgtcaaaatttataaatcttctgtttctaagagtttatcctaaggaataacaagaactgagaaaaatagatgaatataaattatttataataaaaaatggaaaacaacttagataataatcggctattgataaaattggagtacagtataaccaccggaaaaaatgttatttatcagtgcttttccaattgcacttcccacagtgacccccagagggctcattttgctggcctgctccagggcagatctgggagctgctggctgtctcagtttcaaccaaagatacctgagctttcaacactttcctatactgggttcgagataaaattatatttgaagaaaggatcttactacctgaaaaaaaatgccacttgtacaagttcatatttcttaacaggaaaagatggtcataacaggcttttcagaggaaaaaaattaggttacaaggtaggatctgtatcatgatctcatttctaaatgtgtaatctatgtattatatatgcatgcgttatcaaaatttaagcttgacgtttctggttggtgccactatggatttttttccttttcgcttatttttatttgctagtgtttctataatcaatgtgtgtaacttatacgattttataaaaaggtaaatgagttacatggaaaagaggaaggctaagcggcagagaagtacagaaatggacaagagcacatggaagagggagtctgactttccgggggtgagaaacatgagacagtctaccttatatgcaaacctgcatttcatctgttcgatcctggatccttcagccctgagaagtcactcttcatggcaaatgggctcagctgcctggaaggccatcagtctccctgctgtggcagctgcgtcacagcctaagagagtggggctttcacagtgtagccccagcaccagtaagagaaaaacagctccaagcctataccctgctgggcctttggggaaacatcttcggagaccttggtagaattgattctaaagtatgattctatactctcttccctgtccatccctctcaaaagtgaatgcactgttcatccatcaagtggaaactttgtggggcccaggagtcacacgttgcccttaagagtgccctggaacctatagaagaagatgacattagaattctaaccattgaaagaacaagagcacagggggcagctactaatgtcacccaggaccatagaaacccatcaggcttcacacccccaccatcaagaagaaccccctccagtcctattacctgccctcttcatcccaccctgcttctttcttaatctgaactcccactgcttctctccacgcctggtttttctctgttcctttagggaccagctcccctatgccatcaaactcctaatgggaggctccctccacctcctgcctcctgaggtggctgattcccttaagacatctcaagaggatgctcctcaaccttcctttttaggtacctcatggcctggtggtggtgaagacattttcctaccgttctagaccacctccaggccttttctccttcagtattgcatacgcatgccctctctgtttctctgatgctcatgctatcagtctagactaccttctacaacacctccccatatgtctgtgcccatatggccatggccctcatccagtgaagatgttgacactattctcagtctctcacctttcatactgtcctaatcacaggtgactcgatgtctacataaactaagtctagtgagagggcttcacatgatgactcacagagcttgacatgttccctgcatttgaaagcattgtggaccaatgactacatcacacctgagaatgttgaagagaagctggctggcgctgtggtcctagcccagggggctgatgtaaaacctgcatcatgaagtttgcctgaggctgtgttaaaaaagatcctgccccagacatttctgccagagaaagaggcctccctga

SEQ ID NO: 2 - DNA, 1567 bp, Homo sapiens
  CDS (129)..(1241)
gggctcagggagggctctgtgcctccgttcagcagagctgcagctgctgcccagctctcaggaggcaagctggactccctcactcagctgcaggagcaaggacagtgaggctcaaccccgcctgagccatgccagccaacttcacagagggcagcttcgattccagtgggaccgggcagacgctggattcttccccagtggcttgcactgaaacagtgacttttactgaagtggtggaaggaaaggaatggggttccttctactactcctttaagactgagcaattgataactctgtgggtcctctttgtttttaccattgttggaaactccgttgtgcttttttccacatggaggagaaagaagaagtcaagaatgaccttctttgtgactcagctggccatcacagattctttcacaggactggtcaacatcttgacagatattaattggcgattcactggagacttcacggcacctgacctggtttgccgagtggtccgctatttgcaggttgtgctgctctacgcctctacctacgtcctggtgtccctcagcatagacagataccatgccatcgtctaccccatgaagttccttcaaggagaaaagcaagccagggtcctcattgtgatcgcctggagcctgtcttttctgttctccattcccaccctgatcatatttgggaagaggacactgtccaacggtgaagtgcagtgctgggccctgtggcctgacgactcctactggaccccatacatgaccatcgtggccttcctggtgtacttcatccctctgacaatcatcagcatcatgtatggcattgtgatccgaactatttggattaaaagcaaaacctacgaaacagtgatttccaactgctcagatgggaaactgtgcagcagctataaccgaggactcatctcaaaggcaaaaatcaaggctatcaagtatagcatcatcatcattcttgccttcatctgctgttggagtccatacttcctgtttgacattttggacaatttcaacctccttccagacacccaggagcgtttctatgcctctgtgatcattcagaacctgccagcattgaatagtgccatcaaccccctcatctactgtgtcttcagcagctccatctctttcccctgcagggagcaaagatcacaggattccagaatgacgttccgggagagaactgagaggcatgagatgcagattctgtccaagccagaattcatctagaccctagggcagtgccagtgctaggctgagcaccatcagctctcccaggtccttgtcacctgcttgggcacgtgcatggaacccgagccaacttcaccccaccctcgtcattacctgggagatgcacaagacaaatgttctaatgactgcatgcactgcttaagtattggccaacacgaactccccagttattcatgccagccaggaaggaaacgccttccttccccaccattcccagccctccttcccactggccagcacctgaacccagtgaacacaggcattagtggtccagggtcctggcttggagccagtgagtagac

SEQ ID NO: 3 - protein, 371 aa, Homo sapiens
MPANFTEGSFDSSGTGQTLDSSPVACTETVTFTEVVEGKEWGSFYYSFKTEQLITLWVLFVFTIVGNSVVLFSTWRRKKKSRMTFFVTQLAITDSFTGLVNILTDINWRFTGDFTAPDLVCRVVRYLQVVLLYASTYVLVSLSIDRYHAIVYPMKFLQGEKQARVLIVIAWSLSFLFSIPTLIIFGKRTLSNGEVQCWALWPDDSYWTPYMTIVAFLVYFIPLTIISIMYGIVIRTIWIKSKTYETVISNCSDGKLCSSYNRGLISKAKIKAIKYSIIIILAFICCWSPYFLFDILDNFNLLPDTQERFYASVIIQNLPALNSAINPLIYCVFSSSISFPCREQRSQDSRMTFRERTERHEMQILSKPEFI

SEQ ID NO: 4 - DNA, 1410 bp, Homo sapiens
  CDS (129)..(1259)
gggctcagggagggctctgtgcctccgttcagcagagctgcagctgctgcccagctctcaggaggcaagctggactccctcactcagctgcaggagcaaggacagtgaggctcaaccccgcctgagccatgccagccaacttcacagagggcagcttcgattccagtgggaccgggcagacgctggattcttccccagtggcttgcactgaaacagtgacttttactgaagtggtggaaggaaaggaatggggttccttctactactcctttaagactgagcaattgataactctgtgggtcctctttgtttttaccattgttggaaactccgttgtgcttttttccacatggaggagaaagaagaagtcaagaatgaccttctttgtgactcagctggccatcacagattctttcacaggactggtcaacatcttgacagatattaattggcgattcactggagacttcacggcacctgacctggtttgccgagtggtccgctatttgcaggttgtgctgctctacgcctctacctacgtcctggtgtccctcagcatagacagataccatgccatcgtctaccccatgaagttccttcaaggagaaaagcaagccagggtcctcattgtgatcgcctggagcctgtcttttctgttctccattcccaccctgatcatatttgggaagaggacactgtccaacggtgaagtgcagtgctgggccctgtggcctgacgactcctactggaccccatacatgaccatcgtggccttcctggtgtacttcatccctctgacaatcatcagcatcatgtatggcattgtgatccgaactatttggattaaaagcaaaacctacgaaacagtgatttccaactgctcagatgggaaactgtgcagcagctataaccgaggactcatctcaaaggcaaaaatcaaggctatcaagtatagcatcatcatcattcttgccttcatctgctgttggagtccatacttcctgtttgacattttggacaatttcaacctccttccagacacccaggagcgtttctatgcctctgtgatcattcagaacctgccagcattgaatagtgccatcaaccccctcatctactgtgtcttcagcagctccatctctttcccctgcagggtcatccgtctccgtcagctccaggaggctgcgctaatgctctgccctcaacgagagaactggaagggtacttggccaggtgtaccttcctgggctcttccaaggtgacagctctcaccctgtgctgcaggtggccctgtgcctggtgccacttctcactgcttaccagggcacaaggacaccagtggttcccaaaatgggtcacagcaggatggcctgcatcagattcaccagggagggctataagaaggcagac

SEQ ID NO: 5 - protein, 377 aa, Homo sapiens
MPANFTEGSFDSSGTGQTLDSSPVACTETVTFTEVVEGKEWGSFYYSFKTEQLITLWVLFVFTIVGNSVVLFSTWRRKKKSRMTFFVTQLAITDSFTGLVNILTDINWRFTGDFTAPDLVCRVVRYLQVVLLYASTYVLVSLSIDRYHAIVYPMKFLQGEKQARVLIVIAWSLSFLFSIPTLIIFGKRTLSNGEVQCWALWPDDSYWTPYMTIVAFLVYFIPLTIISIMYGIVIRTIWIKSKTYETVISNCSDGKLCSSYNRGLISKAKIKAIKYSIIIILAFICCWSPYFLFDILDNFNLLPDTQERFYASVIIQNLPALNSAINPLIYCVFSSSISFPCRVIRLRQLQEAALMLCPQRENWKGTWPGVPSWALPR

SEQ ID NO: 6 - DNA, 1377 bp, Homo sapiens
  CDS (129)..(1226)
gggctcagggagggctctgtgcctccgttcagcagagctgcagctgctgcccagctctcaggaggcaagctggactccctcactcagctgcaggagcaaggacagtgaggctcaaccccgcctgagccatgccagccaacttcacagagggcagcttcgattccagtgggaccgggcagacgctggattcttccccagtggcttgcactgaaacagtgacttttactgaagtggtggaaggaaaggaatggggttccttctactactcctttaagactgagcaattgataactctgtgggtcctctttgtttttaccattgttggaaactccgttgtgcttttttccacatggaggagaaagaagaagtcaagaatgaccttctttgtgactcagctggccatcacagatattaattggcgattcactggagacttcacggcacctgacctggtttgccgagtggtccgctatttgcaggttgtgctgctctacgcctctacctacgtcctggtgtccctcagcatagacagataccatgccatcgtctaccccatgaagttccttcaaggagaaaagcaagccagggtcctcattgtgatcgcctggagcctgtcttttctgttctccattcccaccctgatcatatttgggaagaggacactgtccaacggtgaagtgcagtgctgggccctgtggcctgacgactcctactggaccccatacatgaccatcgtggccttcctggtgtacttcatccctctgacaatcatcagcatcatgtatggcattgtgatccgaactatttggattaaaagcaaaacctacgaaacagtgatttccaactgctcagatgggaaactgtgcagcagctataaccgaggactcatctcaaaggcaaaaatcaaggctatcaagtatagcatcatcatcattcttgccttcatctgctgttggagtccatacttcctgtttgacattttggacaatttcaacctccttccagacacccaggagcgtttctatgcctctgtgatcattcagaacctgccagcattgaatagtgccatcaaccccctcatctactgtgtcttcagcagctccatctctttcccctgcagggtcatccgtctccgtcagctccaggaggctgcgctaatgctctgccctcaacgagagaactggaagggtacttggccaggtgtaccttcctgggctcttccaaggtgacagctctcaccctgtgctgcaggtggccctgtgcctggtgccacttctcactgcttaccagggcacaaggacaccagtggttcccaaaatgggtcacagcaggatggcctgcatcagattcaccagggagggctataagaaggcagac

SEQ ID NO: 7 - protein, 366 aa, Homo sapiens
MPANFTEGSFDSSGTGQTLDSSPVACTETVTFTEVVEGKEWGSFYYSFKTEQLITLWVLFVFTIVGNSVVLFSTWRRKKKSRMTFFVTQLAITDINWRFTGDFTAPDLVCRVVRYLQVVLLYASTYVLVSLSIDRYHAIVYPMKFLQGEKQARVLIVIAWSLSFLFSIPTLIIFGKRTLSNGEVQCWALWPDDSYWTPYMTIVAFLVYFIPLTIISIMYGIVIRTIWIKSKTYETVISNCSDGKLCSSYNRGLISKAKIKAIKYSIIIILAFICCWSPYFLFDILDNFNLLPDTQERFYASVIIQNLPALNSAINPLIYCVFSSSISFPCRVIRLRQLQEAALMLCPQRENWKGTWPGVPSWALPR

SEQ ID NO: 8 - DNA, 814 bp, Homo sapiens
  CDS (129)..(410)
gggctcagggagggctctgtgcctccgttcagcagagctgcagctgctgcccagctctcaggaggcaagctggactccctcactcagctgcaggagcaaggacagtgaggctcaaccccgcctgagccatgccagccaacttcacagagggcagcttcgattccagtgggaccgggcagacgctggattcttccccagtggcttgcactgaaacagtgacttttactgaagtggtggaaggaaaggaatggggttccttctactactcctttaagactgagcaattgataactctgtgggtcctctttgtttttaccattgttggaaactccgttgtgcttttttccacatggaggagaaagaagaagtcaagaatgaccttctttgtgactcagctggccatcacagtataacaagcccacctgcttgagctgggctgcagtggccagggtaaacatccaaggcaccagtgaaaaatacagagaaggtaaaaggagcaagagttctgaagatggaacctgggatgggggaaagtttcttcaatctttcctaccaacaagaactccaatttttcactcctataaccgtagaagtagaggtaattaggatcatccagcaaatgcttagaggcaaatatccctggatgaggatgccacagcttattttcattatatttcttcgattacagtgtggtaatgcatgttgtatggaactacatattctttcagaatgaaaggatttagaggtggcaagaatatcagcttgaaatttaaagttttttcataaacaataaacaaatgataattgaaaattc

SEQ ID NO: 9 - protein, 94 aa, Homo sapiens
MPANFTEGSFDSSGTGQTLDSSPVACTETVTFTEVVEGKEWGSFYYSFKTEQLITLWVLFVFTIVGNSVVLFSTWRRKKKSRMTFFVTQLAITV

SEQ ID NO: 10 - DNA, 1463 bp, Homo sapiens
  CDS (129)..(602)
gggctcagggagggctctgtgcctccgttcagcagagctgcagctgctgcccagctctcaggaggcaagctggactccctcactcagctgcaggagcaaggacagtgaggctcaaccccgcctgagccatgccagccaacttcacagagggcagcttcgattccagtgggaccgggcagacgctggattcttccccagtggcttgcactgaaacagtgacttttactgaagtggtggaaggaaaggaatggggttccttctactactcctttaagactgagcaattgataactctgtgggtcctctttgtttttaccattgttggaaactccgttgtgcttttttccacatggaggagaaagaagaagtcaagaatgaccttctttgtgactcagctggccatcacaggttgtgctgctctacgcctctacctacgtcctggtgtccctcagcatagacagataccatgccatcgtctaccccatgaagttccttcaaggagaaaagcaagccagggtcctcattgtgatcgcctggagcctgtcttttctgttctccattcccaccctgatcatatttgggaagaggacactgtccaacggtgaagtgcagtgctgggccctgtggcctgacgactcctactggaccccatacatgaccatcgtggccttcctggtgtacttcatccctctgacaatcatcagcatcatgtatggcattgtgatccgaactatttggattaaaagcaaaacctacgaaacagtgatttccaactgctcagatgggaaactgtgcagcagctataaccgaggactcatctcaaaggcaaaaatcaaggctatcaagtatagcatcatcatcattcttgccttcatctgctgttggagtccatacttcctgtttgacattttggacaatttcaacctccttccagacacccaggagcgtttctatgcctctgtgatcattcagaacctgccagcattgaatagtgccatcaaccccctcatctactgtgtcttcagcagctccatctctttcccctgcagggagcaaagatcacaggattccagaatgacgttccgggagagaactgagaggcatgagatgcagattctgtccaagccagaattcatctagaccctagggcagtgccagtgctaggctgagcaccatcagctctcccaggtccttgtcacctgcttgggcacgtgcatggaacccgagccaacttcaccccaccctcgtcattacctgggagatgcacaagacaaatgttctaatgactgcatgcactgcttaagtattggccaacacgaactccccagttattcatgccagccaggaaggaaacgccttccttccccaccattcccagccctccttcccactggccagcacctgaacccagtgaacacaggcattagtggtccagggtcctggcttggagccagtgagtagac

SEQ ID NO: 11 - protein, 158 aa, Homo sapiens
MPANFTEGSFDSSGTGQTLDSSPVACTETVTFTEVVEGKEWGSFYYSFKTEQLITLWVLFVFTIVGNSVVLFSTWRRKKKSRMTFFVTQLAITGCAALRLYLRPGVPQHRQIPCHRLPHEVPSRRKASQGPHCDRLEPVFSVLHSHPDHIWEEDTVQR

SEQ ID NO: 12 - DNA, 1473 bp, Homo sapiens
  CDS (129)..(536)
gggctcagggagggctctgtgcctccgttcagcagagctgcagctgctgcccagctctcaggaggcaagctggactccctcactcagctgcaggagcaaggacagtgaggctcaaccccgcctgagccatgccagccaacttcacagagggcagcttcgattccagtgggaccgggcagacgctggattcttccccagtggcttgcactgaaacagtgacttttactgaagtggtggaaggaaaggaatggggttccttctactactcctttaagactgagcaattgataactctgtgggtcctctttgtttttaccattgttggaaactccgttgtgcttttttccacatggaggagaaagaagaagtcaagaatgaccttctttgtgactcagctggccatcacagattctttcacaggactggtcaacatcttgacagatattaattggcgattcactggagacttcacggcacctgacctggtttgccgagtggtccgctatttgcagaaaagcaagccagggtcctcattgtgatcgcctggagcctgtcttttctgttctccattcccaccctgatcatatttgggaagaggacactgtccaacggtgaagtgcagtgctgggccctgtggcctgacgactcctactggaccccatacatgaccatcgtggccttcctggtgtacttcatccctctgacaatcatcagcatcatgtatggcattgtgatccgaactatttggattaaaagcaaaacctacgaaacagtgatttccaactgctcagatgggaaactgtgcagcagctataaccgaggactcatctcaaaggcaaaaatcaaggctatcaagtatagcatcatcatcattcttgccttcatctgctgttggagtccatacttcctgtttgacattttggacaatttcaacctccttccagacacccaggagcgtttctatgcctctgtgatcattcagaacctgccagcattgaatagtgccatcaaccccctcatctactgtgtcttcagcagctccatctctttcccctgcagggagcaaagatcacaggattccagaatgacgttccgggagagaactgagaggcatgagatgcagattctgtccaagccagaattcatctagaccctagggcagtgccagtgctaggctgagcaccatcagctctcccaggtccttgtcacctgcttgggcacgtgcatggaacccgagccaacttcaccccaccctcgtcattacctgggagatgcacaagacaaatgttctaatgactgcatgcactgcttaagtattggccaacacgaactccccagttattcatgccagccaggaaggaaacgccttccttccccaccattcccagccctccttcccactggccagcacctgaacccagtgaacacaggcattagtggtccagggtcctggcttggagccagtgagtagac

SEQ ID NO: 13 - protein, 136 aa, Homo sapiens
MPANFTEGSFDSSGTGQTLDSSPVACTETVTFTEVVEGKEWGSFYYSFKTEQLITLWVLFVFTIVGNSVVLFSTWRRKKKSRMTFFVTQLAITDSFTGLVNILTDINWRFTGDFTAPDLVCRVVRYLQKSKPGSSL

SEQ ID NO: 14 - DNA, 1369 bp, Homo sapiens
  CDS (129)..(1043)
gggctcagggagggctctgtgcctccgttcagcagagctgcagctgctgcccagctctcaggaggcaagctggactccctcactcagctgcaggagcaaggacagtgaggctcaaccccgcctgagccatgccagccaacttcacagagggcagcttcgattccagtgggaccgggcagacgctggattcttccccagtggcttgcactgaaacagtgacttttactgaagtggtggaaggaaaggaatggggttccttctactactcctttaagactgagcaattgataactctgtgggtcctctttgtttttaccattgttggaaactccgttgtgcttttttccacatggaggagaaagaagaagtcaagaatgaccttctttgtgactcagctggccatcacagaaaagcaagccagggtcctcattgtgatcgcctggagcctgtcttttctgttctccattcccaccctgatcatatttgggaagaggacactgtccaacggtgaagtgcagtgctgggccctgtggcctgacgactcctactggaccccatacatgaccatcgtggccttcctggtgtacttcatccctctgacaatcatcagcatcatgtatggcattgtgatccgaactatttggattaaaagcaaaacctacgaaacagtgatttccaactgctcagatgggaaactgtgcagcagctataaccgaggactcatctcaaaggcaaaaatcaaggctatcaagtatagcatcatcatcattcttgccttcatctgctgttggagtccatacttcctgtttgacattttggacaatttcaacctccttccagacacccaggagcgtttctatgcctctgtgatcattcagaacctgccagcattgaatagtgccatcaaccccctcatctactgtgtcttcagcagctccatctctttcccctgcagggagcaaagatcacaggattccagaatgacgttccgggagagaactgagaggcatgagatgcagattctgtccaagccagaattcatctagaccctagggcagtgccagtgctaggctgagcaccatcagctctcccaggtccttgtcacctgcttgggcacgtgcatggaacccgagccaacttcaccccaccctcgtcattacctgggagatgcacaagacaaatgttctaatgactgcatgcactgcttaagtattggccaacacgaactccccagttattcatgccagccaggaaggaaacgccttccttccccaccattcccagccctccttcccactggccagcacctgaacccagtgaacacaggcattagtggtccagggtcctggcttggagccagtgagtagac

SEQ ID NO: 15 - protein, 305 aa, Homo sapiens
MPANFTEGSFDSSGTGQTLDSSPVACTETVTFTEVVEGKEWGSFYYSFKTEQLITLWVLFVFTIVGNSVVLFSTWRRKKKSRMTFFVTQLAITEKQARVLIVIAWSLSFLFSIPTLIIFGKRTLSNGEVQCWALWPDDSYWTPYMTIVAFLVYFIPLTIISIMYGIVIRTIWIKSKTYETVISNCSDGKLCSSYNRGLISKAKIKAIKYSIIIILAFICCWSPYFLFDILDNFNLLPDTQERFYASVIIQNLPALNSAINPLIYCVFSSSISFPCREQRSQDSRMTFRERTERHEMQILSKPEFI

SEQ ID NO: 16 - DNA, 1532 bp, Homo sapiens
  CDS (231)..(383)
gacacagagaagaccactcccacctccccgagtgcaaggtgtgaagggacagatcttttaaccatgcctgcccccttatacttgctgttcatagaattgcaactgaaagtgaccatgaggatccactggatggagttacttctttcttaagtgaggaggctaagatctggagtgacttctccccagatttttgtatacctgactctgtttcagcatccgcttcccaaagaatgcagtgtgaagcaggagcttatgtgagaagaaacgcagggagacagttcagtcactgcaatcttcatgcccatcagtttcttgtgagaagaaaacaagagctccaatctgttagatggtattttgaagcaggtctttgggtaaaggacacctagacccagtgaaggtcatggtgattattattggacaatgggacatcactctgctatttgaacaaataagactttttcctgacatgcatctggaggcaggtcaaagctccaggccaactccaagtttctgatggggtctctagccaatggaaggcttcttctccttcaattgcctgactcttcaggactcttaaatactgcaaagtgagaaaatgagacaggttgcactgagggctgttagccagacagagtctcgaactggaagtccatctagatgttttgcataagagaatggaaacaatctgtctgtgatttagggacatactctggcagcaatatgggaatacagtttcaatcctcattaacaaaacaggtatgaaatacatatttatttagtaaggtgccagctgtatgaaaaatccatttcttatttcccataatgtttctgaaatgtcttagcagtgcatagagacagcatgtcatcattttctagggactgtgtgttattgcatttttcctagggaagatcttttctaggtcacctgctccttcgctaaagctctgaccaatctagcttgctaacctgtgactccattttcctaagtcctgagagagaaaaacgctttgcagcaaattatgccaggcatccttgtgtctaaatgaaaaaaggaaaaagcctccttccttccctctgttgagaagtgcacggtccacatatgcatgcacagcatatactgtgagggtatttgcagtcccttgggttgctttgataactggccaggttgctgttctatttttccacattctattaatcctcctacaggcagttattaggtattgagtgctcacacacccctggcatagtcaccacatgccattagctccagataaacttccagaaaaaagtccatcccccacttctctcagctgcctgccaacgctggacaccttctcaccaagccagcaggacagagaaaagcctgggctttaagatcaaacaaacacagcttcaaattaggactctgtcacttcctgtgtactgggcactttgctgagtatgtggtttctcatctgtaaaacagagaaagatgattatctcccaatctttctatgttatatgtttgaattaaataaggtactctccatgaa

SEQ ID NO: 17 - protein, 51 aa, Homo sapiens
MQCEAGAYVRRNAGRQFSHCNLHAHQFLVRRKQELQSVRWYFEAGLWVKDT

SEQ ID NO: 18 - DNA, 1407 bp, Homo sapiens
  CDS (244)..(402)
gacacagagaagaccactcccacctccccgagtgcaaggtgtgaagggacagatcttttaaccatgcctgcccccttatacttgctgttcatagaattgcaactgaaagtgaccatgaggatccactggatggagttacttctttcttaagtgaggaggctaagatctgagttcttcacatctctctgtagataaaatttccggtctggtttcacattcctctgtcagaagaactttctttaatgtttcttaaagtacaggtctgctgcttatgtgagaagaaacgcagggagacagttcagtcactgcaatcttcatgcccatcagtttcttgtgagaagaaaacaagagctccaatctgttagatggtattttgaagcaggtctttgggtaaaggacacctagacccagtgaaggtcatggtgattattattggacaatgggacatcactctgctattaagtgagaaaatgagacaggttgcactgagggctgttagccagacagagtctcgaactggaagtccatctagatgttttgcataagagaatggaaacaatctgtctgtgatttagggacatactctggcagcaatatgggaatacagtttcaatcctcattaacaaaacaggtatgaaatacatatttatttagtaaggtgccagctgtatgaaaaatccatttcttatttcccataatgtttctgaaatgtcttagcagtgcatagagacagcatgtcatcattttctagggactgtgtgttattgcatttttcctagggaagatcttttctaggtcacctgctccttcgctaaagctctgaccaatctagcttgctaacctgtgactccattttcctaagtcctgagagagaaaaacgctttgcagcaaattatgccaggcatccttgtgtctaaatgaaaaaaggaaaaagcctccttccttccctctgttgagaagtgcacggtccacatatgcatgcacagcatatactgtgagggtatttgcagtcccttgggttgctttgataactggccaggttgctgttctatttttccacattctattaatcctcctacaggcagttattaggtattgagtgctcacacacccctggcatagtcaccacatgccattagctccagataaacttccagaaaaaagtccatcccccacttctctcagctgcctgccaacgctggacaccttctcaccaagccagcaggacagagaaaagcctgggctttaagatcaaacaaacacagcttcaaattaggactctgtcacttcctgtgtactgggcactttgctgagtatgtggtttctcatctgtaaaacagagaaagatgattatctcccaatctttctatgttatatgtttgaattaaataaggtactctccatgaa

SEQ ID NO: 19 - protein, 53 aa, Homo sapiens
CFLKYRSAAYVRRNAGRQFSHCNLHAHQFLVRRKQELQSVRWYFEAGLWVKDT

SEQ ID NO: 20 - DNA, 341 bp, Homo sapiens
  CDS (146)..(328)
gctgatggtggaaggagaatgagtctctgatgcctttggacttgatgctggaaagacttaagactttgggggactactggaaaggagtgacttctccccagatttttgtatacctgactctgtttcagcatccgcttcccaaagaatgcagtgtgaagcaggagcttatgtgagaagaaacgcagggagacagttcagtcactgcaatcttcatgcccatcagtttcttgtgagaagaaaacaagtggatatacactgttccaagcagcatgtgttgaaaagatttgtcttttccccatttaatggtcttggtacctttctcaaaaattgaccatatatga

SEQ ID NO: 21 - protein, 61 aa, Homo sapiens
MQCEAGAYVRRNAGRQFSHCNLHAHQFLVRRKQVDIHCSKQHVLKRFVFSPFNGLGTFLKN

SEQ ID NO: 22 - DNA, 710 bp, Homo sapiens
  CDS (175)..(291)
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaagaaaacaaggactgaaatccacacaggaaggtggcagtgaactccacagacggacctggacgcctcaacactcctggccttacctcccttgctgaacgtctcaagtttctctgcgttcaggactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtgacgctgggcttctttaacaaattaatcccaagtgcaagacatttatttcttctgtacctaatgacctgagcaatccttctctgctgaacctggtagtgtcatctttagaagtgaagacacaattaacacatggtcatttcttcattatatcgttgttact

SEQ ID NO: 23 - protein, 39 aa, Homo sapiens
MKKLKHRPAYVRRNAGRQFSHCNLHAHQFLVRRKQENKD

SEQ ID NO: 24 - DNA, 949 bp, Homo sapiens
  CDS (175)..(366)
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaagactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtagcaaactgctatgcatccttcagctgcaagggattgaatgctatcaacaaccatacaagtggagaagcagatgcttccctagctgagcctcaggctttttgatggaattgctacaacttggtgcatgcctgctcctaaaagaaatactcaggaattgtctcataaagtcctcacctactggcaaaaacaagatgttctactcccaggttgactttttcaagccccaagatgttgagtcagccattctccaaggatctcgatttccttttaatggaaaataacattaaacaccaaatataagcctcgctgtcccacatgcgtattggggacaagatgaaacctgcttccaggctactttggcagcagaactgaaaaaggctttttttccagatatatgatttctcatcgacagggttgcacagccctctttattgttcgtgtaaatgacacccttggatctgaacaatacacaccaggacaattgtgtgcaacagttctacaaactgatatttctaatta

SEQ ID NO: 25 - protein, 64 aa, Homo sapiens
MKKLKHRPAYVRRNAGRQFSHCNLHAHQFLVRRKQDWQRLLPPLSCQVGSPGCSARKRSHVQEH

SEQ ID NO: 26 - DNA, 1068 bp, Homo sapiens
  CDS (176)..(367)
gtctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaagactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtagcaaactgctatgcatccttcagctgcaagggattgaatgctatcaacaaccatacaagtggagaagcagatgcttccctagctgagcctcagctctgttttcctcagtcctaggggcagattctaatttggtacagaaaaaaaaagtactcctgccaattcccatcctggaatgactgaagaaaagaaaatgcatgtgattattttaaaggctttttgatggaattgctacaacttggtgcatgcctgctcctaaaagaaatactcaggaattgtctcataaagtcctcacctactggcaaaaacaagatgttctactcccaggttgactttttcaagccccaagatgttgagtcagccattctccaaggatctcgatttccttttaatggaaaataacattaaacaccaaatataagcctcgctgtcccacatgcgtattggggacaagatgaaacctgcttccaggctactttggcagcagaactgaaaaaggctttttttccagatatatgatttctcatcgacagggttgcacagccctctttattgttcgtgtaaatgacacccttggatctgaacaatacacaccaggacaattgtgtgcaacagttctacaaactgatatttctaatta

SEQ ID NO: 27 - protein, 64 aa, Homo sapiens; identical to SEQ ID NO: 25

SEQ ID NO: 28 - DNA, 799 bp, Homo sapiens
  CDS (175)..(363)
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaaagcaaactgctatgcatccttcagctgcaagggattgaatgctatcaacaaccatacaagtggagaagcagatgcttccctagctgagcctcaggctttttgatggaattgctacaacttggtgcatgcctgctcctaaaagaaatactcaggaattgtctcataaagtcctcacctactggcaaaaacaagatgttctactcccaggttgactttttcaagccccaagatgttgagtcagccattctccaaggatctcgatttccttttaatggaaaataacattaaacaccaaatataagcctcgctgtcccacatgcgtattggggacaagatgaaacctgcttccaggctactttggcagcagaactgaaaaaggctttttttccagatatatgatttctcatcgacagggttgcacagccctctttattgttcgtgtaaatgacacccttggatctgaacaatacacaccaggacaattgtgtgcaacagttctacaaactgatatttctaatta

SEQ ID NO: 29 - protein, 63 aa, Homo sapiens
MKKLKHRPAYVRRNAGRQFSHCNLHAHQFLVRRKQSKLLCILQLQGIECYQQPYKWRSRCFPS

SEQ ID NO: 30 - DNA, 834 bp, Homo sapiens
  CDS (30)..(251)
gctgatggtggaaggagaatgagtctctgatgcctttggacttgatgctggaaagacttaagactttgggggactactggaaagcttatgtgagaagaaacgcagggagacagttcagtcactgcaatcttcatgcccatcagtttcttgtgagaagaaaacaagactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtagcaaactgctatgcatccttcagctgcaagggattgaatgctatcaacaaccatacaagtggagaagcagatgcttccctagctgagcctcaggctttttgatggaattgctacaacttggtgcatgcctgctcctaaaagaaatactcaggaattgtctcataaagtcctcacctactggcaaaaacaagatgttctactcccaggttgactttttcaagccccaagatgttgagtcagccattctccaaggatctcgatttccttttaatggaaaataacattaaacaccaaatataagcctcgctgtcccacatgcgtattggggacaagatgaaacctgcttccaggctactttggcagcagaactgaaaaaggctttttttccagatatatgatttctcatcgacagggttgcacagccctctttattgttcgtgtaaatgacacccttggatctgaacaatacacaccaggacaattgtgtgcaacagttctacaaactgatatttctaatta

SEQ ID NO: 31 - protein, 74 aa, Homo sapiens
MPLDLMLERLKTLGDYWKAYVRRNAGRQFSHCNLHAHQFLVRRKQDWQRLLPPLSCQVGSPGCSARKRSHVQEH

SEQ ID NO: 32 - DNA, 550 bp, Homo sapiens
  CDS (175)..(366)
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaagactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtgccatgttttgaaccactagattagagggtcaagcaatttcttggaattttactctgaattctacgtagaccattttcatgtgtatacctcctctgagtcaccctcaggtagggacatttt

SEQ ID NO: 33 - protein, 64 aa, Homo sapiens; identical to SEQ ID NO: 25

SEQ ID NO: 34 - DNA, 376 bp, Homo sapiens
  CDS (175)..(363)
tctaggactcagaaatatagatgttagtaagagcaaacagacataacagataacacatacaaagtgcctaccacatgctaaccactgctgcaggcactttctatagaagaactaatttaatcatcaccataaccctatggggtagatgatatttttacaacctccattttacagatgaagaaactgaagcatagacctgcttatgtgagaagaaacgcagggagacagttcagtcactgcaatcttcatgcccatcagtttcttgtgagaagaaaacaagtggatatacactgttccaagcagcatgtgttgaaaagatttgtcttttccccatttaatggtcttggtacctttctcaaaaattgaccatatatga

SEQ ID NO: 35 - protein, 63 aa, Homo sapiens
MKKLKHRPAYVRRNAGRQFSHCNLHAHQFLVRRKQVDIHCSKQHVLKRFVFSPFNGLGTFLKN

SEQ ID NO: 36 - DNA, 369 bp, Homo sapiens
  CDS (146)..(247)
gctgatggtggaaggagaatgagtctctgatgcctttggacttgatgctggaaagacttaagactttgggggactactggaaaggagtgacttctccccagatttttgtatacctgactctgtttcagcatccgcttcccaaagaatgcagtgtgaagcaggagcttatgtgagaagaaacgcagggagacagttcagtcactgcaatcttcatgcccatcagtttcttgtgagaagaaaacaagtttaggaaaacttcctacaccttctttgttgggatgttctctggactaatgactccaggcgagaccaccgttgatcatgaactcactttgaaacagaagctgggttggtaagactggagctact

SEQ ID NO: 37 - protein, 34 aa, Homo sapiens
MQCEAGAYVRRNAGRQFSHCNLHAHQFLVRRKQV

SEQ ID NO: 38 - DNA, 512 bp, Homo sapiens
  CDS (62)..(172)
gagtgacttctccccagatttttgtatacctgactctgtttcagcatccgcttcccaaagaatgcagtgtgaagcaggagcttatgtgagaagaaacgcagggagacagttcagtcactgcaatcttcatgcccatcagtttcttgtgagaagaaaacaagaaaacaaggactgaaatccacacaggaaggtggcagtgaactccacagacggacctggacgcctcaacactcctggccttacctcccttgctgaacgtctcaagtttctctgcgttcaggtaatgtataggagggttatgagggcagagaattcctaagctcattagtaaattgctcttcagaaaagtgctttgaagcaaagctaatttcctttcccaatatgagaagatttggccctaccagaaaaaggaaatgatttgaatgtgcgccaaaaaatatgttttctttcttttctttgttgaacactcatcgggagttactcttattagttccgcatttttattgccattt

SEQ ID NO: 39 - protein, 37 aa, Homo sapiens [sequence omitted]

SEQ ID NO: 40 - DNA, 830 bp, Homo sapiens
  CDS (62)..(247)
gagtgacttctccccagatttttgtatacctgactctgtttcagcatccgcttcccaaagaatgcagtgtgaagcaggagcttatgtgagaagaaacgcagggagacagttcagtcactgcaatcttcatgcccatcagtttcttgtgagaagaaaacaagactggcaacgcctgcttcctcctctgagctgtcaagtaggaagtccgggctgctctgctagaaagagaagtcatgtgcaggagcactgaggcatcccaggtgtgacactcttccacctagagcattccgtctctcatcctctgccatgtagcaaactgctatgcatccttcagctgcaagggattgaatgctatcaacaaccatacaagtggagaagcagatgcttccctagctgagcctcaggctttttgatggaattgctacaacttggtgcatgcctgctcctaaaagaaatactcaggaattgtctcataaagtcctcacctactggcaaaaacaagatgttctactcccaggttgactttttcaagccccaagatgttgagtcagccattctccaaggatctcgatttccttttaatggaaaataacattaaacaccaaatataagcctcgctgtcccacatgcgtattggggacaagatgaaacctgcttccaggctactttggcagcagaactgaaaaaggctttttttccagatatatgatttctcatcgacagggttgcacagccctctttattgttcgtgtaaatgacacccttggatctgaacaatacacaccaggacaattgtgtgcaacagttctacaaactgatatttctaatta

SEQ ID NO: 41 - protein, 62 aa, Homo sapiens
MQCEAGAYVRRNAGRQFSHCNLHAHQFLVRRKQDWQRLLPPLSCQVGSPGCSARKRSHVQEH

SEQ ID NO: 42 - protein, 13 aa, Artificial Sequence
  Description of Artificial Sequence Synthetic peptide
YVRRNAGRQFSHC

SEQ ID NO: 43 - DNA, 20 bp, Artificial Sequence
  Description of Artificial Sequence Synthetic oligonucleotide
ccccctgctctctctctctt

SEQ ID NO: 44 - DNA, 21 bp, Artificial Sequence
  Description of Artificial Sequence Synthetic oligonucleotide
gatcttccatttcaccaagca

SEQ ID NO: 45 - DNA, 21 bp, Artificial Sequence
  Description of Artificial Sequence Synthetic oligonucleotide
tgagttgatgaatctgctgga

SEQ ID NO: 46 - DNA, 20 bp, Artificial Sequence
  Description of Artificial Sequence Synthetic oligonucleotide
caatggatcactgcagccta

SEQ ID NO: 47 - DNA, 20 bp, Artificial Sequence
  Description of Artificial Sequence Synthetic oligonucleotide
aacacagccctccagacact

SEQ ID NO: 48 - DNA, 20 bp, Artificial Sequence
  Description of Artificial Sequence Synthetic oligonucleotide
ctattgggccctggagactt

SEQ ID NO: 49 - DNA, 20 bp, Artificial Sequence
  Description of Artificial Sequence Synthetic oligonucleotide
agggatcatgtctccagcac

SEQ ID NO: 50 - DNA, 24 bp, Artificial Sequence
  Description of Artificial Sequence Synthetic oligonucleotide
tgtgtctccttctttgtgttcata

SEQ ID NO: 51 - DNA, 20 bp, Artificial Sequence
  Description of Artificial Sequence Synthetic oligonucleotide
cacagggaggaaaggaacag

SEQ ID NO: 52 - DNA, 20 bp, Artificial Sequence
  Description of Artificial Sequence Synthetic oligonucleotide
ggccatctgataaagcagga

SEQ ID NO: 53 - DNA, 21 bp, Artificial Sequence
  Description of Artificial Sequence Synthetic oligonucleotide
gatgaaaagggaaccattcct

SEQ ID NO: 54 - DNA, 20 bp, Artificial Sequence
  Description of Artificial Sequence Synthetic oligonucleotide
tgacatgttccctgcatttg

SEQ ID NO: 55 - DNA, 21 bp, Artificial Sequence
  Description of Artificial Sequence Synthetic oligonucleotide
gggatttatccatttcctcca

SEQ ID NO: 56 - DNA, 23 bp, Artificial Sequence
  Description of Artificial Sequence Synthetic oligonucleotide
gatggatatttagctccccctta

SEQ ID NO: 57 - DNA, 20 bp, Artificial Sequence
  Description of Artificial Sequence Synthetic oligonucleotide
ggcaatgccaattaagagga

SEQ ID NO: 58 - DNA, 21 bp, Artificial Sequence
  Description of Artificial Sequence Synthetic oligonucleotide
ttcccacccattagtttttcc

SEQ ID NO: 59 - DNA, 19 bp, Artificial Sequence
  Description of Artificial Sequence Synthetic oligonucleotide
atacctggccagcggttta

SEQ ID NO: 60 - DNA, 20 bp, Artificial Sequence
  Description of Artificial Sequence Synthetic oligonucleotide
atgcagcttgcattgttcac

SEQ ID NO: 61 - DNA, 20 bp, Artificial Sequence
  Description of Artificial Sequence Synthetic oligonucleotide
ttaaatgcccactagcacca

SEQ ID NO: 62 - DNA, 20 bp, Artificial Sequence
  Description of Artificial Sequence Synthetic oligonucleotide
tgaaagcagggctaccattt

SEQ ID NO: 63 - DNA, 20 bp, Artificial Sequence
  Description of Artificial Sequence Synthetic oligonucleotide
gatgggattgaggagcttga

SEQ ID NO: 64 - DNA, 20 bp, Artificial Sequence
  Description of Artificial Sequence Synthetic oligonucleotide
tgatgcataggaatgcaagg

SEQ ID NO: 65 - DNA, 20 bp, Artificial Sequence
  Description of Artificial Sequence Synthetic oligonucleotide
tgcttttcctgtgtccattg

SEQ ID NO: 66 - DNA, 20 bp, Artificial Sequence
  Description of Artificial Sequence Synthetic oligonucleotide
agagggctttgtgctctctg

SEQ ID NO: 67 - DNA, 21 bp, Artificial Sequence
  Description of Artificial Sequence Synthetic oligonucleotide
atgccagcatcatacttcctg

SEQ ID NO: 68 - DNA, 22 bp, Artificial Sequence
  Description of Artificial Sequence Synthetic oligonucleotide
cacactagcaaagtcaggcaat

SEQ ID NO: 69 - DNA, 21 bp, Artificial Sequence
  Description of Artificial Sequence Synthetic oligonucleotide
cacgagccctggattacctac

SEQ ID NO: 70 - DNA, 23 bp, Artificial Sequence
  Description of Artificial Sequence Synthetic oligonucleotide
agaacaagctatgccagagactg

SEQ ID NO: 71 - DNA, 24 bp, Artificial Sequence
  Description of Artificial Sequence Synthetic oligonucleotide
tggaaaaatttgaacattattggg

SEQ ID NO: 72 - DNA, 18 bp, Artificial Sequence
  Description of Artificial Sequence Synthetic oligonucleotide
ggtctctgggaccccttc

SEQ ID NO: 73 - DNA, 22 bp, Artificial Sequence
  Description of Artificial Sequence Synthetic oligonucleotide
gagcagagatcagaaacaggct

SEQ ID NO: 74 - DNA, 23 bp, Artificial Sequence
  Description of Artificial Sequence Synthetic oligonucleotide
gcaggaacagaaaacgaaatacc

SEQ ID NO: 75 - DNA, 20 bp, Artificial Sequence
  Description of Artificial Sequence Synthetic oligonucleotide
acagtggatggtttcttggc

SEQ ID NO: 76 - DNA, 20 bp, Artificial Sequence
  Description of Artificial Sequence Synthetic oligonucleotide
agtctccagtgaatcgccaa

SEQ ID NO: 77 - DNA, 24 bp, Artificial Sequence
  Description of Artificial Sequence Synthetic oligonucleotide
atgccctaattctaaaaccaatga

SEQ ID NO: 78 - DNA, 18 bp, Artificial Sequence
  Description of Artificial Sequence Synthetic oligonucleotide
caccctccctggtgacct

SEQ ID NO: 79 - DNA, 20 bp, Artificial Sequence
  Description of Artificial Sequence Synthetic oligonucleotide
gcctgccctagagacatcag

SEQ ID NO: 80 - DNA, 20 bp, Artificial Sequence
  Description of Artificial Sequence Synthetic oligonucleotide
gcctgccctagagacatcag

SEQ ID NO: 81 - DNA, 20 bp, Artificial Sequence
  Description of Artificial Sequence Synthetic oligonucleotide
ccaattctcatccaaaaggc

SEQ ID NO: 82 - DNA, 49 bp, Artificial Sequence
  Description of Artificial Sequence Synthetic oligonucleotide
aactgactaaactaggtgccacgtcgtatcctgacacagggatttaagc

SEQ ID NO: 83 - DNA, 21 bp, Artificial Sequence
  Description of Artificial Sequence Synthetic oligonucleotide
ggaaactggaagtcaacacca

SEQ ID NO: 84 - DNA, 27 bp, Artificial Sequence
  Description of Artificial Sequence Synthetic oligonucleotide
cctgcaataaacattctcataattaaa

SEQ ID NO: 85 - DNA, 24 bp, Artificial Sequence
  Description of Artificial Sequence Synthetic oligonucleotide
gaagtggacttactgcatcaaaga

SEQ ID NO: 86 - DNA, 20 bp, Artificial Sequence
  Description of Artificial Sequence Synthetic oligonucleotide
gctctagaacccgctagcat

SEQ ID NO: 87 - DNA, 20 bp, Artificial Sequence
  Description of Artificial Sequence Synthetic oligonucleotide
gcaaaagttgagggggattc

SEQ ID NO: 88 - DNA, 18 bp, Artificial Sequence
  Description of Artificial Sequence Synthetic oligonucleotide
aggaacccttcccagcag

SEQ ID NO: 89 - DNA, 20 bp, Artificial Sequence
  Description of Artificial Sequence Synthetic oligonucleotide
tgcaccagctttttgaggta

SEQ ID NO: 90 - DNA, 20 bp, Artificial Sequence
  Description of Artificial Sequence Synthetic oligonucleotide
ctttagtgggaaggctgtgg

SEQ ID NO: 91 - DNA, 21 bp, Artificial Sequence
  Description of Artificial Sequence Synthetic oligonucleotide
tgctctttttgacagccaatc

SEQ ID NO: 92 - DNA, 20 bp, Artificial Sequence
  Description of Artificial Sequence Synthetic oligonucleotide
ccataagcctcgcctatttt

SEQ ID NO: 93 - DNA, 20 bp, Artificial Sequence
  Description of Artificial Sequence Synthetic oligonucleotide
caaatcctcgttctcctgct

SEQ ID NO: 94 - DNA, 20 bp, Artificial Sequence
  Description of Artificial Sequence Synthetic oligonucleotide
cctttctcagcctctgggta

SEQ ID NO: 95 - DNA, 20 bp, Artificial Sequence
  Description of Artificial Sequence Synthetic oligonucleotide
ctcagaacactgctgccaga

SEQ ID NO: 96 - DNA, 20 bp, Artificial Sequence
  Description of Artificial Sequence Synthetic oligonucleotide
ttgatctggccaaatggaac

SEQ ID NO: 97 - DNA, 21 bp, Artificial Sequence
  Description of Artificial Sequence Synthetic oligonucleotide
gcatgtgaatacagcacttgg

SEQ ID NO: 98 - DNA, 21 bp, Artificial Sequence
  Description of Artificial Sequence Synthetic oligonucleotide
ccctacaagttaccctgcaca

SEQ ID NO: 99 - DNA, 20 bp, Artificial Sequence
  Description of Artificial Sequence Synthetic oligonucleotide
tcaagctcctcaatcccatc

SEQ ID NO: 100 - DNA, 20 bp, Artificial Sequence
  Description of Artificial Sequence Synthetic oligonucleotide
aaaggcagccagtgtctgtt

SEQ ID NO: 101 - DNA, 20 bp, Artificial Sequence
  Description of Artificial Sequence Synthetic oligonucleotide
tctcagcctgctacccctta

SEQ ID NO: 102 - DNA, 20 bp, Artificial Sequence
  Description of Artificial Sequence Synthetic oligonucleotide
tcaacagaggcatgattgga

SEQ ID NO: 103 - DNA, 50 bp, Artificial Sequence
  Description of Artificial Sequence Synthetic oligonucleotide
aactgactaaactaggtgccacgtcgtccatacatgaccatcgtgctctt

SEQ ID NO: 104 - DNA, 20 bp, Artificial Sequence
  Description of Artificial Sequence Synthetic oligonucleotide
aggcaagtcacctgctcttc

SEQ ID NO: 105 - DNA, 20 bp, Artificial Sequence
  Description of Artificial Sequence Synthetic oligonucleotide
tgtctacctgttggcctgtg

SEQ ID NO: 106 - DNA, 20 bp, Artificial Sequence
  Description of Artificial Sequence Synthetic oligonucleotide
catggagaaggaaggtcagg

SEQ ID NO: 107 - DNA, 20 bp, Artificial Sequence
  Description of Artificial Sequence Synthetic oligonucleotide
atgactgcatgcactgctta

SEQ ID NO: 108 - DNA, 21 bp, Artificial Sequence
  Description of Artificial Sequence Synthetic oligonucleotide
tggctgccttcttcatctttg

SEQ ID NO: 109 - DNA, 21 bp, Artificial Sequence
  Description of Artificial Sequence Synthetic oligonucleotide
tggctgccttcttcatctttg

SEQ ID NO: 110 - DNA, 37 bp, Artificial Sequence
  Description of Artificial Sequence Synthetic oligonucleotide
taaactaggtgcgtcacatttgctcactggagatggc

SEQ ID NO: 111 - DNA, 22 bp, Artificial Sequence
  Description of Artificial Sequence Synthetic oligonucleotide
tggatttgcatttcgtctaatg

SEQ ID NO: 112 - DNA, 20 bp, Artificial Sequence
  Description of Artificial Sequence Synthetic oligonucleotide
tctccctcagaaaagccaga

SEQ ID NO: 113 - DNA, 20 bp, Artificial Sequence
  Description of Artificial Sequence Synthetic oligonucleotide
ggtgggattacaggcatgag

SEQ ID NO: 114 - DNA, 19 bp, Artificial Sequence
  Description of Artificial Sequence Synthetic oligonucleotide
ggggtttcaccgtgttagc

SEQ ID NO: 115 - DNA, 20 bp, Artificial Sequence
  Description of Artificial Sequence Synthetic oligonucleotide
gcctcagcctctggagtagc

SEQ ID NO: 116 - DNA, 20 bp, Artificial Sequence
  Description of Artificial Sequence Synthetic oligonucleotide
accatgcctcgccatagtaa

SEQ ID NO: 117 - DNA, 20 bp, Artificial Sequence
  Description of Artificial Sequence Synthetic oligonucleotide
cccaaagccctgagaaaaat

SEQ ID NO: 118 - DNA, 20 bp, Artificial Sequence
  Description of Artificial Sequence Synthetic oligonucleotide
tgtaacatcccccagggact

SEQ ID NO: 119 - DNA, 22 bp, Artificial Sequence
  Description of Artificial Sequence Synthetic oligonucleotide
aagccactaatatttgggatgg

SEQ ID NO: 120 - DNA, 20 bp, Artificial Sequence
  Description of Artificial Sequence Synthetic oligonucleotide
ggctgctttgatctgaggac

SEQ ID NO: 121 - DNA, 20 bp, Artificial Sequence
  Description of Artificial Sequence Synthetic oligonucleotide
gtgggcactgaggcatagat

SEQ ID NO: 122 - DNA, 20 bp, Artificial Sequence
  Description of Artificial Sequence Synthetic oligonucleotide
tacccagaggctgagaaagg

SEQ ID NO: 123 - DNA, 20 bp, Artificial Sequence
  Description of Artificial Sequence Synthetic oligonucleotide
gtgagctgagttcgcgctat

SEQ ID NO: 124 - DNA, 20 bp, Artificial Sequence
  Description of Artificial Sequence Synthetic oligonucleotide
gctctgggatgcgttaactt

SEQ ID NO: 125 - DNA, 21 bp, Artificial Sequence
  Description of Artificial Sequence Synthetic oligonucleotide
ctaaacaaaaaggggctgcaa

SEQ ID NO: 126 - DNA, 20 bp, Artificial Sequence
  Description of Artificial Sequence Synthetic oligonucleotide
ctcccgagttcaagcaattc

SEQ ID NO: 127 - DNA, 20 bp, Artificial Sequence
  Description of Artificial Sequence Synthetic oligonucleotide
cagaggtttggatgggattg

SEQ ID NO: 128 - DNA, 20 bp, Artificial Sequence
  Description of Artificial Sequence Synthetic oligonucleotide
gtggaggaagtgccttttca

SEQ ID NO: 129 - DNA, 20 bp, Artificial Sequence
  Description of Artificial Sequence Synthetic oligonucleotide
ggcagagagtcccacagtga

SEQ ID NO: 130 - DNA, 20 bp, Artificial Sequence
  Description of Artificial Sequence Synthetic oligonucleotide
aagcacaagcatcaggttca

SEQ ID NO: 131 - DNA, 22 bp, Artificial Sequence
  Description of Artificial Sequence Synthetic oligonucleotide
gcagatggaggtagagctgttt

SEQ ID NO: 132 - DNA, 20 bp, Artificial Sequence
  Description of Artificial Sequence Synthetic oligonucleotide
tagcacaatgcctgccctat

SEQ ID NO: 133 - DNA, 20 bp, Artificial Sequence
  Description of Artificial Sequence Synthetic oligonucleotide
gctgcagaaagaaccaaagg

SEQ ID NO: 134 - DNA, 20 bp, Artificial Sequence
  Description of Artificial Sequence Synthetic oligonucleotide
gtcttgtgcatctcccaggt

SEQ ID NO: 135 - DNA, 20 bp, Artificial Sequence
  Description of Artificial Sequence Synthetic oligonucleotide
ctagcactggcactgcccta

SEQ ID NO: 136 - DNA, 20 bp, Artificial Sequence
  Description of Artificial Sequence Synthetic oligonucleotide
tctgcaaaccgaggctatct

SEQ ID NO: 137 - DNA, 20 bp, Artificial Sequence
  Description of Artificial Sequence Synthetic oligonucleotide
tctgtgcctccgttcagcag

SEQ ID NO: 138 - DNA, 22 bp, Artificial Sequence
  Description of Artificial Sequence Synthetic oligonucleotide
aagctggactccctcactcagc

SEQ ID NO: 139 - DNA, 23 bp, Artificial Sequence
  Description of Artificial Sequence Synthetic oligonucleotide
agcaaggacagtgaggctcaacc

SEQ ID NO: 140 - DNA, 24 bp, Artificial Sequence
  Description of Artificial Sequence Synthetic oligonucleotide
ctggcatgaataactggggagttc

SEQ ID NO: 141 - DNA, 24 bp, Artificial Sequence
  Description of Artificial Sequence Synthetic oligonucleotide
tttgtcttgtgcatctcccaggta

SEQ ID NO: 142 - DNA, 24 bp, Artificial Sequence
  Description of Artificial Sequence Synthetic oligonucleotide
tatagccctccctggtgaatctga

SEQ ID NO: 143 - DNA, 24 bp, Artificial Sequence
  Description of Artificial Sequence Synthetic oligonucleotide
gtgccctggtaagcagtgagaagt

SEQ ID NO: 144 - DNA, 28 bp, Artificial Sequence
  Description of Artificial Sequence Synthetic oligonucleotide
tgagcaattgataactctgtgggtcctc

SEQ ID NO: 145 - DNA, 26 bp, Artificial Sequence
  Description of Artificial Sequence Synthetic oligonucleotide
aaataagctgtggcatcctcatccag

SEQ ID NO: 146 - DNA, 26 bp, Artificial Sequence
  Description of Artificial Sequence Synthetic oligonucleotide
tggcatcctcatccagggatatttgc

SEQ ID NO: 147 - DNA, 25 bp, Artificial Sequence
  Description of Artificial Sequence Synthetic oligonucleotide
ggaaggccacgatggtcatgtatgg

SEQ ID NO: 148 - DNA, 24 bp, Artificial Sequence
  Description of Artificial Sequence Synthetic oligonucleotide
atgacgagggtggggtgaagttgg

SEQ ID NO: 149 - DNA, 24 bp, Artificial Sequence
  Description of Artificial Sequence Synthetic oligonucleotide
gaatggtggggaaggaaggcgttt

SEQ ID NO: 150 - DNA, 24 bp, Artificial Sequence
  Description of Artificial Sequence Synthetic oligonucleotide
ggccatcctgctgtgacccatttt

SEQ ID NO: 151 - DNA, 23 bp, Artificial Sequence
  Description of Artificial Sequence Synthetic oligonucleotide
atgagatgcagattctgtccaag

SEQ ID NO: 152 - DNA, 24 bp, Artificial Sequence
  Description of Artificial Sequence Synthetic oligonucleotide
tgcacaagacaaatgttctaatga

SEQ ID NO: 153 - DNA, 24 bp, Artificial Sequence
  Description of Artificial Sequence Synthetic oligonucleotide
cagctataaccgaggactcatctc

SEQ ID NO: 154 - DNA, 23 bp, Artificial Sequence
  Description of Artificial Sequence Synthetic oligonucleotide
tgttggagtccatacttcctgtt

SEQ ID NO: 155 - DNA, 25 bp, Artificial Sequence
  Description of Artificial Sequence Synthetic oligonucleotide
aaaagacaggctccaggcgatcaca

SEQ ID NO: 156 - DNA, 27 bp, Artificial Sequence
  Description of Artificial Sequence Synthetic oligonucleotide
gattcttccccagtggcttgcactgaa

SEQ ID NO: 157 - DNA, 27 bp, Artificial Sequence
  Description of Artificial Sequence Synthetic oligonucleotide
tgatggccagctgagtcacaaagaagg

SEQ ID NO: 158 - DNA, 19 bp, Artificial Sequence
  Description of Artificial Sequence Synthetic oligonucleotide
actcagctgcaggagcaag

SEQ ID NO: 159 - DNA, 20 bp, Artificial Sequence
  Description of Artificial Sequence Synthetic oligonucleotide
aggaggaagaaatccagcct

SEQ ID NO: 160 - DNA, 25 bp, Artificial Sequence
  Description of Artificial Sequence Synthetic oligonucleotide
gccatacattgttagtaacctgaaa

SEQ ID NO: 161 - DNA, 22 bp, Artificial Sequence
  Description of Artificial Sequence Synthetic oligonucleotide
ttcctaccaacaagaactccaa

SEQ ID NO: 162 - DNA, 24 bp, Artificial Sequence
  Description of Artificial Sequence Synthetic oligonucleotide
taagtcaaagaactcctaccttgc

SEQ ID NO: 163 - DNA, 20 bp, Artificial Sequence
  Description of Artificial Sequence Synthetic oligonucleotide
ctgccctctttcacccagta

SEQ ID NO: 164 - DNA, 22 bp, Artificial Sequence
  Description of Artificial Sequence Synthetic oligonucleotide
gcttctgttcaagcttcccttt

SEQ ID NO: 165 - DNA, 21 bp, Artificial Sequence
  Description of Artificial Sequence Synthetic oligonucleotide
tggatcctcatggtcactttc

SEQ ID NO: 166 - DNA, 22 bp, Artificial Sequence
  Description of Artificial Sequence Synthetic oligonucleotide
ttgagagagtctgagcattcca

SEQ ID NO: 167 - DNA, 21 bp, Artificial Sequence
  Description of Artificial Sequence Synthetic oligonucleotide
tgacatcaatgctccaaacaa

SEQ ID NO: 168 - DNA, 24 bp, Artificial Sequence
  Description of Artificial Sequence Synthetic oligonucleotide
ttaacatgtctacttgccttttca

SEQ ID NO: 169 - DNA, 20 bp, Artificial Sequence
  Description of Artificial Sequence Synthetic oligonucleotide
gcagagctgtcacccaaaat

SEQ ID NO: 170 - DNA, 24 bp, Artificial Sequence
  Description of Artificial Sequence Synthetic oligonucleotide
tgacactcttaagttccagcagtc

SEQ ID NO: 171 - DNA, 20 bp, Artificial Sequence
  Description of Artificial Sequence Synthetic oligonucleotide
tgaccgatgcgttacatttt

SEQ ID NO: 172 - DNA, 20 bp, Artificial Sequence
  Description of Artificial Sequence Synthetic oligonucleotide
cctcatccagggatatttgc

SEQ ID NO: 173 - DNA, 27 bp, Artificial Sequence
  Description of Artificial Sequence Synthetic oligonucleotide
cgatgatgaattagaacatacaacttt

SEQ ID NO: 174 - DNA, 27 bp, Artificial Sequence
  Description of Artificial Sequence Synthetic oligonucleotide
agcaaaggaaatacattaaaaatcaaa

SEQ ID NO: 175 - DNA, 20 bp, Artificial Sequence
  Description of Artificial Sequence Synthetic oligonucleotide
agccacccaccttccttagt

SEQ ID NO: 176 - DNA, 19 bp, Artificial Sequence
  Description of Artificial Sequence Synthetic oligonucleotide
gtgtggtcctgtcctgacg

SEQ ID NO: 177 - DNA, 20 bp, Artificial Sequence
  Description of Artificial Sequence Synthetic oligonucleotide
tctctgctggcatagcttga

SEQ ID NO: 178 - DNA, 22 bp, Artificial Sequence
  Description of Artificial Sequence Synthetic oligonucleotide
ccaaattattcaacccatagcc

SEQ ID NO: 179 - DNA, 21 bp, Artificial Sequence
  Description of Artificial Sequence Synthetic oligonucleotide
tgtcatgattaaggcggtttc

SEQ ID NO: 180 - DNA, 20 bp, Artificial Sequence
  Description of Artificial Sequence Synthetic oligonucleotide
tctgcaaaccgaggctatct

SEQ ID NO: 181 - DNA, 20 bp, Artificial Sequence
  Description of Artificial Sequence Synthetic oligonucleotide
agcctgggcaacaagagtaa

SEQ ID NO: 182 - DNA, 20 bp, Artificial Sequence
  Description of Artificial Sequence Synthetic oligonucleotide
ggccatctgataaagcagga

SEQ ID NO: 183 - DNA, 20 bp, Artificial Sequence
  Description of Artificial Sequence Synthetic oligonucleotide
tgatgcataggaatgcaagg

SEQ ID NO: 184 - DNA, 20 bp, Artificial Sequence
  Description of Artificial Sequence Synthetic oligonucleotide
agtctccagtgaatcgccaa

SEQ ID NO: 185 - DNA, 50 bp, Artificial Sequence; identical to SEQ ID NO: 103
  Description of Artificial Sequence Synthetic oligonucleotide

SEQ ID NO: 186 - DNA, 20 bp, Artificial Sequence
  Description of Artificial Sequence Synthetic oligonucleotide
tgtctacctgttggcctgtg

SEQ ID NO: 187 - DNA, 20 bp, Artificial Sequence
  Description of Artificial Sequence Synthetic oligonucleotide
catggagaaggaaggtcagg

SEQ ID NO: 188 - DNA, 20 bp, Artificial Sequence
  Description of Artificial Sequence Synthetic oligonucleotide
atgactgcatgcactgctta

SEQ ID NO: 189 - DNA, 20 bp, Artificial Sequence
  Description of Artificial Sequence Synthetic oligonucleotide
tagcacaatgcctgccctat

SEQ ID NO: 190 - DNA, 20 bp, Artificial Sequence
  Description of Artificial Sequence Synthetic oligonucleotide
gtcttgtgcatctcccaggt

SEQ ID NO: 191 - DNA, 20 bp, Artificial Sequence
  Description of Artificial Sequence Synthetic oligonucleotide
ctagcactggcactgcccta

SEQ ID NO: 192 - DNA, 20 bp, Artificial Sequence
  Description of Artificial Sequence Synthetic oligonucleotide
tctgcaaaccgaggctatct

SEQ ID NO: 193 - DNA, 30 bp, Artificial Sequence
  Description of Artificial Sequence Synthetic oligonucleotide
tttttcactcctataatcgtagaagtagag

SEQ ID NO: 194 - DNA, 30 bp, Artificial Sequence
  Description of Artificial Sequence Synthetic oligonucleotide
cctggatgaggatgccccagcttattttca

SEQ ID NO: 195 - DNA, 36 bp, Artificial Sequence
  Description of Artificial Sequence Synthetic oligonucleotide
ttttcattatatttcttcaattacagtgtggtaatg

SEQ ID NO: 196 - DNA, 27 bp, Artificial Sequence
  Description of Artificial Sequence Synthetic oligonucleotide
ttgacagatattatttggcgattcact

SEQ ID NO: 197 - DNA, 41 bp, Artificial Sequence
  Description of Artificial Sequence Synthetic oligonucleotide
ctgtctttaggttgtgctgctgtacgcctctacctacgtcc

SEQ ID NO: 198 - DNA, 40 bp, Artificial Sequence
  Description of Artificial Sequence Synthetic oligonucleotide
tacatgaccatcgtggcctttctggtgtacttcatccctc

SEQ ID NO: 199 - DNA, 33 bp, Artificial Sequence
  Description of Artificial Sequence Synthetic oligonucleotide
tatttggattaaaaggaaaacctacgaaacagt

SEQ ID NO: 200 - DNA, 31 bp, Artificial Sequence
  Description of Artificial Sequence Synthetic oligonucleotide
cagggagcgaagatcacaggattccagaatg

SEQ ID NO: 201 - DNA, 41 bp, Artificial Sequence
  Description of Artificial Sequence Synthetic oligonucleotide
gacgttccgggagagaaccgagaggcatgagatgcagattc

SEQ ID NO: 202 - DNA, 39 bp, Artificial Sequence
  Description of Artificial Sequence Synthetic oligonucleotide
ccagtgaacacaggcatcagtggtccagggtcctggctt

SEQ ID NO: 203 - DNA, 37 bp, Artificial Sequence
  Description of Artificial Sequence Synthetic oligonucleotide
ctaatgctctgccctcaaagagagaactggaagggta

SEQ ID NO: 204 - DNA, 40 bp, Artificial Sequence
  Description of Artificial Sequence Synthetic oligonucleotide
agagaactggaagggtatttggccaggtgtaccttcctgg

SEQ ID NO: 205 - DNA, 30 bp, Artificial Sequence
  Description of Artificial Sequence Synthetic oligonucleotide
tctcactgcttaccagggcacatggacacc

SEQ ID NO: 206 - DNA, 27 bp, Artificial Sequence
  Description of Artificial Sequence Synthetic oligonucleotide
ggacaccggtggttcccaaaatgggtc

SEQ ID NO: 207 - DNA, 22 bp, Artificial Sequence
  Description of Artificial Sequence Synthetic oligonucleotide
agaatgagtctctgatgacttt

SEQ ID NO: 208 - DNA, 23 bp, Artificial Sequence
  Description of Artificial Sequence Synthetic oligonucleotide
acttgctgttcatagaattgcaa

SEQ ID NO: 209 - DNA, 24 bp, Artificial Sequence
  Description of Artificial Sequence Synthetic oligonucleotide
tgacttctccccagatttttgtat

SEQ ID NO: 210 - DNA, 24 bp, Artificial Sequence
  Description of Artificial Sequence Synthetic oligonucleotide
acacatacaaagtgcctaccacat

SEQ ID NO: 211 - DNA, 24 bp, Artificial Sequence
  Description of Artificial Sequence Synthetic oligonucleotide
ttgaaactgtatttcccatattgc

SEQ ID NO: 212 - DNA, 24 bp, Artificial Sequence
  Description of Artificial Sequence Synthetic oligonucleotide
aaatgcaataaaaatgcggaacta

SEQ ID NO: 213 - DNA, 22 bp, Artificial Sequence
  Description of Artificial Sequence Synthetic oligonucleotide
gagtcattagtccagagaacat

SEQ ID NO: 214 - DNA, 21 bp, Artificial Sequence
  Description of Artificial Sequence Synthetic oligonucleotide
ctgcttggaacagtgtatatc

SEQ ID NO: 215 - DNA, 23 bp, Artificial Sequence
  Description of Artificial Sequence Synthetic oligonucleotide
tggtctacgtagaattcagagta

SEQ ID NO: 216 - DNA, 23 bp, Artificial Sequence
  Description of Artificial Sequence Synthetic oligonucleotide
catgtgttaattgtgtcttcact

SEQ ID NO: 217 - DNA, 24 bp, Artificial Sequence
  Description of Artificial Sequence Synthetic oligonucleotide
gggtgtcatttacacgaacaataa

SEQ ID NO: 218 - DNA, 24 bp, Artificial Sequence
  Description of Artificial Sequence Synthetic oligonucleotide
cagttcagtcactgcaatcttcat

SEQ ID NO: 219 - DNA, 24 bp, Artificial Sequence
  Description of Artificial Sequence Synthetic oligonucleotide
ttatgagctaaagtgccaattaaa

SEQ ID NO: 220 - DNA, 21 bp, Artificial Sequence
  Description of Artificial Sequence Synthetic oligonucleotide
tccccgtctcctctagtcttc

SEQ ID NO: 221 - DNA, 20 bp, Artificial Sequence
  Description of Artificial Sequence Synthetic oligonucleotide
tgttggttcgatgagagcat

SEQ ID NO: 222 - DNA, 20 bp, Artificial Sequence
  Description of Artificial Sequence Synthetic oligonucleotide
aggaaggtggcagtgaactc

SEQ ID NO: 223 - DNA, 27 bp, Artificial Sequence
  Description of Artificial Sequence Synthetic oligonucleotide
tgagatgctgtctctaaaataaataga

SEQ ID NO: 224 - DNA, 20 bp, Artificial Sequence
  Description of Artificial Sequence Synthetic oligonucleotide
aagtggggaacaaacactgg

SEQ ID NO: 225 - DNA, 21 bp, Artificial Sequence
  Description of Artificial Sequence Synthetic oligonucleotide
ccctcctatacattacctgaa

SEQ ID NO: 226 - DNA, 20 bp, Artificial Sequence
  Description of Artificial Sequence Synthetic oligonucleotide
ttctctctttgtacccacac

SEQ ID NO: 227 - DNA, 24 bp, Artificial Sequence
  Description of Artificial Sequence Synthetic oligonucleotide
gtacctatatttgtattgcactta

SEQ ID NO: 228 - DNA, 20 bp, Artificial Sequence
  Description of Artificial Sequence Synthetic oligonucleotide
tactggaaaggtatgattgt

SEQ ID NO: 229 - DNA, 20 bp, Artificial Sequence
  Description of Artificial Sequence Synthetic oligonucleotide
ctaagacctggtaaagttat

SEQ ID NO: 230 - DNA, 20 bp, Artificial Sequence
  Description of Artificial Sequence Synthetic oligonucleotide
aggtctgctggtaaggaatt

SEQ ID NO: 231 - DNA, 20 bp, Artificial Sequence
  Description of Artificial Sequence Synthetic oligonucleotide
gaagcaggaggtaagggagg

SEQ ID NO: 232 - DNA, 20 bp, Artificial Sequence
  Description of Artificial Sequence Synthetic oligonucleotide
catagacctggtgagtaact

SEQ ID NO: 233 - DNA, 20 bp, Artificial Sequence
  Description of Artificial Sequence Synthetic oligonucleotide
aagaaaacaagtacgtatag

SEQ ID NO: 234 - DNA, 20 bp, Artificial Sequence
  Description of Artificial Sequence Synthetic oligonucleotide
ctctgctattgtaagtaatt

SEQ ID NO: 235 - DNA, 20 bp, Artificial Sequence
  Description of Artificial Sequence Synthetic oligonucleotide
aaatactgcagttaagtaca

SEQ ID NO: 236 - DNA, 20 bp, Artificial Sequence
  Description of Artificial Sequence Synthetic oligonucleotide
tctccatgaagtgcttggac

SEQ ID NO: 237 - DNA, 20 bp, Artificial Sequence
  Description of Artificial Sequence Synthetic oligonucleotide
ctgcgttcaggtaatgtata

SEQ ID NO: 238 - DNA, 10 bp, Artificial Sequence
  Description of Artificial Sequence Synthetic oligonucleotide
tattgcattt

SEQ ID NO: 239 - DNA, 20 bp, Artificial Sequence
  Description of Artificial Sequence Synthetic oligonucleotide
agctgggttggtaagactgg

SEQ ID NO: 240 - DNA, 20 bp, Artificial Sequence
  Description of Artificial Sequence Synthetic oligonucleotide
ccatatatgagtggatcata

SEQ ID NO: 241 - DNA, 20 bp, Artificial Sequence
  Description of Artificial Sequence Synthetic oligonucleotide
tctgccatgtgtaagtcttc

SEQ ID NO: 242 - DNA, 20 bp, Artificial Sequence
  Description of Artificial Sequence Synthetic oligonucleotide
gggacattttgttttccatt

SEQ ID NO: 243 - DNA, 20 bp, Artificial Sequence
  Description of Artificial Sequence Synthetic oligonucleotide
atgacctgaggtgggtatta

SEQ ID NO: 244 - DNA, 20 bp, Artificial Sequence
  Description of Artificial Sequence Synthetic oligonucleotide
cgttgttactaaaaaagaaa

SEQ ID NO: 245 - DNA, 20 bp, Artificial Sequence
  Description of Artificial Sequence Synthetic oligonucleotide
tgagcctcaggtaagaaccc

SEQ ID NO: 246 - DNA, 20 bp, Artificial Sequence
  Description of Artificial Sequence Synthetic oligonucleotide
tattttaaaggtacatgtat

SEQ ID NO: 247 - DNA, 20 bp, Artificial Sequence
  Description of Artificial Sequence Synthetic oligonucleotide
tttctaattaaaaaataaaa

SEQ ID NO: 248 - DNA, 20 bp, Artificial Sequence
  Description of Artificial Sequence Synthetic oligonucleotide
gattttacaggctgatggtg

SEQ ID NO: 249 - DNA, 20 bp, Artificial Sequence
  Description of Artificial Sequence Synthetic oligonucleotide
ctactgtgaggacacagaga

SEQ ID NO: 250 - DNA, 20 bp, Artificial Sequence
  Description of Artificial Sequence Synthetic oligonucleotide
ctattttcagagttcttcac

SEQ ID NO: 251 - DNA, 20 bp, Artificial Sequence
  Description of Artificial Sequence Synthetic oligonucleotide
gctttcacaggagtgacttc

SEQ ID NO: 252 - DNA, 20 bp, Artificial Sequence
  Description of Artificial Sequence Synthetic oligonucleotide
ctctggggaggtctaggact

SEQ ID NO: 253 - DNA, 20 bp, Artificial Sequence
  Description of Artificial Sequence Synthetic oligonucleotide
ccatttacagcttatgtgag

SEQ ID NO: 254 - DNA, 20 bp, Artificial Sequence
  Description of Artificial Sequence Synthetic oligonucleotide
gttttgacaggagctccaat

SEQ ID NO: 255 - DNA, 20 bp, Artificial Sequence
  Description of Artificial Sequence Synthetic oligonucleotide
cattgtacagtgaacaaata

SEQ ID NO: 256 - DNA, 20 bp, Artificial Sequence
  Description of Artificial Sequence Synthetic oligonucleotide
catcttctagaagtgagaaa

SEQ ID NO: 257 - DNA, 20 bp, Artificial Sequence
  Description of Artificial Sequence Synthetic oligonucleotide
caaaatccaggaaaacaagg

SEQ ID NO: 258 - DNA, 22 bp, Artificial Sequence
  Description of Artificial Sequence Synthetic oligonucleotide
taaaaattagaggtttaggaaa

SEQ ID NO: 259 - DNA, 20 bp, Artificial Sequence
  Description of Artificial Sequence Synthetic oligonucleotide
ttttttgcaggtggatatac

SEQ ID NO: 260 - DNA, 20 bp, Artificial Sequence
  Description of Artificial Sequence Synthetic oligonucleotide
cctttcgcaggactggcaac

SEQ ID NO: 261 - DNA, 20 bp, Artificial Sequence
  Description of Artificial Sequence Synthetic oligonucleotide
gattctgaaggccatgtttt

SEQ ID NO: 262 - DNA, 20 bp, Artificial Sequence
  Description of Artificial Sequence Synthetic oligonucleotide
tgaattttaggacgctgggc

SEQ ID NO: 263 - DNA, 20 bp, Artificial Sequence
  Description of Artificial Sequence Synthetic oligonucleotide
ctgtccacagcaatccttct

SEQ ID NO: 264 - DNA, 20 bp, Artificial Sequence
  Description of Artificial Sequence Synthetic oligonucleotide
ttctcaacagagcaaactgc

SEQ ID NO: 265 - DNA, 20 bp, Artificial Sequence
  Description of Artificial Sequence Synthetic oligonucleotide
ttgtttttaggctttttgat

SEQ ID NO: 266 - DNA, 20 bp, Artificial Sequence
  Description of Artificial Sequence Synthetic oligonucleotide
ttctccccagctctgttttc